SEQUENCE LISTING (398 sequences)

SEQ ID NO: 1 - protein, 1004 aa, synthetic construct
QPESVRLFGKLGVRRYLVLGLDPGVASCGFSLIDLHNHEILEMGSRLFDSPVVPKTQQSKAAVRRGFRSTRRNLDRTQNRLKHCLRLLKEQGLVPAGATPEFFHTAKGDQPPIRLRVAGLDRLLTDREWAIVLYSLCKRRGYIPHGEGGEGDVDSASEDGKVLKAISQNKKLLAESGARTVGEWLATQPRSRNRSGEYDKCVTHAQLTDEVRALFSAQRSFGSTSATEDLEDAYLEVFDWEKPRDAFDRRSYELVGNCVYFPGEKRAARCTLTSELVSAYGALGNVTIQFPDGTTRPLTAKERDTFIDTLFSPSALKGNKECKVKFSDIRRRLDLDARTSFKGISSDDEKNREVYRPQGWRTLRKTLGSEGTALLTRFLADRDLADATMEAVAYSSSLPVLSSRLEELELTDDEVALVERLPYSSRALNGYGSRSKKALDLLLGAFEEPEVLTLTDAETATGLDELRKHGSAGIERSTKLMPYAAWIALTGRTNNNPVVLRAMAQMRKVVNAVCGEWGVPNEIHVELARELALPKKAKDSIARANRQNEKDNERIRKQISELTGRDHDRIKNSLVAKWRLWEEQGNCDIYTGERIDALRLVNDDTYTQVDHILPFSRTGDNSRHNKVLVLAHSNQLKRERSPFEWMTSEEAGAPDWNSFVASVQENRRISPRKRGFLLERDLMSKEGDFQSRNLTDTAYMAREVCAYLSDCLAFPNDGRKVHVVPTKGTASAWLRRSWGLNFGMAGEKDRSDDRHHATDACVIAACSRSLVIQTAKLSEHHYTLNEQGRDEALLGSMPWPSFADDVRQMREKVIPTRFVPRVGSGELFEQTIYSYVGTDDKGKDLLSAKGGKPKPAGNAVVSDDEKSAIKVGGMICLRLWHDVDARKGRGQWYADPIYYADLPALRNGTYVPRIAQAHKGRKTWKPIPERVLSGKPICLYLGDAVRVDHEIGRFSGFDIDAANWSLKSIYSGEKICFPSIGKIDNITFPIKITEDILGRCWGNL

SEQ ID NO: 2 - protein, 1005 aa, synthetic construct
MQPESVRLFGKLGVRRYLVLGLDPGVASCGFSLIDLHNHEILEMGSRLFDSPVVPKTQQSKAAVRRGFRSTRRNLDRTQNRLKHCLRLLKEQGLVPAGATPEFFHTAKGDQPPIRLRVAGLDRLLTDREWAIVLYSLCKRRGYIPHGEGGEGDVDSASEDGKVLKAISQNKKLLAESGARTVGEWLATQPRSRNRSGEYDKCVTHAQLTDEVRALFSAQRSFGSTSATEDLEDAYLEVFDWEKPRDAFDRRSYELVGNCVYFPGEKRAARCTLTSELVSAYGALGNVTIQFPDGTTRPLTAKERDTFIDTLFSPSALKGNKECKVKFSDIRRRLDLDARTSFKGISSDDEKNREVYRPQGWRTLRKTLGSEGTALLTRFLADRDLADATMEAVAYSSSLPVLSSRLEELELTDDEVALVERLPYSSRALNGYGSRSKKALDLLLGAFEEPEVLTLTDAETATGLDELRKHGSAGIERSTKLMPYAAWIALTGRTNNNPVVLRAMAQMRKVVNAVCGEWGVPNEIHVELARELALPKKAKDSIARANRQNEKDNERIRKQISELTGRDHDRIKNSLVAKWRLWEEQGNCDIYTGERIDALRLVNDDTYTQVDHILPFSRTGDNSRHNKVLVLAHSNQLKRERSPFEWMTSEEAGAPDWNSFVASVQENRRISPRKRGFLLERDLMSKEGDFQSRNLTDTAYMAREVCAYLSDCLAFPNDGRKVHVVPTKGTASAWLRRSWGLNFGMAGEKDRSDDRHHATDACVIAACSRSLVIQTAKLSEHHYTLNEQGRDEALLGSMPWPSFADDVRQMREKVIPTRFVPRVGSGELFEQTIYSYVGTDDKGKDLLSAKGGKPKPAGNAVVSDDEKSAIKVGGMICLRLWHDVDARKGRGQWYADPIYYADLPALRNGTYVPRIAQAHKGRKTWKPIPERVLSGKPICLYLGDAVRVDHEIGRFSGFDIDAANWSLKSIYSGEKICFPSIGKIDNITFPIKITEDILGRCWGNL

SEQ ID NO: 3 - protein, 1055 aa, synthetic construct
MGKPIPNPLLGLDSTKRTADGSEFESPKKKRKVQPESVRLFGKLGVRRYLVLGLDPGVASCGFSLIDLHNHEILEMGSRLFDSPVVPKTQQSKAAVRRGFRSTRRNLDRTQNRLKHCLRLLKEQGLVPAGATPEFFHTAKGDQPPIRLRVAGLDRLLTDREWAIVLYSLCKRRGYIPHGEGGEGDVDSASEDGKVLKAISQNKKLLAESGARTVGEWLATQPRSRNRSGEYDKCVTHAQLTDEVRALFSAQRSFGSTSATEDLEDAYLEVFDWEKPRDAFDRRSYELVGNCVYFPGEKRAARCTLTSELVSAYGALGNVTIQFPDGTTRPLTAKERDTFIDTLFSPSALKGNKECKVKFSDIRRRLDLDARTSFKGISSDDEKNREVYRPQGWRTLRKTLGSEGTALLTRFLADRDLADATMEAVAYSSSLPVLSSRLEELELTDDEVALVERLPYSSRALNGYGSRSKKALDLLLGAFEEPEVLTLTDAETATGLDELRKHGSAGIERSTKLMPYAAWIALTGRTNNNPVVLRAMAQMRKVVNAVCGEWGVPNEIHVELARELALPKKAKDSIARANRQNEKDNERIRKQISELTGRDHDRIKNSLVAKWRLWEEQGNCDIYTGERIDALRLVNDDTYTQVDHILPFSRTGDNSRHNKVLVLAHSNQLKRERSPFEWMTSEEAGAPDWNSFVASVQENRRISPRKRGFLLERDLMSKEGDFQSRNLTDTAYMAREVCAYLSDCLAFPNDGRKVHVVPTKGTASAWLRRSWGLNFGMAGEKDRSDDRHHATDACVIAACSRSLVIQTAKLSEHHYTLNEQGRDEALLGSMPWPSFADDVRQMREKVIPTRFVPRVGSGELFEQTIYSYVGTDDKGKDLLSAKGGKPKPAGNAVVSDDEKSAIKVGGMICLRLWHDVDARKGRGQWYADPIYYADLPALRNGTYVPRIAQAHKGRKTWKPIPERVLSGKPICLYLGDAVRVDHEIGRFSGFDIDAANWSLKSIYSGEKICFPSIGKIDNITFPIKITEDILGRCWGNLKRTADGSEFESPKKKRKV

SEQ ID NO: 4 - DNA, 3018 bp, synthetic construct
  source 1..3018: mol_type = other DNA
atgcagcctgagtccgttcgcctctttggaaagcttggggttcgccgctacctcgtccttggactcgatcccggggtcgccagctgcggcttttctctcatcgacctccacaaccacgagattctcgagatggggtcccgtctcttcgactcgcccgtcgtccccaagacgcagcagagcaaggcggcggtgcgacggggcttccgctcaacccgtcgcaacctcgacaggacgcagaatcgtctgaagcactgccttcgcctcctcaaggagcagggcctcgtccccgccggcgccacccccgagttcttccacaccgcaaaaggcgaccaaccgcccatcaggctgagggtcgccggactcgaccggcttctcaccgaccgcgagtgggcgatcgtcctgtactcgctctgcaagcgccgcggctacataccccacggtgagggcggggagggtgacgtcgacagcgcaagcgaggacggaaaggtcctcaaggccataagccagaacaagaagcttctcgcggagagcggtgccagaaccgtcggtgagtggcttgctacgcagccgcgcagcaggaaccgctccggggagtacgacaagtgcgtcacccacgcacagctcaccgatgaggtgcgcgcgctgttcagcgcgcagaggtccttcggaagcacgtctgccacggaggatctcgaggacgcctatcttgaggtcttcgactgggagaagccccgcgacgcgttcgacaggcgaagctacgagctcgtcggcaactgcgtgtacttccccggcgagaagcgcgcggcgcgctgcacgctcacgagcgagctcgtctccgcatacggcgccctcggcaacgtcacaattcagttccccgacggcacgacgcgcccgctcaccgcaaaggagcgcgacaccttcatcgacacgctcttctccccctccgcgctcaagggcaacaaggagtgcaaggtcaagttctccgacatcagaaggcgcctcgacctcgacgcccggacctcgttcaagggcatctcgtctgatgacgagaagaaccgcgaggtataccggccgcaaggctggagaacgctgcgaaagacgctcggctccgaggggacggccctgctcacgaggtttctcgccgaccgcgacctcgcggacgcgacgatggaggccgtcgcgtactcctcgtccctccccgtcttgagctcgcgtctcgaggagcttgagctcaccgatgacgaggtcgccctcgtcgagcgcctcccctactcgagccgtgccctcaacggatacgggtcccgctccaagaaggcgcttgacctgctgctgggcgccttcgaggaacccgaggtactcactctcacggacgccgagacggccactggccttgatgagcttcgcaagcacggctcagccggcatcgagcggtcgacaaagctgatgccgtacgccgcatggatcgccctcaccggccgcaccaacaacaaccccgtcgtcctcagggcgatggctcagatgcgcaaggtcgtcaacgccgtctgtggtgaatggggcgtccccaacgagattcacgtcgagctcgcccgcgagctcgcgctacccaagaaggccaaggactccatcgcccgtgccaaccgtcagaacgagaaggataacgagcgcatcaggaagcagatctccgaactcacggggcgagaccacgaccggatcaagaactcacttgtggcaaagtggcgcctctgggaggagcaggggaactgcgacatctacacgggcgagagaatcgacgccctgcggctcgtcaatgacgacacgtacacccaggtcgaccatatcctccccttctctcgcacgggagacaactcgcgccacaacaaggtgctcgtccttgcccacagcaaccagctcaagcgcgagcggtctcccttcgagtggatgacctccgaggaggcgggcgcgcccgactggaactcctttgtcgcatccgtccaagagaaccgcaggatcagcccgcggaagcgtggcttcctcctcgagcgcgacctcatgtctaaggagggcgacttccaaagcaggaacctgacggacacggcctacatggcccgcgaggtctgcgcctatctctcggactgcctcgccttcccgaacgacggcaggaaggttcacgtcgtgccgaccaagggcaccgcctcggcgtggctcagaaggtcctgggggctcaacttcggcatggcgggggagaaggaccgctcggacgaccgtcaccacgcaacggatgcgtgcgtgattgccgcgtgctctcgctcgctcgtcattcagacggcaaagctctccgagcaccactacacgctcaacgagcagggtcgcgacgaggcgcttcttgggtctatgccctggccatcctttgccgatgacgtccgacagatgcgcgagaaggtcatcccgaccaggtttgtcccccgcgtcgggtcgggcgagctctttgagcagacgatctactcctatgtcggaaccgacgacaagggcaaggaccttctctcggcaaagggcggcaagccgaagcccgctggcaacgcagttgtctccgatgacgagaagtccgccatcaaggtcggaggaatgatctgcctgcgcctctggcacgatgtcgatgccagaaaggggcgtggccagtggtacgccgacccgatctactatgccgacctaccggctctgcgaaacgggacgtatgtgccgcggattgcccaagcgcataaggggagaaagacttggaagcccattcctgagcgggtcctttccggcaaaccgatttgtctctatctcggagatgcggtgcgggttgaccatgagatcggcagattttctggctttgacatcgatgcagccaattggtcactaaagagtatttactctggggagaagatttgttttccttctattggaaagattgataatataacttttccaattaaaatcacagaggatatccttggtagatgttggggaaatttgtag

SEQ ID NO: 5 - DNA, 3072 bp, synthetic construct
  source 1..3072: mol_type = other DNA
atgcagcctgagagcgtgcgactgttcggaaagctgggagtgagaagatacctggtgctgggtctggaccctggcgtggccagctgcggctttagccttatcgacctgcacaatcacgagatcctggaaatgggctctcggctgttcgacagccctgtggtgcctaagacccagcagtctaaggccgctgtgcggcggggcttcagatctaccagacggaatctggacagaacacaaaacagactgaagcactgcctgagactgctgaaggaacagggcctggtccctgccggggcaacccctgagttcttccacaccgccaaaggcgatcagccccccatcagactgcgggtggccggcctggaccgtctgctgaccgacagagagtgggccatcgtgctttatagcttgtgcaagcggagaggctacatcccgcacggcgagggcggcgaaggcgatgtggattctgccagcgaggatggcaaggtgctgaaagccatcagccagaacaaaaaactgctggccgagagcggcgctagaaccgtgggcgagtggctggctacccagcctagatcacggaaccggagcggcgagtacgacaagtgcgtgacacacgcccaactgaccgacgaggtgagagccctgttttctgcccagagatcctttggtagcacatccgccaccgaggacctggaagatgcctacctggaagtgttcgattgggagaagccacgcgacgccttcgacagacggtcctacgagctggtcggcaactgcgtgtacttcccaggcgaaaaaagagccgcccggtgcacattaacaagcgagctggtgtccgcttacggtgccctgggcaacgtgaccatccaatttccagacggcaccacccgcccactgacggccaaagagagagataccttcatcgacaccctcttcagcccatccgccctgaagggaaacaaagaatgcaaagtgaagttctctgacatcagaaggagactggacctcgacgccaggaccagcttcaagggaatctccagcgacgacgagaagaaccgggaggtgtaccggcctcagggatggcggaccctgagaaagacactgggcagcgaaggcaccgcgctgctgacacggttcctggctgatcgagatctggccgatgctacaatggaggccgtggcttacagctccagccttcctgtgctgtctagcagactggaagagctggaactaaccgatgatgaagtggccctcgttgagcgcctgccttacagcagcagagccctgaacggctatggaagccggagcaagaaggccctggatctgctgctgggcgccttcgaggaacctgaggtgctgacactgaccgacgccgaaaccgctaccggcctggacgagctgagaaagcacggcagcgccggcatcgagagaagcaccaagctgatgccctacgccgcctggatcgccctgacaggaagaacaaacaacaaccccgttgtgctgagagctatggcccagatgcggaaagtggtgaacgccgtttgtggcgagtggggcgtgcccaacgagatccacgtggaacttgcccgggagctggccctgcctaagaaggctaaggactctatcgccagggccaatagacagaacgagaaggataacgagagaatccggaagcagatctcggagctgacaggacgggaccacgatcggattaagaacagcctggtggctaagtggagactgtgggaagaacagggaaattgcgacatctacaccggcgagcggatcgatgctttgcgcctggtcaacgatgacacgtacactcaggtggaccacatcctgcctttcagcagaaccggagataatagcagacacaacaaggtgctggtgctggctcacagcaaccagctgaagagagaaagatctccttttgagtggatgacctccgaggaagccggagcccctgactggaactcgtttgtggccagcgtgcaggagaaccggagaatctctcccagaaagcgcggcttcctgctggaaagagatctgatgagcaaggaaggcgatttccagagcagaaatctgacagacaccgcctacatggccagagaggtctgcgcctacctgtctgattgcctggctttccctaacgacggcagaaaggtgcacgtggtgcctaccaagggcaccgcctctgcatggctgcggagaagctggggactgaacttcggcatggccggcgagaaggaccggagcgacgatcggcaccatgccaccgacgcttgtgtgatcgccgcctgtagcagaagcctggtcatccagaccgccaagctgtccgagcaccactacacactgaacgagcagggccgggacgaggccctactgggctctatgccttggccttctttcgccgatgacgtgagacagatgagagaaaaagtgatcccaacaagattcgtgcctcgggtaggcagtggcgagctgttcgagcaaacaatctacagctacgtgggcactgacgataagggcaaggacctcctgagcgccaaaggaggcaaacccaagcccgccggcaatgccgtggtcagcgacgacgagaaatcagcaatcaaggtgggcggcatgatctgtctgagactgtggcacgatgtggacgctcggaagggcagaggccagtggtatgccgaccccatctactacgccgacctgccagccctgagaaacggcacctacgtgcctagaatcgcccaggcccacaagggcagaaaaacctggaagcccatccctgaaagagtgctgagcggcaagcccatctgtctgtacctcggcgacgccgtgcgcgtggaccatgaaatcggcagattcagcggctttgacatagatgccgctaactggagcctgaaatccatttacagcggagagaagatctgcttccctagcattggcaagatcgacaacatcaccttccctatcaagatcacagaggacatcctgggaagatgctggggcaacctgaagcggacagcagacggctccgaatttgaaagccctaagaaaaagagaaaggtgtga

SEQ ID NO: 6 - DNA, 3168 bp, synthetic construct
  source 1..3168: mol_type = other DNA
atgggcaaacctatccccaatcccctgctgggcctggacagcaccaagcggactgccgacggcagtgagttcgagtctcccaagaaaaagaggaaagtgcagcctgagagcgtgcgactgttcggaaagctgggagtgagaagatacctggtgctgggtctggaccctggcgtggccagctgcggctttagccttatcgacctgcacaatcacgagatcctggaaatgggctctcggctgttcgacagccctgtggtgcctaagacccagcagtctaaggccgctgtgcggcggggcttcagatctaccagacggaatctggacagaacacaaaacagactgaagcactgcctgagactgctgaaggaacagggcctggtccctgccggggcaacccctgagttcttccacaccgccaaaggcgatcagccccccatcagactgcgggtggccggcctggaccgtctgctgaccgacagagagtgggccatcgtgctttatagcttgtgcaagcggagaggctacatcccgcacggcgagggcggcgaaggcgatgtggattctgccagcgaggatggcaaggtgctgaaagccatcagccagaacaaaaaactgctggccgagagcggcgctagaaccgtgggcgagtggctggctacccagcctagatcacggaaccggagcggcgagtacgacaagtgcgtgacacacgcccaactgaccgacgaggtgagagccctgttttctgcccagagatcctttggtagcacatccgccaccgaggacctggaagatgcctacctggaagtgttcgattgggagaagccacgcgacgccttcgacagacggtcctacgagctggtcggcaactgcgtgtacttcccaggcgaaaaaagagccgcccggtgcacattaacaagcgagctggtgtccgcttacggtgccctgggcaacgtgaccatccaatttccagacggcaccacccgcccactgacggccaaagagagagataccttcatcgacaccctcttcagcccatccgccctgaagggaaacaaagaatgcaaagtgaagttctctgacatcagaaggagactggacctcgacgccaggaccagcttcaagggaatctccagcgacgacgagaagaaccgggaggtgtaccggcctcagggatggcggaccctgagaaagacactgggcagcgaaggcaccgcgctgctgacacggttcctggctgatcgagatctggccgatgctacaatggaggccgtggcttacagctccagccttcctgtgctgtctagcagactggaagagctggaactaaccgatgatgaagtggccctcgttgagcgcctgccttacagcagcagagccctgaacggctatggaagccggagcaagaaggccctggatctgctgctgggcgccttcgaggaacctgaggtgctgacactgaccgacgccgaaaccgctaccggcctggacgagctgagaaagcacggcagcgccggcatcgagagaagcaccaagctgatgccctacgccgcctggatcgccctgacaggaagaacaaacaacaaccccgttgtgctgagagctatggcccagatgcggaaagtggtgaacgccgtttgtggcgagtggggcgtgcccaacgagatccacgtggaacttgcccgggagctggccctgcctaagaaggctaaggactctatcgccagggccaatagacagaacgagaaggataacgagagaatccggaagcagatctcggagctgacaggacgggaccacgatcggattaagaacagcctggtggctaagtggagactgtgggaagaacagggaaattgcgacatctacaccggcgagcggatcgatgctttgcgcctggtcaacgatgacacgtacactcaggtggaccacatcctgcctttcagcagaaccggagataatagcagacacaacaaggtgctggtgctggctcacagcaaccagctgaagagagaaagatctccttttgagtggatgacctccgaggaagccggagcccctgactggaactcgtttgtggccagcgtgcaggagaaccggagaatctctcccagaaagcgcggcttcctgctggaaagagatctgatgagcaaggaaggcgatttccagagcagaaatctgacagacaccgcctacatggccagagaggtctgcgcctacctgtctgattgcctggctttccctaacgacggcagaaaggtgcacgtggtgcctaccaagggcaccgcctctgcatggctgcggagaagctggggactgaacttcggcatggccggcgagaaggaccggagcgacgatcggcaccatgccaccgacgcttgtgtgatcgccgcctgtagcagaagcctggtcatccagaccgccaagctgtccgagcaccactacacactgaacgagcagggccgggacgaggccctactgggctctatgccttggccttctttcgccgatgacgtgagacagatgagagaaaaagtgatcccaacaagattcgtgcctcgggtaggcagtggcgagctgttcgagcaaacaatctacagctacgtgggcactgacgataagggcaaggacctcctgagcgccaaaggaggcaaacccaagcccgccggcaatgccgtggtcagcgacgacgagaaatcagcaatcaaggtgggcggcatgatctgtctgagactgtggcacgatgtggacgctcggaagggcagaggccagtggtatgccgaccccatctactacgccgacctgccagccctgagaaacggcacctacgtgcctagaatcgcccaggcccacaagggcagaaaaacctggaagcccatccctgaaagagtgctgagcggcaagcccatctgtctgtacctcggcgacgccgtgcgcgtggaccatgaaatcggcagattcagcggctttgacatagatgccgctaactggagcctgaaatccatttacagcggagagaagatctgcttccctagcattggcaagatcgacaacatcaccttccctatcaagatcacagaggacatcctgggaagatgctggggcaacctgaagcggacagcagacggctccgaatttgaaagccctaagaaaaagagaaaggtgtga

SEQ ID NO: 7 - protein, 18 aa, synthetic construct
KRTADGSEFESPKKKRKV

SEQ ID NO: 8 - protein, 7 aa, synthetic construct
PKKKRKV

SEQ ID NO: 9 - protein, 7 aa, synthetic construct
PKKKRRV

SEQ ID NO: 10 - protein, 16 aa, synthetic construct
KRPAATKKAGQAKKKK

SEQ ID NO: 11 - protein, 11 aa, synthetic construct
YGRKKRRQRRR

SEQ ID NO: 12 - protein, 9 aa, synthetic construct
RKKRRQRRR

SEQ ID NO: 13 - protein, 9 aa, synthetic construct
PAAKRVKLD

SEQ ID NO: 14 - protein, 11 aa, synthetic construct
RQRRNELKRSP

SEQ ID NO: 15 - protein, 8 aa, synthetic construct
VSRKRPRP

SEQ ID NO: 16 - protein, 8 aa, synthetic construct
PPKKARED

SEQ ID NO: 17 - protein, 8 aa, synthetic construct
PQPKKKPL

SEQ ID NO: 18 - protein, 12 aa, synthetic construct
SALIKKKKKMAP

SEQ ID NO: 19 - protein, 7 aa, synthetic construct
PKQKKRK

SEQ ID NO: 20 - protein, 10 aa, synthetic construct
RKLKKKIKKL

SEQ ID NO: 21 - protein, 10 aa, synthetic construct
REKKKFLKRR

SEQ ID NO: 22 - protein, 20 aa, synthetic construct
KRKGDEVDGVDEVAKKKSKK

SEQ ID NO: 23 - protein, 17 aa, synthetic construct
RKCLQAGMNLEARKTKK

SEQ ID NO: 24 - protein, 38 aa, synthetic construct
NQSSNFGPMKGGNFGGRSSGPYGGGGQYFAKPRNQGGY

SEQ ID NO: 25 - protein, 42 aa, synthetic construct
RMRIZFKNKGKDTAELRRRRVEVSVELRKAKKDEQILKRRNV

SEQ ID NO: 26 - protein, 14 aa, synthetic construct
GKPIPNPLLGLDST

SEQ ID NO: 27 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 28 - RNA, 36 nt, synthetic construct
  source 1..36: mol_type = other RNA
gtcttgagcacgcacccttccccaaggtgatacgct

SEQ ID NO: 29 - RNA, 104 nt, synthetic construct
  source 1..104: mol_type = other RNA
tcaccttggggaagggtgcggctccagacaagggaagtcagctatctgacttacccgtaaagttacccccgcaccgtcctcggacgatgcggggcgaacttttt

SEQ ID NO: 30-33 - skipped (4 entries)

SEQ ID NO: 34 - RNA, 24 nt, synthetic construct
  source 1..24: mol_type = other RNA
cttgtggctgacccgyggctgctc

SEQ ID NO: 35 - RNA, 23 nt, synthetic construct
  source 1..23: mol_type = other RNA
cttgggtgggagcagccrcgggt

SEQ ID NO: 36 - RNA, 24 nt, synthetic construct
  source 1..24: mol_type = other RNA
ggagcagccrcgggtcagccacaa

SEQ ID NO: 37 - RNA, 24 nt, synthetic construct
  source 1..24: mol_type = other RNA
cttgtggctgacccgtggctgctc

SEQ ID NO: 38 - RNA, 23 nt, synthetic construct
  source 1..23: mol_type = other RNA
cttgggtgggagcagccacgggt

SEQ ID NO: 39 - RNA, 24 nt, synthetic construct
  source 1..24: mol_type = other RNA
ggagcagccacgggtcagccacaa

SEQ ID NO: 40 - RNA, 24 nt, synthetic construct
  source 1..24: mol_type = other RNA
cttgtggctgacccgcggctgctc

SEQ ID NO: 41 - RNA, 23 nt, synthetic construct
  source 1..23: mol_type = other RNA
cttgggtgggagcagccgcgggt

SEQ ID NO: 42 - RNA, 24 nt, synthetic construct
  source 1..24: mol_type = other RNA
ggagcagccgcgggtcagccacaa

SEQ ID NO: 43 - RNA, 23 nt, synthetic construct
  source 1..23: mol_type = other RNA
gtgccagaaacaggggtgacggg

SEQ ID NO: 44 - RNA, 133 nt, synthetic construct
  source 1..133: mol_type = other RNA
gtcttgagcacgcacccttccccaaggtgagaaatcaccttggggaagggtgcggctccagacaagggaagtcagctatctgacttacccgtaaagttacccccgcaccgtcctcggacgatgcggggcgaac

SEQ ID NO: 45 - RNA, 99 nt, synthetic construct
  source 1..99: mol_type = other RNA
gtcttgagcacgcgaaagcggctccagacaagggaagtcagctatctgacttacccgtaaagttacccccgcaccgtcctcggacgatgcggggcgaac

SEQ ID NO: 46 - RNA, 139 nt, synthetic construct
  source 1..139: mol_type = other RNA
gtcttgagcacgcacccttccccaaggtgagaaatcaccttggggaagggtgcggctccagacaagggaagtcagctatctgacttacccgtaaagttacccccgcaccgtcctcggacgatgcggggcgaactttttt

SEQ ID NO: 47 - RNA, 105 nt, synthetic construct
  source 1..105: mol_type = other RNA
gtcttgagcacgcgaaagcggctccagacaagggaagtcagctatctgacttacccgtaaagttacccccgcaccgtcctcggacgatgcggggcgaactttttt

SEQ ID NO: 48 - DNA, 23 bp, synthetic construct
  source 1..23: mol_type = other DNA
ggcgatgccacctacggcaagct

SEQ ID NO: 49 - DNA, 37 bp, synthetic construct
  source 1..37: mol_type = other DNA
gagggcgatgccacctacggcaagctgaccctgaagt

SEQ ID NO: 50 - DNA, 27 bp, synthetic construct
  source 1..27: mol_type = other DNA
caccggcgatgccacctacggcaagct

SEQ ID NO: 51 - DNA, 27 bp, synthetic construct
  source 1..27: mol_type = other DNA
agacagcttgccgtaggtggcatcgcc

SEQ ID NO: 52 - DNA, 23 bp, synthetic construct
  source 1..23: mol_type = other DNA
acggcgtgcagtgcttcagccgc

SEQ ID NO: 53 - DNA, 37 bp, synthetic construct
  source 1..37: mol_type = other DNA
cctacggcgtgcagtgcttcagccgctaccccgacca

SEQ ID NO: 54 - DNA, 28 bp, synthetic construct
  source 1..28: mol_type = other DNA
caccgacggcgtgcagtgcttcagccgc

SEQ ID NO: 55 - DNA, 28 bp, synthetic construct
  source 1..28: mol_type = other DNA
agacgcggctgaagcactgcacgccgtc

SEQ ID NO: 56 - DNA, 24 bp, synthetic construct
  source 1..24: mol_type = other DNA
cttgtggctgacccgtggctgctc

SEQ ID NO: 57 - DNA, 38 bp, synthetic construct
  source 1..38: mol_type = other DNA
gcccttgtggctgacccgtggctgctcccacccaagaa

SEQ ID NO: 58 - DNA, 29 bp, synthetic construct
  source 1..29: mol_type = other DNA
caccgcttgtggctgacccgtggctgctc

SEQ ID NO: 59 - DNA, 29 bp, synthetic construct
  source 1..29: mol_type = other DNA
agacgagcagccacgggtcagccacaagc

SEQ ID NO: 60 - DNA, 23 bp, synthetic construct
  source 1..23: mol_type = other DNA
cttgggtgggagcagccacgggt

SEQ ID NO: 61 - DNA, 37 bp, synthetic construct
  source 1..37: mol_type = other DNA
attcttgggtgggagcagccacgggtcagccacaagg

SEQ ID NO: 62 - DNA, 28 bp, synthetic construct
  source 1..28: mol_type = other DNA
caccgcttgggtgggagcagccacgggt

SEQ ID NO: 63 - DNA, 28 bp, synthetic construct
  source 1..28: mol_type = other DNA
agacacccgtggctgctcccacccaagc

SEQ ID NO: 64 - DNA, 24 bp, synthetic construct
  source 1..24: mol_type = other DNA
ggagcagccacgggtcagccacaa

SEQ ID NO: 65 - DNA, 38 bp, synthetic construct
  source 1..38: mol_type = other DNA
gtgggagcagccacgggtcagccacaagggccacagcc

SEQ ID NO: 66 - DNA, 28 bp, synthetic construct
  source 1..28: mol_type = other DNA
caccggagcagccacgggtcagccacaa

SEQ ID NO: 67 - DNA, 28 bp, synthetic construct
  source 1..28: mol_type = other DNA
agacttgtggctgacccgtggctgctcc

SEQ ID NO: 68 - DNA, 23 bp, synthetic construct
  source 1..23: mol_type = other DNA
gtgccagaaacaggggtgacggg

SEQ ID NO: 69 - DNA, 37 bp, synthetic construct
  source 1..37: mol_type = other DNA
ctggtgccagaaacaggggtgacgggagggcagaact

SEQ ID NO: 70 - DNA, 27 bp, synthetic construct
  source 1..27: mol_type = other DNA
caccgtgccagaaacaggggtgacggg

SEQ ID NO: 71 - DNA, 27 bp, synthetic construct
  source 1..27: mol_type = other DNA
agaccccgtcacccctgtttctggcac

SEQ ID NO: 72 - DNA, 59 bp, synthetic construct
  source 1..59: mol_type = other DNA
tcgtcggcagcgtcagatgtgtataagagacagaaaacacaccgcatacgtacgattta

SEQ ID NO: 73 - DNA, 61 bp, synthetic construct
  source 1..61: mol_type = other DNA
tcgtcggcagcgtcagatgtgtataagagacagtttaatcacaccgcatacgtacgattta

SEQ ID NO: 74 - DNA, 59 bp, synthetic construct
  source 1..59: mol_type = other DNA
gtctcgtgggctcggagatgtgtataagagacagcgttctgatttaatctgtatcaggc

SEQ ID NO: 75 - DNA, 62 bp, synthetic construct
  source 1..62: mol_type = other DNA
tcgtcggcagcgtcagatgtgtataagagacagtttttggttacgcatctgtgcggtatttc

SEQ ID NO: 76 - DNA, 58 bp, synthetic construct
  source 1..58: mol_type = other DNA
tcgtcggcagcgtcagatgtgtataagagacagcccttacgcatctgtgcggtatttc

SEQ ID NO: 77 - DNA, 59 bp, synthetic construct
  source 1..59: mol_type = other DNA
tcgtcggcagcgtcagatgtgtataagagacagtatcgatttaaataggcctgactcac

SEQ ID NO: 78 - DNA, 62 bp, synthetic construct
  source 1..62: mol_type = other DNA
tcgtcggcagcgtcagatgtgtataagagacagatttcgatttaaataggcctgactcacta

SEQ ID NO: 79 - DNA, 55 bp, synthetic construct
  source 1..55: mol_type = other DNA
gtctcgtgggctcggagatgtgtataagagacagaatcttctctcatccgccaaa

SEQ ID NO: 80 - DNA, 31 bp, synthetic construct
  source 1..31: mol_type = other DNA
cggcattcctgctgaaccgctcttccgatct

SEQ ID NO: 81 - DNA, 30 bp, synthetic construct
  source 1..30: mol_type = other DNA
gatcggaagagcggttcagcaggaatgccg

SEQ ID NO: 82 - DNA, 55 bp, synthetic construct
  source 1..55: mol_type = other DNA
tcgtcggcagcgtcagatgtgtataagagacagctgctgaaccgctcttccgatc

SEQ ID NO: 83 - DNA, 60 bp, synthetic construct
  source 1..60: mol_type = other DNA
tcgtcggcagcgtcagatgtgtataagagacagtaagactgctgaaccgctcttccgatc

SEQ ID NO: 84 - DNA, 72 bp, synthetic construct
  source 1..72: mol_type = other DNA
tcgtcggcagcgtcagatgtgtataagagacaggctagacctaatgtgatctgctgaaccgctcttccgatc

SEQ ID NO: 85 - DNA, 63 bp, synthetic construct
  source 1..63: mol_type = other DNA
gtctcgtgggctcggagatgtgtataagagacagtctgcgttctgatttaatctgtatcaggc

SEQ ID NO: 86 - DNA, 20 bp, synthetic construct
  source 1..20: mol_type = other DNA
gggaacgaacaggtaagggg

SEQ ID NO: 87 - DNA, 20 bp, synthetic construct
  source 1..20: mol_type = other DNA
cagcaccccatctgttttcc

SEQ ID NO: 88 - DNA, 28 bp, synthetic construct
  source 1..28: mol_type = other DNA
gtcttaatttccactcatacagtggtag

SEQ ID NO: 89 - DNA, 22 bp, synthetic construct
  source 1..22: mol_type = other DNA
cgttttgggctctgggactcag

SEQ ID NO: 90 - DNA, 20 bp, synthetic construct
  source 1..20: mol_type = other DNA
agcgtgaggaagttgatggg

SEQ ID NO: 91 - RNA, 81 nt, synthetic construct
  source 1..81: mol_type = other RNA
gtcttgagcacgcgaaagcggctccagacaagggaagtcagctatctgacttacccgtaaagttaccccgaaagggcgaac

SEQ ID NO: 92 - RNA, 87 nt, synthetic construct
  source 1..87: mol_type = other RNA
gtcttgagcacgcgaaagcggctccagacaagggaagtcagctatctgacttacccgtaaagttaccccgaaagggcgaactttttt

SEQ ID NO: 93 - RNA, 24 nt, synthetic construct
  source 1..24: mol_type = other RNA
ggcccttgtggctgacccgyggct

SEQ ID NO: 94 - RNA, 24 nt, synthetic construct
  source 1..24: mol_type = other RNA
ggcccttgtggctgacccgtggct

SEQ ID NO: 95 - RNA, 24 nt, synthetic construct
  source 1..24: mol_type = other RNA
ggcccttgtggctgacccgcggct

SEQ ID NO: 96 - RNA, 20 nt, synthetic construct
  source 1..20: mol_type = other RNA
gggtgggagcagccrcgggt

SEQ ID NO: 97 - RNA, 21 nt, synthetic construct
  source 1..21: mol_type = other RNA
tgggtgggagcagccrcgggt

SEQ ID NO: 98 - RNA, 22 nt, synthetic construct
  source 1..22: mol_type = other RNA
ttgggtgggagcagccrcgggt

SEQ ID NO: 99 - RNA, 24 nt, synthetic construct
  source 1..24: mol_type = other RNA
tcttgggtgggagcagccrcgggt

SEQ ID NO: 100 - RNA, 25 nt, synthetic construct
  source 1..25: mol_type = other RNA
ttcttgggtgggagcagccrcgggt

SEQ ID NO: 101 - RNA, 20 nt, synthetic construct
  source 1..20: mol_type = other RNA
gggtgggagcagccacgggt

SEQ ID NO: 102 - RNA, 21 nt, synthetic construct
  source 1..21: mol_type = other RNA
tgggtgggagcagccacgggt

SEQ ID NO: 103 - RNA, 22 nt, synthetic construct
  source 1..22: mol_type = other RNA
ttgggtgggagcagccacgggt

SEQ ID NO: 104 - RNA, 24 nt, synthetic construct
  source 1..24: mol_type = other RNA
tcttgggtgggagcagccacgggt

SEQ ID NO: 105 - RNA, 25 nt, synthetic construct
  source 1..25: mol_type = other RNA
ttcttgggtgggagcagccacgggt

SEQ ID NO: 106 - RNA, 20 nt, synthetic construct
  source 1..20: mol_type = other RNA
gggtgggagcagccgcgggt

SEQ ID NO: 107 - RNA, 21 nt, synthetic construct
  source 1..21: mol_type = other RNA
tgggtgggagcagccgcgggt

SEQ ID NO: 108 - RNA, 22 nt, synthetic construct
  source 1..22: mol_type = other RNA
ttgggtgggagcagccgcgggt

SEQ ID NO: 109 - RNA, 24 nt, synthetic construct
  source 1..24: mol_type = other RNA
tcttgggtgggagcagccgcgggt

SEQ ID NO: 110 - RNA, 25 nt, synthetic construct
  source 1..25: mol_type = other RNA
ttcttgggtgggagcagccgcgggt

SEQ ID NO: 111 - RNA, 23 nt, synthetic construct
  source 1..23: mol_type = other RNA
catgctcccgggctcctgcacac

SEQ ID NO: 112 - RNA, 23 nt, synthetic construct
  source 1..23: mol_type = other RNA
cctccatgctcccgggctcctgc

SEQ ID NO: 113 - DNA, 23 bp, synthetic construct
  source 1..23: mol_type = other DNA
agccaccaccgccaagcccggga

SEQ ID NO: 114 - RNA, 23 nt, synthetic construct
  source 1..23: mol_type = other RNA
tcccttctcctgtcctgtcaatg

SEQ ID NO: 115 - RNA, 23 nt, synthetic construct
  source 1..23: mol_type = other RNA
cccttctcctgtcctgtcaatgt

SEQ ID NO: 116 - RNA, 23 nt, synthetic construct
  source 1..23: mol_type = other RNA
tgtcctgtcaatgttatccaaag

SEQ ID NO: 117 - RNA, 23 nt, synthetic construct
  source 1..23: mol_type = other RNA
atccaaagccctcatatattcag

SEQ ID NO: 118 - RNA, 23 nt, synthetic construct
  source 1..23: mol_type = other RNA
aaggcagtgttcagtgccagccc

SEQ ID NO: 119 - RNA, 23 nt, synthetic construct
  source 1..23: mol_type = other RNA
tcaaggcagtgttcagtgccagc

SEQ ID NO: 120 - RNA, 23 nt, synthetic construct
  source 1..23: mol_type = other RNA
gtgaaattagacaagcgcatatt

SEQ ID NO: 121 - RNA, 23 nt, synthetic construct
  source 1..23: mol_type = other RNA
ttggagcaatatgcgcttgtcta

SEQ ID NO: 122 - RNA, 24 nt, synthetic construct
  source 1..24: mol_type = other RNA
gttttcttgctgtgaaattagaca

SEQ ID NO: 123 - RNA, 23 nt, synthetic construct
  source 1..23: mol_type = other RNA
tcacagcaagaaaactgagctga

SEQ ID NO: 124 - RNA, 23 nt, synthetic construct
  source 1..23: mol_type = other RNA
aagaagtcaagcgccctgctggg

SEQ ID NO: 125 - RNA, 23 nt, synthetic construct
  source 1..23: mol_type = other RNA
gaagtcaagcgccctgctggggc

SEQ ID NO: 126 - RNA, 23 nt, synthetic construct
  source 1..23: mol_type = other RNA
aactctgcacccgtccctgtgtg

SEQ ID NO: 127 - RNA, 23 nt, synthetic construct
  source 1..23: mol_type = other RNA
tgctggggcgtcacacagggacg

SEQ ID NO: 128 - RNA, 23 nt, synthetic construct
  source 1..23: mol_type = other RNA
aacatggcccgagatagatgcgg

SEQ ID NO: 129 - RNA, 23 nt, synthetic construct
  source 1..23: mol_type = other RNA
acagaggcttggtgctgcaaaca

SEQ ID NO: 130 - RNA, 23 nt, synthetic construct
  source 1..23: mol_type = other RNA
tccaagggaaacagaggcttggt

SEQ ID NO: 131 - RNA, 23 nt, synthetic construct
  source 1..23: mol_type = other RNA
gactcagcacagctgctccaagg

SEQ ID NO: 132 - RNA, 23 nt, synthetic construct
  source 1..23: mol_type = other RNA
gcctgggtctgactcagcacagc

SEQ ID NO: 133 - RNA, 23 nt, synthetic construct
  source 1..23: mol_type = other RNA
cccttggagcagctgtgctgagt

SEQ ID NO: 134 - RNA, 23 nt, synthetic construct
  source 1..23: mol_type = other RNA
tcagtgcccagcctgggtctgac

SEQ ID NO: 135 - RNA, 23 nt, synthetic construct
  source 1..23: mol_type = other RNA
atacacatcagaatccttacttt

SEQ ID NO: 136 - RNA, 24 nt, synthetic construct
  source 1..24: mol_type = other RNA
gtaaggattctgatgtgtatatca

SEQ ID NO: 137 - RNA, 23 nt, synthetic construct
  source 1..23: mol_type = other RNA
gagcaacagtgctgtggcctgga

SEQ ID NO: 138 - RNA, 23 nt, synthetic construct
  source 1..23: mol_type = other RNA
ggagcaacaaatctgactttgca

SEQ ID NO: 139 - RNA, 23 nt, synthetic construct
  source 1..23: mol_type = other RNA
tgctgttgttgaaggcgtttgca

SEQ ID NO: 140 - RNA, 23 nt, synthetic construct
  source 1..23: mol_type = other RNA
agcattattccagaagacacctt

SEQ ID NO: 141 - RNA, 23 nt, synthetic construct
  source 1..23: mol_type = other RNA
tattccagaagacaccttcttcc

SEQ ID NO: 142 - RNA, 23 nt, synthetic construct
  source 1..23: mol_type = other RNA
acaaagtaaggattctgatgtgt

SEQ ID NO: 143 - RNA, 23 nt, synthetic construct
  source 1..23: mol_type = other RNA
gtctagcacagttttgtctgtga

SEQ ID NO: 144 - RNA, 23 nt, synthetic construct
  source 1..23: mol_type = other RNA
cttgaagtccatagacctcatgt

SEQ ID NO: 145 - RNA, 23 nt, synthetic construct
  source 1..23: mol_type = other RNA
ggccacagcactgttgctcttga

SEQ ID NO: 146 - RNA, 23 nt, synthetic construct
  source 1..23: mol_type = other RNA
tgcaaagtcagatttgttgctcc

SEQ ID NO: 147 - RNA, 23 nt, synthetic construct
  source 1..23: mol_type = other RNA
gaataatgctgttgttgaaggcg

SEQ ID NO: 148 - RNA, 23 nt, synthetic construct
  source 1..23: mol_type = other RNA
taaggccacggagcgagacatct

SEQ ID NO: 149 - RNA, 23 nt, synthetic construct
  source 1..23: mol_type = other RNA
caggccagaaagagagagtagcg

SEQ ID NO: 150 - RNA, 24 nt, synthetic construct
  source 1..24: mol_type = other RNA
gcctggaggctatccagcgtgagt

SEQ ID NO: 151 - RNA, 23 nt, synthetic construct
  source 1..23: mol_type = other RNA
ggccgccagcccagttgtagcac

SEQ ID NO: 152 - RNA, 23 nt, synthetic construct
  source 1..23: mol_type = other RNA
ctggccgccagcccagttgtagc

SEQ ID NO: 153 - RNA, 23 nt, synthetic construct
  source 1..23: mol_type = other RNA
gccatccccgttttacctggagc

SEQ ID NO: 154 - RNA, 23 nt, synthetic construct
  source 1..23: mol_type = other RNA
cgccatccccgttttacctggag

SEQ ID NO: 155 - RNA, 23 nt, synthetic construct
  source 1..23: mol_type = other RNA
ccgccatccccgttttacctgga

SEQ ID NO: 156 - RNA, 23 nt, synthetic construct
  source 1..23: mol_type = other RNA
gggatggcgggagggttgacctc

SEQ ID NO: 157 - RNA, 24 nt, synthetic construct
  source 1..24: mol_type = other RNA
gagctaaggacctcaggacctttg

SEQ ID NO: 158 - RNA, 24 nt, synthetic construct
  source 1..24: mol_type = other RNA
gagaggctttcggggtggaaggac

SEQ ID NO: 159 - RNA, 23 nt, synthetic construct
  source 1..23: mol_type = other RNA
gagaggctttcggggtggaagga

SEQ ID NO: 160 - RNA, 23 nt, synthetic construct
  source 1..23: mol_type = other RNA
agagaggctttcggggtggaagg

SEQ ID NO: 161 - DNA, 22 bp, synthetic construct
  source 1..22: mol_type = other DNA
cagtgatagagatctccctatc

SEQ ID NO: 162 - DNA, 24 bp, synthetic construct
  source 1..24: mol_type = other DNA
ggcccttgtggctgacccgtggct

SEQ ID NO: 163 - DNA, 38 bp, synthetic construct
  source 1..38: mol_type = other DNA
tgtggcccttgtggctgacccgtggctgctcccaccca

SEQ ID NO: 164 - DNA, 28 bp, synthetic construct
  source 1..28: mol_type = other DNA
caccggcccttgtggctgacccgtggct

SEQ ID NO: 165 - DNA, 28 bp, synthetic construct
  source 1..28: mol_type = other DNA
agacagccacgggtcagccacaagggcc

SEQ ID NO: 166 - DNA, 20 bp, synthetic construct
  source 1..20: mol_type = other DNA
gggtgggagcagccacgggt

SEQ ID NO: 167 - DNA, 34 bp, synthetic construct
  source 1..34: mol_type = other DNA
cttgggtgggagcagccacgggtcagccacaagg

SEQ ID NO: 168 - DNA, 24 bp, synthetic construct
  source 1..24: mol_type = other DNA
caccgggtgggagcagccacgggt

SEQ ID NO: 169 - DNA, 24 bp, synthetic construct
  source 1..24: mol_type = other DNA
agacacccgtggctgctcccaccc

SEQ ID NO: 170 - DNA, 21 bp, synthetic construct
  source 1..21: mol_type = other DNA
tgggtgggagcagccacgggt

SEQ ID NO: 171 - DNA, 35 bp, synthetic construct
  source 1..35: mol_type = other DNA
tcttgggtgggagcagccacgggtcagccacaagg

SEQ ID NO: 172 - DNA, 26 bp, synthetic construct
  source 1..26: mol_type = other DNA
caccgtgggtgggagcagccacgggt

SEQ ID NO: 173 - DNA, 26 bp, synthetic construct
  source 1..26: mol_type = other DNA
agacacccgtggctgctcccacccac

SEQ ID NO: 174 - DNA, 22 bp, synthetic construct
  source 1..22: mol_type = other DNA
ttgggtgggagcagccacgggt

SEQ ID NO: 175 - DNA, 36 bp, synthetic construct
  source 1..36: mol_type = other DNA
ttcttgggtgggagcagccacgggtcagccacaagg

SEQ ID NO: 176 - DNA, 27 bp, synthetic construct
  source 1..27: mol_type = other DNA
caccgttgggtgggagcagccacgggt

SEQ ID NO: 177 - DNA, 27 bp, synthetic construct
  source 1..27: mol_type = other DNA
agacacccgtggctgctcccacccaac

SEQ ID NO: 178 - DNA, 24 bp, synthetic construct
  source 1..24: mol_type = other DNA
tcttgggtgggagcagccacgggt

SEQ ID NO: 179 - DNA, 38 bp, synthetic construct
  source 1..38: mol_type = other DNA
cattcttgggtgggagcagccacgggtcagccacaagg

SEQ ID NO: 180 - DNA, 29 bp, synthetic construct
  source 1..29: mol_type = other DNA
caccgtcttgggtgggagcagccacgggt

SEQ ID NO: 181 - DNA, 29 bp, synthetic construct
  source 1..29: mol_type = other DNA
agacacccgtggctgctcccacccaagac

SEQ ID NO: 182 - DNA, 25 bp, synthetic construct
  source 1..25: mol_type = other DNA
ttcttgggtgggagcagccacgggt

SEQ ID NO: 183 - DNA, 39 bp, synthetic construct
  source 1..39: mol_type = other DNA
gcattcttgggtgggagcagccacgggtcagccacaagg

SEQ ID NO: 184 - DNA, 30 bp, synthetic construct
  source 1..30: mol_type = other DNA
caccgttcttgggtgggagcagccacgggt

SEQ ID NO: 185 - DNA, 30 bp, synthetic construct
  source 1..30: mol_type = other DNA
agacacccgtggctgctcccacccaagaac

SEQ ID NO: 186 - DNA, 21 bp, synthetic construct
  source 1..21: mol_type = other DNA
tgggtgggagcagccgcgggt

SEQ ID NO: 187 - DNA, 35 bp, synthetic construct
  source 1..35: mol_type = other DNA
tcttgggtgggagcagccgcgggtcagccacaagg

SEQ ID NO: 188 - DNA, 26 bp, synthetic construct
  source 1..26: mol_type = other DNA
caccgtgggtgggagcagccgcgggt

SEQ ID NO: 189 - DNA, 26 bp, synthetic construct
  source 1..26: mol_type = other DNA
agacacccgcggctgctcccacccac

SEQ ID NO: 190 - DNA, 23 bp, synthetic construct
  source 1..23: mol_type = other DNA
catgctcccgggctcctgcacac

SEQ ID NO: 191 - DNA, 37 bp, synthetic construct
  source 1..37: mol_type = other DNA
ctccatgctcccgggctcctgcacaccccacccacac

SEQ ID NO: 192 - DNA, 28 bp, synthetic construct
  source 1..28: mol_type = other DNA
caccgcatgctcccgggctcctgcacac

SEQ ID NO: 193 - DNA, 28 bp, synthetic construct
  source 1..28: mol_type = other DNA
agacgtgtgcaggagcccgggagcatgc

SEQ ID NO: 194 - DNA, 23 bp, synthetic construct
  source 1..23: mol_type = other DNA
cctccatgctcccgggctcctgc

SEQ ID NO: 195 - DNA, 37 bp, synthetic construct
  source 1..37: mol_type = other DNA
acccctccatgctcccgggctcctgcacaccccaccc

SEQ ID NO: 196 - DNA, 28 bp, synthetic construct
  source 1..28: mol_type = other DNA
caccgcctccatgctcccgggctcctgc

SEQ ID NO: 197 - DNA, 28 bp, synthetic construct
  source 1..28: mol_type = other DNA
agacgcaggagcccgggagcatggaggc

SEQ ID NO: 198 - DNA, 37 bp, synthetic construct
  source 1..37: mol_type = other DNA
ctcagccaccaccgccaagcccgggactctcccagac

SEQ ID NO: 199 - DNA, 28 bp, synthetic construct
  source 1..28: mol_type = other DNA
caccgagccaccaccgccaagcccggga

SEQ ID NO: 200 - DNA, 28 bp, synthetic construct
  source 1..28: mol_type = other DNA
agactcccgggcttggcggtggtggctc

SEQ ID NO: 201 - DNA, 23 bp, synthetic construct
  source 1..23: mol_type = other DNA
tcccttctcctgtcctgtcaatg

SEQ ID NO: 202 - DNA, 37 bp, synthetic construct
  source 1..37: mol_type = other DNA
ttctcccttctcctgtcctgtcaatgttatccaaagc

SEQ ID NO: 203 - DNA, 28 bp, synthetic construct
  source 1..28: mol_type = other DNA
caccgtcccttctcctgtcctgtcaatg

SEQ ID NO: 204 - DNA, 28 bp, synthetic construct
  source 1..28: mol_type = other DNA
agaccattgacaggacaggagaagggac

SEQ ID NO: 205 - DNA, 23 bp, synthetic construct
  source 1..23: mol_type = other DNA
cccttctcctgtcctgtcaatgt

SEQ ID NO: 206 - DNA, 37 bp, synthetic construct
  source 1..37: mol_type = other DNA
tctcccttctcctgtcctgtcaatgttatccaaagcc

SEQ ID NO: 207 - DNA, 28 bp, synthetic construct
  source 1..28: mol_type = other DNA
caccgcccttctcctgtcctgtcaatgt

SEQ ID NO: 208 - DNA, 28 bp, synthetic construct
  source 1..28: mol_type = other DNA
agacacattgacaggacaggagaagggc

SEQ ID NO: 209 - DNA, 23 bp, synthetic construct
  source 1..23: mol_type = other DNA
tgtcctgtcaatgttatccaaag

SEQ ID NO: 210 - DNA, 37 bp, synthetic construct
  source 1..37: mol_type = other DNA
tcctgtcctgtcaatgttatccaaagccctcatatat

SEQ ID NO: 211 - DNA, 28 bp, synthetic construct
  source 1..28: mol_type = other DNA
caccgtgtcctgtcaatgttatccaaag

SEQ ID NO: 212 - DNA, 28 bp, synthetic construct
  source 1..28: mol_type = other DNA
agacctttggataacattgacaggacac

SEQ ID NO: 213 - DNA, 23 bp, synthetic construct
  source 1..23: mol_type = other DNA
atccaaagccctcatatattcag

SEQ ID NO: 214 - DNA, 37 bp, synthetic construct
  source 1..37: mol_type = other DNA
gttatccaaagccctcatatattcagtcaacaaacac

SEQ ID NO: 215 - DNA, 28 bp, synthetic construct
  source 1..28: mol_type = other DNA
caccgatccaaagccctcatatattcag

SEQ ID NO: 216 - DNA, 28 bp, synthetic construct
  source 1..28: mol_type = other DNA
agacctgaatatatgagggctttggatc

SEQ ID NO: 217 - DNA, 23 bp, synthetic construct
  source 1..23: mol_type = other DNA
aaggcagtgttcagtgccagccc

SEQ ID NO: 218 - DNA, 37 bp, synthetic construct
  source 1..37: mol_type = other DNA
atcaaggcagtgttcagtgccagccctgcacaaacag

SEQ ID NO: 219 - DNA, 28 bp, synthetic construct
  source 1..28: mol_type = other DNA
caccgaaggcagtgttcagtgccagccc

SEQ ID NO: 220 - DNA, 28 bp, synthetic construct
  source 1..28: mol_type = other DNA
agacgggctggcactgaacactgccttc

SEQ ID NO: 221 - DNA, 23 bp, synthetic construct
  source 1..23: mol_type = other DNA
tcaaggcagtgttcagtgccagc

SEQ ID NO: 222 - DNA, 37 bp, synthetic construct
  source 1..37: mol_type = other DNA
agatcaaggcagtgttcagtgccagccctgcacaaac

SEQ ID NO: 223 - DNA, 28 bp, synthetic construct
  source 1..28: mol_type = other DNA
caccgtcaaggcagtgttcagtgccagc

SEQ ID NO: 224 - DNA, 28 bp, synthetic construct
  source 1..28: mol_type = other DNA
agacgctggcactgaacactgccttgac

SEQ ID NO: 225 - DNA, 23 bp, synthetic construct
  source 1..23: mol_type = other DNA
gtgaaattagacaagcgcatatt

SEQ ID NO: 226 - DNA, 37 bp, synthetic construct
  source 1..37: mol_type = other DNA
gctgtgaaattagacaagcgcatattgctccaaataa

SEQ ID NO: 227 - DNA, 27 bp, synthetic construct
  source 1..27: mol_type = other DNA
caccgtgaaattagacaagcgcatatt

SEQ ID NO: 228 - DNA, 27 bp, synthetic construct
  source 1..27: mol_type = other DNA
agacaatatgcgcttgtctaatttcac

SEQ ID NO: 229 - DNA, 23 bp, synthetic construct
  source 1..23: mol_type = other DNA
ttggagcaatatgcgcttgtcta

SEQ ID NO: 230 - DNA, 37 bp, synthetic construct
  source 1..37: mol_type = other DNA
tatttggagcaatatgcgcttgtctaatttcacagca

SEQ ID NO: 231 - DNA, 28 bp, synthetic construct
  source 1..28: mol_type = other DNA
caccgttggagcaatatgcgcttgtcta

SEQ ID NO: 232 - DNA, 28 bp, synthetic construct
  source 1..28: mol_type = other DNA
agactagacaagcgcatattgctccaac

SEQ ID NO: 233 - DNA, 24 bp, synthetic construct
  source 1..24: mol_type = other DNA
gttttcttgctgtgaaattagaca

SEQ ID NO: 234 - DNA, 38 bp, synthetic construct
  source 1..38: mol_type = other DNA
tcagttttcttgctgtgaaattagacaagcgcatattg

SEQ ID NO: 235 - DNA, 28 bp, synthetic construct
  source 1..28: mol_type = other DNA
caccgttttcttgctgtgaaattagaca

SEQ ID NO: 236 - DNA, 28 bp, synthetic construct
  source 1..28: mol_type = other DNA
agactgtctaatttcacagcaagaaaac

SEQ ID NO: 237 - DNA, 23 bp, synthetic construct
  source 1..23: mol_type = other DNA
tcacagcaagaaaactgagctga

SEQ ID NO: 238 - DNA, 37 bp, synthetic construct
  source 1..37: mol_type = other DNA
atttcacagcaagaaaactgagctgaggctcaaagaa

SEQ ID NO: 239 - DNA, 28 bp, synthetic construct
  source 1..28: mol_type = other DNA
caccgtcacagcaagaaaactgagctga

SEQ ID NO: 240 - DNA, 28 bp, synthetic construct
  source 1..28: mol_type = other DNA
agactcagctcagttttcttgctgtgac

SEQ ID NO: 241 - DNA, 23 bp, synthetic construct
  source 1..23: mol_type = other DNA
aagaagtcaagcgccctgctggg

SEQ ID NO: 242 - DNA, 37 bp, synthetic construct
  source 1..37: mol_type = other DNA
tcaaagaagtcaagcgccctgctggggcgtcacacag

SEQ ID NO: 243 - DNA, 28 bp, synthetic construct
  source 1..28: mol_type = other DNA
caccgaagaagtcaagcgccctgctggg

SEQ ID NO: 244 - DNA, 28 bp, synthetic construct
  source 1..28: mol_type = other DNA
agaccccagcagggcgcttgacttcttc

SEQ ID NO: 245 - DNA, 23 bp, synthetic construct
  source 1..23: mol_type = other DNA
gaagtcaagcgccctgctggggc

SEQ ID NO: 246 - DNA, 37 bp, synthetic construct
  source 1..37: mol_type = other DNA
aaagaagtcaagcgccctgctggggcgtcacacaggg

SEQ ID NO: 247 - DNA, 27 bp, synthetic construct
  source 1..27: mol_type = other DNA
caccgaagtcaagcgccctgctggggc

SEQ ID NO: 248 - DNA, 27 bp, synthetic construct
  source 1..27: mol_type = other DNA
agacgccccagcagggcgcttgacttc

SEQ ID NO: 249 - DNA, 23 bp, synthetic construct
  source 1..23: mol_type = other DNA
aactctgcacccgtccctgtgtg

SEQ ID NO: 250 - DNA, 37 bp, synthetic construct
  source 1..37: mol_type = other DNA
ctcaactctgcacccgtccctgtgtgacgccccagca

SEQ ID NO: 251 - DNA, 28 bp, synthetic construct
  source 1..28: mol_type = other DNA
caccgaactctgcacccgtccctgtgtg

SEQ ID NO: 252 - DNA, 28 bp, synthetic construct
  source 1..28: mol_type = other DNA
agaccacacagggacgggtgcagagttc

SEQ ID NO: 253 - DNA, 23 bp, synthetic construct
  source 1..23: mol_type = other DNA
tgctggggcgtcacacagggacg

SEQ ID NO: 254 - DNA, 37 bp, synthetic construct
  source 1..37: mol_type = other DNA
ccctgctggggcgtcacacagggacgggtgcagagtt

SEQ ID NO: 255 - DNA, 28 bp, synthetic construct
  source 1..28: mol_type = other DNA
caccgtgctggggcgtcacacagggacg

SEQ ID NO: 256 - DNA, 28 bp, synthetic construct
  source 1..28: mol_type = other DNA
agaccgtccctgtgtgacgccccagcac

SEQ ID NO: 257 - DNA, 23 bp, synthetic construct
  source 1..23: mol_type = other DNA
aacatggcccgagatagatgcgg

SEQ ID NO: 258 - DNA, 37 bp, synthetic construct
  source 1..37: mol_type = other DNA
gcaaacatggcccgagatagatgcgggcttccaactc

SEQ ID NO: 259 - DNA, 28 bp, synthetic construct
  source 1..28: mol_type = other DNA
caccgaacatggcccgagatagatgcgg

SEQ ID NO: 260 - DNA, 28 bp, synthetic construct
  source 1..28: mol_type = other DNA
agacccgcatctatctcgggccatgttc

SEQ ID NO: 261 - DNA, 23 bp, synthetic construct
  source 1..23: mol_type = other DNA
acagaggcttggtgctgcaaaca

SEQ ID NO: 262 - DNA, 37 bp, synthetic construct
  source 1..37: mol_type = other DNA
gaaacagaggcttggtgctgcaaacatggcccgagat

SEQ ID NO: 263 - DNA, 28 bp, synthetic construct
  source 1..28: mol_type = other DNA
caccgacagaggcttggtgctgcaaaca

SEQ ID NO: 264 - DNA, 28 bp, synthetic construct
  source 1..28: mol_type = other DNA
agactgtttgcagcaccaagcctctgtc

SEQ ID NO: 265 - DNA, 23 bp, synthetic construct
  source 1..23: mol_type = other DNA
tccaagggaaacagaggcttggt

SEQ ID NO: 266 - DNA, 37 bp, synthetic construct
  source 1..37: mol_type = other DNA
tgctccaagggaaacagaggcttggtgctgcaaacat

SEQ ID NO: 267 - DNA, 28 bp, synthetic construct
  source 1..28: mol_type = other DNA
caccgtccaagggaaacagaggcttggt

SEQ ID NO: 268 - DNA, 28 bp, synthetic construct
  source 1..28: mol_type = other DNA
agacaccaagcctctgtttcccttggac

SEQ ID NO: 269 - DNA, 23 bp, synthetic construct
  source 1..23: mol_type = other DNA
gactcagcacagctgctccaagg

SEQ ID NO: 270 - DNA, 37 bp, synthetic construct
  source 1..37: mol_type = other DNA
tctgactcagcacagctgctccaagggaaacagaggc

SEQ ID NO: 271 - DNA, 27 bp, synthetic construct
  source 1..27: mol_type = other DNA
caccgactcagcacagctgctccaagg

SEQ ID NO: 272 - DNA, 27 bp, synthetic construct
  source 1..27: mol_type = other DNA
agacccttggagcagctgtgctgagtc

SEQ ID NO: 273 - DNA, 23 bp, synthetic construct
  source 1..23: mol_type = other DNA
gcctgggtctgactcagcacagc

SEQ ID NO: 274 - DNA, 37 bp, synthetic construct
  source 1..37: mol_type = other DNA
ccagcctgggtctgactcagcacagctgctccaaggg

SEQ ID NO: 275 - DNA, 27 bp, synthetic construct
  source 1..27: mol_type = other DNA
caccgcctgggtctgactcagcacagc

SEQ ID NO: 276 - DNA, 27 bp, synthetic construct
  source 1..27: mol_type = other DNA
agacgctgtgctgagtcagacccaggc

SEQ ID NO: 277 - DNA, 23 bp, synthetic construct
  source 1..23: mol_type = other DNA
cccttggagcagctgtgctgagt

SEQ ID NO: 278 - DNA, 37 bp, synthetic construct
  source 1..37: mol_type = other DNA
tttcccttggagcagctgtgctgagtcagacccaggc

SEQ ID NO: 279 - DNA, 28 bp, synthetic construct
  source 1..28: mol_type = other DNA
caccgcccttggagcagctgtgctgagt

SEQ ID NO: 280 - DNA, 28 bp, synthetic construct
  source 1..28: mol_type = other DNA
agacactcagcacagctgctccaagggc

SEQ ID NO: 281 - DNA, 23 bp, synthetic construct
  source 1..23: mol_type = other DNA
tcagtgcccagcctgggtctgac

SEQ ID NO: 282 - DNA, 37 bp, synthetic construct
  source 1..37: mol_type = other DNA
ccctcagtgcccagcctgggtctgactcagcacagct

SEQ ID NO: 283 - DNA, 28 bp, synthetic construct
  source 1..28: mol_type = other DNA
caccgtcagtgcccagcctgggtctgac

SEQ ID NO: 284 - DNA, 28 bp, synthetic construct
  source 1..28: mol_type = other DNA
agacgtcagacccaggctgggcactgac

SEQ ID NO: 285 - DNA, 20 bp, synthetic construct
  source 1..20: mol_type = other DNA
gagatagatgcgggcttcca

SEQ ID NO: 286 - DNA, 23 bp, synthetic construct
  source 1..23: mol_type = other DNA
atacacatcagaatccttacttt

SEQ ID NO: 287 - DNA, 37 bp, synthetic construct
  source 1..37: mol_type = other DNA
gatatacacatcagaatccttactttgtgacacattt

SEQ ID NO: 288 - DNA, 28 bp, synthetic construct
  source 1..28: mol_type = other DNA
caccgatacacatcagaatccttacttt

SEQ ID NO: 289 - DNA, 28 bp, synthetic construct
  source 1..28: mol_type = other DNA
agacaaagtaaggattctgatgtgtatc

SEQ ID NO: 290 - DNA, 24 bp, synthetic construct
  source 1..24: mol_type = other DNA
gtaaggattctgatgtgtatatca

SEQ ID NO: 291 - DNA, 38 bp, synthetic construct
  source 1..38: mol_type = other DNA
aaagtaaggattctgatgtgtatatcacagacaaaact

SEQ ID NO: 292 - DNA, 28 bp, synthetic construct
  source 1..28: mol_type = other DNA
caccgtaaggattctgatgtgtatatca

SEQ ID NO: 293 - DNA, 28 bp, synthetic construct
  source 1..28: mol_type = other DNA
agactgatatacacatcagaatccttac

SEQ ID NO: 294 - DNA, 23 bp, synthetic construct
  source 1..23: mol_type = other DNA
gagcaacagtgctgtggcctgga

SEQ ID NO: 295 - DNA, 37 bp, synthetic construct
  source 1..37: mol_type = other DNA
caagagcaacagtgctgtggcctggagcaacaaatct

SEQ ID NO: 296 - DNA, 27 bp, synthetic construct
  source 1..27: mol_type = other DNA
caccgagcaacagtgctgtggcctgga

SEQ ID NO: 297 - DNA, 27 bp, synthetic construct
  source 1..27: mol_type = other DNA
agactccaggccacagcactgttgctc

SEQ ID NO: 298 - DNA, 23 bp, synthetic construct
  source 1..23: mol_type = other DNA
ggagcaacaaatctgactttgca

SEQ ID NO: 299 - DNA, 37 bp, synthetic construct
  source 1..37: mol_type = other DNA
cctggagcaacaaatctgactttgcatgtgcaaacgc

SEQ ID NO: 300 - DNA, 27 bp, synthetic construct
  source 1..27: mol_type = other DNA
caccggagcaacaaatctgactttgca

SEQ ID NO: 301 - DNA, 27 bp, synthetic construct
  source 1..27: mol_type = other DNA
agactgcaaagtcagatttgttgctcc

SEQ ID NO: 302 - DNA, 23 bp, synthetic construct
  source 1..23: mol_type = other DNA
tgctgttgttgaaggcgtttgca

SEQ ID NO: 303 - DNA, 37 bp, synthetic construct
  source 1..37: mol_type = other DNA
taatgctgttgttgaaggcgtttgcacatgcaaagtc

SEQ ID NO: 304 - DNA, 28 bp, synthetic construct
  source 1..28: mol_type = other DNA
caccgtgctgttgttgaaggcgtttgca

SEQ ID NO: 305 - DNA, 28 bp, synthetic construct
  source 1..28: mol_type = other DNA
agactgcaaacgccttcaacaacagcac

SEQ ID NO: 306 - DNA, 23 bp, synthetic construct
  source 1..23: mol_type = other DNA
agcattattccagaagacacctt

SEQ ID NO: 307 - DNA, 37 bp, synthetic construct
  source 1..37: mol_type = other DNA
aacagcattattccagaagacaccttcttccccagcc

SEQ ID NO: 308 - DNA, 28 bp, synthetic construct
  source 1..28: mol_type = other DNA
caccgagcattattccagaagacacctt

SEQ ID NO: 309 - DNA, 28 bp, synthetic construct
  source 1..28: mol_type = other DNA
agacaaggtgtcttctggaataatgctc

SEQ ID NO: 310 - DNA, 23 bp, synthetic construct
  source 1..23: mol_type = other DNA
tattccagaagacaccttcttcc

SEQ ID NO: 311 - DNA, 37 bp, synthetic construct
  source 1..37: mol_type = other DNA
cattattccagaagacaccttcttccccagcccaggt

SEQ ID NO: 312 - DNA, 28 bp, synthetic construct
  source 1..28: mol_type = other DNA
caccgtattccagaagacaccttcttcc

SEQ ID NO: 313 - DNA, 28 bp, synthetic construct
  source 1..28: mol_type = other DNA
agacggaagaaggtgtcttctggaatac

SEQ ID NO: 314 - DNA, 23 bp, synthetic construct
  source 1..23: mol_type = other DNA
acaaagtaaggattctgatgtgt

SEQ ID NO: 315 - DNA, 37 bp, synthetic construct
  source 1..37: mol_type = other DNA
gtcacaaagtaaggattctgatgtgtatatcacagac

SEQ ID NO: 316 - DNA, 28 bp, synthetic construct
  source 1..28: mol_type = other DNA
caccgacaaagtaaggattctgatgtgt

SEQ ID NO: 317 - DNA, 28 bp, synthetic construct
  source 1..28: mol_type = other DNA
agacacacatcagaatccttactttgtc

SEQ ID NO: 318 - DNA, 23 bp, synthetic construct
  source 1..23: mol_type = other DNA
gtctagcacagttttgtctgtga

SEQ ID NO: 319 - DNA, 37 bp, synthetic construct
  source 1..37: mol_type = other DNA
catgtctagcacagttttgtctgtgatatacacatca

SEQ ID NO: 320 - DNA, 27 bp, synthetic construct
  source 1..27: mol_type = other DNA
caccgtctagcacagttttgtctgtga

SEQ ID NO: 321 - DNA, 27 bp, synthetic construct
  source 1..27: mol_type = other DNA
agactcacagacaaaactgtgctagac

SEQ ID NO: 322 - DNA, 23 bp, synthetic construct
  source 1..23: mol_type = other DNA
cttgaagtccatagacctcatgt

SEQ ID NO: 323 - DNA, 37 bp, synthetic construct
  source 1..37: mol_type = other DNA
gctcttgaagtccatagacctcatgtctagcacagtt

SEQ ID NO: 324 - DNA, 28 bp, synthetic construct
  source 1..28: mol_type = other DNA
caccgcttgaagtccatagacctcatgt

SEQ ID NO: 325 - DNA, 28 bp, synthetic construct
  source 1..28: mol_type = other DNA
agacacatgaggtctatggacttcaagc

SEQ ID NO: 326 - DNA, 23 bp, synthetic construct
  source 1..23: mol_type = other DNA
ggccacagcactgttgctcttga

SEQ ID NO: 327 - DNA, 37 bp, synthetic construct
  source 1..37: mol_type = other DNA
ccaggccacagcactgttgctcttgaagtccatagac

SEQ ID NO: 328 - DNA, 27 bp, synthetic construct
  source 1..27: mol_type = other DNA
caccggccacagcactgttgctcttga

SEQ ID NO: 329 - DNA, 27 bp, synthetic construct
  source 1..27: mol_type = other DNA
agactcaagagcaacagtgctgtggcc

SEQ ID NO: 330 - DNA, 23 bp, synthetic construct
  source 1..23: mol_type = other DNA
tgcaaagtcagatttgttgctcc

SEQ ID NO: 331 - DNA, 37 bp, synthetic construct
  source 1..37: mol_type = other DNA
acatgcaaagtcagatttgttgctccaggccacagca

SEQ ID NO: 332 - DNA, 28 bp, synthetic construct
  source 1..28: mol_type = other DNA
caccgtgcaaagtcagatttgttgctcc

SEQ ID NO: 333 - DNA, 28 bp, synthetic construct
  source 1..28: mol_type = other DNA
agacggagcaacaaatctgactttgcac

SEQ ID NO: 334 - DNA, 23 bp, synthetic construct
  source 1..23: mol_type = other DNA
gaataatgctgttgttgaaggcg

SEQ ID NO: 335 - DNA, 37 bp, synthetic construct
  source 1..37: mol_type = other DNA
ctggaataatgctgttgttgaaggcgtttgcacatgc

SEQ ID NO: 336 - DNA, 27 bp, synthetic construct
  source 1..27: mol_type = other DNA
caccgaataatgctgttgttgaaggcg

SEQ ID NO: 337 - DNA, 27 bp, synthetic construct
  source 1..27: mol_type = other DNA
agaccgccttcaacaacagcattattc

SEQ ID NO: 338 - DNA, 23 bp, synthetic construct
  source 1..23: mol_type = other DNA
taaggccacggagcgagacatct

SEQ ID NO: 339 - DNA, 37 bp, synthetic construct
  source 1..37: mol_type = other DNA
agctaaggccacggagcgagacatctcggcccgaatg

SEQ ID NO: 340 - DNA, 28 bp, synthetic construct
  source 1..28: mol_type = other DNA
caccgtaaggccacggagcgagacatct

SEQ ID NO: 341 - DNA, 28 bp, synthetic construct
  source 1..28: mol_type = other DNA
agacagatgtctcgctccgtggccttac

SEQ ID NO: 342 - DNA, 23 bp, synthetic construct
  source 1..23: mol_type = other DNA
caggccagaaagagagagtagcg

SEQ ID NO: 343 - DNA, 37 bp, synthetic construct
  source 1..37: mol_type = other DNA
ctccaggccagaaagagagagtagcgcgagcacagct

SEQ ID NO: 344 - DNA, 28 bp, synthetic construct
  source 1..28: mol_type = other DNA
caccgcaggccagaaagagagagtagcg

SEQ ID NO: 345 - DNA, 28 bp, synthetic construct
  source 1..28: mol_type = other DNA
agaccgctactctctctttctggcctgc

SEQ ID NO: 346 - DNA, 24 bp, synthetic construct
  source 1..24: mol_type = other DNA
gcctggaggctatccagcgtgagt

SEQ ID NO: 347 - DNA, 38 bp, synthetic construct
  source 1..38: mol_type = other DNA
ctggcctggaggctatccagcgtgagtctctcctaccc

SEQ ID NO: 348 - DNA, 28 bp, synthetic construct
  source 1..28: mol_type = other DNA
caccgcctggaggctatccagcgtgagt

SEQ ID NO: 349 - DNA, 28 bp, synthetic construct
  source 1..28: mol_type = other DNA
agacactcacgctggatagcctccaggc

SEQ ID NO: 350 - DNA, 23 bp, synthetic construct
  source 1..23: mol_type = other DNA
ggccgccagcccagttgtagcac

SEQ ID NO: 351 - DNA, 37 bp, synthetic construct
  source 1..37: mol_type = other DNA
cctggccgccagcccagttgtagcaccgcccagacga

SEQ ID NO: 352 - DNA, 27 bp, synthetic construct
  source 1..27: mol_type = other DNA
caccggccgccagcccagttgtagcac

SEQ ID NO: 353 - DNA, 27 bp, synthetic construct
  source 1..27: mol_type = other DNA
agacgtgctacaactgggctggcggcc

SEQ ID NO: 354 - DNA, 23 bp, synthetic construct
  source 1..23: mol_type = other DNA
ctggccgccagcccagttgtagc

SEQ ID NO: 355 - DNA, 37 bp, synthetic construct
  source 1..37: mol_type = other DNA
atcctggccgccagcccagttgtagcaccgcccagac

SEQ ID NO: 356 - DNA, 28 bp, synthetic construct
  source 1..28: mol_type = other DNA
caccgctggccgccagcccagttgtagc

SEQ ID NO: 357 - DNA, 28 bp, synthetic construct
  source 1..28: mol_type = other DNA
agacgctacaactgggctggcggccagc

SEQ ID NO: 358 - DNA, 23 bp, synthetic construct
  source 1..23: mol_type = other DNA
gccatccccgttttacctggagc

SEQ ID NO: 359 - DNA, 37 bp, synthetic construct
  source 1..37: mol_type = other DNA
cccgccatccccgttttacctggagccacccaaagcg

SEQ ID NO: 360 - DNA, 27 bp, synthetic construct
  source 1..27: mol_type = other DNA
caccgccatccccgttttacctggagc

SEQ ID NO: 361 - DNA, 27 bp, synthetic construct
  source 1..27: mol_type = other DNA
agacgctccaggtaaaacggggatggc

SEQ ID NO: 362 - DNA, 23 bp, synthetic construct
  source 1..23: mol_type = other DNA
cgccatccccgttttacctggag

SEQ ID NO: 363 - DNA, 37 bp, synthetic construct
  source 1..37: mol_type = other DNA
tcccgccatccccgttttacctggagccacccaaagc

SEQ ID NO: 364 - DNA, 28 bp, synthetic construct
  source 1..28: mol_type = other DNA
caccgcgccatccccgttttacctggag

SEQ ID NO: 365 - DNA, 28 bp, synthetic construct
  source 1..28: mol_type = other DNA
agacctccaggtaaaacggggatggcgc

SEQ ID NO: 366 - DNA, 23 bp, synthetic construct
  source 1..23: mol_type = other DNA
ccgccatccccgttttacctgga

SEQ ID NO: 367 - DNA, 37 bp, synthetic construct
  source 1..37: mol_type = other DNA
ctcccgccatccccgttttacctggagccacccaaag

SEQ ID NO: 368 - DNA, 28 bp, synthetic construct
  source 1..28: mol_type = other DNA
caccgccgccatccccgttttacctgga

SEQ ID NO: 369 - DNA, 28 bp, synthetic construct
  source 1..28: mol_type = other DNA
agactccaggtaaaacggggatggcggc

SEQ ID NO: 370 - DNA, 23 bp, synthetic construct
  source 1..23: mol_type = other DNA
gggatggcgggagggttgacctc

SEQ ID NO: 371 - DNA, 37 bp, synthetic construct
  source 1..37: mol_type = other DNA
acggggatggcgggagggttgacctccagccccacag

SEQ ID NO: 372 - DNA, 27 bp, synthetic construct
  source 1..27: mol_type = other DNA
caccgggatggcgggagggttgacctc

SEQ ID NO: 373 - DNA, 27 bp, synthetic construct
  source 1..27: mol_type = other DNA
agacgaggtcaaccctcccgccatccc

SEQ ID NO: 374 - DNA, 24 bp, synthetic construct
  source 1..24: mol_type = other DNA
gagctaaggacctcaggacctttg

SEQ ID NO: 375 - DNA, 38 bp, synthetic construct
  source 1..38: mol_type = other DNA
acagagctaaggacctcaggacctttgtggccacagag

SEQ ID NO: 376 - DNA, 28 bp, synthetic construct
  source 1..28: mol_type = other DNA
caccgagctaaggacctcaggacctttg

SEQ ID NO: 377 - DNA, 28 bp, synthetic construct
  source 1..28: mol_type = other DNA
agaccaaaggtcctgaggtccttagctc

SEQ ID NO: 378 - DNA, 24 bp, synthetic construct
  source 1..24: mol_type = other DNA
gagaggctttcggggtggaaggac

SEQ ID NO: 379 - DNA, 38 bp, synthetic construct
  source 1..38: mol_type = other DNA
ccagagaggctttcggggtggaaggactgcccaaaaaa

SEQ ID NO: 380 - DNA, 28 bp, synthetic construct
  source 1..28: mol_type = other DNA
caccgagaggctttcggggtggaaggac

SEQ ID NO: 381 - DNA, 28 bp, synthetic construct
  source 1..28: mol_type = other DNA
agacgtccttccaccccgaaagcctctc

SEQ ID NO: 382 - DNA, 23 bp, synthetic construct
  source 1..23: mol_type = other DNA
gagaggctttcggggtggaagga

SEQ ID NO: 383 - DNA, 37 bp, synthetic construct
  source 1..37: mol_type = other DNA
ccagagaggctttcggggtggaaggactgcccaaaaa

SEQ ID NO: 384 - DNA, 27 bp, synthetic construct
  source 1..27: mol_type = other DNA
caccgagaggctttcggggtggaagga

SEQ ID NO: 385 - DNA, 27 bp, synthetic construct
  source 1..27: mol_type = other DNA
agactccttccaccccgaaagcctctc

SEQ ID NO: 386 - DNA, 23 bp, synthetic construct
  source 1..23: mol_type = other DNA
agagaggctttcggggtggaagg

SEQ ID NO: 387 - DNA, 37 bp, synthetic construct
  source 1..37: mol_type = other DNA
cccagagaggctttcggggtggaaggactgcccaaaa

SEQ ID NO: 388 - DNA, 28 bp, synthetic construct
  source 1..28: mol_type = other DNA
caccgagagaggctttcggggtggaagg

SEQ ID NO: 389 - DNA, 28 bp, synthetic construct
  source 1..28: mol_type = other DNA
agacccttccaccccgaaagcctctctc

SEQ ID NO: 390 - DNA, 20 bp, synthetic construct
  source 1..20: mol_type = other DNA
catcacgagcagctggtttc

SEQ ID NO: 391 - DNA, 20 bp, synthetic construct
  source 1..20: mol_type = other DNA
tggcaatggataaggccgag

SEQ ID NO: 392 - DNA, 20 bp, synthetic construct
  source 1..20: mol_type = other DNA
ctctaacctggcactgcgtc

SEQ ID NO: 393 - DNA, 20 bp, synthetic construct
  source 1..20: mol_type = other DNA
cgaagtccacagctctccag

SEQ ID NO: 394 - DNA, 20 bp, synthetic construct
  source 1..20: mol_type = other DNA
cactgcctctgtcactctcg

SEQ ID NO: 395 - DNA, 20 bp, synthetic construct
  source 1..20: mol_type = other DNA
gacttgggccaggggaggag

SEQ ID NO: 396 - DNA, 20 bp, synthetic construct
  source 1..20: mol_type = other DNA
ccccttcctccacctccctc

SEQ ID NO: 397 - DNA, 20 bp, synthetic construct
  source 1..20: mol_type = other DNA
ggttgagtgctggattcgga

SEQ ID NO: 398 - DNA, 101 bp, synthetic construct
  source 1..101: mol_type = other DNA
cctgagtggctgagctcaggccttcgcagcattcttgggtgggagcagccacgggtcagccacaagggccacagccatgaatggcacagaaggccctaact

(19) United States
(12) Patent Application Publication
(10) Pub. No.: US 2026/0014279 A1
(43) Pub. Date: Jan. 15, 2026

(54) ENQP TYPE CAS PROTEINS AND APPLICATIONS THEREOF

(71) Applicant: Alia Therapeutics Srl, Milano (IT)

(72) Inventors: Antonio CASINI, Trento (IT); Laura Pezzè, Trento (IT); Matteo CICIANI, Schio (IT); Nicola SEGATA, Trento (IT); Anna CERESETO, Trento (IT)

(73) Assignee: Alia Therapeutics Srl, Milano (IT)

(21) Appl. No.: 19/112,164

(22) PCT Filed: Sep. 15, 2023
(86) PCT No.: PCT/EP2023/075483

§ 371 Date: Mar. 14, 2025

Related U.S. Application Data
(60) Provisional application No. 63/407,255, filed on Sep. 16, 2022.
(60) Provisional application No. 63/430,891, filed on Dec. 7, 2022.

(51) Int. Cl. A61K 48/00; C12N 9/22; C12N 15/11

(52) U.S. Cl. CPC A61K 48/0058; C12N 9/226; C12N 15/11; C12N 2310/20; C12N 2310/531

(57) ABSTRACT

Type II Cas proteins referred to as ENQP Type II Cas proteins; gRNAs for Type II Cas proteins; systems comprising Type II Cas proteins and gRNAs; nucleic acids encoding the Type II Cas proteins, gRNAs and systems; particles comprising the foregoing; pharmaceutical compositions of the foregoing; and uses of the foregoing, for example to alter the genomic DNA of a cell.

### 1. CROSS-REFERENCE TO RELATED APPLICATIONS

This application claims the priority benefit of U.S. provisional application Nos. 63/407,255, filed Sep. 16, 2022, and 63/430,891, filed Dec. 7, 2023, the contents of which are incorporated herein in their entireties by reference thereto

### 2. SEQUENCE LISTING

The instant application contains a Sequence Listing which has been submitted electronically in XML format and is hereby incorporated by reference in its entirety. Said XML Sequence Listing, created on Aug. 25, 2023, is named ALA-009WO_SL.xml and is 364,410 bytes in size.

### 3. BACKGROUND

CRISPR-Cas genome editing with Type II Cas proteins and associated guide RNAs (gRNAs) is a powerful tool with the potential to treat a variety of genetic diseases. Adeno-associated viral vectors (AAVs) are commonly used to deliver Cas proteins, for example Streptococcus pyogenes Cas9 (SpCas9), and their guide RNAs (gRNAs). However, packaging a large Cas protein such as SpCas9 together with a guide RNA into a single AAV vector can be challenging due to the limited packaging capacity of AAVs. Thus, there is a need for Type II Cas nucleases with smaller sizes that can be packaged together with a gRNA in a single AAV. In addition, the discovery of novel nucleases with new PAM specificities can broaden the range of targetable sites in the cell genome, making genome editing more flexible and efficient.

### 4. SUMMARY

This disclosure is based, in part, on the discovery of a Type II Cas protein from Thermophilibacter mediterraneus (referred to herein as “wild-type ENQP Type II Cas”). Wild-type ENQP Type II Cas protein is approximately 1000 amino acids in length, significantly shorter than SpCas9.

In one aspect, the disclosure provides Type II Cas proteins whose amino acid sequence comprises an amino acid sequence that is at least 50% identical (e.g., at least 55%, at least 60%, at least 65%, at least 70%, at least 75%, at least 85%, at least 90%, at least 95% identical, or more) to SEQ ID NO: 1 (such proteins referred to herein as “ENQP Type II Cas proteins”). Exemplary ENQP Type II Cas protein sequences are set forth in SEQ ID NO:1, SEQ ID NO:2, and SEQ ID NO:3.

In another aspect, the disclosure provides Type II Cas proteins comprising an amino acid sequence having at least 50% (e.g., at least 55%, at least 60%, at least 65%, at least 70%, at least 75%, at least 85%, at least 90%, at least 95%, or more) sequence identity to a RuvC-I domain, RuvC-II domain, RuvC-III domain, BH domain, REC domain, HNH domain, WED domain, or PID domain of an ENQP Type II Cas protein. In some embodiments, a Type II Cas protein of the disclosure is a chimeric Type II Cas protein, for example, comprising one or more domains from an ENQP Type II Cas protein and one or more domains from a different Type II Cas protein such as SpCas9.

In some embodiments, the Type II Cas proteins of the disclosure are in the form of a fusion protein, for example, comprising a ENQP Type II Cas protein sequence fused to one or more additional amino acid sequences, for example, one or more nuclear localization signals and/or one or more tags. Other exemplary fusion partners can enable base editing (e.g., where the fusion partner is nucleoside deaminase) or prime editing (e.g., where the fusion partner is a reverse transcriptase).

Exemplary features of Type II Cas proteins of the disclosure are described in Section 6.2 and specific embodiments 1 to 181 and 561 to 567, infra.

In further aspects, the disclosure provides guide (gRNA) molecules, for example single guide RNAs (sgRNAs), and combinations of two or more gRNA molecules (e.g., combinations of sgRNA molecules). In various embodiments, the disclosure provides gRNAs that can be used with the ENQP Type II Cas proteins of the disclosure. Exemplary features of the gRNAs of the disclosure and combinations of gRNAs of the disclosure are described in Section 6.3 and specific embodiments 182 to 509, infra.

In further aspects, the disclosure provides systems comprising a Type II Cas protein of the disclosure and one or more gRNAs, e.g., sgRNAs. For example, a system can comprise a ribonucleoprotein (RNP) comprising a Type II Cas protein complexed with a gRNA, e.g., an sgRNA or separate crRNA and tracrRNA. Exemplary features of systems are described in Section 6.4 and specific embodiments 510 to 512, infra.

In another aspect, the disclosure provides nucleic acids and pluralities of nucleic acids encoding a Type II Cas protein of the disclosure and, optionally, a guide RNA, for example a sgRNA. In some embodiments, the nucleic acids comprise a Type II Cas protein of the disclosure operably linked to a heterologous promoter, e.g., a mammalian promoter, for example a human promoter.

In another aspect, the disclosure provides nucleic acids encoding a gRNA of the disclosure, for example a sgRNA, and, optionally, a Type II Cas protein.

In another aspect, the disclosure provides nucleic acids encoding combinations of gRNAs of the disclosure, for example a combination of two gRNAs, and, optionally, a Type II Cas protein.

Exemplary features of nucleic and pluralities of nucleic acids of the disclosure are described in Section 6.5 and specific embodiments 513 to 560, infra.

In further aspects, the disclosure provides particles comprising the Type II Cas proteins, gRNAs, nucleic acids, and systems of the disclosure. Exemplary features of particles of the disclosure are described in Section 6.6 and specific embodiments 568 to 583, infra.

In another aspect, the disclosure provides cells and populations of cells containing or contacted with a Type II Cas protein, gRNA, nucleic acid, plurality of nucleic acids, system, or particle of the disclosure. Exemplary features of such cells and cell populations are described in Section 6.6 and specific embodiments 585 to 594 and 633, infra.

In another aspect, the disclosure provides pharmaceutical compositions comprising a Type II Cas protein, gRNA, nucleic acid, plurality of nucleic acids, system, particle, cell, or population of cells together with one or more excipients. Exemplary features of pharmaceutical compositions are described in Section 6.7 and specific embodiment 584, infra.

In another aspect, the disclosure provides methods of altering cells (e.g., editing the genome of a cell) using the Type II Cas proteins, gRNAs, nucleic acids, systems, particles, and pharmaceutical compositions of the disclosure. Cells altered according to the methods of the disclosure can be used, for example, to treat subjects having a disease or disorder, e.g., genetic disease or disorder, for example retinitis pigmentosa caused by a RHO mutation. Features of exemplary methods of altering cells are described in Section 6.8 and specific embodiments 595 to 632, infra.

### 5. BRIEF DESCRIPTION OF THE FIGURES

FIGS. 1A-1B show ENQP Type II Cas sgRNA scaffolds. FIGS. 1A-1B show schematic representation of the hairpin structure generated for visualization after in silico folding using RNA folding form v2.3 (www.unafold.org) of exemplary sgRNA scaffolds (not including the spacer sequence) designed from crRNAs and tracrRNAs identified for ENQP Type II Cas. FIG. 1A shows a standard full length sgRNA scaffold obtained by fusion of ENQP Type II Cas crRNA and tracrRNA, while FIG. 1B shows a trimmed version of the same scaffold. Scaffold sequences are shown in Table 6 (SEQ ID NOS 46-47, respectively in order of appearance).

FIGS. 2A-2B illustrate the determination of ENQP Type II Cas PAM specificity. FIG. 2A shows a PAM sequence logo for ENQP Type II Cas obtained using an in vitro PAM discovery assay. FIG. 2B shows a PAM enrichment heatmap for ENQP Type II Cas showing the nucleotide preferences at position 5,6,7 and 8 of the PAM.

FIG. 3 shows the activity of ENQP Type II Cas against an EGFP reporter gene. EGFP disruption was measured by cytofluorimetry after nucleofection of U2OS-EGFP cells with ENQP Type II Cas and two different sgRNAs targeting the EGFP coding sequence. Data are presented as mean±SEM for N≥2 independent replicates.

FIG. 4 shows activity of ENQP Type II Cas on endogenous genomic loci (RHO, DNMT1) after transient transfection in HEK293T cells. Data are presented as mean±SEM for N=2 independent replicates.

FIG. 5 shows allele-specificity of ENQP Type II Cas measured on the rs7984 RHO SNP after transient transfection in HEK293T cells (rs7984A allele) or HEK293-RHO-GFP cells (rs7984G allele). Data presented as mean±SEM for N≥2 independent replicates.

FIG. 6 shows an exemplary ENQP Type II Cas sgRNA scaffold (sgRNAtrimV2) (SEQ ID NO:92). The scaffold is based on the ENQP trimmed scaffold sgRNAtrimV1 and includes an additionally trimmed stem-loop (substitution with a GAAA tetraloop).

FIG. 7 shows a side-by-side comparison of indel formation by ENQP Type II Cas and RHO SNP rs7984 guide RNAs having the sgRNAtrimV1 and the sgRNAtrimV2 scaffolds.

FIG. 8 shows a schematic representation of the rs7984 locus with the position of ENQP Type II Cas sgRNAs which were evaluated for editing activity towards the SNP (Example 3). The rs7984A allele is shown in bold. Figure discloses SEQ ID NO: 398.

FIG. 9 shows the levels of indel generated by ENQP Type II Cas in combination with different guide RNAs targeting the rs7984 SNP after transient plasmid transfection of HEK293T cells (Example 3).

FIG. 10 shows the editing observed when evaluating ENQP Type II Cas in conjunction with different versions of a gRNA characterized by varying spacer lengths (20-25 nt) after transient plasmid transfection of HEK293T cells (Example 3).

FIG. 11 shows the allele specificity of gRNA5 when targeting the rs7984A or rs7984G allele in HEK293-RHO-P23H minigene expressing cells (Example 3). Indels are measured after transient plasmid transfection both on the target allele (either at the endogenous RHO locus or at the integrated minigene, depending on the version of the guide used) and the counter-allele (either at the integrated minigene or at the endogenous RHO locus, respectively).

FIG. 12 shows the on-target editing levels obtained after transient transfection of HEK293T cells with different versions of gRNA5, having both different spacer length (21 nt vs 23 nt) and also exploiting two different scaffolds (trimV1 vs trimV2) (Example 3).

Data in FIGS. 9-12 is presented as mean±SEM for n≥2 independent runs, except for FIG. 10 where single data points are shown for the 20 and 23-nucleotide spacers.

FIG. 13 shows on-target editing obtained with ENQP Type II Cas and guide RNAs targeting RHO intron 1 after transient plasmid transfection of HEK293T cells (Example 3). Data presented as mean±SEM for n≥2 independent runs.

FIG. 14 shows deletion formation using combinations of the sgRNAs targeting the RHO rs7984 SNP and RHO intron 1 after transient plasmid transfection of HEK293-RHO-P23H cells (Example 3).

FIGS. 15A-15D show the editing activity of ENQP Type II Cas in combination with panels of sgRNAs targeting TRAC (FIG. 15A), B2M (FIG. 15B), PD-1 (FIG. 15C) and LAG3 (FIG. 15D) loci after transient plasmid transfection in HEK293T cells. Data are presented as mean±SEM for n≥2 independent runs, except for FIG. 15C, where single data points are shown.

### 6. DETAILED DESCRIPTION

In one aspect, the disclosure provides ENQP Type II Cas proteins. Type II Cas proteins of the disclosure can be in the form of fusion proteins. Unless required otherwise by context, disclosures relating to Type II Cas proteins encompass Type II Cas proteins which are not fusion proteins and Type II Cas proteins which are in the form of fusion proteins (e.g., Type II Cas protein comprising one or more nuclear localization signals and/or one or more tags).

In some embodiments, a Type II Cas protein of the disclosure comprises an amino acid sequence having at least 50% (e.g., at least 55%, at least 60%, at least 65%, at least 70%, at least 75%, at least 85%, at least 90%, at least 95%, or more) sequence identity to a RuvC-I domain, RuvC-II domain, RuvC-III domain, BH domain, REC domain, HNH domain, WED domain, or PID domain of wild-type ENQP Type II Cas protein. In some embodiments, a Type II Cas protein of the disclosure is a chimeric Type II Cas protein, for example, comprising one or more domains from an ENQP Type II Cas protein and one or more domains from a different Type II Cas protein such as SpCas9.

Exemplary features of Type II Cas proteins of the disclosure are described in Section 6.2.

In further aspects, the disclosure provides guide (gRNA) molecules, for example single guide RNAs (sgRNAs), and combinations of guide RNA molecules, for example combinations of two or more sgRNAs. Combinations of gRNAs can include, for example, a gRNA targeting the RHO rs7984 SNP and a second gRNA targeting RHO intron 1. Combinations of gRNAs targeting the RHO rs7984 SNP and RHO intron 1 can be used to selectively edit RHO alleles having pathogenic mutations. This dual targeting approach is further described Section 6.8 and Example 3. Exemplary features of the gRNAs and combinations of gRNAs of the disclosure are further described in Section 6.3.

In further aspects, the disclosure provides systems comprising a Type II Cas protein of the disclosure and one or more gRNAs, e.g., sgRNAs. Exemplary features of systems are described in Section 6.4.

In further aspects, the disclosure provides nucleic acids and pluralities of nucleic acids encoding a Type II Cas protein of the disclosure and, optionally, a guide RNA, for example a sgRNA, and provides nucleic acids encoding a gRNA, for example a sgRNA, of the disclosure and, optionally, a Type II Cas protein. Exemplary features of nucleic and pluralities of nucleic acids of the disclosure are described in Section 6.5.

In further aspects, the disclosure provides particles comprising the Type II Cas proteins, gRNAs, nucleic acids, and systems of the disclosure. Exemplary features of particles of the disclosure are described in Section 6.6.

In another aspect, the disclosure provides cells and populations of cells containing or contacted with a Type II Cas protein, gRNA, nucleic acid, plurality of nucleic acids, system, or particle of the disclosure. Exemplary features of such cells and cell populations are described in Section 6.6.

In another aspect, the disclosure provides pharmaceutical compositions comprising a Type II Cas protein, gRNA, nucleic acid, plurality of nucleic acids, system, particle, cell, or population of cells together with one or more excipients. Exemplary features of pharmaceutical compositions are described in Section 6.7.

In another aspect, the disclosure provides methods of altering cells (e.g., editing the genome of a cell) using the Type II Cas proteins, gRNAs, nucleic acids, systems, particles, and pharmaceutical compositions of the disclosure. Features of exemplary methods of altering cells are described in Section 6.8.

Those skilled in the relevant art will recognize and appreciate that many changes can be made to the various embodiments described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### 6.1. Definitions

Unless defined otherwise, all technical and scientific terms used herein have the same meaning as commonly understood to one of ordinary skill in the art to which this invention belongs. The following definitions are provided for the full understanding of terms used in this specification.

As used in the specification and claim, the singular form “a,” “an,” and “the” include plural references unless the context clearly dictates otherwise. For example, the term “an agent” includes a plurality of agents, including mixtures thereof.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

A Type II Cas protein refers to a wild-type or engineered Type II Cas protein. Engineered Type II Cas proteins can also be referred to as Type II Cas variants. For the avoidance of doubt, any disclosure pertaining to a “Type II Cas” or “Type II Cas protein” pertains to wild-type Type II Cas proteins and Type II Cas variants, unless the context dictates otherwise. A Type II Cas protein can have nuclease activity or be catalytically inactive (e.g., as in a dCas).

As used herein, the percentage identity between two nucleotide sequences or between two amino acid sequences is calculated by multiplying the number of matches between a pair of aligned sequences by 100, and dividing by the length of the aligned region. Identity scoring only counts perfect matches and does not consider the degree of similarity of amino acids to one another, nor does it consider substitutions or deletions as matches. Alignment for purposes of determining percent sequence identity can be achieved in various ways that are within the skill in the art, for instance, by manual alignment or using publicly available computer software such as BLAST, BLAST-2, ALIGN, ALIGN-2 or Megalign (DNASTAR) software. Those skilled in the art can determine appropriate parameters for achieving maximum alignment.

Guide RNA molecule (gRNA) refers to an RNA capable of forming a complex with a Type II Cas protein and which can direct the Type II Cas protein to a target DNA. gRNAs typically comprise a spacer of 15 to 30 nucleotides in length. gRNAs of the disclosure are in some embodiments single guide RNAs (sgRNAs), which typically comprise a spacer at the 5′ end of the molecule and a 3′ sgRNA scaffold. Various non-limiting examples of 3′ sgRNA scaffolds are described in Section 6.3.

An sgRNA can in some embodiments comprise no uracil base at the 3′ end of the sgRNA sequence. Alternatively, a sgRNA can comprise one or more uracil bases at the 3′ end of the sgRNA sequence. For example, a sgRNA can comprise 1 uracil (U) at the 3′ end of the sgRNA sequence, 2 uracil (UU) at the 3′ end of the sgRNA sequence, 3 uracil (UUU) at the 3′ end of the sgRNA sequence, 4 uracil (UUUU) at the 3′ end of the sgRNA sequence, 5 uracil (UUUUU) at the 3′ end of the sgRNA sequence, 6 uracil (UUUUUU) at the 3′ end of the sgRNA sequence, 7 uracil (UUUUUUU) at the 3′ end of the sgRNA sequence, or 8 uracil (UUUUUUUU) at the 3′ end of the sgRNA sequence. Different length stretches of uracil can be appended at the 3′ end of a sgRNA as terminators. Thus, for example, the 3′ sgRNA scaffolds set forth in Section 6.3 can be modified by adding or removing one or more uracils at the end of the sequence.

Peptide, protein, and polypeptide are used interchangeably to refer to a natural or synthetic molecule comprising two or more amino acids linked by the carboxyl group of one amino acid to the alpha amino group of another. The amino acids may be natural or synthetic, and can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance molecules required for function. Examples of molecules which may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is expressly no requirement that a polypeptide must contain an intended function; a polypeptide can be functional, non-functional, function for unexpected/unintended purposes, or have unknown function. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Polynucleotide and oligonucleotide are used interchangeably and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, primers and gRNAs. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C); guanine (G); thymine (T); and uracil (U) for thymine (T) when the polynucleotide is RNA. Thus, the term “nucleotide sequence” is the alphabetical representation of a polynucleotide molecule. The letters used in polynucleotide sequences described herein correspond to IUPAC notation. For example, the letter “N” in a nucleotide sequence represents a nucleotide which can be A, T, C, or G in a DNA sequence, or A, U, C, or G in a RNA sequence; the letter “R” in a nucleotide sequence represents a nucleotide which can be A or G; and the letter “V” in a nucleotide sequence represents a nucleotide which can be “A, C, or G.

Protospacer adjacent motif (PAM) refers to a DNA sequence downstream (e.g., immediately downstream) of a target sequence on the non-target strand recognized by a Type II Cas protein. A PAM sequence is located 3′ of the target sequence on the non-target strand.

Spacer refers to a region of a gRNA molecule which is partially or fully complementary to a target sequence found in the + or − strand of genomic DNA. When complexed with a Type II Cas protein, the gRNA directs the Type II Cas to the target sequence in the genomic DNA. A spacer of a Type II Cas gRNA is typically 15 to 30 nucleotides in length (e.g., 20-25 nucleotides). The nucleotide sequence of a spacer can be, but is not necessarily, fully complementary to the target sequence. For example, a spacer can contain one or more mismatches with a target sequence, e.g., the spacer can comprise one, two, or three mismatches with the target sequence.

### 6.2. Type II Cas Proteins

### 6.2.1. ENQP Type II Cas Proteins

In one aspect, the disclosure provides ENQP Type II Cas proteins. The ENQP Type II Cas proteins typically comprise an amino acid sequence that is at least 50%, at least 55%, at least 60%, at least 65%, at least 70%, at least 75%, at least 85%, at least 90%, or at least 95% identical to SEQ ID NO: 1. In some embodiments, the ENQP Type II Cas proteins comprise an amino acid sequence that is at least 95%, at least 96%, at least 97%, at least 98%, or at least 99% identical to SEQ ID NO:1. In some embodiments, a ENQP Type II Cas protein comprises an amino acid sequence that is identical to SEQ ID NO: 1.

Exemplary ENQP Type II Cas protein sequences and nucleotide sequences encoding exemplary ENQP Type II Cas proteins are set forth in Table 1.

TABLE 1
ENQP Type II Cas Sequences

| | | SEQ ID |
|---|---|---|
| Name | Sequence | NO |
| ENQP Type II | QPESVRLFGKLGVRRYLVLGLDPGVASCGFSLIDLHNHEILEMGSRLFD | 1 |
| Cas coding | SPVVPKTQQSKAAVRRGFRSTRRNLDRTQNRLKHCLRLLKEQGLVPAGA | |
| sequence | TPEFFHTAKGDQPPIRLRVAGLDRLLTDREWAIVLYSLCKRRGYIPHGE | |
| (aa) (without | GGEGDVDSASEDGKVLKAISQNKKLLAESGARTVGEWLATQPRSRNRSG | |
| N-terminal | EYDKCVTHAQLTDEVRALFSAQRSFGSTSATEDLEDAYLEVFDWEKPRD | |
| methionine) | AFDRRSYELVGNCVYFPGEKRAARCTLTSELVSAYGALGNVTIQFPDGT | |
| | TRPLTAKERDTFIDTLFSPSALKGNKECKVKFSDIRRRLDLDARTSFKG | |
| | ISSDDEKNREVYRPQGWRTLRKTLGSEGTALLTRFLADRDLADATMEAV | |
| | AYSSSLPVLSSRLEELELTDDEVALVERLPYSSRALNGYGSRSKKALDL | |
| | LLGAFEEPEVLTLTDAETATGLDELRKHGSAGIERSTKLMPYAAWIALT | |
| | GRTNNNPVVLRAMAQMRKVVNAVCGEWGVPNEIHVELARELALPKKAKD | |
| | SIARANRQNEKDNERIRKQISELTGRDHDRIKNSLVAKWRLWEEQGNCD | |
| | IYTGERIDALRLVNDDTYTQVDHILPFSRTGDNSRHNKVLVLAHSNQLK | |
| | RERSPFEWMTSEEAGAPDWNSFVASVQENRRISPRKRGFLLERDLMSKE | |
| | GDFQSRNLTDTAYMAREVCAYLSDCLAFPNDGRKVHVVPTKGTASAWLR | |
| | RSWGLNFGMAGEKDRSDDRHHATDACVIAACSRSLVIQTAKLSEHHYTL | |
| | NEQGRDEALLGSMPWPSFADDVRQMREKVIPTRFVPRVGSGELFEQTIY | |
| | SYVGTDDKGKDLLSAKGGKPKPAGNAVVSDDEKSAIKVGGMICLRLWHD | |
| | VDARKGRGQWYADPIYYADLPALRNGTYVPRIAQAHKGRKTWKPIPERV | |
| | LSGKPICLYLGDAVRVDHEIGRFSGFDIDAANWSLKSIYSGEKICFPSI | |
| | GKIDNITFPIKITEDILGRCWGNL | |
| ENQP Type II | MQPESVRLFGKLGVRRYLVLGLDPGVASCGFSLIDLHNHEILEMGSRLF | 2 |
| Cas coding | DSPVVPKTQQSKAAVRRGFRSTRRNLDRTQNRLKHCLRLLKEQGLVPAG | |
| sequence | ATPEFFHTAKGDQPPIRLRVAGLDRLLTDREWAIVLYSLCKRRGYIPHG | |
| (aa) | EGGEGDVDSASEDGKVLKAISQNKKLLAESGARTVGEWLATQPRSRNRS | |
| | GEYDKCVTHAQLTDEVRALFSAQRSFGSTSATEDLEDAYLEVFDWEKPR | |
| | DAFDRRSYELVGNCVYFPGEKRAARCTLTSELVSAYGALGNVTIQFPDG | |
| | TTRPLTAKERDTFIDTLFSPSALKGNKECKVKFSDIRRRLDLDARTSFK | |
| | GISSDDEKNREVYRPQGWRTLRKTLGSEGTALLTRFLADRDLADATMEA | |
| | VAYSSSLPVLSSRLEELELTDDEVALVERLPYSSRALNGYGSRSKKALD | |
| | LLLGAFEEPEVLTLTDAETATGLDELRKHGSAGIERSTKLMPYAAWIAL | |
| | TGRTNNNPVVLRAMAQMRKVVNAVCGEWGVPNEIHVELARELALPKKAK | |
| | DSIARANRQNEKDNERIRKQISELTGRDHDRIKNSLVAKWRLWEEQGNC | |
| | DIYTGERIDALRLVNDDTYTQVDHILPFSRTGDNSRHNKVLVLAHSNQL | |
| | KRERSPFEWMTSEEAGAPDWNSFVASVQENRRISPRKRGFLLERDLMSK | |
| | EGDFQSRNLTDTAYMAREVCAYLSDCLAFPNDGRKVHVVPTKGTASAWL | |
| | RRSWGLNFGMAGEKDRSDDRHHATDACVIAACSRSLVIQTAKLSEHHYT | |
| | LNEQGRDEALLGSMPWPSFADDVRQMREKVIPTRFVPRVGSGELFEQTI | |
| | YSYVGTDDKGKDLLSAKGGKPKPAGNAVVSDDEKSAIKVGGMICLRLWH | |
| | DVDARKGRGQWYADPIYYADLPALRNGTYVPRIAQAHKGRKTWKPIPER | |
| | VLSGKPICLYLGDAVRVDHEIGRFSGFDIDAANWSLKSIYSGEKICFPS | |
| | IGKIDNITFPIKITEDILGRCWGNL | |
| ENQP Type II | MGKPIPNPLLGLDSTKRTADGSEFESPKKKRKVQPESVRLFGKLGVRRY | 3 |
| Cas | LVLGLDPGVASCGFSLIDLHNHEILEMGSRLFDSPVVPKTQQSKAAVRR | |
| mammalian | GFRSTRRNLDRTQNRLKHCLRLLKEQGLVPAGATPEFFHTAKGDQPPIR | |
| expression | LRVAGLDRLLTDREWAIVLYSLCKRRGYIPHGEGGEGDVDSASEDGKVL | |
| construct | KAISQNKKLLAESGARTVGEWLATQPRSRNRSGEYDKCVTHAQLTDEVR | |
| (includes N- | ALFSAQRSFGSTSATEDLEDAYLEVFDWEKPRDAFDRRSYELVGNCVYF | |
| terminal SV5 | PGEKRAARCTLTSELVSAYGALGNVTIQFPDGTTRPLTAKERDTFIDTL | |
| tag and NLS | FSPSALKGNKECKVKFSDIRRRLDLDARTSFKGISSDDEKNREVYRPQG | |
| and C- | WRTLRKTLGSEGTALLTRFLADRDLADATMEAVAYSSSLPVLSSRLEEL | |
| terminal NLS) | ELTDDEVALVERLPYSSRALNGYGSRSKKALDLLLGAFEEPEVLTLTDA | |
| (aa) | ETATGLDELRKHGSAGIERSTKLMPYAAWIALTGRTNNNPVVLRAMAQM | |
| | RKVVNAVCGEWGVPNEIHVELARELALPKKAKDSIARANRQNEKDNERI | |
| | RKQISELTGRDHDRIKNSLVAKWRLWEEQGNCDIYTGERIDALRLVNDD | |
| | TYTQVDHILPFSRTGDNSRHNKVLVLAHSNQLKRERSPFEWMTSEEAGA | |
| | PDWNSFVASVQENRRISPRKRGFLLERDLMSKEGDFQSRNLTDTAYMAR | |
| | EVCAYLSDCLAFPNDGRKVHVVPTKGTASAWLRRSWGLNFGMAGEKDRS | |
| | DDRHHATDACVIAACSRSLVIQTAKLSEHHYTLNEQGRDEALLGSMPWP | |
| | SFADDVRQMREKVIPTRFVPRVGSGELFEQTIYSYVGTDDKGKDLLSAK | |
| | GGKPKPAGNAVVSDDEKSAIKVGGMICLRLWHDVDARKGRGQWYADPIY | |
| | YADLPALRNGTYVPRIAQAHKGRKTWKPIPERVLSGKPICLYLGDAVRV | |
| | DHEIGRFSGFDIDAANWSLKSIYSGEKICFPSIGKIDNITFPIKITEDI | |
| | LGRCWGNLKRTADGSEFESPKKKRKV | |
| ENQP Type II | ATGCAGCCTGAGTCCGTTCGCCTCTTTGGAAAGCTTGGGGTTCGCCGCT | 4 |
| Cas coding | ACCTCGTCCTTGGACTCGATCCCGGGGTCGCCAGCTGCGGCTTTTCTCT | |
| sequence (nt) | CATCGACCTCCACAACCACGAGATTCTCGAGATGGGGTCCCGTCTCTTC | |
| (not codon | GACTCGCCCGTCGTCCCCAAGACGCAGCAGAGCAAGGCGGCGGTGCGAC | |
| optimized) | GGGGCTTCCGCTCAACCCGTCGCAACCTCGACAGGACGCAGAATCGTCT | |
| | GAAGCACTGCCTTCGCCTCCTCAAGGAGCAGGGCCTCGTCCCCGCCGGC | |
| | GCCACCCCCGAGTTCTTCCACACCGCAAAAGGCGACCAACCGCCCATCA | |
| | GGCTGAGGGTCGCCGGACTCGACCGGCTTCTCACCGACCGCGAGTGGGC | |
| | GATCGTCCTGTACTCGCTCTGCAAGCGCCGCGGCTACATACCCCACGGT | |
| | GAGGGGGGGGAGGGTGACGTCGACAGCGCAAGCGAGGACGGAAAGGTCC | |
| | TCAAGGCCATAAGCCAGAACAAGAAGCTTCTCGCGGAGAGCGGTGCCAG | |
| | AACCGTCGGTGAGTGGCTTGCTACGCAGCCGCGCAGCAGGAACCGCTCC | |
| | GGGGAGTACGACAAGTGCGTCACCCACGCACAGCTCACCGATGAGGTGC | |
| | GCGCGCTGTTCAGCGCGCAGAGGTCCTTCGGAAGCACGTCTGCCACGGA | |
| | GGATCTCGAGGACGCCTATCTTGAGGTCTTCGACTGGGAGAAGCCCCGC | |
| | GACGCGTTCGACAGGCGAAGCTACGAGCTCGTCGGCAACTGCGTGTACT | |
| | TCCCCGGCGAGAAGCGCGCGGCGCGCTGCACGCTCACGAGCGAGCTCGT | |
| | CTCCGCATACGGCGCCCTCGGCAACGTCACAATTCAGTTCCCCGACGGC | |
| | ACGACGCGCCCGCTCACCGCAAAGGAGCGCGACACCTTCATCGACACGC | |
| | TCTTCTCCCCCTCCGCGCTCAAGGGCAACAAGGAGTGCAAGGTCAAGTT | |
| | CTCCGACATCAGAAGGCGCCTCGACCTCGACGCCCGGACCTCGTTCAAG | |
| | GGCATCTCGTCTGATGACGAGAAGAACCGCGAGGTATACCGGCCGCAAG | |
| | GCTGGAGAACGCTGCGAAAGACGCTCGGCTCCGAGGGGACGGCCCTGCT | |
| | CACGAGGTTTCTCGCCGACCGCGACCTCGCGGACGCGACGATGGAGGCC | |
| | GTCGCGTACTCCTCGTCCCTCCCCGTCTTGAGCTCGCGTCTCGAGGAGC | |
| | TTGAGCTCACCGATGACGAGGTCGCCCTCGTCGAGCGCCTCCCCTACTC | |
| | GAGCCGTGCCCTCAACGGATACGGGTCCCGCTCCAAGAAGGCGCTTGAC | |
| | CTGCTGCTGGGCGCCTTCGAGGAACCCGAGGTACTCACTCTCACGGACG | |
| | CCGAGACGGCCACTGGCCTTGATGAGCTTCGCAAGCACGGCTCAGCCGG | |
| | CATCGAGCGGTCGACAAAGCTGATGCCGTACGCCGCATGGATCGCCCTC | |
| | ACCGGCCGCACCAACAACAACCCCGTCGTCCTCAGGGCGATGGCTCAGA | |
| | TGCGCAAGGTCGTCAACGCCGTCTGTGGTGAATGGGGCGTCCCCAACGA | |
| | GATTCACGTCGAGCTCGCCCGCGAGCTCGCGCTACCCAAGAAGGCCAAG | |
| | GACTCCATCGCCCGTGCCAACCGTCAGAACGAGAAGGATAACGAGCGCA | |
| | TCAGGAAGCAGATCTCCGAACTCACGGGGCGAGACCACGACCGGATCAA | |
| | GAACTCACTTGTGGCAAAGTGGCGCCTCTGGGAGGAGCAGGGGAACTGC | |
| | GACATCTACACGGGCGAGAGAATCGACGCCCTGCGGCTCGTCAATGACG | |
| | ACACGTACACCCAGGTCGACCATATCCTCCCCTTCTCTCGCACGGGAGA | |
| | CAACTCGCGCCACAACAAGGTGCTCGTCCTTGCCCACAGCAACCAGCTC | |
| | AAGCGCGAGCGGTCTCCCTTCGAGTGGATGACCTCCGAGGAGGCGGGCG | |
| | CGCCCGACTGGAACTCCTTTGTCGCATCCGTCCAAGAGAACCGCAGGAT | |
| | CAGCCCGCGGAAGCGTGGCTTCCTCCTCGAGCGCGACCTCATGTCTAAG | |
| | GAGGGCGACTTCCAAAGCAGGAACCTGACGGACACGGCCTACATGGCCC | |
| | GCGAGGTCTGCGCCTATCTCTCGGACTGCCTCGCCTTCCCGAACGACGG | |
| | CAGGAAGGTTCACGTCGTGCCGACCAAGGGCACCGCCTCGGCGTGGCTC | |
| | AGAAGGTCCTGGGGGCTCAACTTCGGCATGGCGGGGGAGAAGGACCGCT | |
| | CGGACGACCGTCACCACGCAACGGATGCGTGCGTGATTGCCGCGTGCTC | |
| | TCGCTCGCTCGTCATTCAGACGGCAAAGCTCTCCGAGCACCACTACACG | |
| | CTCAACGAGCAGGGTCGCGACGAGGCGCTTCTTGGGTCTATGCCCTGGC | |
| | CATCCTTTGCCGATGACGTCCGACAGATGCGCGAGAAGGTCATCCCGAC | |
| | CAGGTTTGTCCCCCGCGTCGGGTCGGGCGAGCTCTTTGAGCAGACGATC | |
| | TACTCCTATGTCGGAACCGACGACAAGGGCAAGGACCTTCTCTCGGCAA | |
| | AGGGCGGCAAGCCGAAGCCCGCTGGCAACGCAGTTGTCTCCGATGACGA | |
| | GAAGTCCGCCATCAAGGTCGGAGGAATGATCTGCCTGCGCCTCTGGCAC | |
| | GATGTCGATGCCAGAAAGGGGCGTGGCCAGTGGTACGCCGACCCGATCT | |
| | ACTATGCCGACCTACCGGCTCTGCGAAACGGGACGTATGTGCCGCGGAT | |
| | TGCCCAAGCGCATAAGGGGAGAAAGACTTGGAAGCCCATTCCTGAGCGG | |
| | GTCCTTTCCGGCAAACCGATTTGTCTCTATCTCGGAGATGCGGTGCGGG | |
| | TTGACCATGAGATCGGCAGATTTTCTGGCTTTGACATCGATGCAGCCAA | |
| | TTGGTCACTAAAGAGTATTTACTCTGGGGAGAAGATTTGTTTTCCTTCT | |
| | ATTGGAAAGATTGATAATATAACTTTTCCAATTAAAATCACAGAGGATA | |
| | TCCTTGGTAGATGTTGGGGAAATTTGTAG | |
| ENQP Type II | ATGCAGCCTGAGAGCGTGCGACTGTTCGGAAAGCTGGGAGTGAGAAGAT | 5 |
| Cas coding | ACCTGGTGCTGGGTCTGGACCCTGGCGTGGCCAGCTGCGGCTTTAGCCT | |
| sequence (nt) | TATCGACCTGCACAATCACGAGATCCTGGAAATGGGCTCTCGGCTGTTC | |
| (human | GACAGCCCTGTGGTGCCTAAGACCCAGCAGTCTAAGGCCGCTGTGCGGC | |
| codon- | GGGGCTTCAGATCTACCAGACGGAATCTGGACAGAACACAAAACAGACT | |
| optimized) | GAAGCACTGCCTGAGACTGCTGAAGGAACAGGGCCTGGTCCCTGCCGGG | |
| | GCAACCCCTGAGTTCTTCCACACCGCCAAAGGCGATCAGCCCCCCATCA | |
| | GACTGCGGGTGGCCGGCCTGGACCGTCTGCTGACCGACAGAGAGTGGGC | |
| | CATCGTGCTTTATAGCTTGTGCAAGCGGAGAGGCTACATCCCGCACGGC | |
| | GAGGGCGGCGAAGGCGATGTGGATTCTGCCAGCGAGGATGGCAAGGTGC | |
| | TGAAAGCCATCAGCCAGAACAAAAAACTGCTGGCCGAGAGCGGCGCTAG | |
| | AACCGTGGGCGAGTGGCTGGCTACCCAGCCTAGATCACGGAACCGGAGC | |
| | GGCGAGTACGACAAGTGCGTGACACACGCCCAACTGACCGACGAGGTGA | |
| | GAGCCCTGTTTTCTGCCCAGAGATCCTTTGGTAGCACATCCGCCACCGA | |
| | GGACCTGGAAGATGCCTACCTGGAAGTGTTCGATTGGGAGAAGCCACGC | |
| | GACGCCTTCGACAGACGGTCCTACGAGCTGGTCGGCAACTGCGTGTACT | |
| | TCCCAGGCGAAAAAAGAGCCGCCCGGTGCACATTAACAAGCGAGCTGGT | |
| | GTCCGCTTACGGTGCCCTGGGCAACGTGACCATCCAATTTCCAGACGGC | |
| | ACCACCCGCCCACTGACGGCCAAAGAGAGAGATACCTTCATCGACACCC | |
| | TCTTCAGCCCATCCGCCCTGAAGGGAAACAAAGAATGCAAAGTGAAGTT | |
| | CTCTGACATCAGAAGGAGACTGGACCTCGACGCCAGGACCAGCTTCAAG | |
| | GGAATCTCCAGCGACGACGAGAAGAACCGGGAGGTGTACCGGCCTCAGG | |
| | GATGGCGGACCCTGAGAAAGACACTGGGCAGCGAAGGCACCGCGCTGCT | |
| | GACACGGTTCCTGGCTGATCGAGATCTGGCCGATGCTACAATGGAGGCC | |
| | GTGGCTTACAGCTCCAGCCTTCCTGTGCTGTCTAGCAGACTGGAAGAGC | |
| | TGGAACTAACCGATGATGAAGTGGCCCTCGTTGAGCGCCTGCCTTACAG | |
| | CAGCAGAGCCCTGAACGGCTATGGAAGCCGGAGCAAGAAGGCCCTGGAT | |
| | CTGCTGCTGGGCGCCTTCGAGGAACCTGAGGTGCTGACACTGACCGACG | |
| | CCGAAACCGCTACCGGCCTGGACGAGCTGAGAAAGCACGGCAGCGCCGG | |
| | CATCGAGAGAAGCACCAAGCTGATGCCCTACGCCGCCTGGATCGCCCTG | |
| | ACAGGAAGAACAAACAACAACCCCGTTGTGCTGAGAGCTATGGCCCAGA | |
| | TGCGGAAAGTGGTGAACGCCGTTTGTGGCGAGTGGGGCGTGCCCAACGA | |
| | GATCCACGTGGAACTTGCCCGGGAGCTGGCCCTGCCTAAGAAGGCTAAG | |
| | GACTCTATCGCCAGGGCCAATAGACAGAACGAGAAGGATAACGAGAGAA | |
| | TCCGGAAGCAGATCTCGGAGCTGACAGGACGGGACCACGATCGGATTAA | |
| | GAACAGCCTGGTGGCTAAGTGGAGACTGTGGGAAGAACAGGGAAATTGC | |
| | GACATCTACACCGGCGAGCGGATCGATGCTTTGCGCCTGGTCAACGATG | |
| | ACACGTACACTCAGGTGGACCACATCCTGCCTTTCAGCAGAACCGGAGA | |
| | TAATAGCAGACACAACAAGGTGCTGGTGCTGGCTCACAGCAACCAGCTG | |
| | AAGAGAGAAAGATCTCCTTTTGAGTGGATGACCTCCGAGGAAGCCGGAG | |
| | CCCCTGACTGGAACTCGTTTGTGGCCAGCGTGCAGGAGAACCGGAGAAT | |
| | CTCTCCCAGAAAGCGCGGCTTCCTGCTGGAAAGAGATCTGATGAGCAAG | |
| | GAAGGCGATTTCCAGAGCAGAAATCTGACAGACACCGCCTACATGGCCA | |
| | GAGAGGTCTGCGCCTACCTGTCTGATTGCCTGGCTTTCCCTAACGACGG | |
| | CAGAAAGGTGCACGTGGTGCCTACCAAGGGCACCGCCTCTGCATGGCTG | |
| | CGGAGAAGCTGGGGACTGAACTTCGGCATGGCCGGCGAGAAGGACCGGA | |
| | GCGACGATCGGCACCATGCCACCGACGCTTGTGTGATCGCCGCCTGTAG | |
| | CAGAAGCCTGGTCATCCAGACCGCCAAGCTGTCCGAGCACCACTACACA | |
| | CTGAACGAGCAGGGCCGGGACGAGGCCCTACTGGGCTCTATGCCTTGGC | |
| | CTTCTTTCGCCGATGACGTGAGACAGATGAGAGAAAAAGTGATCCCAAC | |
| | AAGATTCGTGCCTCGGGTAGGCAGTGGCGAGCTGTTCGAGCAAACAATC | |
| | TACAGCTACGTGGGCACTGACGATAAGGGCAAGGACCTCCTGAGCGCCA | |
| | AAGGAGGCAAACCCAAGCCCGCCGGCAATGCCGTGGTCAGCGACGACGA | |
| | GAAATCAGCAATCAAGGTGGGCGGCATGATCTGTCTGAGACTGTGGCAC | |
| | GATGTGGACGCTCGGAAGGGCAGAGGCCAGTGGTATGCCGACCCCATCT | |
| | ACTACGCCGACCTGCCAGCCCTGAGAAACGGCACCTACGTGCCTAGAAT | |
| | CGCCCAGGCCCACAAGGGCAGAAAAACCTGGAAGCCCATCCCTGAAAGA | |
| | GTGCTGAGCGGCAAGCCCATCTGTCTGTACCTCGGCGACGCCGTGCGCG | |
| | TGGACCATGAAATCGGCAGATTCAGCGGCTTTGACATAGATGCCGCTAA | |
| | CTGGAGCCTGAAATCCATTTACAGCGGAGAGAAGATCTGCTTCCCTAGC | |
| | ATTGGCAAGATCGACAACATCACCTTCCCTATCAAGATCACAGAGGACA | |
| | TCCTGGGAAGATGCTGGGGCAACCTGAAGCGGACAGCAGACGGCTCCGA | |
| | ATTTGAAAGCCCTAAGAAAAAGAGAAAGGTGTGA | |
| ENQP Type II | ATGGGCAAACCTATCCCCAATCCCCTGCTGGGCCTGGACAGCACCAAGC | 6 |
| Cas | GGACTGCCGACGGCAGTGAGTTCGAGTCTCCCAAGAAAAAGAGGAAAGT | |
| mammalian | GCAGCCTGAGAGCGTGCGACTGTTCGGAAAGCTGGGAGTGAGAAGATAC | |
| expression | CTGGTGCTGGGTCTGGACCCTGGCGTGGCCAGCTGCGGCTTTAGCCTTA | |
| construct | TCGACCTGCACAATCACGAGATCCTGGAAATGGGCTCTCGGCTGTTCGA | |
| (includes N- | CAGCCCTGTGGTGCCTAAGACCCAGCAGTCTAAGGCCGCTGTGCGGCGG | |
| terminal SV5 | GGCTTCAGATCTACCAGACGGAATCTGGACAGAACACAAAACAGACTGA | |
| tag and NLS | AGCACTGCCTGAGACTGCTGAAGGAACAGGGCCTGGTCCCTGCCGGGGC | |
| and C- | AACCCCTGAGTTCTTCCACACCGCCAAAGGCGATCAGCCCCCCATCAGA | |
| terminal NLS) | CTGCGGGTGGCCGGCCTGGACCGTCTGCTGACCGACAGAGAGTGGGCCA | |
| (nt) | TCGTGCTTTATAGCTTGTGCAAGCGGAGAGGCTACATCCCGCACGGCGA | |
| | GGGCGGCGAAGGCGATGTGGATTCTGCCAGCGAGGATGGCAAGGTGCTG | |
| | AAAGCCATCAGCCAGAACAAAAAACTGCTGGCCGAGAGCGGCGCTAGAA | |
| | CCGTGGGCGAGTGGCTGGCTACCCAGCCTAGATCACGGAACCGGAGCGG | |
| | CGAGTACGACAAGTGCGTGACACACGCCCAACTGACCGACGAGGTGAGA | |
| | GCCCTGTTTTCTGCCCAGAGATCCTTTGGTAGCACATCCGCCACCGAGG | |
| | ACCTGGAAGATGCCTACCTGGAAGTGTTCGATTGGGAGAAGCCACGCGA | |
| | CGCCTTCGACAGACGGTCCTACGAGCTGGTCGGCAACTGCGTGTACTTC | |
| | CCAGGCGAAAAAAGAGCCGCCCGGTGCACATTAACAAGCGAGCTGGTGT | |
| | CCGCTTACGGTGCCCTGGGCAACGTGACCATCCAATTTCCAGACGGCAC | |
| | CACCCGCCCACTGACGGCCAAAGAGAGAGATACCTTCATCGACACCCTC | |
| | TTCAGCCCATCCGCCCTGAAGGGAAACAAAGAATGCAAAGTGAAGTTCT | |
| | CTGACATCAGAAGGAGACTGGACCTCGACGCCAGGACCAGCTTCAAGGG | |
| | AATCTCCAGCGACGACGAGAAGAACCGGGAGGTGTACCGGCCTCAGGGA | |
| | TGGCGGACCCTGAGAAAGACACTGGGCAGCGAAGGCACCGCGCTGCTGA | |
| | CACGGTTCCTGGCTGATCGAGATCTGGCCGATGCTACAATGGAGGCCGT | |
| | GGCTTACAGCTCCAGCCTTCCTGTGCTGTCTAGCAGACTGGAAGAGCTG | |
| | GAACTAACCGATGATGAAGTGGCCCTCGTTGAGCGCCTGCCTTACAGCA | |
| | GCAGAGCCCTGAACGGCTATGGAAGCCGGAGCAAGAAGGCCCTGGATCT | |
| | GCTGCTGGGCGCCTTCGAGGAACCTGAGGTGCTGACACTGACCGACGCC | |
| | GAAACCGCTACCGGCCTGGACGAGCTGAGAAAGCACGGCAGCGCCGGCA | |
| | TCGAGAGAAGCACCAAGCTGATGCCCTACGCCGCCTGGATCGCCCTGAC | |
| | AGGAAGAACAAACAACAACCCCGTTGTGCTGAGAGCTATGGCCCAGATG | |
| | CGGAAAGTGGTGAACGCCGTTTGTGGCGAGTGGGGCGTGCCCAACGAGA | |
| | TCCACGTGGAACTTGCCCGGGAGCTGGCCCTGCCTAAGAAGGCTAAGGA | |
| | CTCTATCGCCAGGGCCAATAGACAGAACGAGAAGGATAACGAGAGAATC | |
| | CGGAAGCAGATCTCGGAGCTGACAGGACGGGACCACGATCGGATTAAGA | |
| | ACAGCCTGGTGGCTAAGTGGAGACTGTGGGAAGAACAGGGAAATTGCGA | |
| | CATCTACACCGGCGAGCGGATCGATGCTTTGCGCCTGGTCAACGATGAC | |
| | ACGTACACTCAGGTGGACCACATCCTGCCTTTCAGCAGAACCGGAGATA | |
| | ATAGCAGACACAACAAGGTGCTGGTGCTGGCTCACAGCAACCAGCTGAA | |
| | GAGAGAAAGATCTCCTTTTGAGTGGATGACCTCCGAGGAAGCCGGAGCC | |
| | CCTGACTGGAACTCGTTTGTGGCCAGCGTGCAGGAGAACCGGAGAATCT | |
| | CTCCCAGAAAGCGCGGCTTCCTGCTGGAAAGAGATCTGATGAGCAAGGA | |
| | AGGCGATTTCCAGAGCAGAAATCTGACAGACACCGCCTACATGGCCAGA | |
| | GAGGTCTGCGCCTACCTGTCTGATTGCCTGGCTTTCCCTAACGACGGCA | |
| | GAAAGGTGCACGTGGTGCCTACCAAGGGCACCGCCTCTGCATGGCTGCG | |
| | GAGAAGCTGGGGACTGAACTTCGGCATGGCCGGCGAGAAGGACCGGAGC | |
| | GACGATCGGCACCATGCCACCGACGCTTGTGTGATCGCCGCCTGTAGCA | |
| | GAAGCCTGGTCATCCAGACCGCCAAGCTGTCCGAGCACCACTACACACT | |
| | GAACGAGCAGGGCCGGGACGAGGCCCTACTGGGCTCTATGCCTTGGCCT | |
| | TCTTTCGCCGATGACGTGAGACAGATGAGAGAAAAAGTGATCCCAACAA | |
| | GATTCGTGCCTCGGGTAGGCAGTGGCGAGCTGTTCGAGCAAACAATCTA | |
| | CAGCTACGTGGGCACTGACGATAAGGGCAAGGACCTCCTGAGCGCCAAA | |
| | GGAGGCAAACCCAAGCCCGCCGGCAATGCCGTGGTCAGCGACGACGAGA | |
| | AATCAGCAATCAAGGTGGGCGGCATGATCTGTCTGAGACTGTGGCACGA | |
| | TGTGGACGCTCGGAAGGGCAGAGGCCAGTGGTATGCCGACCCCATCTAC | |
| | TACGCCGACCTGCCAGCCCTGAGAAACGGCACCTACGTGCCTAGAATCG | |
| | CCCAGGCCCACAAGGGCAGAAAAACCTGGAAGCCCATCCCTGAAAGAGT | |
| | GCTGAGCGGCAAGCCCATCTGTCTGTACCTCGGCGACGCCGTGCGCGTG | |
| | GACCATGAAATCGGCAGATTCAGCGGCTTTGACATAGATGCCGCTAACT | |
| | GGAGCCTGAAATCCATTTACAGCGGAGAGAAGATCTGCTTCCCTAGCAT | |
| | TGGCAAGATCGACAACATCACCTTCCCTATCAAGATCACAGAGGACATC | |
| | CTGGGAAGATGCTGGGGCAACCTGAAGCGGACAGCAGACGGCTCCGAAT | |
| | TTGAAAGCCCTAAGAAAAAGAGAAAGGTGTGA | |

In some embodiments a ENQP Type II Cas protein comprises an amino acid sequence of SEQ ID NO: 1, SEQ ID NO:2, or SEQ ID NO:3. In some embodiments, a ENQP Type II Cas protein has nickase activity, for example resulting from one or more amino acid substitutions relative to the sequence of SEQ ID NO:1, SEQ ID NO:2, or SEQ ID NO:3. In some embodiments, the one or more amino acid substitutions providing nickase activity is a D23A substitution, wherein the position of the D23A substitution is defined with respect to the amino acid numbering of SEQ ID NO:2. In some embodiments, the one or more amino acid substitutions providing nickase activity comprise an H612A substitution, wherein the position of the H612A substitution is defined with respect to the amino acid numbering of SEQ ID NO:2. In some embodiments, an ENQP Type II Cas protein is catalytically inactive, for example due to a D23A substitution in combination with a H612A substitution.

### 6.2.2. Fusion and Chimeric Proteins

The disclosure provides ENQP Type II Cas proteins (e.g., a ENQP Type II Cas protein as described in Section 6.2.1) which are in the form of fusion proteins comprising a Type II Cas protein sequence fused with one or more additional amino acid sequences, such as one or more nuclear localization signals and/or one or more non-native tags. Fusion proteins can also comprise an amino acid sequence of, for example, a nucleoside deaminase, a reverse transcriptase, a transcriptional activator (e.g., VP64), a transcriptional repressor (e.g., Krüppel associated box (KRAB)), a histone-modifying protein, an integrase, or a recombinase.

In some embodiments, a fusion protein of the disclosure comprises a means for localizing the Type II Cas protein to the nucleus, for example a nuclear localization signal.

Non-limiting examples of nuclear localization signals include KRTADGSEFESPKKKRKV (SEQ ID NO: 7), PKKKRKV (SEQ ID NO:8), PKKKRRV (SEQ ID NO:9), KRPAATKKAGQAKKKK (SEQ ID NO: 10), YGRKKRRQRRR (SEQ ID NO:11), RKKRRQRRR (SEQ ID NO:12), PAAKRVKLD (SEQ ID NO: 13), RQRRNELKRSP (SEQ ID NO:14), VSRKRPRP (SEQ ID NO:15), PPKKARED (SEQ ID NO:16), PQPKKKPL (SEQ ID NO:17), SALIKKKKKMAP (SEQ ID NO:18), PKQKKRK (SEQ ID NO:19), RKLKKKIKKL (SEQ ID NO:20), REKKKFLKRR (SEQ ID NO:21), KRKGDEVDGVDEVAKKKSKK (SEQ ID NO: 22), RKCLQAGMNLEARKTKK (SEQ ID NO:23), NQSSNFGPMKGGNFGGRSSGPYGGGGQYFAKPRNQGGY (SEQ ID NO:24), and RMRIZFKNKGKDTAELRRRRVEVSVELRKAKKDEQILKRRNV (SEQ ID NO:25).

Exemplary fusion partners include protein tags (e.g., V5-tag (e.g., having the sequence GKPIPNPLLGLDST (SEQ ID NO:26), FLAG-tag, myc-tag, HA-tag, GST-tag, polyHis-tag, MBP-tag), protein domains, transcription modulators, enzymes acting on small molecule substrates, DNA, RNA and protein modification enzymes (e.g., adenosine deaminase, cytidine deaminase, guanosyl transferase, DNA methyltransferase, RNA methyltransferases, DNA demethylases, RNA demethylases, dioxygenases, polyadenylate polymerases, pseudouridine synthases, acetyltransferases, deacetylase, ubiquitin-ligases, deubiquitinases, kinases, phosphatases, NEDD8-ligases, de-NEDDylases, SUMO-ligases, deSUMOylases, histone deacetylases, histone acetyltransferases histone methyltransferases, histone demethylases), reverse transcriptases, protein DNA binding domains, RNA binding proteins, polypeptide sequences with specific biological functions (e.g., nuclear localization signals, mitochondrial localization signals, plastid localization signals, subcellular localization signals, destabilizing signals, Geminin destruction box motifs), and biological tethering domains (e.g., MS2, Csy4 and lambda N protein). Various Type II Cas fusion proteins are described in Ribeiro et al., 2018, In. J. Genomics, Article ID: 1652567; Jayavaradhan, et al., 2019, Nat Commun 10:2866; Xiao et al., 2019, The CRISPR Journal, 2(1):51-63; Mali et al., 2013, Nat Methods. 10(10):957-63; U.S. Pat. Nos. 9,322,037, and 9,388,430. In some embodiments, a fusion partner is an adenosine deaminase. An exemplary adenosine deaminase is the tRNA adenosine deaminase (TadA) moiety contained in the adenine base editor ABE8e (Richter, 2020, Nature Biotechnology 38:883-891). The TadA moiety of ABE8e comprises the following amino acid sequence:

(SEQ ID NO: 27)
SEVEFSHEYWMRHALTLAKRARDEREVPVGAVLVLNNRVIGEGWNR
AIGLHDPTAHAEIMALRQGGLVMQNYRLIDATLYVTFEPCVMCAGA
MIHSRIGRVVFGVRNSKRGAAGSLMNVLNYPGMNHRVEITEGILAD
ECAALLCDFYRMPRQVFNAQKKAQSSIN

In some embodiments, an adenosine deaminase fusion partner comprises an amino acid sequence having at least 50%, at least 60%, at least 70%, at least 80%, at least 90%, at least 95%, at least 97%, at least 98%, at least 99% amino acid sequence identity with SEQ ID NO:27.

Type II Cas proteins of the disclosure in the form of a fusion protein comprising an adenosine deaminase can be used as an adenine base editor to change an “A” to a “G” in DNA. Type II Cas proteins of the disclosure in the form of a fusion protein comprising a cytidine deaminase can be used as a cytosine base editor to change a “C” to a “T” in DNA.

In some embodiments, a fusion protein of the disclosure comprises a means for deaminating adenosine, for example an adenosine deaminase, e.g., a TadA variant. In some embodiments, a fusion protein of the disclosure comprises a means for deaminating cytidine, for example a cytodine deaminase, e.g., cytidine deaminase 1 (CDA1) or an apolipoprotein B mRNA-editing complex (APOBEC) family deaminase (Cheng et al., 2019, Nat Commun. 10(1):3612; Gehrke et al., 2018, Nat Biotechnol. 36(10):977-982).

In some embodiments, a fusion protein of the disclosure comprises a means for synthesizing DNA from a single-stranded template, for example a reverse transcriptase. Type II Cas proteins of the disclosure in the form of a fusion protein comprising a reverse transcriptase (RT) can be used as a prime editor to carry out precise base editing without double-stranded DNA breaks.

In some embodiments, a fusion protein of the disclosure is a prime editor, e.g., a Type II Cas protein fused to a suitable RT (e.g., Moloney murine leukemia virus (M-MLV) RT or other RT enzyme). Such fusion proteins can be used in conjunction with a prime editing guide RNA (pegRNA) that both specifies the target site and encodes the desired edit (Anzalone et al., 2019, Nature, 576(7785):149-157).

In some embodiments, a fusion protein of the disclosure comprises one or more nuclear localization signals positioned N-terminal and/or C-terminal to a Type II Cas protein sequence (e.g., a ENQP Type II Cas protein having a sequence of SEQ ID NO:1). In some embodiments, a fusion protein of the disclosure comprises an N-terminal and a C-terminal nuclear localization signal, for example each having the sequence KRTADGSEFESPKKKRKV (SEQ ID NO:7).

The disclosure provides chimeric Type II Cas proteins comprising one or more domains of a ENQP Type II Cas protein and one or more domains of one or more different proteins (e.g., one or more different Type II Cas proteins).

The domain structures of wild-type ENQP Type II Cas protein were inferred by multiple alignment with the amino acid sequences of Type II Cas proteins for which the crystal structure is known and for which it is thus possible to define the boundaries of each functional domain. The domains identified in Type II Cas proteins are: the RuvC catalytic domain (discontinuous, represented by RuvC-I, RuvC-II, and RuvC-III domains), bridge helix (BH), recognition (REC) domain, HNH catalytic domain, wedge (WED) domain, and PAM-interacting domain (PID).

Table 2 below reports the amino acid positions corresponding to the boundaries between different functional domains in wild-type ENQP Type II Cas protein (SEQ ID NO:2).

TABLE 2
Amino Acid Positions of ENQP Domains

| Domain | Wild-type ENQP Type II Cas |
|---|---|
| RuvC-I | 1-61 |
| BH | 62-94 |
| REC | 95-471 |
| RuvC-II | 472-531 |
| HNH | 532-695 |
| RuvC-III | 696-832 |
| WED | 833-873 |
| PID | 874-1005 |

A chimeric Type II Cas protein can comprise one of more of the following domains (e.g., one or more, two or more, three or more, four or more, five or more, six or more, seven or more) from a ENQP Type II Cas protein, and one or more domains from one or more other proteins, for example SaCas9, SpCas9 or a Type II Cas protein described in US 2020/0332273, US 2019/0169648, or 2015/0247150 (the contents of each of which are incorporated herein by reference in their entirety): RuvC-I, BH, REC, RuvC-II, HNH, RuvC-III, WED, PID. For example, the PID domain can be swapped between different Type II Cas proteins to change the PAM specificity of the resulting chimeric protein (which is given by the donor PID domain). Swapping of other domains or portions of them is also within the scope of the disclosure (e.g., through protein shuffling).

In some embodiments, a Type II Cas protein of the disclosure comprises one, two, three, four, five, six, seven, or eight of a RuvC-I domain, a BH domain, a REC domain, a RuvC-II domain, a HNH domain, a RuvC-III domain, a WED domain, and a PID domain arranged in the N-terminal to C-terminal direction. In some embodiments, all domains are from an ENQP Type II Cas protein (e.g., a ENQP Type II Cas protein whose amino acid sequence comprises SEQ ID NO:1, 2, or 3). In other embodiments, one or more domains (e.g., one domain), e.g., a PID domain, is from another Type II Cas protein.

In addition, one or more amino acid substitutions can be introduced in one or more domains to modify the properties of the resulting nuclease in terms of editing activity, targeting specificity or PAM recognition specificity. For example, one or more amino acid substitutions can be introduced to provide nickase activity. An exemplary amino acid substitution to provide nickase activity is the D23A substitution, wherein the position of the D23A substitution is defined with respect to the amino acid numbering of SEQ ID NO: 2. Another exemplary amino acid substitution to provide nickase activity is the H612A substitution, wherein the position of the H612A substitution is defined with respect to the amino acid numbering of SEQ ID NO:2. The D23A and H612A substitution can be combined to provide a catalytically inactive Type II Cas protein.

### 6.3. Guide RNAs

The disclosure provides gRNA molecules that can be used with Type II Cas proteins of the disclosure to edit genomic DNA, for example mammalian DNA, e.g., human DNA. gRNAs of the disclosure typically comprise a spacer of 15 to 30 nucleotides in length. The spacer can be positioned 5′ of a crRNA scaffold to form a full crRNA. The crRNA can be used with a tracrRNA to effect cleavage of a target genomic sequence.

An exemplary crRNA scaffold sequence that can be used for ENQP Type II Cas gRNAs comprises GUCUUGAGCACGCACCCUUCCCCAAGGUGAUACGCU (SEQ ID NO:28) and an exemplary tracrRNA sequence that can be used for ENQP Type II Cas gRNAs comprises UCACCUUGGGGAAGGGUGCGGCUCCAGACAAGGGAAGUCAGCUAUCUGACUUACCCGUAAAGUU ACCCCCGCACCGUCCUCGGACGAUGCGGGGCGAACUUUUU (SEQ ID NO:29).

gRNAs of the disclosure are in some embodiments single guide RNAs (sgRNAs), which typically comprise the spacer at the 5′ end of the molecule and a 3′ sgRNA scaffold. Alternatively, gRNAs can comprise separate crRNA and tracrRNA molecules.

Further features of exemplary gRNA spacer sequences are described in Section 6.3.1 and further features of exemplary 3′ sgRNA scaffolds are described in Section 6.3.2.

### 6.3.1. Spacers

The spacer sequence is partially or fully complementary to a target sequence found in a genomic DNA sequence, for example a human genomic DNA sequence. For example, a spacer sequence can be partially or fully complementary to a nucleotide sequence in a gene having a disease-causing mutation. A spacer that is partially complementary to a target sequence can have, for example, one, two, or three mismatches with the target sequence.

gRNAs of the disclosure can comprise a spacer that is 15 to 30 nucleotides in length (e.g., 15 to 25, 16 to 24, 17 to 23, 18 to 22, 19 to 21, 18 to 30, 20 to 28, 22 to 26, or 23 to 25 nucleotides in length). In some embodiments, a spacer is 15 nucleotides in length. In other embodiments, a spacer is 16 nucleotides in length. In other embodiments, a spacer is 17 nucleotides in length. In other embodiments, a spacer is 18 nucleotides in length. In other embodiments, a spacer is 19 nucleotides in length. In other embodiments, a spacer is 20 nucleotides in length. In other embodiments, a spacer is 21 nucleotides in length. In other embodiments, a spacer is 22 nucleotides in length. In other embodiments, a spacer is 23 nucleotides in length. In other embodiments, a spacer is 24 nucleotides in length. In other embodiments, a spacer is 25 nucleotides in length. In other embodiments, a spacer is 26 nucleotides in length. In other embodiments, a spacer is 27 nucleotides in length. In other embodiments, a spacer is 28 nucleotides in length. In other embodiments, a spacer is 29 nucleotides in length. In other embodiments, a spacer is 30 nucleotides in length.

Type II Cas endonucleases require a specific sequence, called a protospacer adjacent motif (PAM) that is downstream (e.g., directly downstream) of the target sequence on the non-target strand. Thus, spacer sequences for targeting a gene of interest can be identified by scanning the gene for PAM sequences recognized by the Type II Cas protein. Exemplary PAM sequences for ENQP Type II Cas proteins are shown in Table 3.

TABLE 3
Exemplary ENQP PAM Sequences
Sequence

N₄CMNA
N₄CCNA
N₄CCGA
N₄CTGA
N = A, T, C, or G
M = C or A

Examples 1 and 3 describe exemplary sequences that can be used to target RHO (Examples 1 and 3) and DNMT1 (Example 1) genomic sequences. Example 4 describes exemplary sequences that can be used to target TRAC, B2M, PD1, and LAG3 genomic sequences. In some embodiments, a gRNA of the disclosure comprises a spacer sequence targeting RHO or DNMT1. In some embodiments, a gRNA of the disclosure comprises a spacer sequence targeting RHO. In some embodiments, a gRNA of the disclosure comprises a spacer sequence targeting DNMT1. In some embodiments, a gRNA of the disclosure comprises a spacer sequence targeting TRAC. In some embodiments, a gRNA of the disclosure comprises a spacer sequence targeting B2M. In some embodiments, a gRNA of the disclosure comprises a spacer sequence targeting PD1. In some embodiments, a gRNA of the disclosure comprises a spacer sequence targeting LAG3.

Additional exemplary spacer sequences that can be used in gRNAs of the disclosure are set forth in Table 4A, Table 4B, Table 4C, and Table 4D.

TABLE 4A
Exemplary spacer sequences (RHO and DNMT1)

| | | | SEQ |
|---|---|---|---|
| | | | ID |
| Target | Spacer Name | Spacer Sequence | NO: |
| RHO | gRNA2_ENQP_ | CUUGUGGCUGACCCGYGGCUGCUC | 34 |
| | RHO_A/G | | |
| RHO | gRNA5_ENQP_ | CUUGGGUGGGAGCAGCCRCGGGU | 35 |
| | RHO_A/G | | |
| RHO | gRNA6_ENQP_ | GGAGCAGCCRCGGGUCAGCCACAA | 36 |
| | RHO_A/G | | |
| RHO | gRNA2_ENQP_ | CUUGUGGCUGACCCGUGGCUGCUC | 37 |
| | RHO_A | | |
| RHO | gRNA5_ENQP_ | CUUGGGUGGGAGCAGCCACGGGU | 38 |
| | RHO_A | | |
| RHO | gRNA6_ENQP_ | GGAGCAGCCACGGGUCAGCCACAA | 39 |
| | RHO_A | | |
| RHO | gRNA2_ENQP_ | CUUGUGGCUGACCCGCGGCUGCUC | 40 |
| | RHO_G | | |
| RHO | gRNA5_ENQP_ | CUUGGGUGGGAGCAGCCGCGGGU | 41 |
| | RHO_G | | |
| RHO | gRNA6_ENQP_ | GGAGCAGCCGCGGGUCAGCCACAA | 42 |
| | RHO_G | | |
| DNMT1 | gRNA1_ENQP_ | GUGCCAGAAACAGGGGUGACGGG | 43 |
| | DNMT1 | | |

Position of RHO rs7984 SNP shown in bold; Y = U or C; R = A or G

TABLE 4B
Exemplary spacer sequences (RHO rs7984 SNP)

| | | | SEQ ID |
|---|---|---|---|
| Target | Spacer Name | Spacer Sequence | NO: |
| RHO | gRNA4_A/G | GGCCCUUGUGGCUGACCC | 93 |
| | | GYGGCU | |
| RHO | gRNA4_A | GGCCCUUGUGGCUGACCC | 94 |
| | | GUGGCU | |
| RHO | gRNA4_G | GGCCCUUGUGGCUGACCC | 95 |
| | | GCGGCU | |
| RHO | gRNA5-20nt_ | GGGUGGGAGCAGCCRCG | 96 |
| | A/G | GGU | |
| RHO | gRNA5-21nt_ | UGGGUGGGAGCAGCCRC | 97 |
| | A/G | GGGU | |
| RHO | gRNA5-22nt_ | UUGGGUGGGAGCAGCCR | 98 |
| | A/G | CGGGU | |
| RHO | gRNA5-24nt_ | UCUUGGGUGGGAGCAGC | 99 |
| | A/G | CRCGGGU | |
| RHO | gRNA5-25nt_ | UUCUUGGGUGGGAGCAG | 100 |
| | A/G | CCRCGGGU | |
| RHO | gRNA5-20nt_A | GGGUGGGAGCAGCCACG | 101 |
| | | GGU | |
| RHO | gRNA5-21nt_A | UGGGUGGGAGCAGCCAC | 102 |
| | | GGGU | |
| RHO | gRNA5-22nt_A | UUGGGUGGGAGCAGCCA | 103 |
| | | CGGGU | |
| RHO | gRNA5-24nt_A | UCUUGGGUGGGAGCAGC | 104 |
| | | CACGGGU | |
| RHO | gRNA5-25nt_A | UUCUUGGGUGGGAGCAG | 105 |
| | | CCACGGGU | |
| RHO | gRNA5-20nt_G | GGGUGGGAGCAGCCGCG | 106 |
| | | GGU | |
| RHO | gRNA5-21nt_G | UGGGUGGGAGCAGCCGC | 107 |
| | | GGGU | |
| RHO | gRNA5-22nt_G | UUGGGUGGGAGCAGCCG | 108 |
| | | CGGGU | |
| RHO | gRNA5-24nt_G | UCUUGGGUGGGAGCAGC | 109 |
| | | CGCGGGU | |
| RHO | gRNA5-25nt_G | UUCUUGGGUGGGAGCAG | 110 |
| | | CCGCGGGU | |

Position of RHO rs7984 SNP shown in bold; Y = U or C; R = A or G

TABLE 4C
Exemplary spacer sequences (RHO intron 1)

| | | | SEQ |
|---|---|---|---|
| | Spacer | | ID |
| Target | Name | Spacer Sequence | NO: |
| RHO | g-int482 | CAUGCUCCCGGGCUCCUGCACAC | 111 |
| RHO | g-int486 | CCUCCAUGCUCCCGGGCUCCUGC | 112 |
| RHO | g-int521 | AGCCACCACCGCCAAGCCCGGGA | 113 |
| RHO | g-int572 | UCCCUUCUCCUGUCCUGUCAAUG | 114 |
| RHO | g-int573 | CCCUUCUCCUGUCCUGUCAAUGU | 115 |
| RHO | g-int582 | UGUCCUGUCAAUGUUAUCCAAAG | 116 |
| RHO | g-int597 | AUCCAAAGCCCUCAUAUAUUCAG | 117 |
| RHO | g-int648 | AAGGCAGUGUUCAGUGCCAGCCC | 118 |
| RHO | g-int650 | UCAAGGCAGUGUUCAGUGCCAGC | 119 |
| RHO | g-int686 | GUGAAAUUAGACAAGCGCAUAUU | 120 |
| RHO | g-int696F | UUGGAGCAAUAUGCGCUUGUCUA | 121 |
| RHO | g-int696R | GUUUUCUUGCUGUGAAAUUAGACA | 122 |
| RHO | g-int722 | UCACAGCAAGAAAACUGAGCUGA | 123 |
| RHO | g-int751 | AAGAAGUCAAGCGCCCUGCUGGG | 124 |
| RHO | g-int753 | GAAGUCAAGCGCCCUGCUGGGGC | 125 |
| RHO | g-int761 | AACUCUGCACCCGUCCCUGUGUG | 126 |
| RHO | g-int767 | UGCUGGGGCGUCACACAGGGACG | 127 |
| RHO | g-int795 | AACAUGGCCCGAGAUAGAUGCGG | 128 |
| RHO | g-int814 | ACAGAGGCUUGGUGCUGCAAACA | 129 |
| RHO | g-int824 | UCCAAGGGAAACAGAGGCUUGGU | 130 |
| RHO | g-int840 | GACUCAGCACAGCUGCUCCAAGG | 131 |
| RHO | g-int850 | GCCUGGGUCUGACUCAGCACAGC | 132 |
| RHO | g-int856 | CCCUUGGAGCAGCUGUGCUGAGU | 133 |
| RHO | g-int860 | UCAGUGCCCAGCCUGGGUCUGAC | 134 |

TABLE 4D
Exemplary spacer sequences
(TRAC, B2M, PD1, and LAG3)

| | | | SEQ |
|---|---|---|---|
| | Spacer | | ID |
| Target | Name | Spacer Sequence | NO: |
| TRAC | TRAC_g709 | AUACACAUCAGAAUCCUUACUUU | 135 |
| TRAC | TRAC_g730 | GUAAGGAUUCUGAUGUGUAUAUCA | 136 |
| TRAC | TRAC_g793 | GAGCAACAGUGCUGUGGCCUGGA | 137 |
| TRAC | TRAC_g813 | GGAGCAACAAAUCUGACUUUGCA | 138 |
| TRAC | TRAC_g821 | UGCUGUUGUUGAAGGCGUUUGCA | 139 |
| TRAC | TRAC_g857 | AGCAUUAUUCCAGAAGACACCUU | 140 |
| TRAC | TRAC_g862 | UAUUCCAGAAGACACCUUCUUCC | 141 |
| TRAC | TRAC_g724 | ACAAAGUAAGGAUUCUGAUGUGU | 142 |
| TRAC | TRAC_g733 | GUCUAGCACAGUUUUGUCUGUGA | 143 |
| TRAC | TRAC_g754 | CUUGAAGUCCAUAGACCUCAUGU | 144 |
| TRAC | TRAC_g772 | GGCCACAGCACUGUUGCUCUUGA | 145 |
| TRAC | TRAC_g796 | UGCAAAGUCAGAUUUGUUGCUCC | 146 |
| TRAC | TRAC_g827 | GAAUAAUGCUGUUGUUGAAGGCG | 147 |
| B2M | B2M_g791 | UAAGGCCACGGAGCGAGACAUCU | 148 |
| B2M | B2M_g824 | CAGGCCAGAAAGAGAGAGUAGCG | 149 |
| B2M | B2M_g860 | GCCUGGAGGCUAUCCAGCGUGAGU | 150 |
| PD1 | PD1_g279 | GGCCGCCAGCCCAGUUGUAGCAC | 151 |
| PD1 | PD1_g281 | CUGGCCGCCAGCCCAGUUGUAGC | 152 |
| LAG3 | LAG3_g636 | GCCAUCCCCGUUUUACCUGGAGC | 153 |
| LAG3 | LAG3_g637 | CGCCAUCCCCGUUUUACCUGGAG | 154 |
| LAG3 | LAG3_g638 | CCGCCAUCCCCGUUUUACCUGGA | 155 |
| LAG3 | LAG3_g668 | GGGAUGGCGGGAGGGUUGACCUC | 156 |
| LAG3 | LAG3_g714 | GAGCUAAGGACCUCAGGACCUUUG | 157 |
| LAG3 | LAG3_g768 | GAGAGGCUUUCGGGGUGGAAGGAC | 158 |
| LAG3 | LAG3_g769 | GAGAGGCUUUCGGGGUGGAAGGA | 159 |
| LAG3 | LAG3_g770 | AGAGAGGCUUUCGGGGUGGAAGG | 160 |

The RHO spacer sequences in Table 4A and Table 4B are useful for targeting a RHO gene in the vicinity of the rs7984 SNP, located in the 5′ untranslated region (UTR) of the RHO gene. Allele specific targeting can be achieved by using a gRNA targeting the SNP variant found in a cell or subject. For example, gRNA2_ENQP_RHO_A, gRNA5_ENQP_RHO_A, and gRNA6_ENQP_RHO_A-based guides can be used when the cell or subject has an “A” at the position of the rs7984 SNP, while gRNA2_ENQP_RHO_G, gRNA5_ENQP_RHO_G, and gRNA6_ENQP_RHO_G-based guides can be used when the cell or subject has a “G” at the position of the rs7984 SNP. Such guides can be used, for example, with a guide RNA targeting RHO intron 1 (for example, having a spacer as shown in Table 4C) to knock-out expression of the mutated protein. Allele-specific targeting of RHO is described further in Example 3. Exemplary combinations of guides include a first guide RNA having a gRNA5 spacer (e.g., a spacer whose sequence is selected from SEQ ID NOS: 35, 38, 41, and 96-110) and a second guide RNA having a g-int648 spacer (SEQ ID NO:118), g-int795 spacer (SEQ ID NO: 128), or a g-int824 spacer (SEQ ID NO:130).

In some embodiments, a gRNA of the disclosure has a spacer whose nucleotide sequence comprises 15 or more consecutive nucleotides from a sequence shown in Table 4A (e.g., SEQ ID NO:35, 38, or 41). In some embodiments, a gRNA of the disclosure has a spacer whose nucleotide sequence comprises 16 or more consecutive nucleotides from a sequence shown in Table 4A (e.g., SEQ ID NO:35, 38, or 41). In some embodiments, a gRNA of the disclosure has a spacer whose nucleotide sequence comprises 17 or more consecutive nucleotides from a sequence shown in Table 4A (e.g., SEQ ID NO:35, 38, or 41). In some embodiments, a gRNA of the disclosure has a spacer whose nucleotide sequence comprises 18 or more consecutive nucleotides from a sequence shown in Table 4A (e.g., SEQ ID NO:35, 38, or 41). In some embodiments, a gRNA of the disclosure has a spacer whose nucleotide sequence comprises 19 or more consecutive nucleotides from a sequence shown in Table 4A (e.g., SEQ ID NO:35, 38, or 41). In some embodiments, a gRNA of the disclosure has a spacer whose nucleotide sequence comprises 20 or more consecutive nucleotides from a sequence shown in Table 4A (e.g., SEQ ID NO:35, 38, or 41). In some embodiments, a gRNA of the disclosure has a spacer whose nucleotide sequence comprises 21 or more consecutive nucleotides from a sequence shown in Table 4A (e.g., SEQ ID NO:35, 38, or 41). In some embodiments, a gRNA of the disclosure has a spacer whose nucleotide sequence comprises 22 or more consecutive nucleotides from a sequence shown in Table 4A (e.g., SEQ ID NO:35, 38, or 41). In some embodiments, a gRNA of the disclosure has a spacer whose nucleotide sequence comprises 23 or more consecutive nucleotides from a sequence shown in Table 4A (e.g., SEQ ID NO:35, 38, or 41). In some embodiments, a gRNA of the disclosure has a spacer whose nucleotide sequence comprises 24 consecutive nucleotides from a sequence shown in Table 4A.

In some embodiments, a gRNA of the disclosure has a spacer whose nucleotide sequence comprises 15 or more consecutive nucleotides from a sequence shown in Table 4B (e.g., any one of SEQ ID NOS: 96-100). In some embodiments, a gRNA of the disclosure has a spacer whose nucleotide sequence comprises 16 or more consecutive nucleotides from a sequence shown in Table 4B (e.g., any one of SEQ ID NOS: 96-100). In some embodiments, a gRNA of the disclosure has a spacer whose nucleotide sequence comprises 17 or more consecutive nucleotides from a sequence shown in Table 4B (e.g., any one of SEQ ID NOS: 96-100). In some embodiments, a gRNA of the disclosure has a spacer whose nucleotide sequence comprises 18 or more consecutive nucleotides from a sequence shown in Table 4B (e.g., any one of SEQ ID NOS: 96-100). In some embodiments, a gRNA of the disclosure has a spacer whose nucleotide sequence comprises 19 or more consecutive nucleotides from a sequence shown in Table 4B (e.g., any one of SEQ ID NOS: 96-100). In some embodiments, a gRNA of the disclosure has a spacer whose nucleotide sequence comprises 20 or more consecutive nucleotides from a sequence shown in Table 4B (e.g., any one of SEQ ID NOS: 96-100). In some embodiments, a gRNA of the disclosure has a spacer whose nucleotide sequence comprises 21 or more consecutive nucleotides from a sequence shown in Table 4B (e.g., any one of SEQ ID NOS: 97-100). In some embodiments, a gRNA of the disclosure has a spacer whose nucleotide sequence comprises 22 or more consecutive nucleotides from a sequence shown in Table 4B (e.g., any one of SEQ ID NOS: 98-100). In some embodiments, a gRNA of the disclosure has a spacer whose nucleotide sequence comprises 23 or more consecutive nucleotides from a sequence shown in Table 4B (e.g., any one of SEQ ID NOS: 99-100). In some embodiments, a gRNA of the disclosure has a spacer whose nucleotide sequence comprises 24 or more consecutive nucleotides from a sequence shown in Table 4B (e.g., any one of SEQ ID NOS: 99-100).

In some embodiments, a gRNA of the disclosure has a spacer whose nucleotide sequence comprises 15 or more consecutive nucleotides from a sequence shown in Table 4C (e.g., any one of SEQ ID NOS: 118, 128 and 130). In some embodiments, a gRNA of the disclosure has a spacer whose nucleotide sequence comprises 16 or more consecutive nucleotides from a sequence shown in Table 4C (e.g., any one of SEQ ID NOS: 118, 128 and 130). In some embodiments, a gRNA of the disclosure has a spacer whose nucleotide sequence comprises 17 or more consecutive nucleotides from a sequence shown in Table 4C (e.g., any one of SEQ ID NOS: 118, 128 and 130). In some embodiments, a gRNA of the disclosure has a spacer whose nucleotide sequence comprises 18 or more consecutive nucleotides from a sequence shown in Table 4C (e.g., any one of SEQ ID NOS: 118, 128 and 130). In some embodiments, a gRNA of the disclosure has a spacer whose nucleotide sequence comprises 19 or more consecutive nucleotides from a sequence shown in Table 4C (e.g., any one of SEQ ID NOS: 118, 128 and 130). In some embodiments, a gRNA of the disclosure has a spacer whose nucleotide sequence comprises 20 or more consecutive nucleotides from a sequence shown in Table 4C (e.g., any one of SEQ ID NOS: 118, 128 and 130). In some embodiments, a gRNA of the disclosure has a spacer whose nucleotide sequence comprises 21 or more consecutive nucleotides from a sequence shown in Table 4C (e.g., any one of SEQ ID NOS: 118, 128 and 130). In some embodiments, a gRNA of the disclosure has a spacer whose nucleotide sequence comprises 22 or more consecutive nucleotides from a sequence shown in Table 4C (e.g., any one of SEQ ID NOS: 118, 128 and 130). In some embodiments, a gRNA of the disclosure has a spacer whose nucleotide sequence comprises 23 or more consecutive nucleotides from a sequence shown in Table 4C (e.g., any one of SEQ ID NOS: 118, 128 and 130). In some embodiments, a gRNA of the disclosure has a spacer whose nucleotide sequence comprises 24 consecutive nucleotides from a sequence shown in Table 4C.

In some embodiments, a gRNA of the disclosure has a spacer whose nucleotide sequence comprises 15 or more consecutive nucleotides from a sequence shown in Table 4D In some embodiments, a gRNA of the disclosure has a spacer whose nucleotide sequence comprises 24 or more consecutive nucleotides from a sequence shown in Table 4D (e.g., any one of SEQ ID NOS: 139, 149, and 152). In some embodiments, a gRNA of the disclosure has a spacer whose nucleotide sequence comprises 16 or more consecutive nucleotides from a sequence shown in Table 4D (e.g., any one of SEQ ID NOS: 139, 149, and 152). In some embodiments, a gRNA of the disclosure has a spacer whose nucleotide sequence comprises 17 or more consecutive nucleotides from a sequence shown in Table 4D (e.g., any one of SEQ ID NOS: 139, 149, and 152). In some embodiments, a gRNA of the disclosure has a spacer whose nucleotide sequence comprises 18 or more consecutive nucleotides from a sequence shown in Table 4D (e.g., any one of SEQ ID NOS: 139, 149, and 152). In some embodiments, a gRNA of the disclosure has a spacer whose nucleotide sequence comprises 19 or more consecutive nucleotides from a sequence shown in Table 4D (e.g., any one of SEQ ID NOS: 139, 149, and 152). In some embodiments, a gRNA of the disclosure has a spacer whose nucleotide sequence comprises 20 or more consecutive nucleotides from a sequence shown in Table 4D (e.g., any one of SEQ ID NOS: 139, 149, and 152). In some embodiments, a gRNA of the disclosure has a spacer whose nucleotide sequence comprises 21 or more consecutive nucleotides from a sequence shown in Table 4D (e.g., any one of SEQ ID NOS: 139, 149, and 152). In some embodiments, a gRNA of the disclosure has a spacer whose nucleotide sequence comprises 22 or more consecutive nucleotides from a sequence shown in Table 4D (e.g., any one of SEQ ID NOS: 139, 149, and 152). In some embodiments, a gRNA of the disclosure has a spacer whose nucleotide sequence comprises 23 or more consecutive nucleotides from a sequence shown in Table 4D (e.g., any one of SEQ ID NOS: 139, 149, and 152). In some embodiments, a gRNA of the disclosure has a spacer whose nucleotide sequence comprises 24 consecutive nucleotides from a sequence shown in Table 4D.

6.3.2. sgRNA Molecules

gRNAs of the disclosure can be single-guide RNA (sgRNA) molecules. A sgRNA can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

The sgRNA can comprise a variable length spacer sequence (e.g., 15 to 30 nucleotides) at the 5′ end of the sgRNA sequence and a 3′ sgRNA segment.

Type II Cas gRNAs typically comprise a repeat-antirepeat duplex and/or one or more stem-loops generated by the gRNA's secondary structure. The length of the repeat-antirepeat duplex and/or one or more stem-loops can be modified in order to modulate (e.g., increase) the editing efficacy of a Type II Cas nuclease, and/or to reduce the size of a guide RNA for easier vectorization in situations in which the cargo size of the vector is limiting (e.g., AAV vectors).

For example, the repeat-antirepeat duplex (which in a sgRNA is fused through a synthetic linker to become an additional stem loop in the structure) can be trimmed at different lengths without generally having detrimental effects on nuclease function and in some cases even producing increased enzymatic activity. If bulges are present within this duplex they generally should be retained in the final guide RNA sequence.

Further optimization of the structure can be obtained by introducing targeted base changes into the stems of the gRNA to increase their stability and folding. Such base changes will preferably correspond to the introduction of G: C couples, which are known to generate the strongest Watson-Crick pairing. For the sake of clarity, these substitutions can consist in the introduction of a G or a C in a specific position of a stem together with a complementary substitution in another position of the gRNA sequence which is predicted to base pair with the former, for example according to available bioinformatic tools for RNA folding such as UNAfold or RNAfold.

Stem-loop trimming can also be exploited to stabilize desired secondary structures by removing portions of the guide RNA producing unwanted secondary structures through annealing with other regions of the RNA molecule.

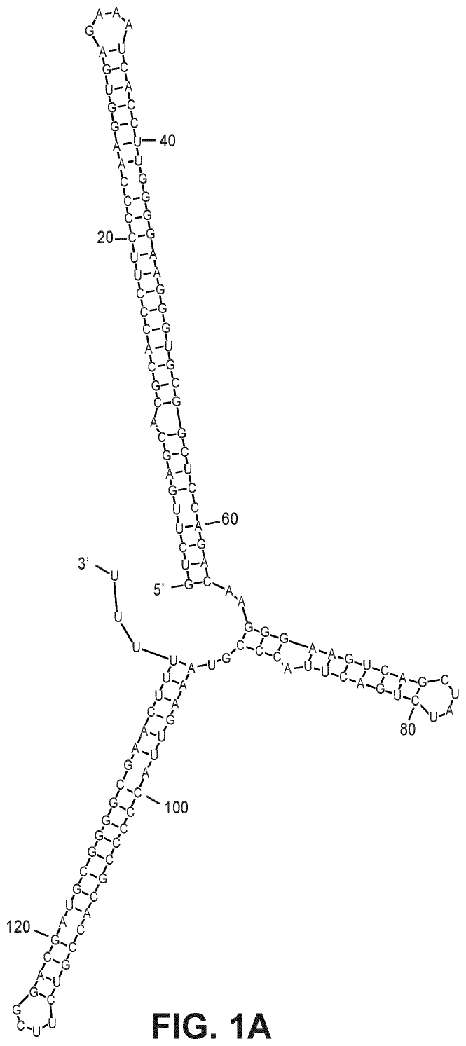

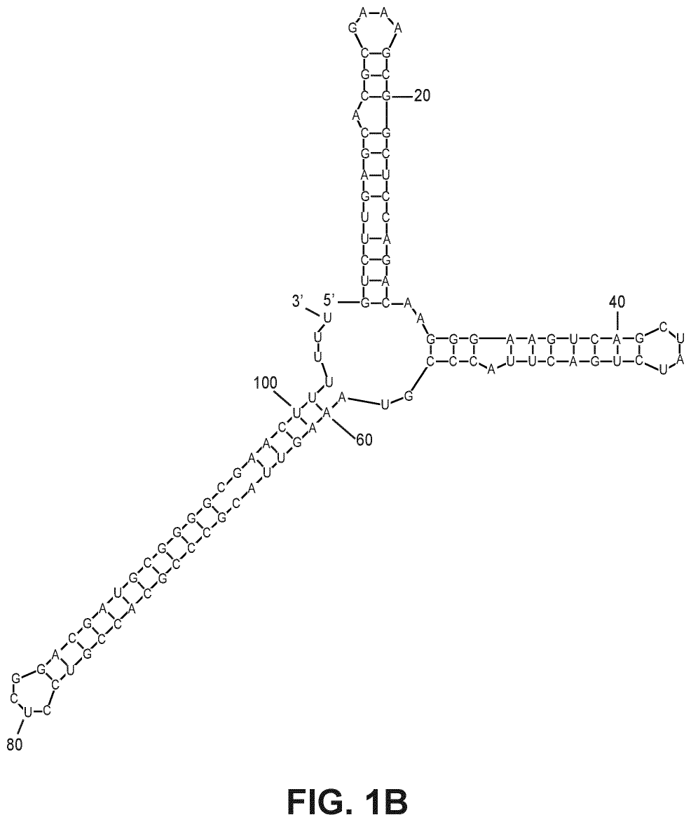

Examples of modifications to that can be made to exemplary ENQP Type II Cas gRNA 3′ scaffolds to make trimmed scaffolds are illustrated in FIG. 1A-1B. For example, the scaffold shown in FIG. 1A can be modified by trimming its first stem-loop to generate a shorter scaffold shown in FIG. 1B.

Further exemplary 3′ sgRNA scaffold sequences for ENQP Type II Cas sgRNAs are shown in Table 5.

TABLE 5
Sequences of sgRNA Scaffolds for ENQP Type II Cas

| | | SEQ |
|---|---|---|
| | | ID |
| Name | Sequence | NO: |
| ENQP full | GUCUUGAGCACGCACCCUUCCCCAAGG | 44 |
| scaffold | UGAGAAAUCACCUUGGGGAAGGGUGCG | |
| ENQP trimmed | GCUCCAGACAAGGGAAGUCAGCUAUCU | |
| | GACUUACCCGUAAAGUUACCCCCGCAC | |
| | CGUCCUCGGACGAUGCGGGGCGAAC | |
| scaffold | GUCUUGAGCACGCGAAAGCGGCUCCAG | 45 |
| (sgRNAtrimV1) | ACAAGGGAAGUCAGCUAUCUGACUUAC | |
| | CCGUAAAGUUACCCCCGCACCGUCCUC | |
| | GGACGAUGCGGGGCGAAC | |
| ENQP super | GUCUUGAGCACGCGAAAGCGGCUCCAG | 91 |
| trimmed | ACAAGGGAAGUCAGCUAUCUGACUUAC | |
| scaffold | CCGUAAAGUUACCCCGAAAGGGCGAAC | |
| (sgRNAtrimV2) | | |

The sgRNA (e.g., for use with an ENQP Type II Cas protein) can comprise no uracil base at the 3′ end of the sgRNA sequence. Typically, however, the sgRNA comprises one or more uracil bases at the 3′ end of the sgRNA sequence, for example to promote correct sgRNA folding. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence. Different length stretches of uracil can be appended at the 3′end of a sgRNA as terminators. Thus, for example, the 3′ sgRNA sequences set forth in Table 5 can be modified by adding (or removing) one or more uracils at the end of the sequence.

In some embodiments, a sgRNA scaffold for use with an ENQP Type II Cas protein comprises the sequence

(SEQ ID NO: 46)
GUCUUGAGCACGCACCCUUCCCCAAGGUGAGAAAUCACCUUGGGG
AAGGGUGCGGCUCCAGACAAGGGAAGUCAGCUAUCUGACUUACCC
GUAAAGUUACCCCCGCACCGUCCUCGGACGAUGCGGGGCGAACUU
UUUU

In some embodiments, a sgRNA scaffold for use with an ENQP Type II Cas protein comprises the sequence

(SEQ ID NO: 47)
GUCUUGAGCACGCGAAAGCGGCUCCAGACAAGGGAAGUCAGCUAU
CUGACUUACCCGUAAAGUUACCCCCGCACCGUCCUCGGACGAUGC
GGGGCGAACUUUUUU

In some embodiments, a sgRNA scaffold for use with an ENQP Type II Cas protein comprises the sequence

(SEQ ID NO: 92)
GUCUUGAGCACGCGAAAGCGGCUCCAGACAAGGGAAGUCAGCUAU
CUGACUUACCCGUAAAGUUACCCCGAAAGGGCGAACUUUUUU

6.3.3. Modified gRNA Molecules

Guide RNAs can be readily synthesized by chemical means, enabling a number of modifications to be readily incorporated, as described in the art. The disclosed gRNA (e.g., sgRNA) molecules can be unmodified or can contain any one or more of an array of chemical modifications.

While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach that can be used for generating chemically modified RNAs of greater length is to produce two or more molecules that are ligated together. Much longer RNAs, such as those encoding a Type II Cas endonuclease, are more readily generated enzymatically. While fewer types of modifications are available for use in enzymatically produced RNAs, there are still modifications that can be used to, for instance, enhance stability, reduce the likelihood or degree of innate immune response, and/or enhance other attributes, as described herein and in the art.

By way of illustration of various types of modifications, especially those used frequently with smaller chemically synthesized RNAs, modifications can comprise one or more nucleotides modified at the 2′ position of the sugar, for instance a 2′-O-alkyl, 2′-O-alkyl-O-alkyl, or 2′-fluoro-modified nucleotide. In some examples, RNA modifications can comprise 2′-fluoro, 2′-amino or 2′-O-methyl modifications on the ribose of pyrimidines, abasic residues, or an inverted base at the 3′ end of the RNA. Such modifications can be routinely incorporated into oligonucleotides and these oligonucleotides have been shown to have a higher Tm (thus, higher target binding affinity) than 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, ˜N(CH₃)—O—CH₂ (known as a methylene (methylimino) or MMI backbone), CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. 1995, Ace. Chem. Res., 28:366-374); morpholino backbone structures (see U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., 1991, Science 254:1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, 2002, Biochemistry, 41(14):4503-4510; Genesis, Volume 30, Issue 3, (2001); Heasman, 2002, Dev. Biol., 243:209-214; Nasevicius et al., 2000, Nat. Genet., 26:216-220; Lacerra et al., 2000, Proc. Natl. Acad. Sci., 97:9591-9596; and U.S. Pat. No. 5,034,506.

Cyclohexenyl nucleic acid oligonucleotide mimetics are described in Wang et al., 2000, J. Am. Chem. Soc., 122:8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

One or more substituted sugar moieties can also be included, e.g., one of the following at the 2′ position: OH, SH, SCH₃, F, OCN, OCH₃, OCH₃ O (CH₂)n CH₃, O (CH₂)n NH₂, or O (CH₂)n CH₃, where n is from 1 to about 10; C₁ to C₁₀ lower alkyl, alkoxyalkoxy, substituted lower alkyl, alkaryl or aralkyl; Cl; Br; CN; CF₃; OCF₃; O—, S—, or bi-alkyl; O—, S—, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some aspects, a modification includes 2′-methoxyethoxy (2′-O—CH₂CH₂OCH₃, also known as 2′-O-(2-methoxyethyl)) (Martin et al., 1995, Helv. Chim. Acta, 78, 486). Other modifications include 2′-methoxy (2′-O—CH₃), 2′-propoxy (2′-OCH₂ CH₂CH₃) and 2′-fluoro (2′-F). Similar modifications can also be made at other positions on the oligonucleotide, particularly the 3′ position of the sugar on the 3′ terminal nucleotide and the 5′ position of 5′ terminal nucleotide. Oligonucleotides can also have sugar mimetics, such as cyclobutyls in place of the pentofuranosyl group.

In some examples, both a sugar and an internucleoside linkage (in the backbone) of the nucleotide units can be replaced with novel groups. The base units can be maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide can be replaced with an amide containing backbone, for example, an aminoethylglycine backbone. The nucleobases can be retained and bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al., 1991, Science, 254:1497-1500.

RNAs such as guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxy cytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine, and 2,6-diaminopurine. Komberg, A., DNA Replication, W. H. Freeman & Co., San Francisco, pp. 75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by about 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

Further, nucleobases can comprise those disclosed in U.S. Pat. No. 3,687,808, those disclosed in ‘The Concise Encyclopedia of Polymer Science and Engineering’, 858-859, Kroschwitz, J. I., ed. John Wiley & Sons, 1990, those disclosed by Englisch et al., Angewandle Chemie, International Edition’, 1991, 30, p. 613, and those disclosed by Sanghvi, Y. S., Chapter 15, Antisense Research and Applications’, 289-302, Crooke, S. T. and Lebleu, B. ea., CRC Press, 1993. Certain of these nucleobases can be useful for increasing the binding affinity of the oligomeric compounds of the invention. These include 5-substituted pyrimidines, 6-azapyrimidines and N-2, N-6 and O-6 substituted purines, comprising 2-aminopropyladenine, 5-propynyluracil and 5-propynylcytosine. 5-methylcytosine substitutions have been shown to increase nucleic acid duplex stability by about 0.6-1.2° C. (Sanghvi, Y. S., Crooke, S. T. and Lebleu, B., eds, ‘Antisense Research and Applications’, CRC Press, Boca Raton, 1993, 276-278) and are aspects of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and U.S. Patent Application Publication 2003/0158403.

Thus, a modified gRNA can include, for example, one or more non-natural sugars, internucleotide linkages and/or bases. It is not necessary for all positions in a given gRNA to be uniformly modified, and in fact more than one of the aforementioned modifications can be incorporated in a single oligonucleotide, or even in a single nucleoside within an oligonucleotide.

The guide RNAs and/or mRNA (or DNA) encoding an endonuclease can be chemically linked to one or more moieties or conjugates that enhance the activity, cellular distribution, or cellular uptake of the oligonucleotide. Such moieties comprise, but are not limited to, lipid moieties such as a cholesterol moiety (Letsinger et al. 1989, Proc. Natl. Acad. Sci. USA, 86:6553-6556); cholic acid (Manoharan et al, 1994, Bioorg. Med. Chem. Let., 4:1053-1060); a thioether, e.g., hexyl-S-tritylthiol (Manoharan et al, 1992, Ann. N. Y. Acad. Sci., 660:306-309; Manoharan et al., 1993, Bioorg. Med. Chem. Let., 3:2765-2770); a thiocholesterol (Oberhauser et al., 1992, Nucl. Acids Res., 20:533-538); an aliphatic chain, e.g., dodecandiol or undecyl residues (Kabanov et al, 1990, FEBS Lett., 259:327-330; Svinarchuk et al, 1993, Biochimie, 75:49-54); a phospholipid, e.g., di-hexadecyl-rac-glycerol or triethylammonium 1,2-di-O-hexadecyl-rac-glycero-3-H-phosphonate (Manoharan et al., 1995, Tetrahedron Lett., 36:3651-3654; and Shea et al, 1990, Nucl. Acids Res., 18:3777-3783); a polyamine or a polyethylene glycol chain (Mancharan et al, 1995, Nucleosides & Nucleotides, 14:969-973); adamantane acetic acid (Manoharan et al, 1995, Tetrahedron Lett., 36:3651-3654); a palmityl moiety (Mishra et al., 1995, Biochim. Biophys. Acta, 1264:229-237); or an octadecylamine or hexylamino-carbonyl-t oxycholesterol moiety (Crooke et al, 1996, J. Pharmacol. Exp. Ther., 277:923-937). See also U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203; 5,451,463; 5,510,475; 5,512,667; 5,514,785; 5,565,552; 5,567,810; 5,574,142; 5,585,481; 5,587,371; 5,595,726; 5,597,696; 5,599,923; 5,599, 928 and 5,688,941.

Sugars and other moieties can be used to target proteins and complexes comprising nucleotides, such as cationic polysomes and liposomes, to particular sites. For example, hepatic cell directed transfer can be mediated via asialoglycoprotein receptors (ASGPRs); see, e.g., Hu, et al., 2014, Protein Pept Lett. 21(10):1025-30. Other systems known in the art and regularly developed can be used to target biomolecules of use in the present case and/or complexes thereof to particular target cells of interest.

Targeting moieties or conjugates can include conjugate groups covalently bound to functional groups, such as primary or secondary hydroxyl groups. Conjugate groups of the present disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this present disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism or excretion of the compounds of the present disclosure. Representative conjugate groups are disclosed in International Patent Application Publication WO1993007883, and U.S. Pat. No. 6,287,860. Conjugate moieties include, but are not limited to, lipid moieties such as a cholesterol moiety, cholic acid, a thioether, e.g., hexyl-5-trityl thiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

A large variety of modifications have been developed and applied to enhance RNA stability, reduce innate immune responses, and/or achieve other benefits that can be useful in connection with the introduction of polynucleotides into human cells, as described herein; see, e.g., the reviews by Whitehead K A et al., 2011, Annual Review of Chemical and Biomolecular Engineering, 2:77-96; Gaglione and Messere, 2010, Mini Rev Med Chem, 10(7):578-95; Chernolovskaya et al, 2010, Curr Opin Mol Ther., 12(2): 158-67; Deleavey et al., 2009, Curr Protoc Nucleic Acid Chem Chapter 16: Unit 16.3; Behlke, 2008, Oligonucleotides 18(4):305-19; Fucini et al, 2012, Nucleic Acid Ther 22(3): 205-210; Bremsen et al, 2012, Front Genet 3:154.

### 6.4. Systems

The disclosure provides systems comprising a Type II Cas protein of the disclosure (e.g., as described in Section 6.2) and a means for targeting the Type II Cas protein to a target genomic sequence. The means for targeting the Type II Cas protein to a target genomic sequence can be a guide RNA (gRNA) (e.g., as described in Section 6.3).

The disclosure also provides systems comprising a Type II Cas protein of the disclosure (e.g., as described in Section 6.2) and a gRNA (e.g., as described in Section 6.3). The systems can comprise a ribonucleoprotein particle (RNP) in which a Type II Cas protein is complexed with a gRNA, for example a sgRNA or separate crRNA and tracrRNA. Systems of the disclosure can in some embodiments further comprise genomic DNA complexed with the Type II Cas protein and the gRNA. Accordingly, the disclosure provides systems comprising a Type II Cas protein, a genomic DNA, and gRNA, all complexed with one another.

The systems of the disclosure can exist within a cell (whether the cell is in vivo, ex vivo, or in vitro) or outside a cell (e.g., in a particle our outside of a particle).

### 6.5. Nucleic Acids

The disclosure provides nucleic acids (e.g., DNA or RNA) encoding Type II Cas proteins (e.g., ENQP Type II Cas proteins), nucleic acids encoding gRNAs of the disclosure (e.g., a single gRNA or combination of gRNAs), nucleic acids encoding both Type II Cas proteins and gRNAs, and pluralities of nucleic acids, for example comprising a nucleic acid encoding a Type II Cas protein and a gRNA.

A nucleic acid encoding a Type II Cas protein and/or gRNA can be, for example, a plasmid or a viral genome (e.g., a lentivirus, retrovirus, adenovirus, or adeno-associated virus genome). Plasmids can be, for example, plasmids for producing virus particles, e.g., lentivirus particles, or plasmids for propagating the Type II Cas and gRNA coding sequences in bacterial (e.g., E. coli) or eukaryotic (e.g., yeast) cells.

A nucleic acid encoding a Type II Cas protein can, in some embodiments, further encode a gRNA. Alternatively, a gRNA can be encoded by a separate nucleic acid (e.g., DNA or mRNA). Nucleic acids encoding a Type II Cas protein can be codon optimized, e.g., where at least one non-common codon or less-common codon has been replaced by a codon that is common in a host cell. For example, a codon optimized nucleic acid can direct the synthesis of an optimized messenger mRNA, e.g., optimized for expression in a mammalian expression system. As an example, if the intended target nucleic acid is within a human cell, a human codon-optimized polynucleotide encoding Type II Cas can be used for producing a Type II Cas polypeptide. Exemplary codon-optimized sequences are shown in Table 1.

Nucleic acids of the disclosure, e.g., plasmids and viral vectors, can comprise one or more regulatory elements such as promoters, enhancers, and other expression control elements (e.g., transcription termination signals, such as polyadenylation signals and poly-U sequences). Such regulatory elements are described, for example, in Goeddel, 1990, GENE EXPRESSION TECHNOLOGY: METHODS IN ENZYMOLOGY 185, Academic Press, San Diego, Calif. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest or in particular cell types. Regulatory elements may also direct expression in a temporal-dependent manner, such as in a cell-cycle dependent or developmental stage-dependent manner, which may or may not also be tissue or cell-type specific. In some embodiments, a nucleic acid of the disclosure comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof, e.g., to express a Type II Cas protein and a gRNA separately. Examples of pol III promoters include, but are not limited to, U6 and H1 promoters. Examples of pol II promoters include, but are not limited to, the retroviral Rous Sarcoma virus (RSV) LTR promoter (optionally with the RSV enhancer), the cytomegalovirus (CMV) promoter (optionally with the CMV enhancer) (see, e.g., Boshart et al, 1985, Cell 41:521-530), the SV40 promoter, the dihydrofolate reductase promoter, the β-actin promoter, the phosphoglycerol kinase (PGK) promoter, and EF1α promoters (for example, full length EF1α promoter and the EFS promoter, which is a short, intron-less form of the full EF1α promoter). Exemplary enhancer elements include WPRE; CMV enhancers; the R-U5′ segment in LTR of HTLV-I; SV40 enhancer; and the intron sequence between exons 2 and 3 of rabbit β-globin. It will be appreciated by those skilled in the art that the design of an expression vector can depend on such factors as the choice of the host cell, the level of expression desired, etc.

The term “vector” refers to a polynucleotide molecule capable of transporting another nucleic acid to which it has been linked. One type of polynucleotide vector includes a “plasmid”, which refers to a circular double-stranded DNA loop into which additional nucleic acid segments are or can be ligated. Another type of polynucleotide vector is a viral vector; wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some examples, vectors can be capable of directing the expression of nucleic acids to which they are operably linked. Such vectors can be referred to herein as “recombinant expression vectors”, or more simply “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, CA (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Vectors can include, but are not limited to, viral vectors based on vaccinia virus, poliovirus, adenovirus, adeno-associated virus (e.g., AAV2, AAV5, AAV7m8, AAV8, AAV9, AAVrh8r, AAVrh10), SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXTI, pSG5, pSVK3, pBPV, pMSG, and pSVLSV40 (Pharmacia). Additional vectors contemplated for eukaryotic target cells include, but are not limited to, the vectors pCTx-I, pCTx-2, and pCTx-3. Other vectors can be used so long as they are compatible with the host cell.

In some examples, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. The vector can be a self-inactivating vector that either inactivates the viral sequences or the components of the CRISPR machinery or other elements.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-l promoters (for example, the full EF1α promoter and the EFS promoter), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-l.

An expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, for example a human RHO promoter or human rhodopsin kinase promoter (hGRK), a cell type specific promoter, etc.).

### 6.6. Particles and Cells

The disclosure further provides particles comprising a Type II Cas protein of the disclosure (e.g., an ENQP Type II Cas protein), particles comprising a gRNA of the disclosure, particles comprising a system of the disclosure, and particles comprising a nucleic acid or plurality of nucleic acids of the disclosure. The particles can in some embodiments comprise or further comprise a gRNA, or a nucleic acid encoding the gRNA (e.g., DNA or mRNA). For example, the particles can comprise a RNP of the disclosure. Exemplary particles include lipid nanoparticles, vesicles, viral-like particles (VLPs) and gold nanoparticles. See, e.g., WO 2020/012335, the contents of which are incorporated herein by reference in their entireties, which describes vesicles that can be used to deliver gRNA molecules and Type II Cas proteins to cells (e.g., complexed together as a RNP).

The disclosure provides particles (e.g., virus particles) comprising a nucleic acid encoding a Type II Cas protein of the disclosure. The particles can further comprise a nucleic acid encoding a gRNA. Alternatively, a nucleic acid encoding a Type II Cas protein can further encode a gRNA.

The disclosure further provides pluralities of particles (e.g., pluralities of virus particles). Such pluralities can include a particle encoding a Type II Cas protein and a different particle encoding a gRNA. For example, a plurality of particles can comprise a virus particle (e.g., a AAV2, AAV5, AAV7m8, AAV8, AAV9, AAVrh8r, or AAVrh10 virus particle) encoding a Type II Cas protein and a second virus particle (e.g., a AAV2, AAV5, AAV7m8, AAV8, AAV9, AAVrh8r, or AAVrh10 virus particle) encoding a gRNA. Alternatively, a plurality of particles can comprise a plurality of virus particles where each particle encodes a Type II Cas protein and a gRNA.

The disclosure further provides cells and populations of cells (e.g., ex vivo cells and populations of cells) that can comprise a Type II Cas protein (e.g., introduced to the cell as a RNP) or a nucleic acid encoding the Type II Cas protein (e.g., DNA or mRNA) (optionally also encoding a gRNA). The disclosure further provides cells and populations of cells comprising a gRNA of the disclosure (optionally complexed with a Type II Cas protein) or a nucleic acid encoding the gRNA (e.g., DNA or mRNA) (optionally also encoding a Type II Cas protein). The cells and populations of cells can be, for example, human cells such as a stem cell, e.g., a hematopoietic stem cell (HSC), a pluripotent stem cell, an induced pluripotent stem cell (iPS), or an embryonic stem cell. In some embodiments, the cells and populations of cells are T cells. Methods for introducing proteins and nucleic acids to cells are known in the art. For example, a RNP can be produced by mixing a Type II Cas protein and one or more guide RNAs in an appropriate buffer. An RNP can be introduced to a cell, for example, via electroporation and other methods known in the art.

The cell populations of the disclosure can be cells in which gene editing by the systems of the disclosure has taken place, or cells in which the components of a system of the disclosure have been introduced or expressed but gene editing has not taken place, or a combination thereof. A cell population can comprise, for example, a population in which at least 1%, at least 5%, at least 10%, at least 15%, at least 20%, at least 30%, at least 40%, at least 50%, at least 60%, or at least 70% of the cells have undergone gene editing by a system of the disclosure.

### 6.7. Pharmaceutical Compositions

Also disclosed herein are pharmaceutical formulations and medicaments comprising a Type II Cas protein, gRNA, nucleic acid or plurality of nucleic acids, system, particle, or plurality of particles of the disclosure together with a pharmaceutically acceptable excipient.

Suitable excipients include, but are not limited to, salts, diluents, (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, antinfective agents, suspending agents, wetting agents, viscosity modifiers, tonicity agents, stabilizing agents, and other components and combinations thereof. Suitable pharmaceutically acceptable excipients can be selected from materials which are generally recognized as safe (GRAS), and may be administered to an individual without causing undesirable biological side effects or unwanted interactions. Suitable excipients and their formulations are described in Remington's Pharmaceutical Sciences, 16th ed. 1980, Mack Publishing Co. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions.

The components of the pharmaceutical formulation can be dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol.

In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers.

In some embodiments, the formulation can be distributed or packaged in a liquid form, or alternatively, as a solid, obtained, for example by lyophilization of a suitable liquid formulation, which can be reconstituted with an appropriate carrier or diluent prior to administration. In some embodiments, the formulations can comprise a guide RNA and a Type II Cas protein in a pharmaceutically effective amount sufficient to edit a gene in a cell. The pharmaceutical compositions can be formulated for medical and/or veterinary use.

### 6.8. Methods of Altering a Cell

The disclosure further provides methods of using the Type II Cas proteins, gRNAs, nucleic acids (including pluralities of nucleic acids), systems, and particles (including pluralities of particles) of the disclosure for altering cells.

In one aspect, a method of altering a cell comprises contacting a eukaryotic cell (e.g., a human cell) with a nucleic acid, particle, system or pharmaceutical composition described herein.

Contacting a cell with a disclosed nucleic acid, particle, system or pharmaceutical composition can be achieved by any method known in the art and can be performed in vivo, ex vivo, or in vitro. In some embodiments, the methods can include obtaining one or more cells from a subject prior to contacting the cell(s) with a herein disclosed nucleic acid, particle, system or pharmaceutical composition. In some embodiments, the methods can further comprise returning or implanting the contacted cell or a progeny thereof to the subject.

Type II Cas and gRNA, as well as nucleic acids encoding Type II Cas and gRNAs can be delivered to a cell by any means known in the art, for example, by viral or non-viral delivery vehicles, electroporation or lipid nanoparticles.

A polynucleotide encoding Type II Cas and a gRNA, can be delivered to a cell (ex vivo or in vivo) by a lipid nanoparticle (LNP). LNPs can have, for example, a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm. LNPs can be made from cationic, anionic, neutral lipids, and combinations thereof. Neutral lipids, such as the fusogenic phospholipid DOPE or the membrane component cholesterol, can be included in LNPs as ‘helper lipids’ to enhance transfection activity and nanoparticle stability.

LNPs can also be comprised of hydrophobic lipids, hydrophilic lipids, or both hydrophobic and hydrophilic lipids. Lipids and combinations of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerCl4, and PEG-CerC20. Lipids can be combined in any number of molar ratios to produce a LNP. In addition, the polynucleotide(s) can be combined with lipid(s) in a wide range of molar ratios to produce a LNP.

Type II Cas and/or gRNAs can be delivered to a cell via an adeno-associated viral vector (e.g., of an AAV2, AAV5, AAV7m8, AAV8, AAV9, AAVrh8r, or AAVrh10 serotype), or by another viral vector. Other viral vectors include, but are not limited to lentivirus, adenovirus, alphavirus, enterovirus, pestivirus, baculovirus, herpesvirus, Epstein Barr virus, papovavirus, poxvirus, vaccinia virus, and herpes simplex virus. In some embodiments, a Type II Cas mRNA is formulated in a lipid nanoparticle, while a sgRNA is delivered to a cell in an AAV or other viral vector. In some embodiments, one or more AAV vectors (e.g., one or more AAV2, AAV5, AAV7m8, AAV8, AAV9, AAVrh8r, or AAVrh10 serotype) are used to deliver both a sgRNA and a Type II Cas. In some embodiments, a Type II Cas and a sgRNA are delivered using separate vectors. In other embodiments, a Type II Cas and a sgRNA are delivered using a single vector. ENQP Type II Cas, with its relatively small size, can be delivered with a gRNA (e.g., sgRNA) using a single AAV vector.

Compositions and methods for delivering Type II Cas and gRNAs to a cell and/or subject are further described in PCT Patent Application Publications WO 2019/102381, WO 2020/012335, and WO 2020/053224, each of which is incorporated by reference herein in its entirety.

DNA cleavage can result in a single-strand break (SSB) or double-strand break (DSB) at particular locations within the DNA molecule. Such breaks can be and regularly are repaired by natural, endogenous cellular processes, such as homology-dependent repair (HDR) and non-homologous end-joining (NHEJ). These repair processes can edit the targeted polynucleotide by introducing a mutation, thereby resulting in a polynucleotide having a sequence which differs from the polynucleotide's sequence prior to cleavage by a Type II Cas.

NHEJ and HDR DNA repair processes consist of a family of alternative pathways. Non-homologous end-joining (NHEJ) refers to the natural, cellular process in which a double-stranded DNA-break is repaired by the direct joining of two non-homologous DNA segments. See, e.g. Cahill et al., 2006, Front. Biosci. 11:1958-1976. DNA repair by non-homologous end-joining is error-prone and frequently results in the untemplated addition or deletion of DNA sequences at the site of repair. Thus, NHEJ repair mechanisms can introduce mutations into the coding sequence which can disrupt gene function. NHEJ directly joins the DNA ends resulting from a double-strand break, sometimes with a modification of the polynucleotide sequence such as a loss of or addition of nucleotides in the polynucleotide sequence. The modification of the polynucleotide sequence can disrupt (or perhaps enhance) gene expression.

Homology-dependent repair (HDR) utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus.

A third repair mechanism includes microhomology-mediated end joining (MMEJ), also referred to as “Alternative NHEJ (ANHEJ)”, in which the genetic outcome is similar to NHEJ in that small deletions and insertions can occur at the cleavage site. MMEJ can make use of homologous sequences of a few base pairs flanking the DNA break site to drive a more favored DNA end joining repair outcome. In some instances, it may be possible to predict likely repair outcomes based on analysis of potential microhomologies at the site of the DNA break.

Modifications of a cleaved polynucleotide by HDR, NHEJ, and/or ANHEJ can result in, for example, mutations, deletions, alterations, integrations, gene correction, gene replacement, gene tagging, transgene insertion, nucleotide deletion, gene disruption, translocations and/or gene mutation. The aforementioned process outcomes are examples of editing a polynucleotide.

Advantages of ex vivo cell therapy approaches include the ability to conduct a comprehensive analysis of the therapeutic prior to administration. Nuclease-based therapeutics can have some level of off-target effects. Performing gene correction ex vivo allows a method user to characterize the corrected cell population prior to implantation, including identifying any undesirable off-target effects. Where undesirable effects are observed, a method user may opt not to implant the cells or cell progeny, may further edit the cells, or may select new cells for editing and analysis. Other advantages include ease of genetic correction in iPSCs compared to other primary cell sources. iPSCs are prolific, making it easy to obtain the large number of cells that will be required for a cell-based therapy. Furthermore, iPSCs are an ideal cell type for performing clonal isolations. This allows screening for the correct genomic correction, without risking a decrease in viability.

Although certain cells present an attractive target for ex vivo treatment and therapy, increased efficacy in delivery may permit direct in vivo delivery to such cells. Ideally the targeting and editing is directed to the relevant cells. Cleavage in other cells can also be prevented by the use of promoters only active in certain cell types and/or developmental stages.

Additional promoters are inducible, and therefore can be temporally controlled if the nuclease is delivered as a plasmid. The amount of time that delivered protein and RNA remain in the cell can also be adjusted using treatments or domains added to change the half-life. In vivo treatment would eliminate a number of treatment steps, but a lower rate of delivery can require higher rates of editing. In vivo treatment can eliminate problems and losses from ex vivo treatment and engraftment.

An advantage of in vivo gene therapy can be the ease of therapeutic production and administration. The same therapeutic approach and therapy has the potential to be used to treat more than one patient, for example a number of patients who share the same or similar genotype or allele. In contrast, ex vivo cell therapy typically requires using a subject's own cells, which are isolated, manipulated and returned to the same patient.

Progenitor cells (also referred to as stem cells herein) are capable of both proliferation and giving rise to more progenitor cells, which in turn have the ability to generate a large number of cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell can derive from a multipotent cell that itself is derived from a multipotent cell, and so on. While each of these multipotent cells can be considered stem cells, the range of cell types that each can give rise to can vary considerably. Some differentiated cells also have the capacity to give rise to cells of greater developmental potential. Such capacity can be natural or can be induced artificially upon treatment with various factors. In many biological instances, stem cells can also be “multipotent” because they can produce progeny of more than one distinct cell type, but this is not required.

Human cells described herein can be induced pluripotent stem cells (iPSCs). An advantage of using iPSCs in the methods of the disclosure is that the cells can be derived from the same subject to which the progenitor cells are to be administered. That is, a somatic cell can be obtained from a subject, reprogrammed to an induced pluripotent stem cell, and then differentiated into a progenitor cell to be administered to the subject (e.g., an autologous cell). Because progenitors are essentially derived from an autologous source, the risk of engraftment rejection or allergic response can be reduced compared to the use of cells from another subject or group of subjects. In addition, the use of iPSCs negates the need for cells obtained from an embryonic source. Thus, in one aspect, the stem cells used in the disclosed methods are not embryonic stem cells.

Methods are known in the art that can be used to generate pluripotent stem cells from somatic cells. Pluripotent stem cells generated by such methods can be used in the method of the disclosure.

Reprogramming methodologies for generating pluripotent cells using defined combinations of transcription factors have been described. Mouse somatic cells can be converted to ES cell-like cells with expanded developmental potential by the direct transduction of Oct4, Sox2, Klf4, and c-Myc; see, e.g., Takahashi and Yamanaka, 2006, Cell 126(4): 663-76. iPSCs resemble ES cells, as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission (see, e.g., Maherali and Hochedlinger, 2008, Cell Stem Cell. 3(6):595-605), and tetraploid complementation.

Human iPSCs can be obtained using similar transduction methods, and the transcription factor trio, OCT4, SOX2, and NANOG, has been established as the core set of transcription factors that govern pluripotency; see, e.g., 2014, Budniatzky and Gepstein, Stem Cells Transl Med. 3(4):448-57; Barrett et al, 2014, Stem Cells Trans Med 3:1-6 sctm.2014-0121; Focosi et al, 2014, Blood Cancer Journal 4: e211. The production of iPSCs can be achieved by the introduction of nucleic acid sequences encoding stem cell-associated genes into an adult, somatic cell, historically using viral vectors.

iPSCs can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., 2010, Cell Stem Cell, 7(5):618-30. Reprogramming can be achieved by introducing a combination of nucleic acids encoding stem cell-associated genes, including, for example, Oct-4 (also known as Oct-3/4 or Pouf5l), SoxI, Sox2, Sox3, Sox 15, Sox 18, NANOG, KlfI, Klf2, Klf4, Klf5, NR5A2, c-Myc, 1-Myc, n-Myc, Rem2, Tert, and LIN28. Reprogramming using the methods and compositions described herein can further comprise introducing one or more of Oct-3/4, a member of the Sox family, a member of the Klf family, and a member of the Myc family to a somatic cell. The methods and compositions described herein can further comprise introducing one or more of each of Oct-4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one aspect the reprogramming is not affected by a method that alters the genome. Thus, in such examples, reprogramming can be achieved, e.g., without the use of viral or plasmid vectors.

Efficiency of reprogramming (the number of reprogrammed cells) derived from a population of starting cells can be enhanced by the addition of various agents, e.g., small molecules, as shown by Shi et al., 2008, Cell-Stem Cell 2:525-528; Huangfu et al., 2008, Nature Biotechnology 26(7):795-797; and Marson et al., 2008, Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HD AC) inhibitors, valproic acid, 5′-azacytidine, dexamethasone, suberoylanilide, hydroxamic acid (SAHA), vitamin C, and trichostatin (TSA), among others. Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-IH,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pi valoyloxy methyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylureido) caproic hydroxamic acid), AOE (2-amino-8-oxo-9, 10-epoxy decanoic acid), CHAP31 and CHAP 50. Other reprogramming enhancing agents include, for example, dominant negative forms of the HDACs (e.g, catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Titan Pharmaceuticals, MethylGene, and Sigma Aldrich.

To confirm the induction of pluripotent stem cells, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbxl5, Ecatl, Esgl, Eras, Gdfi, Fgf4, Cripto, Daxl, Zpf296, Slc2a3, Rexl, Utfl, and Natl. In one case, for example, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. Detection can involve not only RT-PCR, but also detection of protein markers. Intracellular markers can be best identified via RT-PCR, or protein detection methods such as immunocytochemistry, while cell surface markers are readily identified, e.g., by immunocytochemistry.

Pluripotency of isolated cells can be confirmed by tests evaluating the ability of the iPSCs to differentiate into cells of each of the three germ layers. As one example, teratoma formation in nude mice can be used to evaluate the pluripotent character of the isolated clones. The cells can be introduced into nude mice and histology and/or immunohistochemistry can be performed on a tumor arising from the cells. The growth of a tumor comprising cells from all three germ layers, for example, further indicates that the cells are pluripotent stem cells.

Patient-specific iPS cells or cell line can be created. There are many established methods in the art for creating patient specific iPS cells, e.g., as described in Takahashi and Yamanaka 2006; Takahashi, Tanabe et al. 2007. For example, the creating step can comprise: a) isolating a somatic cell, such as a skin cell or fibroblast, from the patient; and b) introducing a set of pluripotency-associated genes into the somatic cell in order to induce the cell to become a pluripotent stem cell. The set of pluripotency-associated genes can be one or more of the genes selected from the group consisting of OCT4, SOX1, SOX2, SOX3, SOX15, SOX18, NANOG, KLF1, KLF2, KLF4, KLF5, c-MYC, n-MYC, REM2, TERT and LIN28.

In some aspects, a biopsy or aspirate of a subject's bone marrow can be performed. A biopsy or aspirate is a sample of tissue or fluid taken from the body. There are many different kinds of biopsies or aspirates. Nearly all of them involve using a sharp tool to remove a small amount of tissue. If the biopsy will be on the skin or other sensitive area, numbing medicine can be applied first. A biopsy or aspirate can be performed according to any of the known methods in the art. For example, in a bone marrow aspirate, a large needle is used to enter the pelvis bone to collect bone marrow.

In some aspects, a mesenchymal stem cell can be isolated from a subject. Mesenchymal stem cells can be isolated according to any method known in the art, such as from a subject's bone marrow or peripheral blood. For example, marrow aspirate can be collected into a syringe with heparin. Cells can be washed and centrifuged on a Percoll™ density gradient. Cells, such as blood cells, liver cells, interstitial cells, macrophages, mast cells, and thymocytes, can be separated using density gradient centrifugation media, Percoll™. The cells can then be cultured in Dulbecco's modified Eagle's medium (DMEM) (low glucose) containing 10% fetal bovine serum (FBS) (Pittinger et. al., 1999, Science 284:143-147).

### 6.8.1. Exemplary Genomic Targets

The Type II Cas proteins and gRNAs of the disclosure can be used to alter various genomic targets. In some aspects, the methods of altering a cell are methods for altering a DNMT1 or RHO genomic sequence. In some aspects, the methods of altering a cell are methods of altering a TRAC, B2M, PD1, or LAG3 genomic sequence. Reference sequences of DNMT1, RHO, TRAC, B2M, PD1, and LAG3 are available in public databases, for example those maintained by NCBI. For example, DNMT1 has the NCBI gene ID 1786; RHO has the NCBI gene ID: 6010; TRAC has the NCBI gene ID: 28755; B2M has the NCBI gene ID: 567; PD1 has the NCBI gene ID: 5133; and LAG3 has the NCBI gene ID: 3902.

In some embodiments, the methods of altering a cell are methods for altering a DNMT1 gene. Mutations in the DNMT1 gene can cause DNMT1-related disorder, which is a degenerative disorder of the central and peripheral nervous systems. DNMT1-related disorder is characterized by sensory impairment, loss of sweating, dementia, and hearing loss.

In some embodiments, the methods of altering a cell are methods for altering a RHO gene. Mutations in the RHO gene can cause retinitis pigmentosa (RP).

Allele specific editing of human RHO alleles having pathogenic mutations (e.g., a P23 mutation such as P23H or a P347 mutation such as P347L, P347S, P347R, P347Q, P347T, or P347A) can be achieved using guide RNA (gRNA) molecules targeting the rs7984 SNP (for example having spacers as shown in Table 4A or Table 4B) located in the 5′ untranslated region (UTR) of the RHO gene. SNPs are very common in the human population, and a significant proportion of subjects are heterozygous for the rs7984 SNP. For a subject heterozygous for the rs7984 SNP and heterozygous for a pathogenic RHO gene mutation, allele specific editing of the RHO allele having the pathogenic mutation can be achieved through the use of a gRNA targeting the SNP variant found in the subject's RHO allele having the pathogenic mutation. This allele-specific editing strategy, which does not directly target a specific pathogenic RHO gene mutation, advantageously allows editing of RHO genes having a variety of different pathogenic mutations. A rs7984 SNP targeting gRNA of the disclosure can be used in combination with a second gRNA targeting a second site in the RHO gene, for example a site in intron 1 (e.g., a gRNA having a spacer as shown in Table 4C), to promote two cuts in the RHO gene having the pathogenic mutation. Cleaving the RHO gene having the pathogenic mutation at two sites can promote a deletion in the RHO gene having the pathogenic mutation, which can result in reduced mutant RHO protein expression.

Editing a subject's RHO allele can comprise editing a RHO allele in one or more cells from the subject (e.g., photoreceptor cells or retinal progenitor cells) or one or more cells derived from a cell of the subject (e.g., an induced pluripotent stem cell (iPSC)). For example, one or more cells from the subject or one or more cells derived from a cell of the subject can be contacted with a nucleic acid, system, or particle of the disclosure ex vivo, and cells having an edited RHO gene or progeny thereof can subsequently be implanted into the subject. Edited iPSCs can be differentiated, for instance into photoreceptor cells or retinal progenitor cells. In some embodiments, resultant differentiated cells can be implanted into the subject. When differentiated cells from the subject are edited, implantation of edited cells can proceed without an intervening differentiation step.

An in vivo method of RHO allele editing can comprise editing a RHO allele having a pathogenic mutation in a cell of a subject, such as photoreceptor cells or retinal progenitor cells. In some embodiments, the in vivo methods comprise administering one or more pharmaceutical compositions of the disclosure to or near the eye of a subject, e.g., by sub-retinal injection or intravitreal injection. For example, a single pharmaceutical composition comprising one or more AAV particles encoding one or more gRNAs (e.g., a gRNA targeting the rs7984 SNP and a gRNA targeting RHO intron 1) and a Type II Cas protein of the disclosure can be used; or alternatively, multiple pharmaceutical compositions can be used, for example a first pharmaceutical composition comprising an AAV particle encoding the gRNA(s) and a second, separate pharmaceutical composition comprising a second AAV particle encoding the Type II Cas protein. When multiple pharmaceutical compositions are used, they are preferably administered sufficiently close in time so that the gRNA(s) and Type II Cas protein provided by the pharmaceutical compositions are present together in vivo.

Targeting of (one or more of) human TRAC, human B2M, human PD1, and human LAG3 genes can be used, for example, in the engineering of chimeric antigen receptor (CAR) T cells. For example, CRISPR/Cas technology has been used to deliver CAR-encoding DNA sequences to loci such as TRAC and PD1 (see, e.g., Eyquem et al., 2017, Nature 543(7643):113-117; Hu et al., 2023, eClinicalMedicine 60:102010), while TRAC, B2M, PD1, and LAG3 knockout CAR T-cells have been reported (see, e.g., Dimitri et al., 2022, Molecular Cancer 21:78; Liu et al., 2016, Cell Research 27:154-157; Ren et al., 2017, Clin Cancer Res. 23(9):2255-2266; Zhang et al., 2017, Front Med. 11(4):554-562). Thus, the Type II Cas proteins and TRAC, B2M, PD1, and LAG3 guides of the disclosure can be used for targeted knock-in of an exogenous DNA sequence to a desired genomic site in a human cell and/or knock-out of TRAC, B2M, PD1, or LAG3 in a human cell, for example a human T cell. In some embodiments, T cells are edited ex vivo to produce CAR-T cells and subsequently administered to a subject in need of CAR-T cell therapy.

### 7. EXAMPLES

### 7.1. Example 1: Identification and Characterization of ENQP Type II Cas Protein

This Example describes studies performed to identify and characterize ENQP Type II Cas protein.

### 7.1.1. Materials and Methods

### 7.1.1.1. Identification of ENQP Type II Cas Protein From Metagenomic Data

154,723 bacterial and archaeal metagenome-assembled genomes (MAGs) reconstructed from the human microbiome (Pasolli, et al., 2019, Cell 176(3):649-662.e20) were screened in order to find new Type II Cas proteins. cas1, cas2 and cas9 genes were identified from the protein annotation, performed with Prokka version 1.12 (Seemann, 2014, Bioinformatics 30(14):2068-2069). CRISPR arrays were identified using MinCED version 0.4.2 (with default parameters) (Bland, et al., 2007, BMC bioinformatics 8:209). Only loci having a CRISPR array and cas1-2-9 genes at a maximum distance of 10 kbp from each other were considered. Loci containing Type II Cas proteins shorter than 950 aa were discarded. The resulting 17,173 CRISPR-Type II loci were filtered by selecting short proteins (less than 1100 aa) from putative unknown species. Type II Cas proteins from the same species, having similar length but slightly different sequence, were compared by multiple sequence alignment. Proteins presenting deletions in nucleasic domains were discarded. The remaining proteins were compared for sequencing coverage and the ortholog with the highest coverage was selected for each species.

### 7.1.1.2. Plasmids

A CAG-driven expression plasmid was used to express the ENQP Type II Cas in mammalian cells. Briefly, a human codon-optimized coding sequence of ENQP Type II Cas and exemplary sgRNA scaffolds (full length or trimmed, reported in Table 6) were cloned into the aforementioned expression plasmid, generating pX-ENQP-full and pX-ENQP-trim. In creating both ENQP sgRNA designs the last six bases of the cRNA were removed and substituted with a GAAA tetraloop to promote folding. Unless otherwise stated the trimmed sgRNA scaffold was used in editing studies. The ENQP Type II Cas coding sequence, modified by the addition of an SV5 tag at the N-terminus and two nuclear localization signals (one at the N-terminus and one at the C-terminus) and human codon-optimized, as well as the sgRNA scaffolds, were obtained as synthetic fragments from Genewiz. Spacer sequences were cloned into the pX-ENQP plasmids as annealed DNA oligonucleotides containing a variable 24-nt spacer sequence using a double BsaI site present in the plasmid. The list of spacer sequences and relative cloning oligonucleotides used in the present Example is reported in Table 7.

TABLE 6
ENQP sgRNA scaffolds

| | | SEQ ID |
|---|---|---|
| Scaffold | Sequence | NO : |
| ENQP full | GUCUUGAGCACGCACCCUU | 46 |
| scaffold | CCCCAAGGUGAGAAAUCAC | |
| (sgRNAFS) | CUUGGGGAAGGGUGCGGCU | |
| (including six | CCAGACAAGGGAAGUCAGC | |
| 3′ uracils) | UAUCUGACUUACCCGUAAA | |
| | GUUACCCCCGCACCGUCCU | |
| | CGGACGAUGCGGGGCGAAC | |
| | UUUUUU | |
| ENQP trimmed | GUCUUGAGCACGCGAAAGC | 47 |
| scaffold | GGCUCCAGACAAGGGAAGU | |
| (sgRNAtrimV1) | CAGCUAUCUGACUUACCCG | |
| (including six | UAAAGUUACCCCCGCACCG | |
| 3′ uracils) | UCCUCGGACGAUGCGGGGC | |
| | GAACUUUUUU | |

TABLE 7
Sequences of the oligonucleotides used for cloning sgRNA
spacers and sequences of their relative target sites

| Spacer sequences used | | | | | Oligo used to clone the | | | |
|---|---|---|---|---|---|---|---|---|
| in reporter assays | | | | | spacer in pX plasmid (**) | | | |
| | | SEQ | | SEQ | | SEQ | | SEQ |
| | | ID | | ID | Oligo 1 | ID | Oligo 2 | ID |
| Name | Protospacer | NO: | Target (*) | NO: | (5′>3′) | NO: | (5′>3′) | NO: |
| gRNA1_ | GGCGATGCC | 48 | gagGGCGATGCC | 49 | caccGGCGA | 50 | agacAGCTTGCCGTAG | 51 |
| ENQP_ | ACCTACGGC | | ACCTACGGCAAG | | TGCCACCTA | | GTGGCATCGCC | |
| EGFP | AAGCT | | CTGACCCTGAagt | | CGGCAAGCT | | | |
| gRNA2_ | ACGGCGTGCAGTG | 52 | cctACGGCGTGC | 53 | caccGACGG | 54 | agacGCGGCTGAAGC | 55 |
| ENQP_ | | | AGTGCTTCAGCC | | CGTGCAGTG | | ACTGCACGCCGTC | |
| EGFP | CTTCAGCCGC | | GCTACCCCGAcca | | CTTCAGCCGC | | | |
| Spacer sequences to | | | | | Oligo used to clone the | | | |
| target endogenous loci | | | | | spacer in pX plasmid (**) | | | |
| | | SEQ | | SEQ | | SEQ | | SEQ |
| | | ID | | ID | Oligo 1 | ID | Oligo 2 | ID |
| Name | Protospacer | NO: | Target (*) | NO: | (5′>3′) | NO: | (5′>3′) | NO: |
| gRNA2_ | CTTGTGGCTGACC | 56 | gccCTTGTGGC | 57 | caccGCTTGTGGCTGA | 58 | agacGAGCAGCCACG | 59 |
| ENQP_ | CGTGGCTGCTC | | TGACCCGTGGCT | | CCCGTGGCTGCTC | | | |
| RHO_A | | | GCTCCCACCCAA | | | | GGTCAGCCACAAGC | |
| | | | gaa | | | | | |
| gRNA5_ | CTTGGGTGGGAGC | 60 | attCTTGGGTG | 61 | caccGCTTGGGTGGGA | 62 | agacACCCGTGGCTG | 63 |
| ENQP_ | AGCCACGGGT | | GGAGCAGCCACG | | GCAGCCACGGGT | | CTCCCACCCAAGC | |
| RHO_A | | | GGTCAGCCACAa | | | | | |
| | | | gg | | | | | |
| gRNA6_ | GGAGCAGCCACG | 64 | gtgGGAGCAGCC | 65 | caccGGAGCAGCCACG | 66 | agacTTGTGGCTGACC | 67 |
| ENQP | GGTCAGCCACAA | | ACGGGTCAGCCA | | GGTCAGCCACAA | | CGTGGCTGCTCC | |
| RHO_A | | | CAAGGGCCACAg | | | | | |
| | | | cc | | | | | |
| gRNA1_ | GTGCCAGAAACAG | 68 | ctgGTGCCAGAA | 69 | caccGTGCCAGAAACA | 70 | agacCCCGTCACCCCT | 71 |
| ENQP_ | GGGTGACGGG | | ACAGGGGTGACG | | GGGGTGACGGG | | GTTTCTGGCAC | |
| DNMT1 | | | GGAGGGCAGAac | | | | | |
| | | | t | | | | | |

(*) The target sequences are reported with three flanking nucleotides on each side.
The PAM sequence is highlighted in bold.
(**) The cloning overhang is reported in lowercase.
Nucleotides highlighted in bold represent 5′-G appended to favor transcription from canonical U6 Pol III promoters.

### 7.1.1.3. Cell Lines

HEK293T cells (obtained from ATCC), U2OS-EGFP cells (harboring a single integrated copy of an EGFP reporter gene) and HEK293-RHO-EGFP cells were cultured in DMEM (Life Technologies) supplemented with 10% FBS (Life Technologies), 2 mM GlutaMax™ (Life Technologies) and penicillin/streptomycin (Life Technologies). HEK293-RHO-EGFP cells were obtained by stable transfection of HEK293 cells with a RHO-EGFP reporter plasmid, obtained by cloning a fragment of the RHO gene up to exon 2 (retaining introns 1 and 2) fused to part of RHO cDNA containing exons 3-5 in frame with the EGFP coding sequence into a CMV-driven expression plasmid. Cells were pool-selected with 5 μg/ml Hygromycin (Invivogen) and single clones were subsequently isolated and expanded. All cells were incubated at 37° C. and 5% CO₂ in a humidified atmosphere. All cells tested mycoplasma negative (PlasmoTest, Invivogen).

7.1.1.4. tracrRNA Identification

Identification of tracrRNAs for ENQP Type II Cas protein was performed with a method based on a work by Chyou and Brown (Chyou and Brown, 2019, RNA biology 16(4):423-434). Starting from unique direct repeats in the CRISPR array, BLAST version 2.2.31 (with parameters-task blastn-short-gapopen 2-gapextend 1-penalty-1-reward 1-evalue 1-word_size 8) was used to identify anti-repeats within a 3000 bp window flanking the CRISPR-Type II Cas locus. A custom version of RNIE (Gardner, et al., 2011, Nucleic Acids Research 39(14):5845-5852) was used to predict Rho-independent transcription terminators (RITs) near anti-repeats. Putative tracrRNA sequences, starting with an anti-repeat and ending with either a RIT (when found) or a poly-T, were combined with directed repeats to form sgRNA scaffolds. The secondary structure of sgRNA scaffolds was predicted using RNAsubopt version 2.4.14 (with parameters—noLP-e 5) (Lorenz, et al., 2011, Algorithms for Molecular Biology 6(1):26). sgRNAs lacking the functional modules identified by (Briner, et al., 2014, Molecular Cell 56(2):333-339), namely the repeat: anti-repeat duplex, nexus and 3′ hairpin-like folds, were discarded.

### 7.1.1.5. Construction of a Randomized PAM Library

A randomized PAM library was prepared as described in Kleinstiver et al., (Kleinstiver, et al., 2015, Nature 523(7561):481-485). Briefly: one synthetic DNA oligonucleotide containing an EcoRI site and a 8-nt randomized sequence (top oligo) was obtained from Eurofins, together with another DNA oligo that anneals to the 3′ region flanking the randomized sequence leaving an SphI-compatible end (bottom oligo). The bottom strand of the annealed oligo duplex was filled-in incubating with Klenow (exo-) and digested with EcoRI for ligation into a suitable destination plasmid. The ligation product was then electroporated into MegaX DH10B™ T1R Electrocompetent Cells (Thermo Scientific) to reach a theoretical library coverage of 100×. Colonies were harvested and the plasmid DNA was purified by maxi-prep (Macherey-Nagel). Two PCR steps (Phusion® HF DNA polymerase, Thermo Fisher Scientific) were performed to prepare the plasmid PAM library for NGS analysis to verify proper complexity: the first, using a set of forward primers and two different reverse primers, to amplify the region containing the protospacer and the randomized PAM and the second to attach the Illumina Nextera™ DNA indexes and adapters (Table 8). PCR products were purified using Agencourt AMPure™ beads in a 1:0.8 ratio. The library was analyzed with a 150-bp single read sequencing, using a v3 flow cell on an Illumina MiSeq sequencer.

TABLE 8
Sequences of the primers used for NGS library
preparation to validate the generated
PAM plasmid library

| | | SEQ |
|---|---|---|
| | | ID |
| Primer name | Sequence (5′ → 3′) | NO: |
| F1a | TCGTCGGCAGCGTCAGATGTGT | 72 |
| | ATAAGAGACAGAAAACACACCG | |
| | CATACGTACGATTTA | |
| F1b | TCGTCGGCAGCGTCAGATGTGT | 73 |
| | ATAAGAGACAGTTTAATCACAC | |
| | CGCATACGTACGATTTA | |
| R1 | GTCTCGTGGGCTCGGAGATGTG | 74 |
| | TATAAGAGACAGCGTTCTGATT | |
| | TAATCTGTATCAGGC | |
| F2a | TCGTCGGCAGCGTCAGATGTGT | 75 |
| | ATAAGAGACAGTTTTTGGTTAC | |
| | GCATCTGTGCGGTATTTC | |
| F2b | TCGTCGGCAGCGTCAGATGTGT | 76 |
| | ATAAGAGACAGCCCTTACGCAT | |
| | CTGTGCGGTATTTC | |
| F3a | TCGTCGGCAGCGTCAGATGTGT | 77 |
| | ATAAGAGACAGTATCGATTTAA | |
| | ATAGGCCTGACTCAC | |
| F3b | TCGTCGGCAGCGTCAGATGTGT | 78 |
| | ATAAGAGACAGATTTCGATTTA | |
| | AATAGGCCTGACTCACTA | |
| R2 | GTCTCGTGGGCTCGGAGATGTG | 79 |
| | TATAAGAGACAGAATCTTCTCT | |
| | CATCCGCCAAA | |

7.1.1.6. In vitro Type II Cas PAM Identification Assay

The in vitro PAM evaluation of ENQP Type II Cas protein was performed according to the protocol from Karvelis et al. (Karvelis, et al., 2019, Methods in Enzymology 616:219-240) with few modifications. In brief: the synthetic DNA encoding the human codon-optimized version of the Type II Cas gene was obtained from Genscript and cloned into an expression vector for in vitro transcription and translation (IVT) (pT7-N-His-GST, Thermo Fisher Scientific). A reaction was performed according to the manufacturer's protocol (1-Step Human High-Yield Mini IVT Kit, Thermo Fisher Scientific). The Cas-guide RNA ribonucleoprotein (RNP) complex was assembled by combining 20 UL of the supernatant containing soluble Type II Cas protein with 1 μL of RiboLock™ RNase Inhibitor (Thermo Fisher Scientific) and 2 μg of guide RNA. The RNP was used to digest 1u g of the randomized PAM plasmid DNA library for 1 hour at 37° C.

A double-stranded DNA adapter (from Karvelis, et al., 2019, Methods in Enzymology 616:219-240) (Table 9) was ligated to the DNA ends generated by the targeted Type II Cas cleavage and the final ligation product was purified using a GeneJet™ PCR Purification Kit (Thermo Fisher Scientific).

TABLE 9
Sequences of the two oligonucleotides
used to prepare the dsDNA adapter
for the in vitro PAM assay

| Oligo | Sequence (5′ → 3′) | SEQ ID NO: |
|---|---|---|
| Oligo UP | CGGCATTCCTGCTGAA | 80 |
| | CCGCTCTTCCGATCT | |
| Oligo BOTTOM | GATCGGAAGAGCGGTT | 81 |
| | CAGCAGGAATGCCG | |

One round of a two-step PCR (Phusion® HF DNA polymerase, Thermo Fisher Scientific) was performed to enrich the sequences that were cut using a set of forward primers annealing on the adapter and a reverse primer designed on the plasmid backbone downstream of the PAM (Table 10). A second round of PCR was performed to attach the Illumina indexes and adapters. PCR products were purified using Agencourt AMPure™ beads in a 1:0.8 ratio.

TABLE 10
Sequences of the primers used for NGS
library preparation in the in vitro
PAM assay

| Primer | | SEQ ID |
|---|---|---|
| name | Sequence (5′ → 3′) | NO: |
| F4a | TCGTCGGCAGCGTCAGATGTGTATA | 82 |
| | AGAGACAGCTGCTGAACCGCTCTTC | |
| | CGATC | |
| F4b | TCGTCGGCAGCGTCAGATGTGTATA | 83 |
| | AGAGACAGTAAGACTGCTGAACCG | |
| | CTCTTCCGATC | |
| F4c | TCGTCGGCAGCGTCAGATGTGTATA | 84 |
| | AGAGACAGGCTAGACCTAATGTGAT | |
| | CTGCTGAACCGCTCTTCCGATC | |
| R3 | GTCTCGTGGGCTCGGAGATGTGTAT | 85 |
| | AAGAGACAGTCTGCGTTCTGATTTA | |
| | ATCTGTATCAGGC | |

The library was analyzed with a 71-bp single read sequencing, using a flow cell v2 micro, on an Illumina MiSeq™ sequencer.

PAM sequences were extracted from Illumina MiSeq™ reads and used to generate PAM sequence logos, using Logomaker version 0.8 (Tareen and Kinney, 2020, Bioinformatics 36(7):2272-2274). PAM heatmaps (Walton, et al., 2021, Nature Protocols 16(3):1511-1547) were used to display PAM enrichment, computed dividing the frequency of PAM sequences in the cleaved library by the frequency of the same sequences in a control uncleaved library.

### 7.1.1.7. Cell Line Transfections

To perform editing studies on EGFP, 200,000 U2OS.EGFP cells were nucleofected with 1 μg of pX-Cas plasmid bearing a sgRNA designed to target EGFP using the 4D-Nucleofector™ X Kit (Lonza), DN100 program, according to the manufacturer's protocol. After electroporation, cells were seeded in a 96-well plate and moved to 24-well plates after 48 hours for expansion.

To perform editing studies on endogenous genomic loci, 100,000 HEK293T cells were seeded in a 24-well plate 24 hours before transfection. Cells were then transfected with 1 μg of pX-ENQP plasmids targeting the locus of interest using the TransIT®-LT1 reagent (Mirus Bio) according to the manufacturer's protocol. Cell pellets were collected 3 day from transfection for indel analysis.

### 7.1.1.8. Evaluation of Gene Editing

Three days after transfection, cells were collected and DNA was extracted using the QuickExtract™ DNA Extraction Solution (Lucigen) according to the manufacturer's instructions. To amplify the target loci, PCR reactions were performed using the HOT FIREPok® polymerase (Solis BioDyne), using the oligonucleotides listed in Table 11. The amplified products were purified, Sanger sequenced (EasyRun service, Microsynth) and analyzed with the TIDE web tool (shinyapps.datacurators.nl/tide/) to quantify indels. The primers used for Sanger sequencing reactions on amplicons are reported in Table 12, associated with their respective target locus.

For EGFP disruption assays, EGFP knock-out was analyzed four days after nucleofection using a BD FACSCanto™ (BD) flow cytometer.

TABLE 11
Oligonucleotides used to amplify genomic
regions and to perform TIDE analysis

| | | SEQ | | SEQ |
|---|---|---|---|---|
| | Forward | ID | Reverse | ID |
| Locus | (5′ → 3′) | NO: | (5′ → 3′) | NO: |
| RHO | GGGAACGAAC | 86 | CAGCACCCCA | 87 |
| | AGGTAAGGGG | | TCTGTTTTCC | |
| RHO- | CAGTGATAGAG | 161 | CAGCACCCCA | 87 |
| GFP | ATCTCCCTATC | | TCTGTTTTCC | |
| DNMT1 | GTCTTAATTTC | 88 | CGTTTTGGGC | 89 |
| | CACTCATACAG | | TCTGGGACTC | |
| | TGGTAG | | AG | |

TABLE 12
Oligonucleotides used for Sanger sequencing
to perform TIDE analysis

| Oligo name | Sequence | SEQ ID NO: |
|---|---|---|
| RHO/RHO-GFP | AGCGTGAGGAAGTTG | 90 |
| | ATGGG | |
| DNMT1 | GTCTTAATTTCCACT | 88 |
| | CATACAGTGGTAG | |

### 7.1.2. Results

7.1.2.1. Identification of a Novel Type II Cas Protein from Metagenomic Data

The great development of the genome editing field, with several upcoming clinical applications already tested in the first patients, and new technologies to modify the cellular DNA going beyond the introduction of double strand breaks, pushes for the discovery of new tools to edit the genetic material of cells. In particular, the discovery of new Type II Cas nucleases with smaller sizes compared to the most widely used SpCas9 and a variety of different PAM specificities is of great interest to the advancement of the field, both for industrial/applied and the basic research. These features will allow on one hand to increase the density of targetable sites in a defined genome (more PAMs) and on the other hand to provide much easier vectorization, especially in AAV vectors which suffer from limitations in cargo size, thanks to the smaller CDS size.

For these studies, a curated collection of assembled bacterial and archaeal metagenome-based genomes (Pasolli, et al., 2019, Cell 176(3):649-662.e20) was explored exploiting a custom-written bioinformatic pipeline to identify novel Type II Cas proteins with extremely low sequence homology to Type II Cas orthologs previously published and characterized. The discovered Type II Cas proteins were filtered based on: i) the length of their coding sequence, discarding those too short (<950 aa) or too long (>1100 aa); ii) their origin from putative unknown species and iii) the presence of intact nucleasic domains. Type II Cas proteins with high sequence similarity were clustered together and the orthologs with the greater sequence representation in the original metagenomic library were selected for each cluster. Among the identified Type II Cas, one was of particular interest: ENQP Type II Cas, originating from Thermophilibacter mediterraneus, 1005 aa long.

Next a search to identify the tracrRNA of this nuclease from the same metagenomic data was performed using a custom-built bioinformatic pipeline and sgRNAs were designed for ENQP Type II Cas by combining the identified tracrRNA with the corresponding crRNAs extracted from the CRISPR arrays of the nuclease. The predicted hairpin structure of an exemplary ENQP Type II Cas sgRNA molecule is represented in FIG. 1A, while the sequence is reported in Table 6. The ENQP Type II Cas sgRNA sequence shown in FIG. 1A was further modified by trimming its first stem-loop generating an alternative shorter scaffold (folded structure illustrated in FIG. 1B and its sequence reported in Table 6).

### 7.1.2.2. Determination of the PAM Specificity of the ENQP Type II Cas Protein

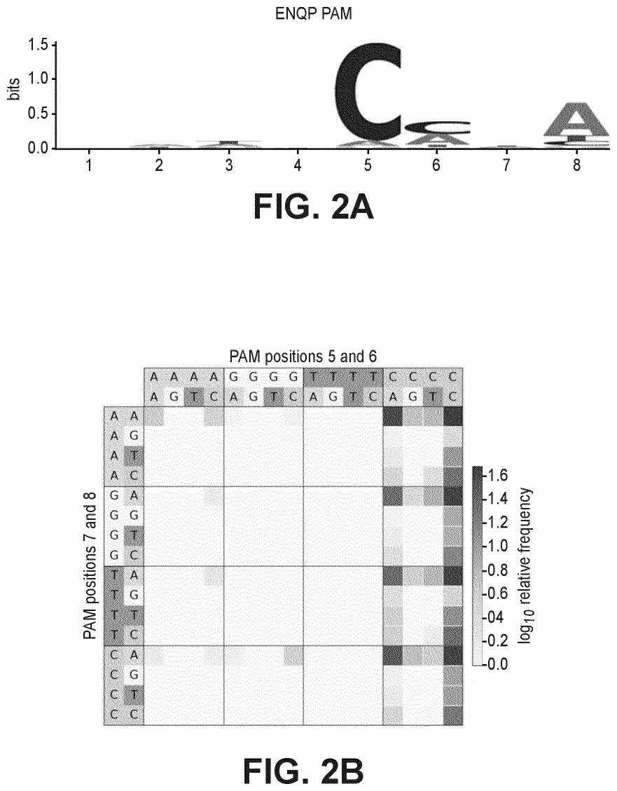

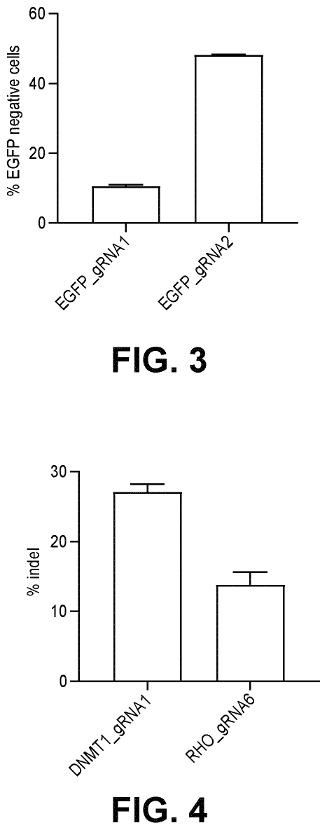

Having determined the sgRNA requirements for ENQP Type II Cas, it was possible to proceed with the discovery of the PAM site recognized by the nuclease. For technical reasons connected to the synthesis of the sgRNA, the trimmed version of the guide scaffold was used for the PAM discovery assay. The ENQP Type II Cas PAM preference was determined exploiting an in vitro assay, resulting in a 3′ N₄CMNA PAM, where M=C or A (FIG. 2A). The visualization of PAM enrichment as heatmaps allowed a more precise evaluation of the PAMs that were better cut by ENQP Type II Cas (FIG. 2B), revealing that it shows a slight preference for an C in position 6 (a more stringent PAM then corresponds to N₄CCNA). This first set of studies also allowed a preliminary validation of the activity of the trimmed sgRNA design generated for ENQP Type II Cas.

### 7.1.2.3. Evaluation of the Editing Activity on an EGFP Reporter

After having determined the PAM preferences of ENQP Type II Cas, the activity of this novel nuclease was assayed in human cells first by performing an EGFP disruption assay in U2OS cells stably expressing EGFP. ENQP Type II Cas was nucleofected in target cells in combination with two different sgRNAs targeting the EGFP coding sequence. EGFP downregulation was measured by cytofluorimetry 4 days after transfection, revealing approximately 50% EGFP for gRNA_2 and only marginal effects on EGFP fluoresce with gRNA_1. Notably, while the PAM sequence associated with gRNA_2 (N₄CCGA) was among the best cleaved in the PAM assay, that is not the case for the PAM of gRNA_1 (N₄CTGA), thus explaining the different efficiencies in indel formation.

### 7.1.2.4. Evaluation of the Editing Activity on Endogenous Genomic Loci

After having evaluated the editing efficacy of ENQP Type II Cas using an EGFP-based reporter system, to further demonstrate the usefulness of the nuclease as a genome editing tool in mammalian cells, its activity was measured on a selection of endogenous genomic loci by transient transfection in HEK293T cells.

The editing activity of ENQP was tested on the RHO and DNMT1 loci demonstrating, as shown in FIG. 4, appreciable indel formation with the evaluated guides (approx. 30% on the DNMT1 locus and approx. 15% on the RHO locus). These data confirmed the activity of ENQP Type II Cas in human cells.

7.1.2.5. Evaluation of ENQP allele-specificity on the RHO rs7984 SNP

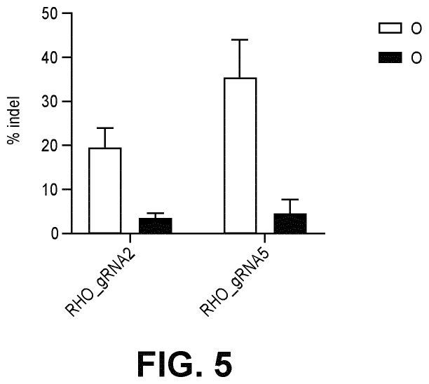

To further characterize the editing activity and specificity of ENQP Type II Cas, two additional guides targeting the rs7984 SNP located in the 5′-UTR of the RHO gene were selected. First, the editing activity of the two guides in combination with the ENQP nuclease was evaluated by transient transfection of HEK293T cells. These cells are homozygous for the rs7984A allele of the SNP. As shown in FIG. 5, both guides showed appreciable levels of indel formation, with the best performing guide gRNA5_ENQP_RHO_A showing almost 40% indel formation.

Next, to verify the targeting specificity of ENQP Type II Cas in combination with the selected guides (targeting the rs7984A SNP allele) an engineered HEK293 cell line expressing a RHO-EGFP minigene characterized by the presence of the rs7984G SNP allele was exploited. After transient transfection of HEK293-RHO-EGFP no editing activity was measured on the integrated minigene (FIG. 5) demonstrating the allele-specificity of ENQP in combination with the two selected guide RNAs, within the limits of sensitivity of the assay used to measure indel formation.

### 7.2. Example 2: “Super Trimmed” sgRNA Scaffold

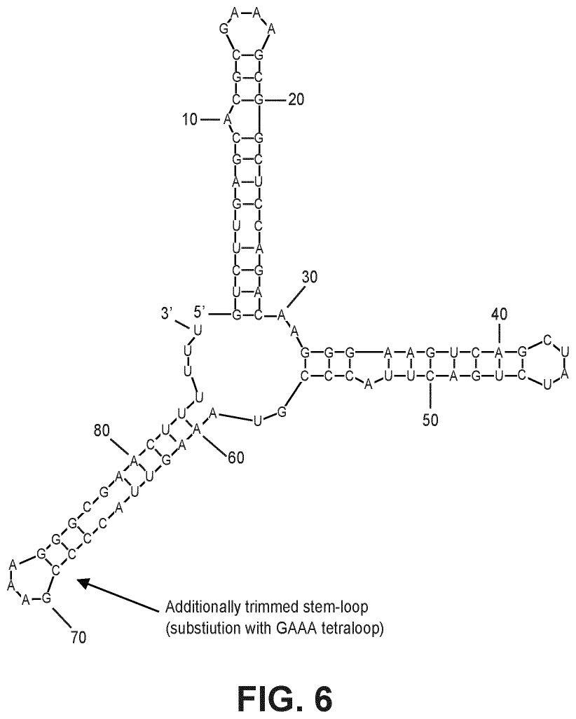

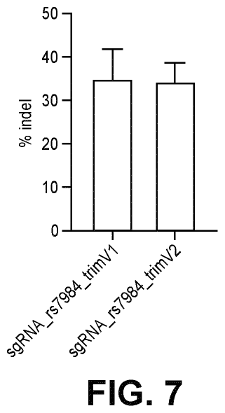

A “super trimmed” scaffold based on the ENQP Type II Cas sgRNAtrimV1 scaffold was designed. The scaffold, sgRNAtrimV2, includes the features of the sgRNAtrimV1 scaffold but includes an additionally trimmed stem-loop (FIG. 6). Indel formation was evaluated as in Example 1 using wild-type ENQP Type II Cas and gRNAs having the gRNA5_ENQP_RHO_A spacer (SEQ ID NO:38) and sgRNAtrimV1 and sgRNAtrimV2 scaffolds (SEQ ID NO:47 and SEQ ID NO:92, respectively). Results are shown in FIG. 7.

### 7.3. Example 3: Allele Specific RHO Editing with ENQP Type II Cas

This Example describes the design and evaluation of a mutation independent allele-specific strategy to selectively inactivate mutated RHO alleles. The RHO gene, which encodes for the photopigment rhodopsin, is one of the most frequently mutated genes in autosomal dominant retinitis pigmentosa and more than one hundred mutations have been described in the art. The great heterogeneity of mutations in affected patients and the overall low prevalence of most of these mutations makes a mutation independent approach to target the disease particularly desirable. Additionally, effective knock-out of diseased alleles can be effectively obtained using gene editing tools, such as Type II Cas enzymes. Key for the success of the approach is the ability to preferentially downregulate RHO mutated alleles while sparing the wild-type counterpart, in order to preserve photoreceptor function.

The strategy described in this Example exploits a commonly occurring non-pathogenic SNP in the RHO gene, rs7984, located in the 5′-UTR of the gene and common in the general population, to selectively target only the one RHO allele containing dominant negative mutations, independently of the exact nature of the mutation. Only patients which are heterozygous for the rs7984 SNP are potentially eligible for this targeting strategy, which is based on the exact knowledge of the phase between the SNP alleles and the mutation affecting each patient. Allele-selectivity is achieved selectively targeting the rs7984 allele which is in phase with the patient's mutation.

Since the rs7984 SNP is located outside the RHO coding sequence, a second cut in RHO intron 1 can be introduced to remove the entire exon 1 and knock-out expression of the mutated protein. This second cut, which has to occur synchronously with the cut on the rs7984 locus to produce the desired deletion, can be bi-allelic, targeting a site present on both RHO alleles.

### 7.3.1. Materials and Methods

### 7.3.1.1. Plasmids

A CAG-driven expression plasmid was used to express the ENQP Type II Cas in mammalian cells. Briefly, a human codon-optimized coding sequence of ENQP Type II Cas and sgRNA scaffolds sgRNAFS (SEQ ID NO:46), sgRNtrimV1 (SEQ ID NO:47) and sgRNAtrimV2 (SEQ ID NO:92) were cloned into the aforementioned expression plasmid, generating pX-ENQP-sgRNAFS, pX-ENQP-sgRNAtrimV1 and pX-ENQP-sgRNAtrimV2. Unless otherwise stated the sgRNAtrimV1 trimmed sgRNA scaffold was used in editing studies. The ENQP Type II Cas coding sequence, modified by the addition of an SV5 tag at the N-terminus and two nuclear localization signals (one at the N-terminus and one at the C-terminus) and human codon-optimized, as well as the sgRNA scaffolds, were obtained as synthetic fragments from Genewiz. Spacer sequences were cloned into the pX-ENQP plasmids as annealed DNA oligonucleotides using a double BsaI site present in the plasmid. The list of spacer sequences and relative cloning oligonucleotides used in the present example is reported in Table 13.

### 7.3.1.2. Cell Lines

HEK293T cells (obtained from ATCC) and HEK293-RHO-P23H cells were cultured in DMEM (Life Technologies) supplemented with 10% FBS (Life Technologies), 2 mM L-Glutamine (Life Technologies) and penicillin/streptomycin (Life Technologies). HEK293-RHO-P23H cells were obtained by stable transfection of HEK293 cells with a tetracycline-inducible CMV-driven RHO minigene (containing the complete exon-intron structure of human RHO except for a truncation in the 3′-UTR of the gene) characterized by the P23H mutation and the rs7984G allele. Cells were pool-selected with 5 μg/ml Hygromycin (Invivogen) and single clones were subsequently isolated and expanded. All cells were incubated at 37° C. and 5% CO₂ in a humidified atmosphere. All cells tested mycoplasma negative (PlasmoTest, Invivogen).

### 7.3.1.3. Cell Line Transfections

To perform editing studies on target genomic loci 100,000 HEK293T or HEK293-RHO-P23H cells were seeded in a 24-well plate 24 hours before transfection. Cells were then transfected with 750 ng of pX-ENQP plasmids targeting the locus of interest using the TransIT®-LT1 reagent (Mirus Bio) according to the manufacturer's protocol. Cell pellets were collected 3 days from transfection for indel analysis.

### 7.3.1.4. Evaluation of Gene Editing

Three days after transfection cells were collected and DNA was extracted using the QuickExtract™ DNA Extraction Solution (Lucigen) according to the manufacturer's instructions. To amplify the target loci, PCR reactions were performed using the HOT FIREPok® polymerase (Solis BioDyne), using the oligonucleotides listed in Table 14. The amplified products were purified, Sanger sequenced (EasyRun service, Microsynth) and analyzed with the TIDE web tool (shinyapps.datacurators.nl/tide/) to quantify indels. The primers used for Sanger sequencing reactions on amplicons are reported in Table 15, associated with their respective target locus. Deletion formation was detected by PCR amplification of the target locus using the primers reported in Table 16 and visualization of the amplified products on agarose gel.

TABLE 13
Sequences of the oligonucleotides used for cloning sgRNA spacers and sequences of their relative target sites

| Spacer sequences | | | | | Oligo used to clone the spacer in pX plasmid ( ** ) | | | |
|---|---|---|---|---|---|---|---|---|
| | | SEQ ID | | SEQ ID | | SEQ ID | | SEQ ID |
| Name | Protospacer | NO: | Target (*) | NO | Oligo 1 (5′>3′) | NO | Oligo 2 (5′>3′) | NO: |
| gRNA2_ | CTTGTGGCTGACC | 56 | gccCTTGTGGCTGACCCGTGGCT | 57 | caccGCTTGTGGCTGA | 58 | agacGAGCAGCCACG | 59 |
| rs7984A | CGTGGCTGCTC | | GCTCCCACCCAAgaa | | CCCGTGGCTGCTC | | GGTCAGCCACAAGC | |
| gRNA4_ | GGCCCTTGTGGCT | 162 | tgtGGCCCTTGTGGCTGACCCGT | 163 | caccGGCCCTTGTGGC | 164 | agacAGCCACGGGTC | 165 |
| rs7984A | GACCCGTGGCT | | GGCTGCTCCCACcca | | TGACCCGTGGCT | | AGCCACAAGGGCC | |
| gRNA5_ | CTTGGGTGGGAGC | 60 | attCTTGGGGGGAGCAGCCACG | 61 | caccGCTTGGGTGGGA | 62 | agacACCCGTGGCTG | 63 |
| rs7984A | AGCCACGGGT | | GGTCAGCCACAagg | | GCAGCCACGGGT | | CTCCCACCCAAGC | |
| gRNA6_ | GGAGCAGCCACG | 64 | gtgGGAGCAGCCACGGGTCAGC | 65 | caccGGAGCAGCCACG | 66 | agacTTGTGGCTGACC | 67 |
| rs7984A | GGTCAGCCACAA | | CACAAGGGCCACAgcc | | GGTCAGCCACAA | | CGTGGCTGCTCC | |
| gRNA5-20nt | GGGTGGGAGCAG | 166 | cttGGGTGGGAGCAGCCACGGGT | 167 | caccGGGTGGGAGCAG | 168 | agacACCCGTGGCTG | 169 |
| | CCACGGGT | | CAGCCACAagg | | CCACGGGT | | CTCCCACCC | |
| gRNA5-21nt | TGGGTGGGAGCAG | 170 | tctTGGGTGGGAGCAGCCACGGG | 171 | caccGTGGGTGGGAGC | 172 | agacACCCGTGGCTG | 173 |
| | CCACGGGT | | TCAGCCACAagg | | AGCCACGGGT | | CTCCCACCCAC | |
| gRNA5-22nt | TTGGGTGGGAGCA | 174 | ttcTTGGGTGGGAGCAGCCACGG | 175 | caccGTTGGGTGGGAG | 176 | agacACCCGTGGCTG | 177 |
| | GCCACGGGT | | GTCAGCCACAagg | | CAGCCACGGGT | | CTCCCACCCAAC | |
| gRNA5-24nt | TCTTGGGTGGGAG | 178 | catTCTTGGGTGGGAGCAGCCAC | 179 | caccGTCTTGGGTGGG | 180 | agacACCCGTGGCTG | 181 |
| | CAGCCACGGGT | | GGGTCAGCCACAagg | | AGCAGCCACGGGT | | CTCCCACCCAAGAC | |
| gRNA5-25nt | TTCTTGGGTGGGA | 182 | gcaTTCTTGGGTGGGAGCAGCCA | 183 | caccGTTCTTGGGTGG | 184 | agacACCCGTGGCTG | 185 |
| | GCAGCCACGGGT | | CGGGTCAGCCACAagg | | GAGCAGCCACGGGT | | CTCCCACCCAAGAAC | |
| gRNA5-21nt_ | TGGGTGGGAGCAG | 186 | tctTGGGTGGGAGCAGCCGCGG | 187 | caccGTGGGTGGGAGC | 188 | agacACCCGCGGCTG | 189 |
| rs7984G | CCGCGGGT | | GTCAGCCACAagg | | AGCCGCGGGT | | CTCCCACCCAC | |
| g-int482 | CATGCTCCCGGGC | 190 | ctcCATGCTCCCGGGCTCCTGCA | 191 | caccGCATGCTCCCGG | 192 | agacGTGTGCAGGAG | 193 |
| | TCCTGCACAC | | CACCCCACCCAcac | | GCTCCTGCACAC | | CCCGGGAGCATGC | |
| g-int486 | CCTCCATGCTCCC | 194 | accCCTCCATGCTCCCGGGCTCC | 195 | caccGCCTCCATGCTC | 196 | agacGCAGGAGCCCG | 197 |
| | GGGCTCCTGC | | TGCACACCCCAccc | | CCGGGCTCCTGC | | GGAGCATGGAGGC | |
| g-int521 | AGCCACCACCGCC | 113 | ctcAGCCACCACCGCCAAGCCCG | 198 | caccGAGCCACCACCG | 199 | agacTCCCGGGCTTG | 200 |
| | AAGCCCGGGA | | GGACTCTCCCAgac | | CCAAGCCCGGGA | | GCGGTGGTGGCTC | |
| g-int572 | TCCCTTCTCCTGTC | 201 | ttcTCCCTTCTCCTGTCCTGTCAA | 202 | caccGTCCCTTCTCCTG | 203 | agacCATTGACAGGAC | 204 |
| | CTGTCAATG | | TGTTATCCAAagc | | TCCTGTCAATG | | AGGAGAAGGGAC | |
| g-int573 | CCCTTCTCCTGTC | 205 | tctCCCTTCTCCTGTCCTGTCAAT | 206 | caccGCCCTTCTCCTGT | 207 | agacACATTGACAGGA | 208 |
| | CTGTCAATGT | | GTTATCCAAAgcc | | CCTGTCAATGT | | CAGGAGAAGGGC | |
| g-int582 | TGTCCTGTCAATGT | 209 | tccTGTCCTGTCAATGTTATCCAA | 210 | caccGTGTCCTGTCAAT | 211 | agacCTTTGGATAACA | 212 |
| | TATCCAAAG | | AGCCCTCATAtat | | GTTATCCAAAG | | TTGACAGGACAC | |
| g-int597 | ATCCAAAGCCCTC | 213 | gttATCCAAAGCCCTCATATATTC | 214 | caccGATCCAAAGCCC | 215 | agacCTGAATATATGA | 216 |
| | ATATATTCAG | | AGTCAACAAAcac | | TCATATATTCAG | | GGGCTTTGGATC | |
| g-int648 | AAGGCAGTGTTCA | 217 | atcAAGGCAGTGTTCAGTGCCAG | 218 | caccGAAGGCAGTGTT | 219 | agacGGGCTGGCACT | 220 |
| | GTGCCAGCCC | | CCCTGCACAAAcag | | CAGTGCCAGCCC | | GAACACTGCCTTC | |
| g-int650 | TCAAGGCAGTGTT | 221 | agaTCAAGGCAGTGTTCAGTGCC | 222 | caccGTCAAGGCAGTG | 223 | agacGCTGGCACTGAA | 224 |
| | CAGTGCCAGC | | AGCCCTGCACAaac | | TTCAGTGCCAGC | | CACTGCCTTGAC | |
| g-int686 | GTGAAATTAGACA | 225 | gctGTGAAATTAGACAAGCGCAT | 226 | caccGTGAAATTAGACA | 227 | agacAATATGCGCTTG | 228 |
| | AGCGCATATT | | ATTGCTCCAAAtaa | | AGCGCATATT | | TCTAATTTCAC | |
| g-int696F | TTGGAGCAATATG | 229 | tatTTGGAGCAATATGCGCTTGTC | 230 | caccGTTGGAGCAATAT | 231 | agacTAGACAAGCGCA | 232 |
| | CGCTTGTCTA | | TAATTTCACAgca | | GCGCTTGTCTA | | TATTGCTCCAAC | |
| g-int696R | GTTTTCTTGCTGTG | 233 | tcaGTTTTCTTGCTGTGAAATTAG | 234 | caccGTTTTCTTGCTGT | 235 | agacTGTCTAATTTCA | 236 |
| | AAATTAGACA | | ACAAGCGCATAttg | | GAAATTAGACA | | CAGCAAGAAAAC | |
| g-int722 | TCACAGCAAGAAA | 237 | attTCACAGCAAGAAAACTGAGCT | 238 | caccGTCACAGCAAGA | 239 | agacTCAGCTCAGTTT | 240 |
| | ACTGAGCTGA | | GAGGCTCAAAgaa | | AAACTGAGCTGA | | TCTTGCTGTGAC | |
| g-int751 | AAGAAGTCAAGCG | 241 | tcaAAGAAGTCAAGCGCCCTGCT | 242 | caccGAAGAAGTCAAG | 243 | agacCCCAGCAGGGC | 244 |
| | CCCTGCTGGG | | GGGGCGTCACAcag | | CGCCCTGCTGGG | | GCTTGACTTCTTC | |
| g-int753 | GAAGTCAAGCGCC | 245 | aaaGAAGTCAAGCGCCCTGCTG | 246 | caccGAAGTCAAGCGC | 247 | agacGCCCCAGCAGG | 248 |
| | CTGCTGGGGC | | GGGCGTCACACAggg | | CCTGCTGGGGC | | GCGCTTGACTTC | |
| g-int761 | AACTCTGCACCCG | 249 | ctcAACTCTGCACCCGTCCCTGT | 250 | caccGAACTCTGCACC | 251 | agacCACACAGGGAC | 252 |
| | TCCCTGTGTG | | GTGACGCCCCAgca | | CGTCCCTGTGTG | | GGGTGCAGAGTTC | |
| g-int767 | TGCTGGGGCGTCA | 253 | cccTGCTGGGGCGTCACACAGG | 254 | caccGTGCTGGGGCGT | 255 | agacCGTCCCTGTGTG | 256 |
| | CACAGGGACG | | GACGGGTGCAGAgtt | | CACACAGGGACG | | ACGCCCCAGCAC | |
| g-int795 | AACATGGCCCGAG | 257 | gcaAACATGGCCCGAGATAGATG | 258 | caccGAACATGGCCCG | 259 | agacCCGCATCTATCT | 260 |
| | ATAGATGCGG | | CGGGCTTCCAActc | | AGATAGATGCGG | | CGGGCCATGTTC | |
| g-int814 | ACAGAGGCTTGGT | 261 | gaaACAGAGGCTTGGTGCTGCAA | 262 | caccGACAGAGGCTTG | 263 | agacTGTTTGCAGCAC | 264 |
| | GCTGCAAACA | | ACATGGCCCGAgat | | GTGCTGCAAACA | | CAAGCCTCTGTC | |
| g-int824 | TCCAAGGGAAACA | 265 | tgcTCCAAGGGAAACAGAGGCTT | 266 | caccGTCCAAGGGAAA | 267 | agacACCAAGCCTCTG | 268 |
| | GAGGCTTGGT | | GGTGCTGCAAAcat | | CAGAGGCTTGGT | | TTTCCCTTGGAC | |
| g-int840 | GACTCAGCACAGC | 269 | tctGACTCAGCACAGCTGCTCCAA | 270 | caccGACTCAGCACAG | 271 | agacCCTTGGAGCAGC | 272 |
| | TGCTCCAAGG | | GGGAAACAGAggc | | CTGCTCCAAGG | | TGTGCTGAGTC | |
| g-int850 | GCCTGGGTCTGAC | 273 | ccaGCCTGGGTCTGACTCAGCAC | 274 | caccGCCTGGGTCTGA | 275 | agacGCTGTGCTGAGT | 276 |
| | TCAGCACAGC | | AGCTGCTCCAAggg | | CTCAGCACAGC | | CAGACCCAGGC | |
| g-int856 | CCCTTGGAGCAGC | 277 | tttCCCTTGGAGCAGCTGTGCTGA | 278 | caccGCCCTTGGAGCA | 279 | agacACTCAGCACAGC | 280 |
| | TGTGCTGAGT | | GTCAGACCCAggc | | GCTGTGCTGAGT | | TGCTCCAAGGGC | |
| g-int860 | TCAGTGCCCAGCC | 281 | cccTCAGTGCCCAGCCTGGGTCT | 282 | caccGTCAGTGCCCAG | 283 | agacGTCAGACCCAG | 284 |
| | TGGGTCTGAC | | GACTCAGCACAgct | | CCTGGGTCTGAC | | GCTGGGCACTGAC | |

(*) The target sequences are reported with three flanking nucleotides on each side.
The PAM sequence is highlighted in bold.
(**) The cloning overhang is reported in lowercase.
Nucleotides highlighted in bold represent 5′-G appended to favor transcription from canonical U6 Pol III promoter

TABLE 14
Oligonucleotides used to amplify target
regions to perform TIDE analysis

| | | SEQ | | SEQ |
|---|---|---|---|---|
| | For | ID | Rev | ID |
| Locus | (5′ → 3′) | NO: | (5′ → 3′) | NO: |
| rs7984 | GGGAACGAAC | 86 | AGCGTGAGGA | 90 |
| endogenous | AGGTAAGGGG | | AGTTGATGGG | |
| rs7984 | CAGTGATAGA | 161 | AGCGTGAGGA | 90 |
| minigene | GATCTCCCTA | | AGTTGATGGG | |
| | TC | | | |
| RHO | GGGAACGAAC | 86 | CAGCACCCCA | 87 |
| intron 1 | AGGTAAGGGG | | TCTGTTTTCC | |

TABLE 15
Oligonucleotides used for Sanger
sequencing to perform TIDE analysis

| | |
|---|---|
| rs7984 | AGCGTGAGGAAGTTGATGGG |
| endogenous | (SEQ ID NO: 90) |
| rs7984 | CAGTGATAGAGATCTCCCTATC |
| minigene | (SEQ ID NO: 161) |
| RHO intron 1 | GAGATAGATGCGGGCTTCCA |
| (up to sg-int650) | (SEQ ID NO: 285) |
| RHO intron 1 | CAGCACCCCATCTGTTTTCC |
| (from g-int686 | (SEQ ID NO: 87) |
| to g-int860) | |

TABLE 16
Oligonucleotides used for
deletion evaluation

| | |
|---|---|
| For (5′ > 3′) | CAGTGATAGAGATCTCCCTATC |
| | (SEQ ID NO: 161) |
| Rev (5′ > 3′) | CAGCACCCCATCTGTTTTCC |
| | (SEQ ID NO: 87) |

### 7.3.2. Results

7.3.2.1. ENQP Type II Cas sgRNAs for the Allele-Specific Targeting of the RHO Rs7984 SNP

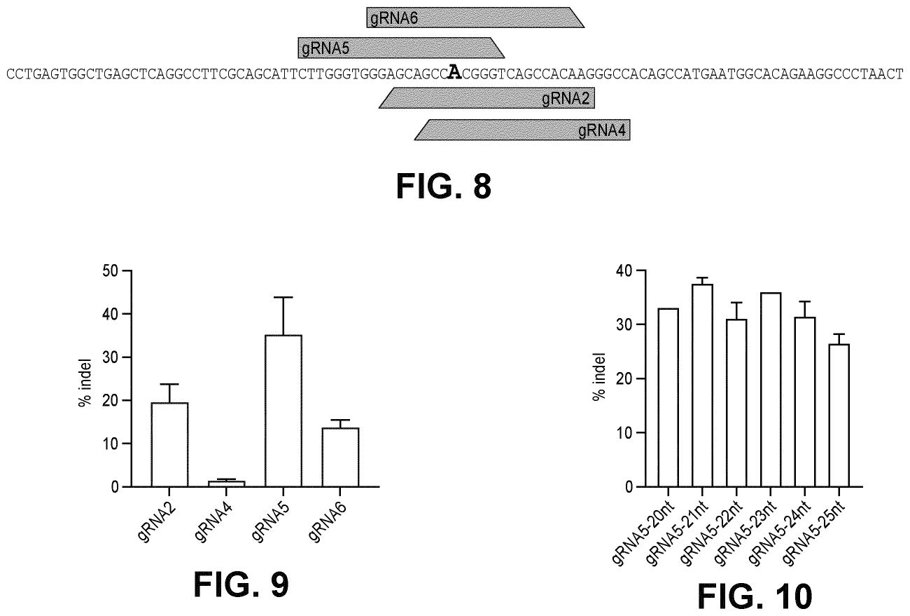

A set of sgRNAs associated with PAMs recognized by ENQP Type II Cas spanning the rs7984 SNP were designed (FIG. 8). The editing activity of the selected guides in combination with ENQP Type II Cas was evaluated by transient transfection of HEK293T cells. These cells are homozygous for the rs7984A allele of the SNP and sgRNAs targeting the rs7984A allele were used. As shown in FIG. 9, some of the guides showed appreciable levels of indel formation, with the best performing gRNA5 showing a striking 40% indel formation. gRNA5 was selected for further characterization given its high editing activity towards the target locus.

To optimize the performance of gRNA5 towards the intended target, different spacer lengths were evaluated spanning from 20 to 25 matching nucleotides (all targeting the rs7984A allele), where the original design was 23 nucleotides long. Where beneficial, a 5′-end non-matching G nucleotide was added to allow efficient transcription for the human U6 promoter, as reported in the art. As shown in FIG. 10, after transient transfection in HEK293T cells, all evaluated lengths demonstrated indel formation, with the 21-nucleotide spacer demonstrating slightly increased activity compared to the others. This spacer length was used in subsequent studies.

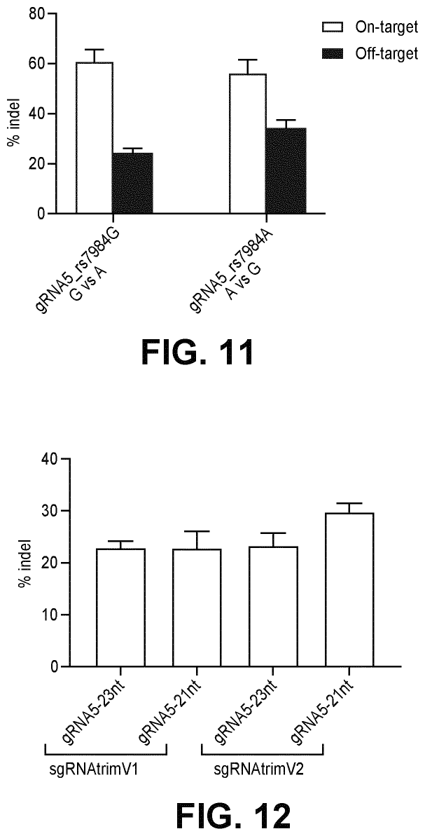

Next, to verify the targeting specificity of ENQP Type II Cas in combination with the selected guide (gRNA5), two parallel approaches were undertaken. A version of gRNA5 targeting the rs7984G allele was transfected together with ENQP Type II Cas in an engineered HEK293 cell line expressing a mutated RHO P23H minigene characterized by the presence of the rs7984G SNP allele. After transient transfection of HEK293-RHO-P23H cells the editing activity was measured both on the integrated minigene (rs7984G allele), to verify on-target cleavage, and on the endogenous locus (rs7984A), to verify the allelic specificity (FIG. 11, left bars). In parallel, the same cells were transfected with a version of gRNA5 targeting the rs7984A allele together with ENQP Type II Cas to measure on-target editing on the endogenous locus and the cleavage specificity towards the integrated rs7984G allele included in the P23H RHO minigene (FIG. 11, right bars). This allowed the complete evaluation of activity and specificity of alternative versions of the guide directed to both alleles of the rs7984 SNP which can be exploited in order to cover the entire putatively eligible patient population. Overall, both versions of gRNA5 show allelic preference towards the intended target, which is more efficiently edited compared to non-target counter allele.

In order to further improve the editing activity of the best performing guide RNA, alternative sgRNA scaffolds for ENQP Type II Cas were evaluated. Side-by-side evaluation of a trimmed scaffold where only the repeat: antirepeat loop is trimmed (sgRNAtrimv1) with an alternative design where the sgRNA is additionally shortened by trimming the stem-loop located at the 3′-end of the guide (sgRNAtrimV2) demonstrated that not only the most trimmed version allowed to reach similar levels of activity against the target locus, but in some conditions showed slightly enhanced indel formation (FIG. 12). Shorter and less complex sgRNA scaffolds can be beneficial when exploiting delivery through viral vectors, particularly AAV vectors, due to their shorter size which is more compatible with vectors with limited payload and the presence of less structured regions (stem-loops, hairpins) which can interfere with correct replication of the vector genome.

7.3.2.2. ENQP Type II Cas sgRNAs to Target RHO Intron 1

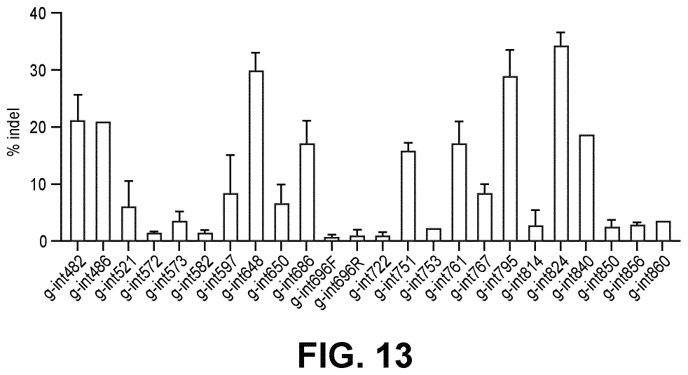

Next, sgRNAs for ENQP Type II Cas targeting RHO intron 1 were screened for cleavage activity with the goal of identifying best performing sgRNAs that can be used in combination with gRNA5 targeting the rs7984 SNP to generate the desired deletion encompassing RHO exon 1, which includes the ATG translation start site, to knock-out mutant protein expression. HEK293T cells were transfected with ENQP Type II Cas together with a panel of sgRNAs targeting the first half of RHO intron 1 to generate a deletion below 1000 bp in size when used in combination with gRNA5 targeting the rs7984 SNP. Variable levels of indel formation were observed with the different guides, with some of the guides failing to generate appreciable editing at the target site (FIG. 13). Interestingly, three sgRNAs showed particularly high levels of modification: g-int648, g-int795 and g-int824, which were selected for further studies.

7.3.2.3. Evaluation of Deletion Formation in the RHO Gene Using Best Performing sgRNA Couples

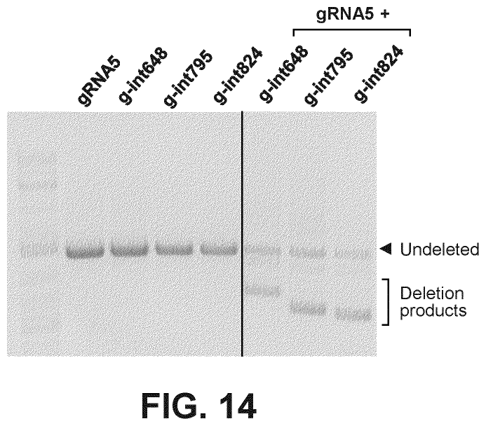

Having identified high performing guides targeting both the rs7984 SNP and RHO intron 1, deletion formation at the integrated RHO minigene was assessed after transient transfection of HEK293-RHO-P23H cells with combinations of gRNA5 (directed towards the rs7984G allele) with the g-int648, g-int795 and g-int824 guides selected to target RHO intron 1. As a control, each guide was also transfected singularly in combination with ENQP Type II Cas. Deletion formation was assessed using a specifically designed PCR assay with primers spanning the deleted region and was visualized using agarose gel electrophoresis, where a low molecular weight band should be present when the desired deletion is correctly formed. As shown in FIG. 14, each of the evaluated combinations was producing the desired deletion in the RHO locus very efficiently, while guides used alone failed to induce such modification.

In summary, sgRNAs targeting the RHO gene that generate high levels of deletion of the first exon of the gene and have selectivity for the desired RHO copy were identified.

7.4. Example 4: Gene Editing with ENQP Type II Cas

To further evaluate the cleavage activity of ENQP Type II Cas on loci of interest, a panel of endogenous loci (B2M, TRAC, LAG3, PD-1) which are commonly targeted to generate allogeneic CAR-T cells (Chimeric Antigen Receptor T cells) were selected for editing studies. For each target locus multiple sgRNAs were designed and evaluated in parallel by transient plasmid transfection in HEK293T cells

### 7.4.1. Materials and Methods

### 7.4.1.1. Plasmids

Spacer sequences were cloned into the pX-ENQP plasmids (described in Example 3) as annealed DNA oligonucleotides using a double BsaI site present in the plasmid. The list of spacer sequences and relative cloning oligonucleotides used in the present example is reported in Table 17.

### 7.4.1.2. Cell Lines

HEK293T cells (obtained from ATCC) were cultured in DMEM (Life Technologies) supplemented with 10% FBS (Life Technologies), 2 mM L-Glutamine (Life Technologies) and penicillin/streptomycin (Life Technologies). Cells were incubated at 37° C. and 5% CO₂ in a humidified atmosphere. All cells tested mycoplasma negative (PlasmoTest, Invivogen).

### 7.4.1.3. Cell Line Transfections

To perform editing experiments on target genomic loci 100,000 HEK293T cells were seeded in a 24-well plate 24 hours before transfection. Cells were then transfected with 750 ng of pX-ENQP plasmids targeting the locus of interest using the TransIT®-LT1 reagent (Mirus Bio) according to the manufacturer's protocol. Cell pellets were collected 3 days from transfection for indel analysis

### 7.4.1.4. Evaluation of Gene Editing

Three days after transfection cells were collected and DNA was extracted using the QuickExtract™ DNA Extraction Solution (Lucigen) according to the manufacturer's instructions. To amplify the target loci, PCR reactions were performed using the HOT FIREPol® polymerase (Solis BioDyne), using the oligonucleotides listed in Table 18. The amplified products were purified, Sanger sequenced (EasyRun service, Microsynth) and analyzed with the TIDE web tool (shinyapps.datacurators.nl/tide/) to quantify indels. Either the forward or reverse primers used for amplification were also used for Sanger sequencing, depending on the position of the guide RNA being evaluated.

TABLE 17
Sequences of the oligonucleotides used for cloning sgRNA spacers and sequences of their relative target sites

| Spacer sequences | | | | | Oligo used to clone the spacer in pX plasmid (**) | | | |
|---|---|---|---|---|---|---|---|---|
| | | SEQ | | SEQ | | SEQ | | SEQ |
| | | ID | | ID | | ID | | ID |
| Name | Protospacer | NO: | Target (*) | NO: | Oligo 1 (5′>3′) | NO: | Oligo 2 (5′>3′) | NO: |
| TRAC_g709 | ATACACATCAGAAT | 286 | gatATACACATCAGAATCCTTACT | 287 | caccGATACACATCAGA | 288 | agacAAAGTAAGGATT | 289 |
| | CCTTACTTT | | TTGTGACACAttt | | ATCCTTACTTT | | CTGATGTGTATC | |
| TRAC_g730 | GTAAGGATTCTGA | 290 | aaaGTAAGGATTCTGATGTGTAT | 291 | caccGTAAGGATTCTGA | 292 | agacTGATATACACAT | 293 |
| | TGTGTATATCA | | ATCACAGACAAAact | | TGTGTATATCA | | CAGAATCCTTAC | |
| TRAC_g793 | GAGCAACAGTGCT | 294 | caaGAGCAACAGTGCTGTGGCCT | 295 | caccGAGCAACAGTGC | 296 | agacTCCAGGCCACAG | 297 |
| | GTGGCCTGGA | | GGAGCAACAAAtct | | TGTGGCCTGGA | | CACTGTTGCTC | |
| TRAC_g813 | GGAGCAACAAATC | 298 | cctGGAGCAACAAATCTGACTTTG | 299 | caccGGAGCAACAAAT | 300 | agacTGCAAAGTCAGA | 301 |
| | TGACTTTGCA | | CATGTGCAAAcgc | | CTGACTTTGCA | | TTTGTTGCTCC | |
| TRAC_g821 | TGCTGTTGTTGAA | 302 | taaTGCTGTTGTTGAAGGCGTTT | 303 | caccGTGCTGTTGTTGA | 304 | agacTGCAAACGCCTT | 305 |
| | GGCGTTTGCA | | GCACATGCAAAgtc | | AGGCGTTTGCA | | CAACAACAGCAC | |
| TRAC_g857 | AGCATTATTCCAGA | 306 | aacAGCATTATTCCAGAAGACAC | 307 | caccGAGCATTATTCCA | 308 | agacAAGGTGTCTTCT | 309 |
| | AGACACCTT | | CTTCTTCCCCAgcc | | GAAGACACCTT | | GGAATAATGCTC | |
| TRAC_g862 | TATTCCAGAAGAC | 310 | catTATTCCAGAAGACACCTTCTT | 311 | caccGTATTCCAGAAGA | 312 | agacGGAAGAAGGTGT | 313 |
| | ACCTTCTTCC | | CCCCAGCCCAggt | | CACCTTCTTCC | | CTTCTGGAATAC | |
| TRAC_g724 | ACAAAGTAAGGAT | 314 | gtcACAAAGTAAGGATTCTGATGT | 315 | caccGACAAAGTAAGG | 316 | agacACACATCAGAAT | 317 |
| | TCTGATGTGT | | GTATATCACAgac | | ATTCTGATGTGT | | CCTTACTTTGTC | |
| TRAC_g733 | GTCTAGCACAGTT | 318 | catGTCTAGCACAGTTTTGTCTGT | 319 | caccGTCTAGCACAGTT | 320 | agacTCACAGACAAAA | 321 |
| | TTGTCTGTGA | | GATATACACAtca | | TTGTCTGTGA | | CTGTGCTAGAC | |
| TRAC_g754 | CTTGAAGTCCATA | 322 | gctCTTGAAGTCCATAGACCTCAT | 323 | caccGCTTGAAGTCCAT | 324 | agacACATGAGGTCTA | 325 |
| | GACCTCATGT | | GTCTAGCACAgtt | | AGACCTCATGT | | TGGACTTCAAGC | |
| TRAC_g772 | GGCCACAGCACTG | 326 | ccaGGCCACAGCACTGTTGCTCT | 327 | caccGGCCACAGCACT | 328 | agacTCAAGAGCAACA | 329 |
| | TTGCTCTTGA | | TGAAGTCCATAgac | | GTTGCTCTTGA | | GTGCTGTGGCC | |
| TRAC_g796 | TGCAAAGTCAGAT | 330 | acaTGCAAAGTCAGATTTGTTGC | 331 | caccGTGCAAAGTCAG | 332 | agacGGAGCAACAAAT | 333 |
| | TTGTTGCTCC | | TCCAGGCCACAgca | | ATTTGTTGCTCC | | CTGACTTTGCAC | |
| TRAC_g827 | GAATAATGCTGTT | 334 | ctgGAATAATGCTGTTGTTGAAGG | 335 | caccGAATAATGCTGTT | 336 | agacCGCCTTCAACAA | 337 |
| | GTTGAAGGCG | | CGTTTGCACAtgc | | GTTGAAGGCG | | CAGCATTATTC | |
| B2M_g791 | TAAGGCCACGGAG | 338 | agcTAAGGCCACGGAGCGAGAC | 339 | caccGTAAGGCCACGG | 340 | agacAGATGTCTCGCT | 341 |
| | CGAGACATCT | | ATCTCGGCCCGAatg | | AGCGAGACATCT | | CCGTGGCCTTAC | |
| B2M_g824 | CAGGCCAGAAAGA | 342 | ctcCAGGCCAGAAAGAGAGAGTA | 343 | caccGCAGGCCAGAAA | 344 | agacCGCTACTCTCTC | 345 |
| | GAGAGTAGCG | | GCGCGAGCACAgct | | GAGAGAGTAGCG | | TTTCTGGCCTGC | |
| B2M_g860 | GCCTGGAGGCTAT | 346 | ctgGCCTGGAGGCTATCCAGCGT | 347 | caccGCCTGGAGGCTA | 348 | agacACTCACGCTGGA | 349 |
| | CCAGCGTGAGT | | GAGTCTCTCCTAccc | | TCCAGCGTGAGT | | TAGCCTCCAGGC | |
| PD1_g279 | GGCCGCCAGCCCA | 350 | cctGGCCGCCAGCCCAGTTGTAG | 351 | caccGGCCGCCAGCCC | 352 | agacGTGCTACAACTG | 353 |
| | GTTGTAGCAC | | CACCGCCCAGAcga | | AGTTGTAGCAC | | GGCTGGCGGCC | |
| PD1_g281 | CTGGCCGCCAGCC | 354 | atcCTGGCCGCCAGCCCAGTTGT | 355 | caccGCTGGCCGCCAG | 356 | agacGCTACAACTGGG | 357 |
| | CAGTTGTAGC | | AGCACCGCCCAgac | | CCCAGTTGTAGC | | CTGGCGGCCAGC | |
| LAG3_g636 | GCCATCCCCGTTT | 358 | cccGCCATCCCCGTTTTACCTGG | 359 | caccGCCATCCCCGTTT | 360 | agacGCTCCAGGTAAA | 361 |
| | TACCTGGAGC | | AGCCACCCAAAgcg | | TACCTGGAGC | | ACGGGGATGGC | |
| LAG3_g637 | CGCCATCCCCGTT | 362 | tccCGCCATCCCCGTTTTACCTG | 363 | caccGCGCCATCCCCG | 364 | agacCTCCAGGTAAAA | 365 |
| | TTACCTGGAG | | GAGCCACCCAAagc | | TTTTACCTGGAG | | CGGGGATGGCGC | |
| LAG3_g638 | CCGCCATCCCCGT | 366 | ctcCCGCCATCCCCGTTTTACCT | 367 | caccGCCGCCATCCCC | 368 | agacTCCAGGTAAAAC | 369 |
| | TTTACCTGGA | | GGAGCCACCCAaag | | GTTTTACCTGGA | | GGGGATGGCGGC | |
| LAG3_g668 | GGGATGGCGGGA | 370 | acgGGGATGGCGGGAGGGTTGA | 371 | caccGGGATGGCGGGA | 372 | agacGAGGTCAACCCT | 373 |
| | GGGTTGACCTC | | CCTCCAGCCCCAcag | | GGGTTGACCTC | | CCCGCCATCCC | |
| LAG3_g714 | GAGCTAAGGACCT | 374 | acaGAGCTAAGGACCTCAGGAC | 375 | caccGAGCTAAGGACC | 376 | agacCAAAGGTCCTGA | 377 |
| | CAGGACCTTTG | | CTTTGTGGCCACAgag | | TCAGGACCTTTG | | GGTCCTTAGCTC | |
| LAG3_g768 | GAGAGGCTTTCGG | 378 | ccaGAGAGGCTTTCGGGGTGGA | 379 | caccGAGAGGCTTTCG | 380 | agacGTCCTTCCACCC | 381 |
| | GGTGGAAGGAC | | AGGACTGCCCAAAaaa | | GGGTGGAAGGAC | | CGAAAGCCTCTC | |
| LAG3_g769 | GAGAGGCTTTCGG | 382 | ccaGAGAGGCTTTCGGGGTGGA | 383 | caccGAGAGGCTTTCG | 384 | agacTCCTTCCACCCC | 385 |
| | GGTGGAAGGA | | AGGACTGCCCAAaaa | | GGGTGGAAGGA | | GAAAGCCTCTC | |
| LAG3_g770 | AGAGAGGCTTTCG | 386 | cccAGAGAGGCTTTCGGGGGG | 387 | caccGAGAGAGGCTTT | 388 | agacCCTTCCACCCCG | 389 |
| | GGGTGGAAGG | | AAGGACTGCCCAaaa | | CGGGGTGGAAGG | | AAAGCCTCTCTC | |

(*) The target sequences are reported with three flanking nucleotides on each side.
The PAM sequence is highlighted in bold.
(**) The cloning overhang is reported in lowercase.
Nucleotides highlighted in bold represent 5′-G appended to favor transcription from canonical U6 Pol III promoter

TABLE 18
Oligonucleotides used to amplify target
regions to perform TIDE analysis

| | | SEQ | | SEQ |
|---|---|---|---|---|
| | For | ID | Rev | ID |
| Locus | (5′ → 3′) | NO: | (5′ → 3′) | NO: |
| TRAC | CATCACGAGCA | 390 | TGGCAATGGA | 391 |
| | GCTGGTTTC | | TAAGGCCGAG | |
| B2M | CTCTAACCTGG | 392 | CGAAGTCCAC | 393 |
| | CACTGCGTC | | AGCTCTCCAG | |
| PD-1 | CACTGCCTCTG | 394 | GACTTGGGCC | 395 |
| | TCACTCTCG | | AGGGGAGGAG | |
| LAG3 | CCCCTTCCTCC | 396 | GGTTGAGTGC | 397 |
| | ACCTCCCTC | | TGGATTCGGA | |

### 7.4.2. Results

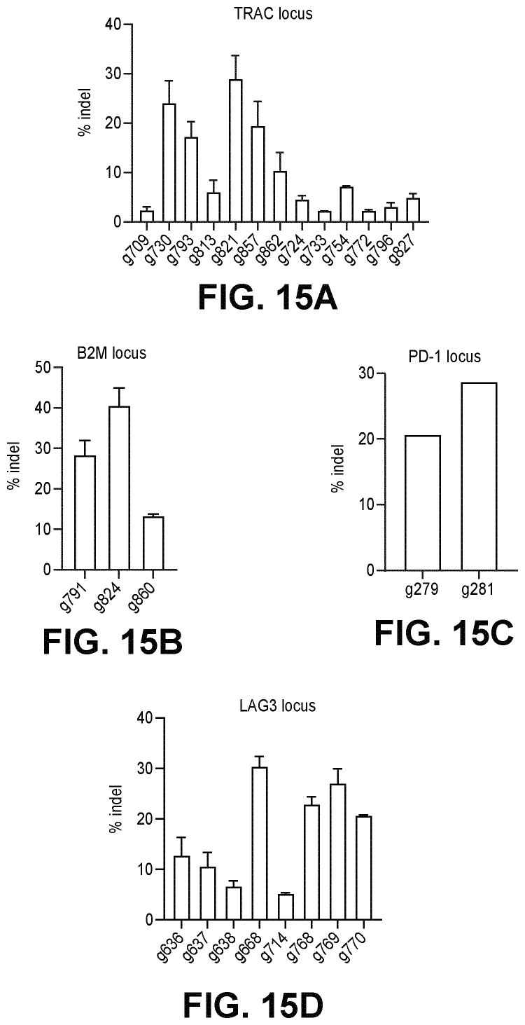

As shown in FIGS. 15A-15D, significant editing activity was observed at each locus, demonstrating that ENQP Type II Cas has the ability to effectively modify various genomic targets of interest.

### 8. SPECIFIC EMBODIMENTS

The present disclosure is exemplified by the specific embodiments below.

1. A Type II Cas protein comprising an amino acid sequence having at least 50% sequence identity to:
(a) the amino acid sequence of a RuvC-I domain of a reference protein sequence;
(b) the amino acid sequence of a RuvC-II domain of a reference protein sequence;
(c) the amino acid sequence of a RuvC-III domain of a reference protein sequence;
(d) the amino acid sequence of a BH domain of a reference protein sequence;
(e) the amino acid sequence of a REC domain of a reference protein sequence;
(f) the amino acid sequence of a HNH domain of a reference protein sequence;
(g) the amino acid sequence of a WED domain of a reference protein sequence;
(h) the amino acid sequence of a PID domain of a reference protein sequence; or
(i) the amino acid sequence of the full length of a reference protein sequence;
wherein the reference protein sequence is SEQ ID NO:1 or SEQ ID NO:2.

2. The Type II Cas protein of embodiment 1, wherein the amino acid sequence of the Type II Cas protein comprises an amino acid sequence that is at least 50% identical to the amino acid sequence of the RuvC-I domain of the reference protein sequence.

3. The Type II Cas protein of embodiment 1, wherein the amino acid sequence of the Type II Cas protein comprises an amino acid sequence that is at least 55% identical to the amino acid sequence of the RuvC-I domain of the reference protein sequence.

4. The Type II Cas protein of embodiment 1, wherein the amino acid sequence of the Type II Cas protein comprises an amino acid sequence that is at least 60% identical to the amino acid sequence of the RuvC-I domain of the reference protein sequence.

5. The Type II Cas protein of embodiment 1, wherein the amino acid sequence of the Type II Cas protein comprises an amino acid sequence that is at least 65% identical to the amino acid sequence of the RuvC-I domain of the reference protein sequence.

6. The Type II Cas protein of embodiment 1, wherein the amino acid sequence of the Type II Cas protein comprises an amino acid sequence that is at least 70% identical to the amino acid sequence of the RuvC-I domain of the reference protein sequence.

7. The Type II Cas protein of embodiment 1, wherein the amino acid sequence of the Type II Cas protein comprises an amino acid sequence that is at least 75% identical to the amino acid sequence of the RuvC-I domain of the reference protein sequence.

8. The Type II Cas protein of embodiment 1, wherein the amino acid sequence of the Type II Cas protein comprises an amino acid sequence that is at least 80% identical to the amino acid sequence of the RuvC-I domain of the reference protein sequence.

9. The Type II Cas protein of embodiment 1, wherein the amino acid sequence of the Type II Cas protein comprises an amino acid sequence that is at least 85% identical to the amino acid sequence of the RuvC-I domain of the reference protein sequence.

10. The Type II Cas protein of embodiment 1, wherein the amino acid sequence of the Type II Cas protein comprises an amino acid sequence that is at least 90% identical to the amino acid sequence of the RuvC-I domain of the reference protein sequence.

11. The Type II Cas protein of embodiment 1, wherein the amino acid sequence of the Type II Cas protein comprises an amino acid sequence that is at least 95% identical to the amino acid sequence of the RuvC-I domain of the reference protein sequence.

12. The Type II Cas protein of embodiment 1, wherein the amino acid sequence of the Type II Cas protein comprises an amino acid sequence that is at least 96% identical to the amino acid sequence of the RuvC-I domain of the reference protein sequence.

13. The Type II Cas protein of embodiment 1, wherein the amino acid sequence of the Type II Cas protein comprises an amino acid sequence that is at least 97% identical to the amino acid sequence of the RuvC-I domain of the reference protein sequence.

14. The Type II Cas protein of embodiment 1, wherein the amino acid sequence of the Type II Cas protein comprises an amino acid sequence that is at least 98% identical to the amino acid sequence of the RuvC-I domain of the reference protein sequence.

15. The Type II Cas protein of embodiment 1, wherein the amino acid sequence of the Type II Cas protein comprises an amino acid sequence that is at least 99% identical to the amino acid sequence of the RuvC-I domain of the reference protein sequence.

16. The Type II Cas protein of embodiment 1, wherein the amino acid sequence of the Type II Cas protein comprises an amino acid sequence that is identical to the amino acid sequence of the RuvC-I domain of the reference protein sequence.

17. The Type II Cas protein of any one of embodiments 1 to 16, wherein the amino acid sequence of the Type II Cas protein comprises an amino acid sequence that is at least 50% identical to the amino acid sequence of the RuvC-II domain of the reference protein sequence.

18. The Type II Cas protein of any one of embodiments 1 to 16, wherein the amino acid sequence of the Type II Cas protein comprises an amino acid sequence that is at least 55% identical to the amino acid sequence of the RuvC-II domain of the reference protein sequence.

19. The Type II Cas protein of any one of embodiments 1 to 16, wherein the amino acid sequence of the Type II Cas protein comprises an amino acid sequence that is at least 60% identical to the amino acid sequence of the RuvC-II domain of the reference protein sequence.

20. The Type II Cas protein of any one of embodiments 1 to 16, wherein the amino acid sequence of the Type II Cas protein comprises an amino acid sequence that is at least 65% identical to the amino acid sequence of the RuvC-II domain of the reference protein sequence.

21. The Type II Cas protein of any one of embodiments 1 to 16, wherein the amino acid sequence of the Type II Cas protein comprises an amino acid sequence that is at least 70% identical to the amino acid sequence of the RuvC-II domain of the reference protein sequence.

22. The Type II Cas protein of any one of embodiments 1 to 16, wherein the amino acid sequence of the Type II Cas protein comprises an amino acid sequence that is at least 75% identical to the amino acid sequence of the RuvC-II domain of the reference protein sequence.

23. The Type II Cas protein of any one of embodiments 1 to 16, wherein the amino acid sequence of the Type II Cas protein comprises an amino acid sequence that is at least 80% identical to the amino acid sequence of the RuvC-II domain of the reference protein sequence.

24. The Type II Cas protein of any one of embodiments 1 to 16, wherein the amino acid sequence of the Type II Cas protein comprises an amino acid sequence that is at least 85% identical to the amino acid sequence of the RuvC-II domain of the reference protein sequence.

25. The Type II Cas protein of any one of embodiments 1 to 16, wherein the amino acid sequence of the Type II Cas protein comprises an amino acid sequence that is at least 90% identical to the amino acid sequence of the RuvC-II domain of the reference protein sequence.

26. The Type II Cas protein of any one of embodiments 1 to 16, wherein the amino acid sequence of the Type II Cas protein comprises an amino acid sequence that is at least 95% identical to the amino acid sequence of the RuvC-II domain of the reference protein sequence.

27. The Type II Cas protein of any one of embodiments 1 to 16, wherein the amino acid sequence of the Type II Cas protein comprises an amino acid sequence that is at least 96% identical to the amino acid sequence of the RuvC-II domain of the reference protein sequence.

28. The Type II Cas protein of any one of embodiments 1 to 16, wherein the amino acid sequence of the Type II Cas protein comprises an amino acid sequence that is at least 97% identical to the amino acid sequence of the RuvC-II domain of the reference protein sequence.

29. The Type II Cas protein of any one of embodiments 1 to 16, wherein the amino acid sequence of the Type II Cas protein comprises an amino acid sequence that is at least 98% identical to the amino acid sequence of the RuvC-II domain of the reference protein sequence.

30. The Type II Cas protein of any one of embodiments 1 to 16, wherein the amino acid sequence of the Type II Cas protein comprises an amino acid sequence that is at least 99% identical to the amino acid sequence of the RuvC-II domain of the reference protein sequence.

31. The Type II Cas protein of any one of embodiments 1 to 16, wherein the amino acid sequence of the Type II Cas protein comprises an amino acid sequence that is identical to the amino acid sequence of the RuvC-II domain of the reference protein sequence.

32. The Type II Cas protein of any one of embodiments 1 to 31, wherein the amino acid sequence of the Type II Cas protein comprises an amino acid sequence that is at least 50% identical to the amino acid sequence of the RuvC-III domain of the reference protein sequence.

33. The Type II Cas protein of any one of embodiments 1 to 31, wherein the amino acid sequence of the Type II Cas protein comprises an amino acid sequence that is at least 55% identical to the amino acid sequence of the RuvC-III domain of the reference protein sequence.

34. The Type II Cas protein of any one of embodiments 1 to 31, wherein the amino acid sequence of the Type II Cas protein comprises an amino acid sequence that is at least 60% identical to the amino acid sequence of the RuvC-III domain of the reference protein sequence.

35. The Type II Cas protein of any one of embodiments 1 to 31, wherein the amino acid sequence of the Type II Cas protein comprises an amino acid sequence that is at least 65% identical to the amino acid sequence of the RuvC-III domain of the reference protein sequence.

36. The Type II Cas protein of any one of embodiments 1 to 31, wherein the amino acid sequence of the Type II Cas protein comprises an amino acid sequence that is at least 70% identical to the amino acid sequence of the RuvC-III domain of the reference protein sequence.

37. The Type II Cas protein of any one of embodiments 1 to 31, wherein the amino acid sequence of the Type II Cas protein comprises an amino acid sequence that is at least 75% identical to the amino acid sequence of the RuvC-III domain of the reference protein sequence.

38. The Type II Cas protein of any one of embodiments 1 to 31, wherein the amino acid sequence of the Type II Cas protein comprises an amino acid sequence that is at least 80% identical to the amino acid sequence of the RuvC-III domain of the reference protein sequence.

39. The Type II Cas protein of any one of embodiments 1 to 31, wherein the amino acid sequence of the Type II Cas protein comprises an amino acid sequence that is at least 85% identical to the amino acid sequence of the RuvC-III domain of the reference protein sequence.

40. The Type II Cas protein of any one of embodiments 1 to 31, wherein the amino acid sequence of the Type II Cas protein comprises an amino acid sequence that is at least 90% identical to the amino acid sequence of the RuvC-III domain of the reference protein sequence.

41. The Type II Cas protein of any one of embodiments 1 to 31, wherein the amino acid sequence of the Type II Cas protein comprises an amino acid sequence that is at least 95% identical to the amino acid sequence of the RuvC-III domain of the reference protein sequence.

42. The Type II Cas protein of any one of embodiments 1 to 31, wherein the amino acid sequence of the Type II Cas protein comprises an amino acid sequence that is at least 96% identical to the amino acid sequence of the RuvC-III domain of the reference protein sequence.

43. The Type II Cas protein of any one of embodiments 1 to 31, wherein the amino acid sequence of the Type II Cas protein comprises an amino acid sequence that is at least 97% identical to the amino acid sequence of the RuvC-III domain of the reference protein sequence.

44. The Type II Cas protein of any one of embodiments 1 to 31, wherein the amino acid sequence of the Type II Cas protein comprises an amino acid sequence that is at least 98% identical to the amino acid sequence of the RuvC-III domain of the reference protein sequence.

45. The Type II Cas protein of any one of embodiments 1 to 31, wherein the amino acid sequence of the Type II Cas protein comprises an amino acid sequence that is at least 99% identical to the amino acid sequence of the RuvC-III domain of the reference protein sequence.

46. The Type II Cas protein of any one of embodiments 1 to 31, wherein the amino acid sequence of the Type II Cas protein comprises an amino acid sequence that is identical to the amino acid sequence of the RuvC-III domain of the reference protein sequence.

47. The Type II Cas protein of any one of embodiments 1 to 46, wherein the amino acid sequence of the Type II Cas protein comprises an amino acid sequence that is at least 50% identical to the amino acid sequence of the BH domain of the reference protein sequence.

48. The Type II Cas protein of any one of embodiments 1 to 46, wherein the amino acid sequence of the Type II Cas protein comprises an amino acid sequence that is at least 55% identical to the amino acid sequence of the BH domain of the reference protein sequence.

49. The Type II Cas protein of any one of embodiments 1 to 46, wherein the amino acid sequence of the Type II Cas protein comprises an amino acid sequence that is at least 60% identical to the amino acid sequence of the BH domain of the reference protein sequence.

50. The Type II Cas protein of any one of embodiments 1 to 46, wherein the amino acid sequence of the Type II Cas protein comprises an amino acid sequence that is at least 65% identical to the amino acid sequence of the BH domain of the reference protein sequence.

51. The Type II Cas protein of any one of embodiments 1 to 46, wherein the amino acid sequence of the Type II Cas protein comprises an amino acid sequence that is at least 70% identical to the amino acid sequence of the BH domain of the reference protein sequence.

52. The Type II Cas protein of any one of embodiments 1 to 46, wherein the amino acid sequence of the Type II Cas protein comprises an amino acid sequence that is at least 75% identical to the amino acid sequence of the BH domain of the reference protein sequence.

53. The Type II Cas protein of any one of embodiments 1 to 46, wherein the amino acid sequence of the Type II Cas protein comprises an amino acid sequence that is at least 80% identical to the amino acid sequence of the BH domain of the reference protein sequence.

54. The Type II Cas protein of any one of embodiments 1 to 46, wherein the amino acid sequence of the Type II Cas protein comprises an amino acid sequence that is at least 85% identical to the amino acid sequence of the BH domain of the reference protein sequence.

55. The Type II Cas protein of any one of embodiments 1 to 46, wherein the amino acid sequence of the Type II Cas protein comprises an amino acid sequence that is at least 90% identical to the amino acid sequence of the BH domain of the reference protein sequence.

56. The Type II Cas protein of any one of embodiments 1 to 46, wherein the amino acid sequence of the Type II Cas protein comprises an amino acid sequence that is at least 95% identical to the amino acid sequence of the BH domain of the reference protein sequence.

57. The Type II Cas protein of any one of embodiments 1 to 46, wherein the amino acid sequence of the Type II Cas protein comprises an amino acid sequence that is at least 96% identical to the amino acid sequence of the BH domain of the reference protein sequence.

58. The Type II Cas protein of any one of embodiments 1 to 46, wherein the amino acid sequence of the Type II Cas protein comprises an amino acid sequence that is at least 97% identical to the amino acid sequence of the BH domain of the reference protein sequence.

59. The Type II Cas protein of any one of embodiments 1 to 46, wherein the amino acid sequence of the Type II Cas protein comprises an amino acid sequence that is at least 98% identical to the amino acid sequence of the BH domain of the reference protein sequence.

60. The Type II Cas protein of any one of embodiments 1 to 46, wherein the amino acid sequence of the Type II Cas protein comprises an amino acid sequence that is at least 99% identical to the amino acid sequence of the BH domain of the reference protein sequence.

61. The Type II Cas protein of any one of embodiments 1 to 46, wherein the amino acid sequence of the Type II Cas protein comprises an amino acid sequence that is identical to the amino acid sequence of the BH domain of the reference protein sequence.

62. The Type II Cas protein of any one of embodiments 1 to 61, wherein the amino acid sequence of the Type II Cas protein comprises an amino acid sequence that is at least 50% identical to the amino acid sequence of the REC domain of the reference protein sequence.

63. The Type II Cas protein of any one of embodiments 1 to 61, wherein the amino acid sequence of the Type II Cas protein comprises an amino acid sequence that is at least 55% identical to the amino acid sequence of the REC domain of the reference protein sequence.

64. The Type II Cas protein of any one of embodiments 1 to 61, wherein the amino acid sequence of the Type II Cas protein comprises an amino acid sequence that is at least 60% identical to the amino acid sequence of the REC domain of the reference protein sequence.

65. The Type II Cas protein of any one of embodiments 1 to 61, wherein the amino acid sequence of the Type II Cas protein comprises an amino acid sequence that is at least 65% identical to the amino acid sequence of the REC domain of the reference protein sequence.

66. The Type II Cas protein of any one of embodiments 1 to 61, wherein the amino acid sequence of the Type II Cas protein comprises an amino acid sequence that is at least 70% identical to the amino acid sequence of the REC domain of the reference protein sequence.

67. The Type II Cas protein of any one of embodiments 1 to 61, wherein the amino acid sequence of the Type II Cas protein comprises an amino acid sequence that is at least 75% identical to the amino acid sequence of the REC domain of the reference protein sequence.

68. The Type II Cas protein of any one of embodiments 1 to 61, wherein the amino acid sequence of the Type II Cas protein comprises an amino acid sequence that is at least 80% identical to the amino acid sequence of the REC domain of the reference protein sequence.

69. The Type II Cas protein of any one of embodiments 1 to 61, wherein the amino acid sequence of the Type II Cas protein comprises an amino acid sequence that is at least 85% identical to the amino acid sequence of the REC domain of the reference protein sequence.

70. The Type II Cas protein of any one of embodiments 1 to 61, wherein the amino acid sequence of the Type II Cas protein comprises an amino acid sequence that is at least 90% identical to the amino acid sequence of the REC domain of the reference protein sequence.

71. The Type II Cas protein of any one of embodiments 1 to 61, wherein the amino acid sequence of the Type II Cas protein comprises an amino acid sequence that is at least 95% identical to the amino acid sequence of the REC domain of the reference protein sequence.

72. The Type II Cas protein of any one of embodiments 1 to 61, wherein the amino acid sequence of the Type II Cas protein comprises an amino acid sequence that is at least 96% identical to the amino acid sequence of the REC domain of the reference protein sequence.

73. The Type II Cas protein of any one of embodiments 1 to 61, wherein the amino acid sequence of the Type II Cas protein comprises an amino acid sequence that is at least 97% identical to the amino acid sequence of the REC domain of the reference protein sequence.

74. The Type II Cas protein of any one of embodiments 1 to 61, wherein the amino acid sequence of the Type II Cas protein comprises an amino acid sequence that is at least 98% identical to the amino acid sequence of the REC domain of the reference protein sequence.

75. The Type II Cas protein of any one of embodiments 1 to 61, wherein the amino acid sequence of the Type II Cas protein comprises an amino acid sequence that is at least 99% identical to the amino acid sequence of the REC domain of the reference protein sequence.

76. The Type II Cas protein of any one of embodiments 1 to 61, wherein the amino acid sequence of the Type II Cas protein comprises an amino acid sequence that is identical to the amino acid sequence of the REC domain of the reference protein sequence.

77. The Type II Cas protein of any one of embodiments 1 to 76, wherein the amino acid sequence of the Type II Cas protein comprises an amino acid sequence that is at least 50% identical to the amino acid sequence of the HNH domain of the reference protein sequence.

78. The Type II Cas protein of any one of embodiments 1 to 76, wherein the amino acid sequence of the Type II Cas protein comprises an amino acid sequence that is at least 55% identical to the amino acid sequence of the HNH domain of the reference protein sequence.

79. The Type II Cas protein of any one of embodiments 1 to 76, wherein the amino acid sequence of the Type II Cas protein comprises an amino acid sequence that is at least 60% identical to the amino acid sequence of the HNH domain of the reference protein sequence.

80. The Type II Cas protein of any one of embodiments 1 to 76, wherein the amino acid sequence of the Type II Cas protein comprises an amino acid sequence that is at least 65% identical to the amino acid sequence of the HNH domain of the reference protein sequence.

81. The Type II Cas protein of any one of embodiments 1 to 76, wherein the amino acid sequence of the Type II Cas protein comprises an amino acid sequence that is at least 70% identical to the amino acid sequence of the HNH domain of the reference protein sequence.

82. The Type II Cas protein of any one of embodiments 1 to 76, wherein the amino acid sequence of the Type II Cas protein comprises an amino acid sequence that is at least 75% identical to the amino acid sequence of the HNH domain of the reference protein sequence.

83. The Type II Cas protein of any one of embodiments 1 to 76, wherein the amino acid sequence of the Type II Cas protein comprises an amino acid sequence that is at least 80% identical to the amino acid sequence of the HNH domain of the reference protein sequence.

84. The Type II Cas protein of any one of embodiments 1 to 76, wherein the amino acid sequence of the Type II Cas protein comprises an amino acid sequence that is at least 85% identical to the amino acid sequence of the HNH domain of the reference protein sequence.

85. The Type II Cas protein of any one of embodiments 1 to 76, wherein the amino acid sequence of the Type II Cas protein comprises an amino acid sequence that is at least 90% identical to the amino acid sequence of the HNH domain of the reference protein sequence.

86. The Type II Cas protein of any one of embodiments 1 to 76, wherein the amino acid sequence of the Type II Cas protein comprises an amino acid sequence that is at least 95% identical to the amino acid sequence of the HNH domain of the reference protein sequence.

87. The Type II Cas protein of any one of embodiments 1 to 76, wherein the amino acid sequence of the Type II Cas protein comprises an amino acid sequence that is at least 96% identical to the amino acid sequence of the HNH domain of the reference protein sequence.

88. The Type II Cas protein of any one of embodiments 1 to 76, wherein the amino acid sequence of the Type II Cas protein comprises an amino acid sequence that is at least 97% identical to the amino acid sequence of the HNH domain of the reference protein sequence.

89. The Type II Cas protein of any one of embodiments 1 to 76, wherein the amino acid sequence of the Type II Cas protein comprises an amino acid sequence that is at least 98% identical to the amino acid sequence of the HNH domain of the reference protein sequence.

90. The Type II Cas protein of any one of embodiments 1 to 76, wherein the amino acid sequence of the Type II Cas protein comprises an amino acid sequence that is at least 99% identical to the amino acid sequence of the HNH domain of the reference protein sequence.

91. The Type II Cas protein of any one of embodiments 1 to 76, wherein the amino acid sequence of the Type II Cas protein comprises an amino acid sequence that is identical to the amino acid sequence of the HNH domain of the reference protein sequence.

92. The Type II Cas protein of any one of embodiments 1 to 91, wherein the amino acid sequence of the Type II Cas protein comprises an amino acid sequence that is at least 50% identical to the amino acid sequence of the WED domain of the reference protein sequence.

93. The Type II Cas protein of any one of embodiments 1 to 91, wherein the amino acid sequence of the Type II Cas protein comprises an amino acid sequence that is at least 55% identical to the amino acid sequence of the WED domain of the reference protein sequence.

94. The Type II Cas protein of any one of embodiments 1 to 91, wherein the amino acid sequence of the Type II Cas protein comprises an amino acid sequence that is at least 60% identical to the amino acid sequence of the WED domain of the reference protein sequence.

95. The Type II Cas protein of any one of embodiments 1 to 91, wherein the amino acid sequence of the Type II Cas protein comprises an amino acid sequence that is at least 65% identical to the amino acid sequence of the WED domain of the reference protein sequence.

96. The Type II Cas protein of any one of embodiments 1 to 91, wherein the amino acid sequence of the Type II Cas protein comprises an amino acid sequence that is at least 70% identical to the amino acid sequence of the WED domain of the reference protein sequence.

97. The Type II Cas protein of any one of embodiments 1 to 91, wherein the amino acid sequence of the Type II Cas protein comprises an amino acid sequence that is at least 75% identical to the amino acid sequence of the WED domain of the reference protein sequence.

98. The Type II Cas protein of any one of embodiments 1 to 91, wherein the amino acid sequence of the Type II Cas protein comprises an amino acid sequence that is at least 80% identical to the amino acid sequence of the WED domain of the reference protein sequence.

99. The Type II Cas protein of any one of embodiments 1 to 91, wherein the amino acid sequence of the Type II Cas protein comprises an amino acid sequence that is at least 85% identical to the amino acid sequence of the WED domain of the reference protein sequence.

100. The Type II Cas protein of any one of embodiments 1 to 91, wherein the amino acid sequence of the Type II Cas protein comprises an amino acid sequence that is at least 90% identical to the amino acid sequence of the WED domain of the reference protein sequence.

101. The Type II Cas protein of any one of embodiments 1 to 91, wherein the amino acid sequence of the Type II Cas protein comprises an amino acid sequence that is at least 95% identical to the amino acid sequence of the WED domain of the reference protein sequence.

102. The Type II Cas protein of any one of embodiments 1 to 91, wherein the amino acid sequence of the Type II Cas protein comprises an amino acid sequence that is at least 96% identical to the amino acid sequence of the WED domain of the reference protein sequence.

103. The Type II Cas protein of any one of embodiments 1 to 91, wherein the amino acid sequence of the Type II Cas protein comprises an amino acid sequence that is at least 97% identical to the amino acid sequence of the WED domain of the reference protein sequence.

104. The Type II Cas protein of any one of embodiments 1 to 91, wherein the amino acid sequence of the Type II Cas protein comprises an amino acid sequence that is at least 98% identical to the amino acid sequence of the WED domain of the reference protein sequence.

105. The Type II Cas protein of any one of embodiments 1 to 91, wherein the amino acid sequence of the Type II Cas protein comprises an amino acid sequence that is at least 99% identical to the amino acid sequence of the WED domain of the reference protein sequence.

106. The Type II Cas protein of any one of embodiments 1 to 91, wherein the amino acid sequence of the Type II Cas protein comprises an amino acid sequence that is identical to the amino acid sequence of the WED domain of the reference protein sequence.

107. The Type II Cas protein of any one of embodiments 1 to 106, wherein the amino acid sequence of the Type II Cas protein comprises an amino acid sequence that is at least 50% identical to the amino acid sequence of the PID domain of the reference protein sequence.

108. The Type II Cas protein of any one of embodiments 1 to 106, wherein the amino acid sequence of the Type II Cas protein comprises an amino acid sequence that is at least 55% identical to the amino acid sequence of the PID domain of the reference protein sequence.

109. The Type II Cas protein of any one of embodiments 1 to 106, wherein the amino acid sequence of the Type II Cas protein comprises an amino acid sequence that is at least 60% identical to the amino acid sequence of the PID domain of the reference protein sequence.

110. The Type II Cas protein of any one of embodiments 1 to 106, wherein the amino acid sequence of the Type II Cas protein comprises an amino acid sequence that is at least 65% identical to the amino acid sequence of the PID domain of the reference protein sequence.

111. The Type II Cas protein of any one of embodiments 1 to 106, wherein the amino acid sequence of the Type II Cas protein comprises an amino acid sequence that is at least 70% identical to the amino acid sequence of the PID domain of the reference protein sequence.

112. The Type II Cas protein of any one of embodiments 1 to 106, wherein the amino acid sequence of the Type II Cas protein comprises an amino acid sequence that is at least 75% identical to the amino acid sequence of the PID domain of the reference protein sequence.

113. The Type II Cas protein of any one of embodiments 1 to 106, wherein the amino acid sequence of the Type II Cas protein comprises an amino acid sequence that is at least 80% identical to the amino acid sequence of the PID domain of the reference protein sequence.

114. The Type II Cas protein of any one of embodiments 1 to 106, wherein the amino acid sequence of the Type II Cas protein comprises an amino acid sequence that is at least 85% identical to the amino acid sequence of the PID domain of the reference protein sequence.

115. The Type II Cas protein of any one of embodiments 1 to 106, wherein the amino acid sequence of the Type II Cas protein comprises an amino acid sequence that is at least 90% identical to the amino acid sequence of the PID domain of the reference protein sequence.

116. The Type II Cas protein of any one of embodiments 1 to 106, wherein the amino acid sequence of the Type II Cas protein comprises an amino acid sequence that is at least 95% identical to the amino acid sequence of the PID domain of the reference protein sequence.

117. The Type II Cas protein of any one of embodiments 1 to 106, wherein the amino acid sequence of the Type II Cas protein comprises an amino acid sequence that is at least 96% identical to the amino acid sequence of the PID domain of the reference protein sequence.

118. The Type II Cas protein of any one of embodiments 1 to 106, wherein the amino acid sequence of the Type II Cas protein comprises an amino acid sequence that is at least 97% identical to the amino acid sequence of the PID domain of the reference protein sequence.

119. The Type II Cas protein of any one of embodiments 1 to 106, wherein the amino acid sequence of the Type II Cas protein comprises an amino acid sequence that is at least 98% identical to the amino acid sequence of the PID domain of the reference protein sequence.

120. The Type II Cas protein of any one of embodiments 1 to 106, wherein the amino acid sequence of the Type II Cas protein comprises an amino acid sequence that is at least 99% identical to the amino acid sequence of the PID domain of the reference protein sequence.

121. The Type II Cas protein of any one of embodiments 1 to 106, wherein the amino acid sequence of the Type II Cas protein comprises an amino acid sequence that is identical to the amino acid sequence of the PID domain of the reference protein sequence.

122. The Type II Cas protein of embodiment 1, wherein the amino acid sequence of the Type II Cas protein comprises an amino acid sequence that is at least 55% identical to the full length of the reference protein sequence.

123. The Type II Cas protein of embodiment 1, wherein the amino acid sequence of the Type II Cas protein comprises an amino acid sequence that is at least 60% identical to the full length of the reference protein sequence.

124. The Type II Cas protein of embodiment 1, wherein the amino acid sequence of the Type II Cas protein comprises an amino acid sequence that is at least 65% identical to the full length of the reference protein sequence.

125. The Type II Cas protein of embodiment 1, wherein the amino acid sequence of the Type II Cas protein comprises an amino acid sequence that is at least 70% identical to the full length of the reference protein sequence.

126. The Type II Cas protein of embodiment 1, wherein the amino acid sequence of the Type II Cas protein comprises an amino acid sequence that is at least 75% identical to the full length of the reference protein sequence.

127. The Type II Cas protein of embodiment 1, wherein the amino acid sequence of the Type II Cas protein comprises an amino acid sequence that is at least 80% identical to the full length of the reference protein sequence.

128. The Type II Cas protein of embodiment 1, wherein the amino acid sequence of the Type II Cas protein comprises an amino acid sequence that is at least 85% identical to the full length of the reference protein sequence.

129. The Type II Cas protein of embodiment 1, wherein the amino acid sequence of the Type II Cas protein comprises an amino acid sequence that is at least 90% identical to the full length of the reference protein sequence.

130. The Type II Cas protein of embodiment 1, wherein the amino acid sequence of the Type II Cas protein comprises an amino acid sequence that is at least 95% identical to the full length of the reference protein sequence.

131. The Type II Cas protein of embodiment 1, wherein the amino acid sequence of the Type II Cas protein comprises an amino acid sequence that is at least 96% identical to the full length of the reference protein sequence.

132. The Type II Cas protein of embodiment 1, wherein the amino acid sequence of the Type II Cas protein comprises an amino acid sequence that is at least 97% identical to the full length of the reference protein sequence.

133. The Type II Cas protein of embodiment 1, wherein the amino acid sequence of the Type II Cas protein comprises an amino acid sequence that is at least 98% identical to the full length of the reference protein sequence.

134. The Type II Cas protein of embodiment 1, wherein the amino acid sequence of the Type II Cas protein comprises an amino acid sequence that is at least 99% identical to the full length of the reference protein sequence.

135. The Type II Cas protein of embodiment 1, wherein the amino acid sequence of the Type II Cas protein comprises an amino acid sequence that is identical to the full length of the reference protein sequence.

136. The Type II Cas protein of any one of embodiments 1 to 135, which is a chimeric Type II Cas protein.

137. The Type II Cas protein of any one of embodiments 1 to 136, which is a fusion protein.

138. The Type II Cas protein of embodiment 137, which comprises one or more nuclear localization signals.

139. The Type II Cas protein of embodiment 138, which comprises two or more nuclear localization signals.

140. The Type II Cas protein of embodiment 138 or embodiment 139, which comprises an N-terminal nuclear localization signal.

141. The Type II Cas protein of any one of embodiments 138 to 140, which comprises a C-terminal nuclear localization signal.

142. The Type II Cas protein of any one of embodiments 138 to 141, which comprises an N-terminal nuclear localization signal and a C-terminal nuclear localization signal.

143. The Type II Cas protein of any one of embodiments 138 to 142, wherein the amino acid sequence of one or more of the nuclear localization signals comprises the amino acid sequence

(SEQ ID NO: 7)
KRTADGSEFESPKKKRKV,
(SEQ ID NO: 8)
PKKKRKV,
(SEQ ID NO: 9)
PKKKRRV,
(SEQ ID NO: 10)
KRPAATKKAGQAKKKK,
(SEQ ID NO: 11)
YGRKKRRQRRR,
(SEQ ID NO: 12)
RKKRRQRRR,
(SEQ ID NO: 13)
PAAKRVKLD,
(SEQ ID NO: 14)
RQRRNELKRSP,
(SEQ ID NO: 15)
VSRKRPRP,
(SEQ ID NO: 16)
PPKKARED,
(SEQ ID NO: 17)
PQPKKKPL,
(SEQ ID NO: 18)
SALIKKKKKMAP,
(SEQ ID NO: 19)
PKQKKRK,
(SEQ ID NO: 20)
RKLKKKIKKL,
(SEQ ID NO: 21)
REKKKFLKRR,
(SEQ ID NO: 22)
KRKGDEVDGVDEVAKKKSKK,
(SEQ ID NO: 23)
RKCLQAGMNLEARKTKK,
(SEQ ID NO: 24)
NQSSNFGPMKGGNFGGRSSGPYGGGGQYFAKPRNQGGY,
or
(SEQ ID NO: 25)
RMRIZFKNKGKDTAELRRRRVEVSVELRKAKKDEQILKRRNV.

144. The Type II Cas protein of embodiment 143, wherein the amino acid sequence of one or more of the nuclear localization signals comprises the amino acid sequence KRTADGSEFESPKKKRKV (SEQ ID NO:7).

145. The Type II Cas protein of embodiment 143, wherein the amino acid sequence of one or more of the nuclear localization signals comprises the amino acid sequence PKKKRKV (SEQ ID NO:8).

146. The Type II Cas protein of embodiment 143, wherein the amino acid sequence of one or more of the nuclear localization signals comprises the amino acid sequence PKKKRRV (SEQ ID NO:9).

147. The Type II Cas protein of embodiment 143, wherein the amino acid sequence of one or more of the nuclear localization signals comprises the amino acid sequence KRPAATKKAGQAKKKK (SEQ ID NO:10).

148. The Type II Cas protein of embodiment 143, wherein the amino acid sequence of one or more of the nuclear localization signals comprises the amino acid sequence YGRKKRRQRRR (SEQ ID NO: 11).

149. The Type II Cas protein of embodiment 143, wherein the amino acid sequence of one or more of the nuclear localization signals comprises the amino acid sequence RKKRRQRRR (SEQ ID NO: 12).

150. The Type II Cas protein of embodiment 143, wherein the amino acid sequence of one or more of the nuclear localization signals comprises the amino acid sequence PAAKRVKLD (SEQ ID NO: 13).

151. The Type II Cas protein of embodiment 143, wherein the amino acid sequence of one or more of the nuclear localization signals comprises the amino acid sequence RQRRNELKRSP (SEQ ID NO: 14).

152. The Type II Cas protein of embodiment 143, wherein the amino acid sequence of one or more of the nuclear localization signals comprises the amino acid sequence VSRKRPRP (SEQ ID NO: 15).

153. The Type II Cas protein of embodiment 143, wherein the amino acid sequence of one or more of the nuclear localization signals comprises the amino acid sequence PPKKARED (SEQ ID NO: 16).

154. The Type II Cas protein of embodiment 143, wherein the amino acid sequence of one or more of the nuclear localization signals comprises the amino acid sequence PQPKKKPL (SEQ ID NO: 17).

155. The Type II Cas protein of embodiment 143, wherein the amino acid sequence of one or more of the nuclear localization signals comprises the amino acid sequence SALIKKKKKMAP (SEQ ID NO: 18).

156. The Type II Cas protein of embodiment 143, wherein the amino acid sequence of one or more of the nuclear localization signals comprises the amino acid sequence PKQKKRK (SEQ ID NO:19).

157. The Type II Cas protein of embodiment 143, wherein the amino acid sequence of one or more of the nuclear localization signals comprises the amino acid sequence RKLKKKIKKL (SEQ ID NO: 20).

158. The Type II Cas protein of embodiment 143, wherein the amino acid sequence of one or more of the nuclear localization signals comprises the amino acid sequence REKKKFLKRR (SEQ ID NO: 21).

159. The Type II Cas protein of embodiment 143, wherein the amino acid sequence of one or more of the nuclear localization signals comprises the amino acid sequence KRKGDEVDGVDEVAKKKSKK (SEQ ID NO:22).

160. The Type II Cas protein of embodiment 143, wherein the amino acid sequence of one or more of the nuclear localization signals comprises the amino acid sequence RKCLQAGMNLEARKTKK (SEQ ID NO:23).

161. The Type II Cas protein of embodiment 143, wherein the amino acid sequence of one or more of the nuclear localization signals comprises the amino acid sequence NQSSNFGPMKGGNFGGRSSGPYGGGGQYFAKPRNQGGY (SEQ ID NO:24).

162. The Type II Cas protein of embodiment 143, wherein the amino acid sequence of one or more of the nuclear localization signals comprises the amino acid sequence RMRIZFKNKGKDTAELRRRRVEVSVELRKAKKDEQILKRRNV (SEQ ID NO:25).

163. The Type II Cas protein of any one of embodiments 138 to 162, wherein the amino acid sequence of each nuclear localization signal is the same.

164. The Type II Cas protein of any one of embodiments 136 to 163, which comprises a fusion partner which is a DNA, RNA or protein modification enzyme, optionally wherein the DNA, RNA or protein modification enzyme is an adenosine deaminase, a cytidine deaminase, a reverse transcriptase, a guanosyl transferase, a DNA methyltransferase, a RNA methyltransferase, a DNA demethylase, a RNA demethylase, a dioxygenase, a polyadenylate polymerase, a pseudouridine synthase, an acetyltransferase, a deacetylase, a ubiquitin-ligase, a deubiquitinase, a kinase, a phosphatase, a NEDD8-ligase, a de-NEDDylase, a SUMO-ligase, a deSUMOylase, a histone deacetylase, a histone acetyltransferase, a histone methyltransferase, or a histone demethylase.

165. The Type II Cas protein of any one of embodiments 136 to 164, which comprises a means for deaminating adenosine, optionally wherein the means for deaminating adenosine is an adenosine deaminase.

166. The Type II Cas protein of any one of embodiments 136 to 164, which comprises a fusion partner which is an adenosine deaminase, optionally wherein the amino acid sequence of the adenosine deaminase comprises an amino acid sequence having at least 50%, at least 60%, at least 70%, at least 80%, at least 90%, at least 95%, at least 97%, at least 98%, at least 99%, or 100% sequence identity with SEQ ID NO:27, optionally wherein the adenosine deaminase is the adenosine deaminase moiety contained in the adenine base editor ABE8e.

167. The Type II Cas protein of any one of embodiments 136 to 164, which comprises a means for deaminating cytidine, optionally wherein the means for deaminating cytidine is a cytodine deaminase.

168. The Type II Cas protein of any one of embodiments 136 to 164, which comprises a fusion partner which is a cytodine deaminase.

169. The Type II Cas protein of any one of embodiments 136 to 164, which comprises a means for synthesizing DNA from a single-stranded template, optionally wherein the means for synthesizing DNA from a single-stranded template is a reverse transcriptase.

170. The Type II Cas protein of any one of embodiments 136 to 164, which comprises a fusion partner which is a reverse transcriptase.

171. The Type II Cas protein of any one of embodiments 136 to 170, which comprises a tag.

172. The Type II Cas protein of embodiment 171, wherein the tag is a SV5 tag, optionally wherein the SV5 tag comprises the amino acid sequence GKPIPNPLLGLDST (SEQ ID NO:26).

173. The Type II Cas protein of any one of embodiments 1 to 172, wherein the reference protein sequence is SEQ ID NO:1.

174. The Type II Cas protein of embodiment 173, whose amino acid sequence comprises the amino acid sequence of SEQ ID NO:1.

175. The Type II Cas protein of any one of embodiments 1 to 172, wherein the reference protein sequence is SEQ ID NO:2.

176. The Type II Cas protein of any one of embodiments 173 to 175, whose amino acid sequence comprises the amino acid sequence of SEQ ID NO:2.

177. The Type II Cas protein of embodiment 1, whose amino acid sequence comprises the amino acid sequence of SEQ ID NO:3.

178. A Type II Cas protein whose amino acid sequence is identical to a Type II Cas protein of any one of embodiments 1 to 177 except for one or more amino acid substitutions relative to the reference sequence that provide nickase activity.

179. The Type II Cas of embodiment 178, wherein the one or more amino acid substitutions relative to the reference sequence that provide nickase activity comprise a D23A substitution, wherein the position of the D23A substitution is defined with respect to the amino acid numbering of SEQ ID NO: 2.

180. The Type II Cas of embodiment 178, wherein the one or more amino acid substitutions relative to the reference sequence that provide nickase activity comprise a H612A substitution, wherein the position of the H612A substitution is defined with respect to the amino acid numbering of SEQ ID NO: 2 181. A Type II Cas protein whose amino acid sequence is identical to a Type II Cas protein of any one of embodiments 1 to 177 except for one or more amino acid substitutions relative to the reference sequence that render the Type II Cas protein catalytically inactive, optionally wherein the one or more amino acid substitutions comprise D23A and H612A substitutions, wherein the position of the D23A and H612A substitutions are defined with respect to the amino acid numbering of SEQ ID NO:2.

182. An ENQP Type II Cas guide RNA (gRNA) molecule for editing a human RHO gene.

183. An ENQP Type II Cas guide RNA (gRNA) molecule for editing a human DNMT1 gene, optionally wherein the gRNA has a spacer sequence comprising 15, 16, 17, 18, 19, 20, 21, 22, or 23 consecutive nucleotides of SEQ ID NO:43.

184. An ENQP Type II Cas guide RNA (gRNA) molecule for editing a human TRAC gene.

185 An ENQP Type II Cas guide RNA (gRNA) molecule for editing a human B2M gene.

186. An ENQP Type II Cas guide RNA (gRNA) molecule for editing a human PD1 gene.

187. An ENQP Type II Cas guide RNA (gRNA) molecule for editing a human LAG3 gene.

188 An ENQP Type II Cas guide RNA (gRNA) molecule for editing a human RHO gene.

189 A guide RNA (gRNA) molecule for editing a human RHO gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 34)
(a) CUUGUGGCUGACCCGYGGCUGCUC, where Y is U or C;
(SEQ ID NO: 35)
(b) CUUGGGUGGGAGCAGCCRCGGGU, where R is A or G;
(SEQ ID NO: 36)
(c) GGAGCAGCCRCGGGUCAGCCACAA, where R is A or G;
(SEQ ID NO: 37)
(d) CUUGUGGCUGACCCGUGGCUGCUC;
(SEQ ID NO: 38)
(e) CUUGGGUGGGAGCAGCCACGGGU;
(SEQ ID NO: 39)
(f) GGAGCAGCCACGGGUCAGCCACAA;
(SEQ ID NO: 40)
(g) CUUGUGGCUGACCCGCGGCUGCUC;
(SEQ ID NO: 41)
(h) CUUGGGUGGGAGCAGCCGCGGGU;
or
(SEQ ID NO: 42)
(i) GGAGCAGCCGCGGGUCAGCCACAA.

190. The gRNA of embodiment 189, which comprises a spacer that is 15 to 30 nucleotides in length.

191. The gRNA of embodiment 189, wherein the spacer is 18 to 30 nucleotides in length.

192. The gRNA of embodiment 189, wherein the spacer is 20 to 28 nucleotides in length.

193 The gRNA of embodiment 189, wherein the spacer is 22 to 26 nucleotides in length.

194 The gRNA of embodiment 189, wherein the spacer is 23 to 25 nucleotides in length.

195 The gRNA of embodiment 189, wherein the spacer is 22 to 25 nucleotides in length.

196 The gRNA of embodiment 189, wherein the spacer is 15 to 25 nucleotides in length.

197 The gRNA of embodiment 189, wherein the spacer is 16 to 24 nucleotides in length. 198 The gRNA of embodiment 189, wherein the spacer is 17 to 23 nucleotides in length.

199 The gRNA of embodiment 189, wherein the spacer is 18 to 22 nucleotides in length.

200. The gRNA of embodiment 189, wherein the spacer is 19 to 21 nucleotides in length.

201. The gRNA of embodiment 189, wherein the spacer is 25 nucleotides in length.

202. The gRNA of embodiment 189, wherein the spacer is 24 nucleotides in length.

203. The gRNA of embodiment 189, wherein the spacer is 23 nucleotides in length.

204. The gRNA of embodiment 189, wherein the spacer is 22 nucleotides in length.

205. The gRNA of embodiment 189, wherein the spacer is 21 nucleotides in length.

206. The gRNA of embodiment 189, wherein the spacer is 20 nucleotides in length.

207. The gRNA of any one of embodiments 189 to 206, wherein the spacer comprises 16 or more consecutive nucleotides of the reference sequence.

208. The gRNA of any one of embodiments 189 to 206, wherein the spacer comprises 17 or more consecutive nucleotides of the reference sequence.

209. The gRNA of any one of embodiments 189 to 206, wherein the spacer comprises 18 or more consecutive nucleotides of the reference sequence.

210. The gRNA of any one of embodiments 189 to 206, wherein the spacer comprises 19 or more consecutive nucleotides of the reference sequence.

211. The gRNA of any one of embodiments 189 to 206, wherein the spacer comprises 20 consecutive nucleotides of the reference sequence.

212. The gRNA of any one of embodiments 189 to 205, wherein the spacer comprises 21 consecutive nucleotides of the reference sequence.

213. The gRNA of any one of embodiments 189 to 204, wherein the spacer comprises 22 consecutive nucleotides of the reference sequence.

214. The gRNA of any one of embodiments 189 to 203, wherein the reference sequence is a reference sequence having at least 23 nucleotides and the spacer comprises 23 consecutive nucleotides of the reference sequence.

215. The gRNA of any one of embodiments 189 to 202, wherein the reference sequence is a reference sequence having at least 24 nucleotides and the spacer comprises 24 consecutive nucleotides of the reference sequence.

216. The gRNA of any one of embodiments 189 to 206, wherein the spacer comprises a nucleotide sequence that is at least 90% identical to the reference sequence.

217. The gRNA of embodiment 216, wherein the spacer comprises a nucleotide sequence that is at least 95% identical to the reference sequence.

218. The gRNA of any one of embodiments 189 to 206, wherein the spacer comprises a nucleotide sequence that has one mismatch relative to the reference sequence.

219. The gRNA of any one of embodiments 189 to 206, wherein the spacer comprises a nucleotide sequence that has two mismatches relative to the reference sequence.

220. The gRNA of any one of embodiments 189 to 206, wherein the spacer comprises the reference sequence.

221. The gRNA of any one of embodiments 189 to 220, wherein the reference sequence is CUUGUGGCUGACCCGYGGCUGCUC (SEQ ID NO:34), where Y is U or C.

222. The gRNA of any one of embodiments 189 to 220, wherein the reference sequence is CUUGGGUGGGAGCAGCCRCGGGU (SEQ ID NO:35), where R is A or G.

223. The gRNA of any one of embodiments 189 to 220, wherein the reference sequence is GGAGCAGCCRCGGGUCAGCCACAA (SEQ ID NO:36), where R is A or G.

224. The gRNA of any one of embodiments 189 to 220, wherein the reference sequence is CUUGUGGCUGACCCGUGGCUGCUC (SEQ ID NO:37).

225. The gRNA of any one of embodiments 189 to 220, wherein the reference sequence is CUUGGGUGGGAGCAGCCACGGGU (SEQ ID NO:38).

226. The gRNA of any one of embodiments 189 to 220, wherein the reference sequence is GGAGCAGCCACGGGUCAGCCACAA (SEQ ID NO:39).

227. The gRNA of any one of embodiments 189 to 220, wherein the reference sequence is CUUGUGGCUGACCCGCGGCUGCUC (SEQ ID NO:40).

228. The gRNA of any one of embodiments 189 to 220, wherein the reference sequence is CUUGGGUGGGAGCAGCCGCGGGU (SEQ ID NO:41).

229. The gRNA of any one of embodiments 189 to 220, wherein the reference sequence is GGAGCAGCCGCGGGUCAGCCACAA (SEQ ID NO:42).

230. A guide RNA (gRNA) molecule for editing a human RHO gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 93)
(a) GGCCCUUGUGGCUGACCCGYGGCU, where Y is U or C;
(SEQ ID NO: 94)
(b) GGCCCUUGUGGCUGACCCGUGGCU;
(SEQ ID NO: 95)
(c) GGCCCUUGUGGCUGACCCGCGGCU;
(SEQ ID NO: 96)
(d) GGGUGGGAGCAGCCRCGGGU, where R is A or G;
(SEQ ID NO: 97)
(e) UGGGUGGGAGCAGCCRCGGGU, where R is A or G;
(SEQ ID NO: 98)
(f) UUGGGUGGGAGCAGCCRCGGGU, where R is A or G;
(SEQ ID NO: 99)
(g) UCUUGGGUGGGAGCAGCCRCGGGU, where R is A or G;
(SEQ ID NO: 100)
(h) UUCUUGGGUGGGAGCAGCCRCGGGU, where R is A or G;
(SEQ ID NO: 101)
(i) GGGUGGGAGCAGCCACGGGU;
(SEQ ID NO: 102)
(j) UGGGUGGGAGCAGCCACGGGU;
(SEQ ID NO: 103)
(k) UUGGGUGGGAGCAGCCACGGGU;
(SEQ ID NO: 104)
(l) UCUUGGGUGGGAGCAGCCACGGGU;
(SEQ ID NO: 105)
(m) UUCUUGGGUGGGAGCAGCCACGGGU;
(SEQ ID NO: 106)
(n) GGGUGGGAGCAGCCGCGGGU;
(SEQ ID NO: 107)
(o) UGGGUGGGAGCAGCCGCGGGU;
(SEQ ID NO: 108)
(p) UUGGGUGGGAGCAGCCGCGGGU;
(SEQ ID NO: 109)
(q) UCUUGGGUGGGAGCAGCCGCGGGU;
or
(SEQ ID NO: 110)
(r) UUCUUGGGUGGGAGCAGCCGCGGGU.

231. The gRNA of embodiment 230, which comprises a spacer that is 15 to 30 nucleotides in length.

232. The gRNA of embodiment 230, wherein the spacer is 18 to 30 nucleotides in length.

233. The gRNA of embodiment 230, wherein the spacer is 20 to 28 nucleotides in length.

234. The gRNA of embodiment 230, wherein the spacer is 22 to 26 nucleotides in length.

235. The gRNA of embodiment 230, wherein the spacer is 23 to 25 nucleotides in length.

236. The gRNA of embodiment 230, wherein the spacer is 22 to 25 nucleotides in length.

237. The gRNA of embodiment 230, wherein the spacer is 15 to 25 nucleotides in length.

238. The gRNA of embodiment 230, wherein the spacer is 16 to 24 nucleotides in length.

239. The gRNA of embodiment 230, wherein the spacer is 17 to 23 nucleotides in length.

240 The gRNA of embodiment 230, wherein the spacer is 18 to 22 nucleotides in length.

241 The gRNA of embodiment 230, wherein the spacer is 19 to 21 nucleotides in length.

242 The gRNA of embodiment 230, wherein the spacer is 25 nucleotides in length.

243 The gRNA of embodiment 230, wherein the spacer is 24 nucleotides in length.

244 The gRNA of embodiment 230, wherein the spacer is 23 nucleotides in length.

245 The gRNA of embodiment 230, wherein the spacer is 22 nucleotides in length.

246 The gRNA of embodiment 230, wherein the spacer is 21 nucleotides in length.

247. The gRNA of embodiment 230, wherein the spacer is 20 nucleotides in length.

248. The gRNA of any one of embodiments 230 to 247, wherein the spacer comprises 16 or more consecutive nucleotides of the reference sequence.

249. The gRNA of any one of embodiments 230 to 247, wherein the spacer comprises 17 or more consecutive nucleotides of the reference sequence.

250. The gRNA of any one of embodiments 230 to 247, wherein the spacer comprises 18 or more consecutive nucleotides of the reference sequence.

251. The gRNA of any one of embodiments 230 to 247, wherein the spacer comprises 19 or more consecutive nucleotides of the reference sequence.

252. The gRNA of any one of embodiments 230 to 247, wherein the spacer comprises 20 or more consecutive nucleotides of the reference sequence.

253. The gRNA of any one of embodiments 230 to 246, wherein the reference sequence is a reference sequence having at least 21 nucleotides and the spacer comprises 21 or more consecutive nucleotides of the reference sequence.

254. The gRNA of any one of embodiments 230 to 245, wherein the reference sequence is a reference sequence having at least 22 nucleotides and the spacer comprises 22 or more consecutive nucleotides of the reference sequence.

255. The gRNA of any one of embodiments 230 to 244, wherein the reference sequence is a reference sequence having at least 23 nucleotides and the spacer comprises 23 or more consecutive nucleotides of the reference sequence.

256. The gRNA of any one of embodiments 230 to 243, wherein the reference sequence is a reference sequence having at least 24 nucleotides and the spacer comprises 24 or more consecutive nucleotides of the reference sequence.

257. The gRNA of any one of embodiments 230 to 242, wherein the reference sequence is a reference sequence having at least 25 nucleotides and the spacer comprises 25 consecutive nucleotides of the reference sequence.

258. The gRNA of any one of embodiments 230 to 257, wherein the spacer comprises a nucleotide sequence that is at least 90% identical to the reference sequence.

259. The gRNA of embodiment 258, wherein the spacer comprises a nucleotide sequence that is at least 95% identical to the reference sequence.

260. The gRNA of any one of embodiments 230 to 257, wherein the spacer comprises a nucleotide sequence that has one mismatch relative to the reference sequence.

261. The gRNA of any one of embodiments 230 to 257, wherein the spacer comprises a nucleotide sequence that has two mismatches relative to the reference sequence.

262. The gRNA of any one of embodiments 230 to 257, wherein the spacer comprises the reference sequence.

263. The gRNA of any one of embodiments 230 to 262, wherein the reference sequence is GGCCCUUGUGGCUGACCCGYGGCU (SEQ ID NO:93), where Y is U or C.

264. The gRNA of any one of embodiments 230 to 262, wherein the reference sequence is GGCCCUUGUGGCUGACCCGUGGCU (SEQ ID NO:94).

265. The gRNA of any one of embodiments 230 to 262, wherein the reference sequence is GGCCCUUGUGGCUGACCCGCGGCU (SEQ ID NO:95).

266. The gRNA of any one of embodiments 230 to 262, wherein the reference sequence is GGGUGGGAGCAGCCRCGGGU (SEQ ID NO:96), where R is A or G.

267. The gRNA of any one of embodiments 230 to 262, wherein the reference sequence is UGGGUGGGAGCAGCCRCGGGU (SEQ ID NO:97), where R is A or G.

268. The gRNA of any one of embodiments 230 to 262, wherein the reference sequence is UUGGGUGGGAGCAGCCRCGGGU (SEQ ID NO:98), where R is A or G.

269. The gRNA of any one of embodiments 230 to 262, wherein the reference sequence is UCUUGGGUGGGAGCAGCCRCGGGU (SEQ ID NO:99), where R is A or G.

270. The gRNA of any one of embodiments 230 to 262, wherein the reference sequence is UUCUUGGGUGGGAGCAGCCRCGGGU (SEQ ID NO:100), where R is A or G.

271. The gRNA of any one of embodiments 230 to 262, wherein the reference sequence is GGGUGGGAGCAGCCACGGGU (SEQ ID NO:101).

272. The gRNA of any one of embodiments 230 to 262, wherein the reference sequence is UGGGUGGGAGCAGCCACGGGU (SEQ ID NO:102).

273. The gRNA of any one of embodiments 230 to 262, wherein the reference sequence is UUGGGUGGGAGCAGCCACGGGU (SEQ ID NO:103).

274. The gRNA of any one of embodiments 230 to 262, wherein the reference sequence is UCUUGGGUGGGAGCAGCCACGGGU (SEQ ID NO:104).

275. The gRNA of any one of embodiments 230 to 262, wherein the reference sequence is UUCUUGGGUGGGAGCAGCCACGGGU (SEQ ID NO:105).

276. The gRNA of any one of embodiments 230 to 262, wherein the reference sequence is GGGUGGGAGCAGCCGCGGGU (SEQ ID NO:106).

277. The gRNA of any one of embodiments 230 to 262, wherein the reference sequence is UGGGUGGGAGCAGCCGCGGGU (SEQ ID NO:107).

278. The gRNA of any one of embodiments 230 to 262, wherein the reference sequence is UUGGGUGGGAGCAGCCGCGGGU (SEQ ID NO:108).

279. The gRNA of any one of embodiments 230 to 262, wherein the reference sequence is UCUUGGGUGGGAGCAGCCGCGGGU (SEQ ID NO:109), or

280. The gRNA of any one of embodiments 230 to 262, wherein the reference sequence is UUCUUGGGUGGGAGCAGCCGCGGGU (SEQ ID NO:110).

281. A guide RNA (gRNA) molecule for editing a human RHO gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 111)
(a) CAUGCUCCCGGGCUCCUGCACAC;
(SEQ ID NO: 112)
(b) CCUCCAUGCUCCCGGGCUCCUGC;
(SEQ ID NO: 113)
(c) AGCCACCACCGCCAAGCCCGGGA;
(SEQ ID NO: 114)
(d) UCCCUUCUCCUGUCCUGUCAAUG;
(SEQ ID NO: 115)
(e) CCCUUCUCCUGUCCUGUCAAUGU;
(SEQ ID NO: 116)
(f) UGUCCUGUCAAUGUUAUCCAAAG;
(SEQ ID NO: 117)
(g) AUCCAAAGCCCUCAUAUAUUCAG;
(SEQ ID NO: 118)
(h) AAGGCAGUGUUCAGUGCCAGCCC;
(SEQ ID NO: 119)
(i) UCAAGGCAGUGUUCAGUGCCAGC;
(SEQ ID NO: 120)
(j) GUGAAAUUAGACAAGCGCAUAUU;
(SEQ ID NO: 121)
(k) UUGGAGCAAUAUGCGCUUGUCUA;
(SEQ ID NO: 122)
(l) GUUUUCUUGCUGUGAAAUUAGACA;
(SEQ ID NO: 123)
(m) UCACAGCAAGAAAACUGAGCUGA;
(SEQ ID NO: 124)
(n) AAGAAGUCAAGCGCCCUGCUGGG;
(SEQ ID NO: 125)
(o) GAAGUCAAGCGCCCUGCUGGGGC;
(SEQ ID NO: 126)
(p) AACUCUGCACCCGUCCCUGUGUG;
(SEQ ID NO: 127)
(q) UGCUGGGGCGUCACACAGGGACG;
(SEQ ID NO: 128)
(r) AACAUGGCCCGAGAUAGAUGCGG;
(SEQ ID NO: 129)
(s) ACAGAGGCUUGGUGCUGCAAACA;
(SEQ ID NO: 130)
(t) UCCAAGGGAAACAGAGGCUUGGU;
(SEQ ID NO: 131)
(u) GACUCAGCACAGCUGCUCCAAGG;
(SEQ ID NO: 132)
(v) GCCUGGGUCUGACUCAGCACAGC;
(SEQ ID NO: 133)
(w) CCCUUGGAGCAGCUGUGCUGAGU;
or
(SEQ ID NO: 134)
(x) UCAGUGCCCAGCCUGGGUCUGAC.

282. The gRNA of embodiment 281, which comprises a spacer that is 15 to 30 nucleotides in length.

283. The gRNA of embodiment 281, wherein the spacer is 18 to 30 nucleotides in length.

284. The gRNA of embodiment 281, wherein the spacer is 20 to 28 nucleotides in length.

285. The gRNA of embodiment 281, wherein the spacer is 22 to 26 nucleotides in length.

286. The gRNA of embodiment 281, wherein the spacer is 23 to 25 nucleotides in length.

287. The gRNA of embodiment 281, wherein the spacer is 22 to 25 nucleotides in length.

288. The gRNA of embodiment 281, wherein the spacer is 15 to 25 nucleotides in length.

289. The gRNA of embodiment 281, wherein the spacer is 16 to 24 nucleotides in length.

290. The gRNA of embodiment 281, wherein the spacer is 17 to 23 nucleotides in length. The gRNA of embodiment 281, wherein the spacer is 18 to 22 nucleotides in length. 291.

292. The gRNA of embodiment 281, wherein the spacer is 19 to 21 nucleotides in length.

293. The gRNA of embodiment 281, wherein the spacer is 25 nucleotides in length.

294. The gRNA of embodiment 281, wherein the spacer is 24 nucleotides in length.

295 The gRNA of embodiment 281, wherein the spacer is 23 nucleotides in length.

296 The gRNA of embodiment 281, wherein the spacer is 22 nucleotides in length.

297. The gRNA of embodiment 281, wherein the spacer is 21 nucleotides in length.

298. The gRNA of embodiment 281, wherein the spacer is 20 nucleotides in length.

299. The gRNA of any one of embodiments 281 to 298, wherein the spacer comprises 16 or more consecutive nucleotides of the reference sequence.

300. The gRNA of any one of embodiments 281 to 298, wherein the spacer comprises 17 or more consecutive nucleotides of the reference sequence.

301. The gRNA of any one of embodiments 281 to 298, wherein the spacer comprises 18 or more consecutive nucleotides of the reference sequence.

302. The gRNA of any one of embodiments 281 to 298, wherein the spacer comprises 19 or more consecutive nucleotides of the reference sequence.

303. The gRNA of any one of embodiments 281 to 298, wherein the spacer comprises 20 or more consecutive nucleotides of the reference sequence.

304. The gRNA of any one of embodiments 281 to 297, wherein the spacer comprises 21 or more consecutive nucleotides of the reference sequence.

305. The gRNA of any one of embodiments 281 to 296, wherein the spacer comprises 22 or more consecutive nucleotides of the reference sequence.

306. The gRNA of any one of embodiments 281 to 295, wherein the spacer comprises 23 or more consecutive nucleotides of the reference sequence.

307. The gRNA of any one of embodiments 281 to 294, wherein the reference sequence is a reference sequence having at least 24 nucleotides and the spacer comprises 24 consecutive nucleotides of the reference sequence.

308. The gRNA of any one of embodiments 281 to 298, wherein the spacer comprises a nucleotide sequence that is at least 90% identical to the reference sequence.

309. The gRNA of embodiment 308, wherein the spacer comprises a nucleotide sequence that is at least 95% identical to the reference sequence.

310. The gRNA of any one of embodiments 281 to 298, wherein the spacer comprises a nucleotide sequence that has one mismatch relative to the reference sequence.

311. The gRNA of any one of embodiments 281 to 298, wherein the spacer comprises a nucleotide sequence that has two mismatches relative to the reference sequence.

312. The gRNA of any one of embodiments 281 to 307, wherein the spacer comprises the reference sequence.

313. The gRNA of any one of embodiments 281 to 312, wherein the reference sequence is CAUGCUCCCGGGCUCCUGCACAC (SEQ ID NO:111).

314. The gRNA of any one of embodiments 281 to 312, wherein the reference sequence is CCUCCAUGCUCCCGGGCUCCUGC (SEQ ID NO:112).

315. The gRNA of any one of embodiments 281 to 312, wherein the reference sequence is AGCCACCACCGCCAAGCCCGGGA (SEQ ID NO:113).

316. The gRNA of any one of embodiments 281 to 312, wherein the reference sequence is UCCCUUCUCCUGUCCUGUCAAUG (SEQ ID NO:114).

317. The gRNA of any one of embodiments 281 to 312, wherein the reference sequence is CCCUUCUCCUGUCCUGUCAAUGU (SEQ ID NO:115).

318. The gRNA of any one of embodiments 281 to 312, wherein the reference sequence is UGUCCUGUCAAUGUUAUCCAAAG (SEQ ID NO:116).

319. The gRNA of any one of embodiments 281 to 312, wherein the reference sequence is AUCCAAAGCCCUCAUAUAUUCAG (SEQ ID NO:117).

320. The gRNA of any one of embodiments 281 to 312, wherein the reference sequence is AAGGCAGUGUUCAGUGCCAGCCC (SEQ ID NO:118).

321. The gRNA of any one of embodiments 281 to 312, wherein the reference sequence is UCAAGGCAGUGUUCAGUGCCAGC (SEQ ID NO:119).

322. The gRNA of any one of embodiments 281 to 312, wherein the reference sequence is GUGAAAUUAGACAAGCGCAUAUU (SEQ ID NO:120).

323. The gRNA of any one of embodiments 281 to 312, wherein the reference sequence is UUGGAGCAAUAUGCGCUUGUCUA (SEQ ID NO:121).

324. The gRNA of any one of embodiments 281 to 312, wherein the reference sequence is GUUUUCUUGCUGUGAAAUUAGACA (SEQ ID NO:122).

325. The gRNA of any one of embodiments 281 to 312, wherein the reference sequence is UCACAGCAAGAAAACUGAGCUGA (SEQ ID NO:123).

326. The gRNA of any one of embodiments 281 to 312, wherein the reference sequence is AAGAAGUCAAGCGCCCUGCUGGG (SEQ ID NO:124).

327. The gRNA of any one of embodiments 281 to 312, wherein the reference sequence is GAAGUCAAGCGCCCUGCUGGGGC (SEQ ID NO:125).

328. The gRNA of any one of embodiments 281 to 312, wherein the reference sequence is AACUCUGCACCCGUCCCUGUGUG (SEQ ID NO:126).

329. The gRNA of any one of embodiments 281 to 312, wherein the reference sequence is UGCUGGGGCGUCACACAGGGACG (SEQ ID NO:127).

330. The gRNA of any one of embodiments 281 to 312, wherein the reference sequence is AACAUGGCCCGAGAUAGAUGCGG (SEQ ID NO:128).

331. The gRNA of any one of embodiments 281 to 312, wherein the reference sequence is ACAGAGGCUUGGUGCUGCAAACA (SEQ ID NO:129).

332. The gRNA of any one of embodiments 281 to 312, wherein the reference sequence is UCCAAGGGAAACAGAGGCUUGGU (SEQ ID NO:130).

333. The gRNA of any one of embodiments 281 to 312, wherein the reference sequence is GACUCAGCACAGCUGCUCCAAGG (SEQ ID NO:131).

334. The gRNA of any one of embodiments 281 to 312, wherein the reference sequence is GCCUGGGUCUGACUCAGCACAGC (SEQ ID NO:132).

335. The gRNA of any one of embodiments 281 to 312, wherein the reference sequence is CCCUUGGAGCAGCUGUGCUGAGU (SEQ ID NO:133).

336. The gRNA of any one of embodiments 281 to 312, wherein the reference sequence is UCAGUGCCCAGCCUGGGUCUGAC (SEQ ID NO:134).

337. A guide RNA (gRNA) molecule for editing a human TRAC gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 135)
(a) AUACACAUCAGAAUCCUUACUUU;
(SEQ ID NO: 136)
(b) GUAAGGAUUCUGAUGUGUAUAUCA;
(SEQ ID NO: 137)
(c) GAGCAACAGUGCUGUGGCCUGGA;
(SEQ ID NO: 138)
(d) GGAGCAACAAAUCUGACUUUGCA;
(SEQ ID NO: 139)
(e) UGCUGUUGUUGAAGGCGUUUGCA;
(SEQ ID NO: 140)
(f) AGCAUUAUUCCAGAAGACACCUU;
(SEQ ID NO: 141)
(g) UAUUCCAGAAGACACCUUCUUCC;
(SEQ ID NO: 142)
(h) ACAAAGUAAGGAUUCUGAUGUGU;
(SEQ ID NO: 143)
(i) GUCUAGCACAGUUUUGUCUGUGA;
(SEQ ID NO: 144)
(j) CUUGAAGUCCAUAGACCUCAUGU;
(SEQ ID NO: 145)
(k) GGCCACAGCACUGUUGCUCUUGA;
(SEQ ID NO: 146)
(l) UGCAAAGUCAGAUUUGUUGCUCC;
or
(SEQ ID NO: 147)
(m) GAAUAAUGCUGUUGUUGAAGGCG.

338. A guide RNA (gRNA) molecule for editing a human B2M gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 148)
(a) UAAGGCCACGGAGCGAGACAUCU;
(SEQ ID NO: 149)
(b) CAGGCCAGAAAGAGAGAGUAGCG;
(SEQ ID NO: 150)
(c) GCCUGGAGGCUAUCCAGCGUGAGU.

339. A guide RNA (gRNA) molecule for editing a human PD1 gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 151)
(a) GGCCGCCAGCCCAGUUGUAGCAC;
or
(SEQ ID NO: 152)
(b) CUGGCCGCCAGCCCAGUUGUAGC.

340. A guide RNA (gRNA) molecule for editing a human LAG3 gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is

(SEQ ID NO: 153)
(a) GCCAUCCCCGUUUUACCUGGAGC;
(SEQ ID NO: 154)
(b) CGCCAUCCCCGUUUUACCUGGAG;
(SEQ ID NO: 155)
(c) CCGCCAUCCCCGUUUUACCUGGA;
(SEQ ID NO: 156)
(d) GGGAUGGCGGGAGGGUUGACCUC;
(SEQ ID NO: 157)
(e) GAGCUAAGGACCUCAGGACCUUUG;
(SEQ ID NO: 158)
(f) GAGAGGCUUUCGGGGUGGAAGGAC;
(SEQ ID NO: 159)
(g) GAGAGGCUUUCGGGGUGGAAGGA;
or
(SEQ ID NO: 160)
(h) AGAGAGGCUUUCGGGGUGGAAGG.

341. The gRNA of any one of embodiments 337 to 340, which comprises a spacer that is 15 to 30 nucleotides in length.

342. The gRNA of any one of embodiments 337 to 340, wherein the spacer is 18 to 30 nucleotides in length.

343. The gRNA of any one of embodiments 337 to 340, wherein the spacer is 20 to 28 nucleotides in length.

344. The gRNA of any one of embodiments 337 to 340, wherein the spacer is 22 to 26 nucleotides in length.

345. The gRNA of any one of embodiments 337 to 340, wherein the spacer is 23 to 25 nucleotides in length.

346. The gRNA of any one of embodiments 337 to 340, wherein the spacer is 22 to 25 nucleotides in length.

347. The gRNA of any one of embodiments 337 to 340, wherein the spacer is 15 to 25 nucleotides in length.

348. The gRNA of any one of embodiments 337 to 340, wherein the spacer is 16 to 24 nucleotides in length.

349. The gRNA of any one of embodiments 337 to 340, wherein the spacer is 17 to 23 nucleotides in length.

350. The gRNA of any one of embodiments 337 to 340, wherein the spacer is 18 to 22 nucleotides in length.

351. The gRNA of any one of embodiments 337 to 340, wherein the spacer is 19 to 21 nucleotides in length.

352. The gRNA of any one of embodiments 337 to 340, wherein the spacer is 25 nucleotides in length.

353. The gRNA of any one of embodiments 337 to 340, wherein the spacer is 24 nucleotides in length.

354. The gRNA of any one of embodiments 337 to 340, wherein the spacer is 23 nucleotides in length.

355. The gRNA of any one of embodiments 337 to 340, wherein the spacer is 22 nucleotides in length.

356. The gRNA of any one of embodiments 337 to 340, wherein the spacer is 21 nucleotides in length.

357. The gRNA of any one of embodiments 337 to 340, wherein the spacer is 20 nucleotides in length.

358. The gRNA of any one of embodiments 337 to 357, wherein the spacer comprises 16 or more consecutive nucleotides of the reference sequence.

359. The gRNA of any one of embodiments 337 to 357, wherein the spacer comprises 17 or more consecutive nucleotides of the reference sequence.

360. The gRNA of any one of embodiments 337 to 357, wherein the spacer comprises 18 or more consecutive nucleotides of the reference sequence.

361. The gRNA of any one of embodiments 337 to 357, wherein the spacer comprises 19 or more consecutive nucleotides of the reference sequence.

362. The gRNA of any one of embodiments 337 to 357, wherein the spacer comprises 20 or more consecutive nucleotides of the reference sequence.

363. The gRNA of any one of embodiments 337 to 356, wherein the spacer comprises 21 or more consecutive nucleotides of the reference sequence.

364. The gRNA of any one of embodiments 337 to 355, wherein the spacer comprises 22 or more consecutive nucleotides of the reference sequence.

365. The gRNA of any one of embodiments 337 to 354, wherein the spacer comprises 23 or more consecutive nucleotides of the reference sequence.

366. The gRNA of any one of embodiments 337 to 353, wherein the reference sequence is a reference sequence having at least 24 nucleotides and the spacer comprises 24 consecutive nucleotides of the reference sequence.

367. The gRNA of any one of embodiments 337 to 357, wherein the spacer comprises a nucleotide sequence that is at least 90% identical to the reference sequence.

368. The gRNA of embodiment 367, wherein the spacer comprises a nucleotide sequence that is at least 95% identical to the reference sequence.

369. The gRNA of any one of embodiments 337 to 357, wherein the spacer comprises a nucleotide sequence that has one mismatch relative to the reference sequence.

370. The gRNA of any one of embodiments 337 to 357, wherein the spacer comprises a nucleotide sequence that has two mismatches relative to the reference sequence.

371. The gRNA of any one of embodiments 337 to 357, wherein the spacer comprises the reference sequence.

372. The gRNA of any one of embodiments 337 and 341 to 371 when depending from embodiment 337, wherein the reference sequence is AUACACAUCAGAAUCCUUACUUU (SEQ ID NO: 135).

373. The gRNA of any one of embodiments 337 and 341 to 371 when depending from embodiment 337, wherein the reference sequence is GUAAGGAUUCUGAUGUGUAUAUCA (SEQ ID NO: 136).

374. The gRNA of any one of embodiments 337 and 341 to 371 when depending from embodiment 337, wherein the reference sequence is GAGCAACAGUGCUGUGGCCUGGA (SEQ ID NO: 137).

375. The gRNA of any one of embodiments 337 and 341 to 371 when depending from embodiment 337, wherein the reference sequence is GGAGCAACAAAUCUGACUUUGCA (SEQ ID NO: 138).

376. The gRNA of any one of embodiments 337 and 341 to 371 when depending from embodiment 337, wherein the reference sequence is UGCUGUUGUUGAAGGCGUUUGCA (SEQ ID NO: 139).

377. The gRNA of any one of embodiments 337 and 341 to 371 when depending from embodiment 337, wherein the reference sequence is AGCAUUAUUCCAGAAGACACCUU (SEQ ID NO: 140).

378. The gRNA of any one of embodiments 337 and 341 to 371 when depending from embodiment 337, wherein the reference sequence is UAUUCCAGAAGACACCUUCUUCC (SEQ ID NO: 141).

379. The gRNA of any one of embodiments 337 and 341 to 371 when depending from embodiment 337, wherein the reference sequence is ACAAAGUAAGGAUUCUGAUGUGU (SEQ ID NO: 142).

380. The gRNA of any one of embodiments 337 and 341 to 371 when depending from embodiment 337, wherein the reference sequence is GUCUAGCACAGUUUUGUCUGUGA (SEQ ID NO: 143).

381. The gRNA of any one of embodiments 337 and 341 to 371 when depending from embodiment 337, wherein the reference sequence is CUUGAAGUCCAUAGACCUCAUGU (SEQ ID NO: 144).

382. The gRNA of any one of embodiments 337 and 341 to 371 when depending from embodiment 337, wherein the reference sequence is GGCCACAGCACUGUUGCUCUUGA (SEQ ID NO: 145).

383. The gRNA of any one of embodiments 337 and 341 to 371 when depending from embodiment 337, wherein the reference sequence is UGCAAAGUCAGAUUUGUUGCUCC (SEQ ID NO: 146).

384. The gRNA of any one of embodiments 337 and 341 to 371 when depending from embodiment 337, wherein the reference sequence is GAAUAAUGCUGUUGUUGAAGGCG (SEQ ID NO: 147).

385. The gRNA of any one of embodiments 338 and 341 to 371 when depending from embodiment 338, wherein the reference sequence is UAAGGCCACGGAGCGAGACAUCU (SEQ ID NO: 148).

386. The gRNA of any one of embodiments 338 and 341 to 371 when depending from embodiment 338, wherein the reference sequence is CAGGCCAGAAAGAGAGAGUAGCG (SEQ ID NO: 149).

387. The gRNA of any one of embodiments 338 and 341 to 371 when depending from embodiment 338, wherein the reference sequence is GCCUGGAGGCUAUCCAGCGUGAGU (SEQ ID NO: 150).

388. The gRNA of any one of embodiments 339 and 341 to 371 when depending from embodiment 339, wherein the reference sequence is GGCCGCCAGCCCAGUUGUAGCAC (SEQ ID NO: 151).

389. The gRNA of any one of embodiments 339 and 341 to 371 when depending from embodiment 339, wherein the reference sequence is CUGGCCGCCAGCCCAGUUGUAGC (SEQ ID NO: 152).

390. The gRNA of any one of embodiments 340 and 341 to 371 when depending from embodiment 340, wherein the reference sequence is GCCAUCCCCGUUUUACCUGGAGC (SEQ ID NO: 153).

391. The gRNA of any one of embodiments 340 and 341 to 371 when depending from embodiment 340, wherein the reference sequence is CGCCAUCCCCGUUUUACCUGGAG (SEQ ID NO: 154).

392. The gRNA of any one of embodiments 340 and 341 to 371 when depending from embodiment 340, wherein the reference sequence is CCGCCAUCCCCGUUUUACCUGGA (SEQ ID NO: 155).

393. The gRNA of any one of embodiments 340 and 341 to 371 when depending from embodiment 340, wherein the reference sequence is GGGAUGGCGGGAGGGUUGACCUC (SEQ ID NO: 156).

394. The gRNA of any one of embodiments 340 and 341 to 371 when depending from embodiment 340, wherein the reference sequence is GAGCUAAGGACCUCAGGACCUUUG (SEQ ID NO: 157).

395. The gRNA of any one of embodiments 340 and 341 to 371 when depending from embodiment 340, wherein the reference sequence is GAGAGGCUUUCGGGGUGGAAGGAC (SEQ ID NO: 158).

396. The gRNA of any one of embodiments 340 and 341 to 371 when depending from embodiment 340, wherein the reference sequence is GAGAGGCUUUCGGGGUGGAAGGA (SEQ ID NO: 159).

397. The gRNA of any one of embodiments 340 and 341 to 371 when depending from embodiment 340, wherein the reference sequence is AGAGAGGCUUUCGGGGUGGAAGG (SEQ ID NO: 160).

398. The gRNA of any one of embodiments 182 to 397, which is a single guide RNA (sgRNA). 399. A gRNA comprising a spacer and a sgRNA scaffold, which is optionally a gRNA according to any one of embodiments 182 to 398, wherein:
(a) the spacer is positioned 5′ to the sgRNA scaffold; and
(b) the nucleotide sequence of the sgRNA scaffold comprises a nucleotide sequence that is at least 50% identical to a reference scaffold sequence, wherein the reference scaffold sequence is SEQ ID NO:44, SEQ ID NO:45, or SEQ ID NO:91.

400. A gRNA comprising a means for binding a target mammalian genomic sequence and a sgRNA scaffold, optionally wherein the means for binding a target mammalian genomic sequence is a spacer, wherein:
(a) the means for binding a target genomic sequence is positioned 5′ to the sgRNA scaffold; and
(b) the nucleotide sequence of the sgRNA scaffold comprises a nucleotide sequence that is at least 50% identical to a reference scaffold sequence, wherein the reference scaffold sequence is SEQ ID NO:44, SEQ ID NO:45, or SEQ ID NO:91.

401. The gRNA of embodiment 399 or embodiment 400, wherein the sgRNA scaffold comprises one or more G: C couples not present in the reference scaffold sequence.

402. The gRNA of any one of embodiments 399 to 400, wherein the sgRNA scaffold comprises one or more U to A substitutions relative to the reference scaffold sequence.

403. The gRNA of any one of embodiments 399 to 402, wherein the sgRNA scaffold comprises one or more trimmed stem loop sequences in place of one or more longer stem loop sequences in the reference scaffold sequence.

404. The gRNA of embodiment 403, wherein the trimmed stem loop sequence comprises a GAAA tetraloop in place of a longer stem loop sequence in the reference scaffold sequence.

405. The gRNA of any one of embodiments 399 to 404, wherein the sgRNA scaffold comprises one or more trimmed loop sequences in place of one or more longer loop sequences in the reference scaffold sequence.

406. The gRNA of embodiment 405, wherein the sgRNA scaffold comprises a GAAA tetraloop in place of a longer loop sequence in the reference scaffold sequence.

407. The gRNA of any one of embodiments 399 to 406, wherein the sgRNA scaffold comprises a nucleotide sequence that is at least 55% identical to the reference scaffold sequence.

408. The gRNA of embodiment 407, wherein the sgRNA scaffold comprises a nucleotide sequence that is at least 60% identical to the reference scaffold sequence.

409. The gRNA of embodiment 407, wherein the sgRNA scaffold comprises a nucleotide sequence that is at least 65% identical to the reference scaffold sequence.

410. The gRNA of embodiment 407, wherein the sgRNA scaffold comprises a nucleotide sequence that is at least 70% identical to the reference scaffold sequence.

411. The gRNA of embodiment 407, wherein the sgRNA scaffold comprises a nucleotide sequence that is at least 75% identical to the reference scaffold sequence.

412. The gRNA of embodiment 407, wherein the sgRNA scaffold comprises a nucleotide sequence that is at least 80% identical to the reference scaffold sequence.

413. The gRNA of embodiment 407, wherein the sgRNA scaffold comprises a nucleotide sequence that is at least 85% identical to the reference scaffold sequence.

414. The gRNA of embodiment 407, wherein the sgRNA scaffold comprises a nucleotide sequence that is at least 90% identical to the reference scaffold sequence.

415. The gRNA of embodiment 407, wherein the sgRNA scaffold comprises a nucleotide sequence that is at least 95% identical to the reference scaffold sequence.

416. The gRNA of embodiment 407, wherein the sgRNA scaffold comprises a nucleotide sequence that is at least 96% identical to the reference scaffold sequence.

417. The gRNA of embodiment 407, wherein the sgRNA scaffold comprises a nucleotide sequence that is at least 97% identical to the reference scaffold sequence.

418. The gRNA of embodiment 407, wherein the sgRNA scaffold comprises a nucleotide sequence that is at least 98% identical to the reference scaffold sequence.

419. The gRNA of embodiment 407, wherein the sgRNA scaffold comprises a nucleotide sequence that is at least 99% identical to the reference scaffold sequence.

420. The gRNA of embodiment 407, wherein the sgRNA scaffold comprises a nucleotide sequence that has no more than 5 nucleotide mismatches with the reference scaffold sequence.

421. The gRNA of embodiment 407, wherein the sgRNA scaffold comprises a nucleotide sequence that has no more than 4 nucleotide mismatches with the reference scaffold sequence.

422. The gRNA of embodiment 407, wherein the sgRNA scaffold comprises a nucleotide sequence that has no more than 3 nucleotide mismatches with the reference scaffold sequence.

423. The gRNA of embodiment 407, wherein the sgRNA scaffold comprises a nucleotide sequence that has no more than 2 nucleotide mismatches with the reference scaffold sequence.

424. The gRNA of embodiment 407, wherein the sgRNA scaffold comprises a nucleotide sequence that has no more than 1 nucleotide mismatches with the reference scaffold sequence.

425. The gRNA of embodiment 182 or embodiment 400, wherein the sgRNA scaffold comprises a nucleotide sequence that is 100% identical to the reference scaffold sequence.

426. The gRNA of any one of embodiments 399 to 425, wherein the reference scaffold sequence is SEQ ID NO:44.

427. The gRNA of any one of embodiments 399 to 425, wherein the reference scaffold sequence is SEQ ID NO:45.

428. The gRNA of any one of embodiments 399 to 425, wherein the reference scaffold sequence is SEQ ID NO:91.

429. The gRNA of embodiment 426, wherein the nucleotide sequence of the sgRNA scaffold comprises the nucleotide sequence of SEQ ID NO:46.

430. The gRNA of embodiment 427, wherein the nucleotide sequence of the sgRNA scaffold comprises the nucleotide sequence of SEQ ID NO:47.

431. The gRNA of embodiment 428, wherein the nucleotide sequence of the sgRNA scaffold comprises the nucleotide sequence of SEQ ID NO:92.

432. The gRNA of any one of embodiments 399 to 431, wherein the sgRNA scaffold comprises 1 to 8 uracils at its 3′ end.

433. The gRNA of embodiment 432, wherein the sgRNA scaffold comprises 1 uracil at its 3′ end.

434. The gRNA of embodiment 432, wherein the sgRNA scaffold comprises 2 uracils at its 3′ end.

435. The gRNA of embodiment 432, wherein the sgRNA scaffold comprises 3 uracils at its 3′ end.

436. The gRNA of embodiment 432, wherein the sgRNA scaffold comprises 4 uracils at its 3′ end.

437. The gRNA of embodiment 432, wherein the sgRNA scaffold comprises 5 uracils at its 3′ end.

438. The gRNA of embodiment 432, wherein the sgRNA scaffold comprises 6 uracils at its 3′ end.

439. The gRNA of embodiment 432, wherein the sgRNA scaffold comprises 7 uracils at its 3′ end.

440. The gRNA of embodiment 432, wherein the sgRNA scaffold comprises 8 uracils at its 3′ end.

441. The gRNA of any one of embodiments 399 to 440, wherein the nucleotide sequence of the spacer is partially or fully complementary to a target mammalian genomic sequence.

442. A gRNA comprising (i) a crRNA comprising a spacer (optionally wherein the spacer is a spacer described in any one of embodiments 189 to 397) and a crRNA scaffold, wherein the spacer is 5′ to the crRNA scaffold, and (ii) a tracrRNA, wherein the nucleotide sequence of the spacer is partially or fully complementary to a target mammalian genomic sequence and the nucleotide sequence of the crRNA scaffold comprises the nucleotide sequence of SEQ ID NO:28.

443. A gRNA comprising (i) a crRNA comprising a means for binding a target mammalian genomic sequence (which is optionally a spacer) and a crRNA scaffold, wherein the means for binding a target mammalian genomic sequence is 5′ to the crRNA scaffold, and (ii) a tracrRNA, wherein the nucleotide sequence of the crRNA scaffold comprises the nucleotide sequence of SEQ ID NO:28.

444. The gRNA of any one of embodiments 442 to 443, wherein the nucleotide sequence of the tracrRNA comprises the nucleotide sequence of SEQ ID NO:29.

445. The gRNA of any one of embodiments 442 to 444 wherein the gRNA comprises separate crRNA and tracrRNA molecules.

446. The gRNA of any one of embodiments 442 to 444, wherein the gRNA is a single guide RNA (sgRNA).

447. The gRNA of any one of embodiments 441 to 446, wherein the target mammalian genomic sequence is a human genomic sequence.

448. The gRNA of embodiment 447, wherein the target mammalian genomic sequence is a RHO or DNMT1 genomic sequence.

449. The gRNA of embodiment 447, wherein the target mammalian genomic sequence is a RHO genomic sequence.

450. The gRNA of embodiment 447, wherein the target mammalian genomic sequence is a TRAC genomic sequence.

451. The gRNA of embodiment 447, wherein the target mammalian genomic sequence is a B2M genomic sequence.

452. The gRNA of embodiment 447, wherein the target mammalian genomic sequence is a PD1 genomic sequence.

453. The gRNA of embodiment 447, wherein the target mammalian genomic sequence is a LAG3 genomic sequence.

454. The gRNA of any one of embodiments 441 to 453, wherein the target mammalian genomic sequence is upstream of a protospacer adjacent motif (PAM) sequence in the non-target strand recognized by a Type II Cas protein, optionally wherein the Type II Cas protein is a Type II Cas protein according to any one of embodiments 1 to 181.

455. The gRNA of embodiment 454, wherein the PAM sequence is N₄CMNA.

456. The gRNA of embodiment 454, wherein the PAM sequence is N₄CCNA.

457. The gRNA of embodiment 454, wherein the PAM sequence is N₄CCGA.

458. The gRNA of embodiment 454, wherein the PAM sequence is N₄CTGA.

459. The gRNA of any one of embodiments 399 to 458, wherein the spacer is 15 to 30 nucleotides in length.

460. The gRNA of embodiment 459, wherein the spacer is 15 to 25 nucleotides in length.

461. The gRNA of embodiment 459, wherein the spacer is 16 to 24 nucleotides in length.

462. The gRNA of embodiment 459, wherein the spacer is 17 to 23 nucleotides in length.

463. The gRNA of embodiment 459, wherein the spacer is 18 to 22 nucleotides in length.

464. The gRNA of embodiment 459, wherein the spacer is 19 to 21 nucleotides in length.

465. The gRNA of embodiment 459, wherein the spacer is 18 to 30 nucleotides in length.

466. The gRNA of embodiment 459, wherein the spacer is 20 to 28 nucleotides in length.

467 The gRNA of embodiment 459, wherein the spacer is 22 to 26 nucleotides in length.

468 The gRNA of embodiment 459, wherein the spacer is 23 to 25 nucleotides in length.

469. The gRNA of embodiment 459, wherein the spacer is 20 nucleotides in length.

470 The gRNA of embodiment 459, wherein the spacer is 21 nucleotides in length.

471. The gRNA of embodiment 459, wherein the spacer is 22 nucleotides in length.

472. The gRNA of embodiment 459, wherein the spacer is 23 nucleotides in length.

473. The gRNA of embodiment 459, wherein the spacer is 24 nucleotides in length.

474. The gRNA of embodiment 459, wherein the spacer is 25 nucleotides in length.

475. The gRNA of embodiment 459, wherein the spacer is 26 nucleotides in length.

476. The gRNA of embodiment 459, wherein the spacer is 27 nucleotides in length.

477. The gRNA of embodiment 459, wherein the spacer is 28 nucleotides in length.

478. A gRNA comprising a spacer sequence of SEQ ID NO:38.

479 A gRNA comprising a spacer whose sequence is the sequence of SEQ ID NO:41.

480. A gRNA comprising a spacer whose sequence is the sequence of SEQ ID NO: 101.

481. A gRNA comprising a spacer whose sequence is the sequence of SEQ ID NO:102.

482. A gRNA comprising a spacer whose sequence is the sequence of SEQ ID NO:103.

483. A gRNA comprising a spacer whose sequence is the sequence of SEQ ID NO:104.

484. A gRNA comprising a spacer whose sequence is the sequence of SEQ ID NO: 105.

485. A gRNA comprising a spacer whose sequence is the sequence of SEQ ID NO: 106.

486. A gRNA comprising a spacer whose sequence is the sequence of SEQ ID NO:107.

487. A gRNA comprising a spacer whose sequence is the sequence of SEQ ID NO:108.

488. A gRNA comprising a spacer whose sequence is the sequence of SEQ ID NO:109.

489 A gRNA comprising a spacer whose sequence is the sequence of SEQ ID NO:110.

490. A gRNA comprising a spacer whose sequence is the sequence of SEQ ID NO:118.

491. A gRNA comprising a spacer whose sequence is the sequence of SEQ ID NO:128.

492. A gRNA comprising a spacer whose sequence is the sequence of SEQ ID NO:130.

493. A gRNA comprising a spacer whose sequence is the sequence of SEQ ID NO:136.

494. A gRNA comprising a spacer whose sequence is the sequence of SEQ ID NO:139.

495. A gRNA comprising a spacer whose sequence is the sequence of SEQ ID NO:140.

496. A gRNA comprising a spacer whose sequence is the sequence of SEQ ID NO:149.

497. A gRNA comprising a spacer whose sequence is the sequence of SEQ ID NO:152.

498. A gRNA comprising a spacer whose sequence is the sequence of SEQ ID NO: 156.

499. A gRNA comprising a spacer whose sequence is the sequence of SEQ ID NO:158.

500. A gRNA comprising a spacer whose sequence is the sequence of SEQ ID NO: 159.

501. A gRNA comprising a spacer whose sequence is the sequence of SEQ ID NO: 160.

502. The gRNA of any one of embodiments 478 to 501, wherein the spacer is positioned 5′ to a scaffold whose sequence comprises the sequence of SEQ ID NO:44.

503. The gRNA of any one of embodiments 478 to 501, wherein the spacer is positioned 5′ to a scaffold whose sequence comprises the sequence of SEQ ID NO:45.

504. The gRNA of any one of embodiments 478 to 501, wherein the spacer is positioned 5′ to a scaffold whose sequence comprises the sequence of SEQ ID NO:46.

505. The gRNA of any one of embodiments 478 to 501, wherein the spacer is positioned 5′ to a scaffold whose sequence comprises the sequence of SEQ ID NO:47.

506. The gRNA of any one of embodiments 478 to 501, wherein the spacer is positioned 5′ to a scaffold whose sequence comprises the sequence of SEQ ID NO:91.

507. The gRNA of any one of embodiments 478 to 501, wherein the spacer is positioned 5′ to a scaffold whose sequence comprises the sequence of SEQ ID NO:92.

508. A combination of gRNAs comprising a first gRNA and a second gRNA independently selected from gRNAs of embodiments 189 to 507.

509. The combination of gRNAs of embodiment 508, wherein the first gRNA is a gRNA targeting the RHO rs7984 SNP and the second gRNA is a gRNA targeting RHO intron 1.

510. A system comprising the Type II Cas protein of any one of embodiments 1 to 181 and a guide RNA (gRNA) comprising a spacer sequence, optionally wherein the gRNA is a gRNA according to any one of embodiments 182 to 507.

511. A system comprising the Type II Cas protein of any one of embodiments 1 to 181 and a means for targeting the Type II Cas protein to a target genomic sequence, optionally wherein the means for targeting the Type II Cas protein to a target genomic sequence is a guide RNA (gRNA) molecule, optionally as described in in any one of embodiments 182 to 507, optionally wherein the gRNA molecule comprises a spacer partially or fully complementary to a target mammalian genomic sequence.

512. The system of embodiment 510 or 511, which is a ribonucleoprotein (RNP) comprising the Type II Cas protein complexed to the gRNA or means for targeting the Type II Cas protein to a target genomic sequence.

513. A nucleic acid encoding the Type II Cas protein of any one of embodiments 1 to 181, optionally wherein the nucleotide sequence encoding the Type II Cas protein is operably linked to a promoter that is heterologous to the Type II Cas protein.

514. The nucleic acid of embodiment 513, wherein the nucleotide sequence encoding the Type II Cas protein is codon optimized for expression in human cells.

515. The nucleic acid of embodiment 514, wherein when the reference protein sequence is SEQ ID NO:1 or SEQ ID NO:2, the nucleotide sequence encoding the Type II Cas protein comprises a nucleotide sequences that is at least 80%, at least 85%, at least 90%, at least 95%, at least 96%, at least 97%, at least 98%, at least 99%, or 100% identical to the nucleotide sequence of SEQ ID NO:5 or SEQ ID NO: 6.

516. The nucleic acid of any one of embodiments embodiment 513 to 515, which is a plasmid.

517. The nucleic acid of any one of embodiments embodiment 513 to 515, which is a viral genome.

518. The nucleic acid of embodiment 517, wherein the viral genome is an adeno-associated virus (AAV) genome.

519. The nucleic acid of embodiment 518, wherein the AAV genome is an AAV2, AAV5, AAV7m8, AAV8, AAV9, AAVrh8r, or AAVrh10 genome.

520. The nucleic acid of embodiment 519, wherein the AAV genome is an AAV2 genome.

521. The nucleic acid of embodiment 519, wherein the AAV genome is an AAV5 genome.

522. The nucleic acid of embodiment 519, wherein the AAV genome is an AAV7m8 genome.

523. The nucleic acid of embodiment 519, wherein the AAV genome is an AAV8 genome.

524 The nucleic acid of embodiment 519, wherein the AAV genome is an AAV9 genome.

525. The nucleic acid of embodiment 519, wherein the AAV genome is an AAVrh8r genome.

526. The nucleic acid of embodiment 519, wherein the AAV genome is an AAVrh10 genome.

527. The nucleic acid of any one of embodiments 513 to 526, further encoding a gRNA, optionally wherein the gRNA is a gRNA according to any one of embodiments 182 to 507.

528. The nucleic acid of any one of embodiments 513 to 526, further encoding a combination of gRNAs, optionally wherein the combination of gRNAs is a combination of gRNAs according to any one of embodiments 508 to 509.

529. A nucleic acid encoding the gRNA of any one of embodiments 182 to 507.

530. A nucleic acid encoding the combination of gRNAs of any one of embodiments 508 to 509.

531. The nucleic acid of embodiment 529 or 530, which is a plasmid.

532. The nucleic acid of embodiment 529 or 530, which is a viral genome.

533. The nucleic acid of embodiment 532, wherein the viral genome is an adeno-associated virus (AAV) genome.

534. The nucleic acid of embodiment 533, wherein the AAV genome is a AAV2, AAV5, AAV7m8, AAV8, AAV9, AAVrh8r, or AAVrh10 genome.

535. The nucleic acid of embodiment 534, wherein the AAV genome is an AAV2 genome. 536. The nucleic acid of embodiment 534, wherein the AAV genome is an AAV5 genome.

537. The nucleic acid of embodiment 534, wherein the AAV genome is an AAV7m8 genome.

538 The nucleic acid of embodiment 534, wherein the AAV genome is an AAV8 genome.

539. The nucleic acid of embodiment 534, wherein the AAV genome is an AAV9 genome.

540 The nucleic acid of embodiment 534, wherein the AAV genome is an AAVrh8r genome.

541. The nucleic acid of embodiment 534, wherein the AAV genome is an AAVrh10 genome.

542. The nucleic acid of any one of embodiments 529 to 541, further encoding a Type II Cas protein, optionally wherein the Type II Cas protein is a Type II Cas protein according to any one of embodiments 1 to 181.

543. A nucleic acid encoding the Type II Cas protein and gRNA of the system of any one of embodiments 510 to 512.

544. The nucleic acid of embodiment 543, wherein the nucleotide sequence encoding the Type II Cas protein is codon optimized for expression in human cells.

545. The nucleic acid of embodiment 543 or embodiment 544, which is a plasmid.

546. The nucleic acid of embodiment 543 or embodiment 544, which is a viral genome.

547. The nucleic acid of embodiment 546, wherein the viral genome is an adeno-associated virus (AAV) genome.

548. The nucleic acid of embodiment 547, wherein the AAV genome is a AAV2, AAV5,

AAV7m8, AAV8, AAV9, AAVrh8r, or AAVrh10 genome.

549. The nucleic acid of embodiment 548, wherein the AAV genome is an AAV2 genome.

550. The nucleic acid of embodiment 548, wherein the AAV genome is an AAV5 genome.

551. The nucleic acid of embodiment 548, wherein the AAV genome is an AAV7m8 genome.

552. The nucleic acid of embodiment 548, wherein the AAV genome is an AAV8 genome.

553. The nucleic acid of embodiment 548, wherein the AAV genome is an AAV9 genome.

554 The nucleic acid of embodiment 548, wherein the AAV genome is an AAVrh8r genome.

555. The nucleic acid of embodiment 548, wherein the AAV genome is an AAVrh10 genome.

556. A plurality of nucleic acids comprising separate nucleic acids encoding the Type II Cas protein and gRNA of the system of any one of embodiments 510 to 512.

557. The plurality of nucleic acid of embodiment 556, wherein the separate nucleic acids encoding the Type II Cas protein and gRNA are plasmids.

558. The plurality of nucleic acids of embodiment 556, wherein the separate nucleic acids encoding the Type II Cas protein and gRNA are viral genomes.

559. The plurality of nucleic acids of embodiment 558, wherein the viral genomes are adeno-associated virus (AAV) genomes.

560. The plurality of nucleic acids of embodiment 559, wherein the AAV genomes the encoding the Type II Cas protein and gRNA are independently an AAV2, AAV5, AAV7m8, AAV8, AAV9, AAVrh8r, or AAVrh10 genome.

561. A Type II Cas protein according to any one of embodiments 1 to 181, a gRNA according to any one of embodiments 182 to 507, a combination of gRNAs according to any one of embodiments 508 to 509, a system according to of any one of embodiments 510 to 512, a nucleic acid according to any one of embodiments 513 to 555, a plurality of nucleic acids according to of any one of embodiments 556 to 560, particle according to any one of embodiments 568 to 583, or a pharmaceutical composition according to embodiment 584 for use in a method of editing a human genomic sequence.

562. The Type II Cas protein, gRNA, combination of gRNAs, system, nucleic acid, a plurality of nucleic acids, particle, or pharmaceutical composition for use according to embodiment 561, wherein the human genomic sequence is a RHO or DNMT1 genomic sequence.

563. The Type II Cas protein, gRNA, combination of gRNAs, system, nucleic acid, a plurality of nucleic acids, particle, or pharmaceutical composition for use according to embodiment 561, wherein the human genomic sequence is a RHO genomic sequence, optionally wherein the RHO genomic sequence has a pathogenic mutation.

564. The Type II Cas protein, gRNA, combination of gRNAs, system, nucleic acid, a plurality of nucleic acids, particle, or pharmaceutical composition for use according to embodiment 561, wherein the human genomic sequence is a TRAC genomic sequence, optionally wherein the human genomic sequence is in a T cell.

565. The Type II Cas protein, gRNA, combination of gRNAs, system, nucleic acid, a plurality of nucleic acids, particle, or pharmaceutical composition for use according to embodiment 561, wherein the human genomic sequence is a B2M genomic sequence, optionally wherein the human genomic sequence is in a T cell.

566. The Type II Cas protein, gRNA, combination of gRNAs, system, nucleic acid, a plurality of nucleic acids, particle, or pharmaceutical composition for use according to embodiment 561, wherein the human genomic sequence is a PD1 genomic sequence, optionally wherein the human genomic sequence is in a T cell.

567. The Type II Cas protein, gRNA, combination of gRNAs, system, nucleic acid, a plurality of nucleic acids, particle, or pharmaceutical composition for use according to embodiment 561, wherein the human genomic sequence is a LAG3 genomic sequence, optionally wherein the human genomic sequence is in a T cell.

568. A particle comprising a Type II Cas protein according to any one of embodiments 1 to

181, a gRNA according to any one of embodiments 182 to 507, a combination of gRNAs according to any one of embodiments 508 to 509, a system according to of any one of embodiments 510 to 512, a nucleic acid according to any one of embodiments 513 to 555, or a plurality of nucleic acids according to of any one of embodiments 556 to 560.

569. The particle of embodiment 568, which is a lipid nanoparticle, a vesicle, a gold nanoparticle, a viral-like particle (VLP) or a viral particle.

570. The particle of embodiment 569, which is a lipid nanoparticle.

571 The particle of embodiment 569, which is a vesicle.

572. The particle of embodiment 569, which is a gold nanoparticle.

573 The particle of embodiment 569, which is a viral-like particle (VLP).

574 The particle of embodiment 569, which is a viral particle.

575. The particle of embodiment 574, which is an adeno-associated virus (AAV) particle.

576. The particle of embodiment 575, wherein the AAV particle is an AAV2, AAV5, AAV7m8, AAV8, AAV9, AAVrh8r, or AAVrh10 particle.

577. The particle of embodiment 576, wherein the AAV particle is an AAV2 particle.

578. The particle of embodiment 576, wherein the AAV particle is an AAV5 particle.

579 The particle of embodiment 576, wherein the AAV particle is an AAV7m8 particle.

580. The particle of embodiment 576, wherein the AAV particle is an AAV8 particle.

581. The particle of embodiment 576, wherein the AAV particle is an AAV9 particle.

582. The particle of embodiment 576, wherein the AAV particle is an AAVrh8r particle.

583. The particle of embodiment 576, wherein the AAV particle is an AAVrh10 particle.

584. A pharmaceutical composition comprising a Type II Cas protein according to any one of embodiments 1 to 181, a gRNA according to any one of embodiments 182 to 507, a combination of gRNAs according to any one of embodiments 508 to 509, a system according to of any one of embodiments 510 to 512, a nucleic acid according to any one of embodiments 513 to 555, or a plurality of nucleic acids according to of any one of embodiments 556 to 560, or a particle according to any one of embodiments 568 to 583 and at least one pharmaceutically acceptable excipient.

585. A cell comprising a Type II Cas protein according to any one of embodiments 1 to 181, a gRNA according to any one of embodiments 182 to 507, a combination of gRNAs according to any one of embodiments 508 to 509, a system according to of any one of embodiments 510 to 512, a nucleic acid according to any one of embodiments 513 to 555, or a plurality of nucleic acids according to of any one of embodiments 556 to 560, or a particle according to any one of embodiments 568 to 583.

586. The cell of embodiment 585, which is a human cell.

587. The cell of embodiment 585 or embodiment 586, wherein the cell is a hematopoietic progenitor cell.

588. The cell of any one of embodiments 585 to 587, which is a stem cell.

589. The cell of embodiment 588, wherein the stem cell is a hematopoietic stem cell (HSC), a pluripotent stem cell, or an induced pluripotent stem cell (iPS).

590. The cell of embodiment 589, wherein the stem cell is an embryonic stem cell.

591. The cell of any one of embodiments 585 to 586, which is a retinal cell.

592. The cell of any one of embodiments 585 to 586, which is a photoreceptor cell.

593. The cell of any one of embodiments 585 to 592, which is an ex vivo cell.

594. A population of cells according to any one of embodiments 585 to 593.

595. A method for altering a cell, the method comprising contacting the cell with a Type II Cas protein according to any one of embodiments 1 to 181, a gRNA according to any one of embodiments 182 to 507, a combination of gRNAs according to any one of embodiments 508 to 509, a system according to of any one of embodiments 510 to 512, a nucleic acid according to any one of embodiments 513 to 555, or a plurality of nucleic acids according to of any one of embodiments 556 to 560, a particle according to any one of embodiments 568 to 583, or a pharmaceutical composition according to embodiment 584.

596. The method of embodiment 595, which comprises contacting the cell with the Type II Cas protein of any one of embodiments 1 to 181.

597. The method of embodiment 595, which comprises contacting the cell with the gRNA of any one of embodiments 182 to 507.

598. The method of embodiment 595, which comprises contacting the cell with the combination of gRNAs according to any one of embodiments 508 to 509.

599. The method of embodiment 595, which comprises contacting the cell with the system of any one of embodiments 478 to 512.

600. The method of embodiment 599, which comprises electroporation of the cell prior to contacting the cell with the system.

601. The method of embodiment 599, which comprises lipid-mediated delivery of the system to the cell, optionally wherein the lipid-mediated delivery is cationic lipid-mediated delivery.

602. The method of embodiment 599, which comprises polymer-mediated delivery of the system to the cell.

603. The method of embodiment 599, which comprises delivery of the system to the cell by lipofection.

604. The method of embodiment 599, which comprises delivery of the system to the cell by nucleofection.

605. The method of embodiment 595, which comprises contacting the cell with the nucleic acid of any one of embodiments 513 to 555.

606. The method of embodiment 595, which comprises contacting the cell with the plurality of nucleic acids of any one of embodiments 556 to 560.

607. The method of embodiment 595, which comprises contacting the cell with the particle of any one of embodiments 568 to 583.

608. The method of embodiment 595, which comprises contacting the cell with the pharmaceutical composition of embodiment 584.

609. The method of any one of embodiments 595 to 608, wherein the contacting alters a RHO or DNMT1 genomic sequence.

610. The method of any one of embodiments 595 to 608, wherein the contacting alters a RHO genomic sequence.

611. The method of embodiment 610, wherein the cell has a RHO allele having a pathogenic mutation and the contacting alters the RHO allele having the pathogenic mutation, optionally wherein the alteration is a deletion.

612. The method of embodiment 611, wherein the cell is a cell from a subject having a RHO allele with the pathogenic mutation or a progeny of such cell.

613. The method of embodiment 612, wherein the subject is heterozygous for the rs7984 SNP and the subject is heterozygous for the pathogenic mutation.

614. The method of embodiment 613, which further comprises a step of genotyping the subject to determine which allele of the rs7984 SNP is in phase with the pathogenic mutation.

615. The method of any one of embodiments 595 to 608, wherein the contacting alters a TRAC genomic sequence.

616. The method of any one of embodiments 595 to 608, wherein the contacting alters a B2M genomic sequence.

617. The method of any one of embodiments 595 to 608, wherein the contacting alters a PD1 genomic sequence.

618. The method of any one of embodiments 595 to 608, wherein the contacting alters a LAG3 genomic sequence.

619. The method of any one of embodiments 595 to 618, wherein the cell is a human cell. 620. The method of any one of embodiments 595 to 619, wherein the cell is a hematopoietic progenitor cell.

621. The method of any one of embodiments 595 to 620, wherein the cell is a stem cell. 622. The method of embodiment 621, wherein the stem cell is a hematopoietic stem cell (HSC), a pluripotent stem cell, or an induced pluripotent stem cell (iPS).

623. The method of embodiment 622, wherein the stem cell is an embryonic stem cell.

624. The method of any one of embodiments 595 to 619, wherein the cell is a retinal cell.

625. The method of any one of embodiments 595 to 619, wherein the cell is a photoreceptor cell.

626. The method of any one of embodiments 595 to 619, wherein the cell is a T cell.

627. The method of any one of embodiments 595 to 626, wherein the contacting is in vitro.

628. The method of embodiment 626, further comprising transplanting the cell to a subject.

629. The method of any one of embodiments 595 to 626, wherein the contacting is in vivo in a subject.

630. The method of embodiment 629, wherein the contacting is performed in or near an eye of the subject.

631. The method of embodiment 630, wherein the contacting comprises delivering the Type II Cas protein, gRNA, combination of gRNAs, system, nucleic acid, plurality of nucleic acids, particle, or pharmaceutical composition to the eye by sub-retinal injection.

632. The method of embodiment 630, wherein the contacting comprises delivering the Type II Cas protein, gRNA, combination of gRNAs, system, nucleic acid, plurality of nucleic acids, particle, or pharmaceutical composition to the eye by intravitreal injection.

633. A cell or population of cells produced by the method of any one of embodiments 595 to 627.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A Type II Cas protein comprising an amino acid sequence having at least 50% sequence identity to:
(a) the amino acid sequence of a RuvC-I domain of a reference protein sequence;
(b) the amino acid sequence of a RuvC-II domain of a reference protein sequence;
(c) the amino acid sequence of a RuvC-III domain of a reference protein sequence;
(d) the amino acid sequence of a BH domain of a reference protein sequence;
(e) the amino acid sequence of a REC domain of a reference protein sequence;
(f) the amino acid sequence of a HNH domain of a reference protein sequence;
(g) the amino acid sequence of a WED domain of a reference protein sequence;
(h) the amino acid sequence of a PID domain of a reference protein sequence; or
(i) the amino acid sequence of the full length of a reference protein sequence;
wherein the reference protein sequence is SEQ ID NO:1 or SEQ ID NO:2.

2. The Type II Cas protein of claim 1, wherein the amino acid sequence of the Type II Cas protein comprises an amino acid sequence that is at least 55% identical, at least 60% identical, at least 65% identical, at least 70% identical, at least 75% identical, at least 80% identical, at least 85% identical, at least 90% identical, at least 95% identical, at least 96% identical, at least 97% identical, at least 98% identical, or is at least 99% identical to the full length of the reference protein sequence.

3. The Type II Cas protein of claim 1, wherein the amino acid sequence of the Type II Cas protein comprises an amino acid sequence that is identical to the full length of the reference protein sequence.

4. The Type II Cas protein of any one of claims 1 to 3, which is a fusion protein.

5. The Type II Cas protein of claim 4, which comprises one or more nuclear localization signals, such as two or more nuclear localization signals, and which optionally comprises an N-terminal nuclear localization signal and/or a C-terminal nuclear localization signal.

6. The Type II Cas protein of any one of claim 5, wherein the amino acid sequence of one or more of the nuclear localization signals comprises the amino acid sequence
(SEQ ID NO: 7)
KRTADGSEFESPKKKRKV,
(SEQ ID NO: 8)
PKKKRKV,
(SEQ ID NO: 9)
PKKKRRV,
(SEQ ID NO: 10)
KRPAATKKAGQAKKKK,
(SEQ ID NO: 11)
YGRKKRRQRRR,
(SEQ ID NO: 12)
RKKRRQRRR,
(SEQ ID NO: 13)
PAAKRVKLD,
(SEQ ID NO: 14)
RQRRNELKRSP,
(SEQ ID NO: 15)
VSRKRPRP,
(SEQ ID NO: 16)
PPKKARED,
(SEQ ID NO: 17)
PQPKKKPL,
(SEQ ID NO: 18)
SALIKKKKKMAP,
(SEQ ID NO: 19)
PKQKKRK,
(SEQ ID NO: 20)
RKLKKKIKKL,
(SEQ ID NO: 21)
REKKKFLKRR,
(SEQ ID NO: 22)
KRKGDEVDGVDEVAKKKSKK,
(SEQ ID NO: 23)
RKCLQAGMNLEARKTKK,
(SEQ ID NO: 24)
NQSSNFGPMKGGNFGGRSSGPYGGGGQYFAKPRNQGGY,
or
(SEQ ID NO: 25)
RMRIZFKNKGKDTAELRRRRVEVSVELRKAKKDEQILKRRNV.

7. The Type II Cas protein of claim 5 or claim 6, wherein the amino acid sequence of each nuclear localization signal is the same.

8. The Type II Cas protein of any one of claims 4 to 7, which comprises a fusion partner which is a DNA, RNA or protein modification enzyme, optionally wherein the DNA, RNA or protein modification enzyme is an adenosine deaminase, a cytidine deaminase, a reverse transcriptase, a guanosyl transferase, a DNA methyltransferase, a RNA methyltransferase, a DNA demethylase, a RNA demethylase, a dioxygenase, a polyadenylate polymerase, a pseudouridine synthase, an acetyltransferase, a deacetylase, a ubiquitin-ligase, a deubiquitinase, a kinase, a phosphatase, a NEDD8-ligase, a de-NEDDylase, a SUMO-ligase, a deSUMOylase, a histone deacetylase, a histone acetyltransferase, a histone methyltransferase, or a histone demethylase.

9. The Type II Cas protein of any one of claims 4 to 8, which comprises a tag, e.g., a SV5 tag, optionally wherein the SV5 tag comprises the amino acid sequence GKPIPNPLLGLDST (SEQ ID NO: 26).

10. The Type II Cas protein of claim 1, whose amino acid sequence comprises the amino acid sequence of SEQ ID NO:3.

11. A Type II Cas protein whose amino acid sequence is identical to a Type II Cas protein of any one of claims 1 to 10 except for one or more amino acid substitutions relative to the reference sequence that provide nickase activity, optionally wherein the one or more amino acid substitutions relative to the reference sequence that provide nickase activity comprise a D23A or H612A substitution, wherein the position of the D23A or H612A substitution is defined with respect to the amino acid numbering of SEQ ID NO:2.

12. A Type II Cas protein whose amino acid sequence is identical to a Type II Cas protein of any one of claims 1 to 10 except for one or more amino acid substitutions relative to the reference sequence that render the Type II Cas protein catalytically inactive, optionally wherein the one or more amino acid substitutions comprise D23A and H612A substitutions, wherein the position of the D23A and H612A substitutions are defined with respect to the amino acid numbering of SEQ ID NO:2.

13. A guide RNA (gRNA) molecule for editing a human RHO gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is
(SEQ ID NO: 97)
(a) UGGGUGGGAGCAGCCRCGGGU, where R is A or G;
(SEQ ID NO: 93)
(b) GGCCCUUGUGGCUGACCCGYGGCU, where Y is U or C;
(SEQ ID NO: 94)
(c) GGCCCUUGUGGCUGACCCGUGGCU;
(SEQ ID NO: 95)
(d) GGCCCUUGUGGCUGACCCGCGGCU;
(SEQ ID NO: 96)
(e) GGGUGGGAGCAGCCRCGGGU, where R is A or G;
(SEQ ID NO: 98)
(f) UUGGGUGGGAGCAGCCRCGGGU, where R is A or G;
(SEQ ID NO: 99)
(g) UCUUGGGUGGGAGCAGCCRCGGGU, where R is A or G;
(SEQ ID NO: 100)
(h) UUCUUGGGUGGGAGCAGCCRCGGGU, where R is A or G;
(SEQ ID NO: 101)
(i) GGGUGGGAGCAGCCACGGGU;
(SEQ ID NO: 102)
(j) UGGGUGGGAGCAGCCACGGGU;
(SEQ ID NO: 103)
(k) UUGGGUGGGAGCAGCCACGGGU;
(SEQ ID NO: 104)
(l) UCUUGGGUGGGAGCAGCCACGGGU;
(SEQ ID NO: 105)
(m) UUCUUGGGUGGGAGCAGCCACGGGU;
(SEQ ID NO: 106)
(n) GGGUGGGAGCAGCCGCGGGU;
(SEQ ID NO: 107)
(o) UGGGUGGGAGCAGCCGCGGGU;
(SEQ ID NO: 108)
(p) UUGGGUGGGAGCAGCCGCGGGU;
(SEQ ID NO: 109)
(q) UCUUGGGUGGGAGCAGCCGCGGGU;
or
(SEQ ID NO: 110)
(r) UUCUUGGGUGGGAGCAGCCGCGGGU.

14. A guide RNA (gRNA) molecule for editing a human RHO gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is
(SEQ ID NO: 35)
(a) CUUGGGUGGGAGCAGCCRCGGGU, where R is A or G;
(SEQ ID NO: 34)
(b) CUUGUGGCUGACCCGYGGCUGCUC, where Y is U or C;
(SEQ ID NO: 36)
(c) GGAGCAGCCRCGGGUCAGCCACAA, where R is A or G;
(SEQ ID NO: 37)
(d) CUUGUGGCUGACCCGUGGCUGCUC;
(SEQ ID NO: 38)
(e) CUUGGGUGGGAGCAGCCACGGGU;
(SEQ ID NO: 39)
(f) GGAGCAGCCACGGGUCAGCCACAA;
(SEQ ID NO: 40)
(g) CUUGUGGCUGACCCGCGGCUGCUC;
(SEQ ID NO: 41)
(h) CUUGGGUGGGAGCAGCCGCGGGU;
or
(SEQ ID NO: 42)
(i) GGAGCAGCCGCGGGUCAGCCACAA.

15. A guide RNA (gRNA) molecule for editing a human RHO gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is
(SEQ ID NO: 111)
(a) CAUGCUCCCGGGCUCCUGCACAC;
(SEQ ID NO: 112)
(b) CCUCCAUGCUCCCGGGCUCCUGC;
(SEQ ID NO: 113)
(c) AGCCACCACCGCCAAGCCCGGGA;
(SEQ ID NO: 114)
(d) UCCCUUCUCCUGUCCUGUCAAUG;
(SEQ ID NO: 115)
(e) CCCUUCUCCUGUCCUGUCAAUGU;
(SEQ ID NO: 116)
(f) UGUCCUGUCAAUGUUAUCCAAAG;
(SEQ ID NO: 117)
(g) AUCCAAAGCCCUCAUAUAUUCAG;
(SEQ ID NO: 118)
(h) AAGGCAGUGUUCAGUGCCAGCCC;
(SEQ ID NO: 119)
(i) UCAAGGCAGUGUUCAGUGCCAGC;
(SEQ ID NO: 120)
(j) GUGAAAUUAGACAAGCGCAUAUU;
(SEQ ID NO: 121)
(k) UUGGAGCAAUAUGCGCUUGUCUA;
(SEQ ID NO: 122)
(l) GUUUUCUUGCUGUGAAAUUAGACA;
(SEQ ID NO: 123)
(m) UCACAGCAAGAAAACUGAGCUGA;
(SEQ ID NO: 124)
(n) AAGAAGUCAAGCGCCCUGCUGGG;
(SEQ ID NO: 125)
(o) GAAGUCAAGCGCCCUGCUGGGGC;
(SEQ ID NO: 126)
(p) AACUCUGCACCCGUCCCUGUGUG;
(SEQ ID NO: 127)
(q) UGCUGGGGCGUCACACAGGGACG;
(SEQ ID NO: 128)
(r) AACAUGGCCCGAGAUAGAUGCGG;
(SEQ ID NO: 129)
(s) ACAGAGGCUUGGUGCUGCAAACA;
(SEQ ID NO: 130)
(t) UCCAAGGGAAACAGAGGCUUGGU;
(SEQ ID NO: 131)
(u) GACUCAGCACAGCUGCUCCAAGG;
(SEQ ID NO: 132)
(v) GCCUGGGUCUGACUCAGCACAGC;
(SEQ ID NO: 133)
(w) CCCUUGGAGCAGCUGUGCUGAGU;
or
(SEQ ID NO: 134)
(x) UCAGUGCCCAGCCUGGGUCUGAC.

16. A guide RNA (gRNA) molecule for editing a human TRAC gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is
(SEQ ID NO: 135)
(a) AUACACAUCAGAAUCCUUACUUU;
(SEQ ID NO: 136)
(b) GUAAGGAUUCUGAUGUGUAUAUCA;
(SEQ ID NO: 137)
(c) GAGCAACAGUGCUGUGGCCUGGA;
(SEQ ID NO: 138)
(d) GGAGCAACAAAUCUGACUUUGCA;
(SEQ ID NO: 139)
(e) UGCUGUUGUUGAAGGCGUUUGCA;
(SEQ ID NO: 140)
(f) AGCAUUAUUCCAGAAGACACCUU;
(SEQ ID NO: 141)
(g) UAUUCCAGAAGACACCUUCUUCC;
(SEQ ID NO: 142)
(h) ACAAAGUAAGGAUUCUGAUGUGU;
(SEQ ID NO: 143)
(i) GUCUAGCACAGUUUUGUCUGUGA;
(SEQ ID NO: 144)
(j) CUUGAAGUCCAUAGACCUCAUGU;
(SEQ ID NO: 145)
(k) GGCCACAGCACUGUUGCUCUUGA;
(SEQ ID NO: 146)
(l) UGCAAAGUCAGAUUUGUUGCUCC;
or
(SEQ ID NO: 147)
(m) GAAUAAUGCUGUUGUUGAAGGCG.

17. A guide RNA (gRNA) molecule for editing a human B2M gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is
(SEQ ID NO: 148)
(a) UAAGGCCACGGAGCGAGACAUCU;
(SEQ ID NO: 149)
(b) CAGGCCAGAAAGAGAGAGUAGCG;
(SEQ ID NO: 150)
(c) GCCUGGAGGCUAUCCAGCGUGAGU.

18. A guide RNA (gRNA) molecule for editing a human PD1 gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is
(SEQ ID NO: 151)
(a) GGCCGCCAGCCCAGUUGUAGCAC;
or
(SEQ ID NO: 152)
(b) CUGGCCGCCAGCCCAGUUGUAGC.

19. A guide RNA (gRNA) molecule for editing a human LAG3 gene, which is optionally an ENQP Type II Cas gRNA, the gRNA comprising a spacer whose nucleotide sequence comprises 15 or more consecutive nucleotides of a reference sequence or comprises a nucleotide sequence that is at least 85% identical to the reference sequence, wherein the reference sequence is
(SEQ ID NO: 153)
(a) GCCAUCCCCGUUUUACCUGGAGC;
(SEQ ID NO: 154)
(b) CGCCAUCCCCGUUUUACCUGGAG;
(SEQ ID NO: 155)
(c) CCGCCAUCCCCGUUUUACCUGGA;
(SEQ ID NO: 156)
(d) GGGAUGGCGGGAGGGUUGACCUC;
(SEQ ID NO: 157)
(e) GAGCUAAGGACCUCAGGACCUUUG;
(SEQ ID NO: 158)
(f) GAGAGGCUUUCGGGGUGGAAGGAC;
(SEQ ID NO: 159)
(g) GAGAGGCUUUCGGGGUGGAAGGA;
or
(SEQ ID NO: 160)
(h) AGAGAGGCUUUCGGGGUGGAAGG.

20. The gRNA of any one of claims 13 to 19, which comprises a spacer that is 15 to 30 nucleotides in length, 18 to 30 nucleotides in length, 20 to 28 nucleotides in length, 22 to 26 nucleotides in length, 23 to 25 nucleotides in length, 22 to 25 nucleotides in length, 15 to 25 nucleotides in length, 16 to 24 nucleotides in length, 17 to 23 nucleotides in length, 18 to 22 nucleotides in length, 19 to 21 nucleotides in length, 25 nucleotides in length, 24 nucleotides in length, 23 nucleotides in length, 22 nucleotides in length, 21 nucleotides in length, or 20 nucleotides in length.

21. The gRNA of any one of claims 13 to 20, wherein the spacer comprises the reference sequence.

22. The gRNA of any one of claims 13 to 21, which is a single guide RNA (sgRNA).

23. A gRNA comprising a spacer and a sgRNA scaffold, wherein:
(a) the spacer is positioned 5′ to the sgRNA scaffold; and
(b) the nucleotide sequence of the sgRNA scaffold comprises a nucleotide sequence that is at least 50% identical to a reference scaffold sequence, wherein the reference scaffold sequence is SEQ ID NO:91, SEQ ID NO:44, or SEQ ID NO:45.

24. The gRNA of claim 23, wherein the sgRNA scaffold comprises a nucleotide sequence that is 100% identical to the reference scaffold sequence.

25. The gRNA of claim 23, wherein the nucleotide sequence of the sgRNA scaffold comprises the nucleotide sequence of SEQ ID NO:92, SEQ ID NO:46, or SEQ ID NO:47.

26. The gRNA of any one of claims 23 to 25, wherein the nucleotide sequence of the spacer is partially or fully complementary to a target mammalian (e.g., human) genomic sequence, for example a human RHO, DNMT1, TRAC, B2M, PD1, or LAG3 genomic sequence.

27. A gRNA comprising a spacer sequence of SEQ ID NO:102, SEQ ID NO: 107, SEQ ID NO: 38, SEQ ID NO:41, SEQ ID NO: 101, SEQ ID NO: 103, SEQ ID NO: 104, SEQ ID NO: 105, SEQ ID NO: 106, SEQ ID NO: 108, SEQ ID NO:109, or SEQ ID NO: 110.

28. A gRNA comprising a spacer sequence of SEQ ID NO:38 or SEQ ID NO:41.

29. A gRNA comprising a spacer whose sequence is the sequence of SEQ ID NO: 118, SEQ ID NO: 128, or SEQ ID NO:130.

30. A gRNA comprising a spacer whose sequence is the sequence of SEQ ID NO: 136, SEQ ID NO: 139, or SEQ ID NO: 140.

31. A gRNA comprising a spacer whose sequence is the sequence of SEQ ID NO: 149.

32. A gRNA comprising a spacer whose sequence is the sequence of SEQ ID NO: 152.

33. A gRNA comprising a spacer whose sequence is the sequence of SEQ ID NO: 156, SEQ ID NO: 158, SEQ ID NO: 159, or SEQ ID NO: 160.

34. The gRNA of any one of claims 27 to 33, wherein the spacer is positioned 5′ to a scaffold whose sequence comprises the sequence of SEQ ID NO:44, SEQ ID NO:45, SEQ ID NO:46, SEQ ID NO: 47, SEQ ID NO:91, or SEQ ID NO:92.

35. A combination of gRNAs comprising (a) a first gRNA comprising a spacer sequence of SEQ ID NO:102, SEQ ID NO: 107, SEQ ID NO:38, SEQ ID NO:41, SEQ ID NO: 101, SEQ ID NO: 103, SEQ ID NO: 104, SEQ ID NO: 105, SEQ ID NO: 106, SEQ ID NO: 108, SEQ ID NO:109, or SEQ ID NO: 110, optionally wherein the spacer of the first gRNA is positioned 5′ to a sgRNA scaffold whose sequence comprises the sequence of SEQ ID NO:44, SEQ ID NO:45, SEQ ID NO:46, SEQ ID NO:47, SEQ ID NO:91, or SEQ ID NO:92 and (b) a second gRNA comprising a spacer whose sequence is the sequence of SEQ ID NO:118, SEQ ID NO: 128, or SEQ ID NO: 130, optionally wherein the spacer of the second gRNA is positioned 5′ to a sgRNA scaffold whose sequence comprises the sequence of SEQ ID NO: 44, SEQ ID NO:45, SEQ ID NO:46, SEQ ID NO:47, SEQ ID NO:91, or SEQ ID NO:92.

36. A system comprising the Type II Cas protein of any one of claims 1 to 12 and a guide RNA (gRNA) molecule comprising a spacer partially or fully complementary to a target mammalian genomic sequence.

37. A nucleic acid encoding the Type II Cas protein of any one of claims 1 to 12, optionally wherein the nucleotide sequence encoding the Type II Cas protein is operably linked to a promoter that is heterologous to the Type II Cas protein.

38. The nucleic acid of claim 37, wherein the nucleotide sequence encoding the Type II Cas protein is codon optimized for expression in human cells.

39. The nucleic acid of claim 38, which comprises a nucleotide sequence that is at least 80%, at least 85%, at least 90%, at least 95%, at least 96%, at least 97%, at least 98%, at least 99%, or 100% identical to the nucleotide sequence of SEQ ID NO:5 or SEQ ID NO:6.

40. The nucleic acid of any one of claims 37 to 39, which is a plasmid or a viral genome, optionally an adeno-associated virus (AAV) genome, e.g., an AAV2, AAV5, AAV7m8, AAV8, AAV9, AAVrh8r, or AAVrh10 genome.

41. The nucleic acid of any one of claims 37 to 40, further encoding a gRNA.

42. A nucleic acid encoding the gRNA of any one of claims 13 to 34 or the combination of gRNAs of claim 34.

43. A plurality of nucleic acids comprising separate nucleic acids encoding the Type II Cas protein and gRNA of the system of claim 36.

44. A particle comprising a Type II Cas protein according to any one of claims 1 to 12, a gRNA according to any one of claims 13 to 34, a combination of gRNAs according to claim 35, a system according to claim 36, a nucleic acid according to any one of claims 37 to 42, or a plurality of nucleic acids according to claim 43.

45. The particle of claim 44, which is a lipid nanoparticle, a vesicle, a gold nanoparticle, a viral-like particle (VLP) or a viral particle, optionally wherein the particle is an AAV particle.

46. A pharmaceutical composition comprising a Type II Cas protein according to any one of claims 1 to 12, a gRNA according to any one of claims 13 to 34, a combination of gRNAs according to claim 35, a system according to claim 36, a nucleic acid according to any one of claims 37 to 42, a plurality of nucleic acids according to claim 43, or a particle according to claim 44 or claim 45 and at least one pharmaceutically acceptable excipient.

47. A Type II Cas protein according to any one of claims 1 to 12, a gRNA according to any one of claims 13 to 34, a combination of gRNAs according to claim 35, a system according to claim 36, a nucleic acid according to any one of claims 37 to 42, a plurality of nucleic acids according to claim 43, a particle according to claim 44 or claim 45, or a pharmaceutical composition according to claim 46 for use in a method of editing a human genomic sequence.

48. The Type II Cas protein, gRNA, combination of gRNAs, system, nucleic acid, a plurality of nucleic acids, particle, or pharmaceutical composition for use according to claim 47, wherein the human genomic sequence is a RHO genomic sequence, optionally wherein the RHO genomic sequence has a pathogenic mutation.

49. The Type II Cas protein, gRNA, combination of gRNAs, system, nucleic acid, a plurality of nucleic acids, particle, or pharmaceutical composition for use according to claim 47, wherein the human genomic sequence is a TRAC, B2M, PD1, or LAG3 genomic sequence, optionally wherein the human genomic sequence is in a T cell.

50. An ex vivo human cell comprising a Type II Cas protein according to any one of claims 1 to 12, a gRNA according to any one of claims 13 to 34, a combination of gRNAs according to claim 35, a system according to claim 36, a nucleic acid according to any one of claims 37 to 42, a plurality of nucleic acids according to claim 43, or a particle according to claim 44 or claim 45.
